(19) United States
(12) Patent Application Publication
(10) Pub. No.: US 2009/0105252 A1
(43) Pub. Date: Apr. 23, 2009

(54) Pyrimidine Derivatives and Their Use in Therapy as well as the Use of Pyrimidine Derivatives in the Manufacture of a Medicament for Prevention and/or Treatment of Alzheimer's Disease

(75) Inventors: Lars Andersson, Sodertalje (SE); Erwan Arzel, Sodertalje (SE); Stefan Berg, Sodertalje (SE); Jeremy Burrows, Sodertalje (SE); Sven Hellberg, Sodertalje (SE); Fernando Huerta, Sodertalje (SE); Torben Pedersen, Sodertalje (SE); Tobias Rein, Sodertalje (SE); Didier Rotticci, Sodertalje (SE); Karin Staaf, Sodertalje (SE); Dominika Turek, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/089,008

(22) PCT Filed: Oct. 2, 2006
(86) PCT No.: PCT/SE2006/001116

§ 371 Date: Apr. 21, 2008

(30) Foreign Application Priority Data
Oct. 3, 2005 (SE) 0502174-6

(51) Int. Cl. A61K 31/5377; C07D 403/02; A61K 31/506; C07D 413/14; A61P 25/28; C07D 403/14; A61K 31/496

(52) U.S. Cl. 514/235.8; 544/331; 514/275; 514/252.19; 544/122

(57) ABSTRACT

Compounds of formula I

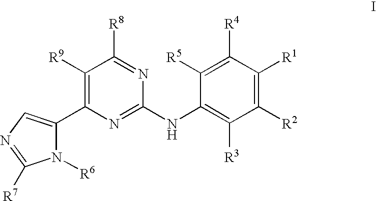

wherein R¹, R², R³, R⁴, R⁵, R⁶, R⁷, R⁸ and R⁹ are as defined in the specification as a base or a pharmaceutically acceptable salt, solvate or solvate of salt thereof, processes for their preparation, new intermediates used therein, pharmaceutical formulations containing said compounds and to the use of said compounds in therapy.

### TECHNICAL FIELD OF INVENTION

The present invention relates to new compounds of formula I, as a free base or a pharmaceutically acceptable salt, solvate or solvate of salt thereof, to pharmaceutical formulations containing said compounds and to the use of said compounds in therapy. The present invention further relates to a process for the preparation of compounds of formula I and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen is synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies

AD is characterized by cognitive decline, cholinergic dysfunction and neuronal death, neurofibrillary tangles and senile plaques consisting of amyloid-β deposits. The sequence of these events in AD is unclear, but they are believed to be related. Glycogen synthase kinase 3β (GSK3β) or Tau (τ) phosphorylating kinase selectively phosphorylates the microtubule associated protein r in neurons at sites that are hyperphosphorylated in AD brains. Hyperphosphorylated protein r has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Imahori and Uchida., J. Biochem 121:179-188, 1997). GSK3, preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3 D inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3, inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK30 may therefore be of therapeutic relevance in the treatment of BD as well as in AD patients that have affective disorders.

### Schizophrenia

GSK3 is involved in signal transduction cascades of multiple cellular processes, particularly during neural development. Kozlovsky et al (Am J Psychiatry 2000 May; 157(5):831-3) found that GSK30 levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

### Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell 1998 Nov. 25; 95 (5):605-14)). The new follicles formed sebaceous glands and dermal papilla, normally established only in embryogenesis. Thus GSK3 inhibition may offer treatment for baldness.

### Oral Contraceptives

Vijajaraghavan et al. (Biol Reprod 2000 June; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### Bone-Related Disorders

It has been shown that GSK3 inhibitors could be used for treatment of bone-related is disorders. This has been discussed in e.g. Tobias et al., Expert Opinion on Therapeutic Targets, February 2002, pp 41-56.

### DISCLOSURE OF THE INVENTION

The object of the present invention is to provide compounds having a selective inhibiting effect at GSK3 as well as having a good bioavailability.

The present invention therefore relates to the use of a compound of the formula I:

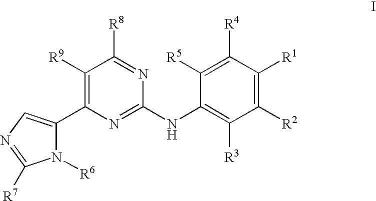

wherein

R¹ is selected from hydrogen, halo, cyano, NO₂, C₁₋₃alkyl, C₁₋₃haloalkyl, OR^{a}, SO₂NR^{b}R^{c}, C₀₋₂alkylC(O)NR^{b}R^{c}, C₁₋₄alkylNR^{b}R^{c}, CH₂OR^{h}, SO₂Rⁱ, C(O)OR^{a}, CH(OH)R^{j} and C(O)R^{j};

R² and R⁴ are independently selected from hydrogen, halo, cyano, NO₂, C₁₋₄alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ, C(O)OR^{a} and C(O)R^{j}; or

R¹ and R², together with the atoms to which they are attached join to form a 5- or 6-membered heterocyclic ring containing at least one N, O or S, in which any of the hydrogens of the CH₂-groups within the said heterocyclic ring can be substituted with oxo, hydroxy or halo and in which any sulphur atom within said heterocyclic ring is optionally oxidised to —SO₂—;

R³ and R⁵ are independently selected from hydrogen, halo, cyano, C₁₋₃alkyl, C₁₋₃haloalkyl and OR^{a};

R⁶ is selected from CH₃, C₆alkyl, C₆alkenyl, C₆alkynyl and C₆haloalkyl; or

R⁶ is a 6-membered heterocyclic ring containing one or more heteroatoms selected from N, O or S, wherein said heterocyclic ring is optionally substituted with one or more C₁₋₃alkyl or C₁₋₃haloalkyl, wherein said C₁₋₃alkyl or C₁₋₃haloalkyl is optionally further substituted with one or more C₁₋₃alkoxy;

R⁷ is selected from hydrogen, C₁₋₃alkyl, cyano, and C₁₋₃haloalkyl, wherein said C₁₋₃alkyl or C₁₋₃haloalkyl is optionally substituted with one or more OR^{a};

R⁸ and R⁹ are independently selected from hydrogen, cyano and halo;

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or

R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};

R^{d} and R^{e} are independently selected from hydrogen, C₁₋₆alkyl and C₁₋₆haloalkyl, wherein said C₁₋₆alkyl or C₁₋₆haloalkyl is optionally substituted with one or more OR^{a}; or

R^{d} and R^{e} may, together with the atom to which they are attached, form a heterocyclic ring wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy;

Rⁱ is selected from C₁₋₆alkyl, heterocyclyl, aryl, heteroaryl and C₁₋₃haloalkyl, wherein said C₁₋₆alkyl, heterocyclyl, aryl, heteroaryl or C₁₋₃haloalkyl is optionally substituted with one or more halo, cyano, di-(C₁₋₄alkyl)amino-, C₁₋₃haloalkyl, C₁₋₃alkyl, heterocyclyl or OR^{a};

R^{j} is an aryl or heteroaryl ring, wherein said aryl or heteroaryl ring is optionally substituted is with one or more C₁₋₃alkyl, OR^{a}, halo or cyano;

as a free base or a pharmaceutically acceptable salt, solvate or solvate of a salt thereof; in the manufacture of a medicament for prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

The present invention also relates to the use of a compound of the formula Ia:

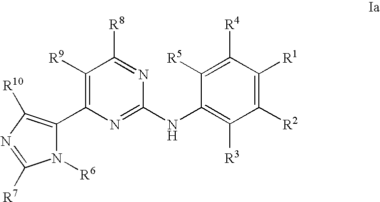

wherein

R¹ is selected from hydrogen, halo, cyano, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};

R² and R⁴ are independently selected from hydrogen, halo, cyano, NO₂, C₁₋₃alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ and C(O)R^{j};

R³ and R⁵ independently are selected from hydrogen, C₁₋₃alkyl, C₁₋₃haloalkyl and OR^{a};

R⁶ is selected from CH₃, C₆alkyl, C₆alkenyl, C₆alkynyl, and C₆haloalkyl; or

R⁶ is a 6-membered heterocyclic ring containing one or more heteroatoms selected from N, O or S, wherein said heterocyclic ring is optionally substituted with one or more C₁₋₃alkyl or C₁₋₃haloalkyl, wherein said C₁₋₃alkyl or C₁₋₃haloalkyl is optionally further substituted with one or more C₁₋₃alkoxy;

R⁷ is selected from C₁₋₃alkyl, cyano, and C₁₋₃haloalkyl, said C₁₋₃alkyl or C₁₋₃haloalkyl is optionally substituted with one or more OR^{a};

R⁸ and R⁹ are independently selected from hydrogen, cyano and halo;

R¹⁰ is hydrogen;

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{c} are independently selected from hydrogen, C₁₋₆alkyl or C₁₋₆haloalkyl, wherein aid C₁₋₆alkyl or C₁₋₆haloalkyl is optionally substituted with one or more OR^{a} or NR^{d}R^{e}; or

R^{b} and R^{c} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy;

R^{d} and R^{e} are independently selected from hydrogen, C₁₋₆alkyl or C₁₋₆haloalkyl, said C₁₋₆alkyl or C₁₋₆haloalkyl is optionally substituted with one or more OR^{a}; or

R^{d} and R^{e} may, together with the atom to which they are attached, form a 4-, 5- or 6-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy

Rⁱ is C₁₋₃alkyl or C₁₋₃haloalkyl, wherein said C₁₋₃alkyl or C₁₋₃haloalkyl is optionally substituted with one or more OR^{a};

R^{j} is an aryl or heteroaryl ring, wherein said aryl or heteroaryl ring is optionally substituted with one or more C₁₋₃alkyl, ORE, halo or cyano;

as a free base or a pharmaceutically acceptable salt, solvate or solvate of a salt thereof; in the manufacture of a medicament for prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

One embodiment of the present invention relates to the use of a compound according to formula I or formula Ia, wherein

R¹ is selected from hydrogen, cyano, C₁₋₃haloalkyl, SO₂NR^{b}R^{c}, C(O)NR^{b}R^{c}, CH₂NR^{b}R^{c}, SO₂Rⁱ and C(O)R^{j};

R² and R⁴ are independently selected from hydrogen, halo, cyano, NO₂, C₁₋₃haloalkyl, ORE, C(O)NR^{b}R^{c}, and SO₂;

R³ and R⁵ independently are selected from hydrogen, C₁₋₃alkyl, and OR^{a};

R⁶ is selected from CH₃, C₆alkyl and C₆haloalkyl; or

R⁶ is a 6-membered heterocyclic ring containing one or more heteroatoms selected from N, O or S, wherein said heterocyclic ring is optionally substituted with one or more C₁₋₃alkyl or C₁₋₃haloalkyl, said C₁₋₃alkyl or C₁₋₃haloalkyl is optionally further substituted with one or more C₁₋₃alkoxy;

R⁷ is selected from C₁₋₃alkyl, cyano, and C₁₋₃haloalkyl;

R¹⁰ is hydrogen;

R⁸ and R⁹ independently are selected from hydrogen, cyano and halo;

R^{a} is selected from hydrogen, C₁₋₃alkyl and C₁₋₃haloalkyl, said C₁₋₃alkyl or C₁₋₃haloalkyl is optionally substituted with one or more C₁₋₃alkoxy;

R^{b} and R^{c} are independently selected from hydrogen, C₁₋₆alkyl or C₁₋₆haloalkyl, said C₁₋₆alkyl or C₁₋₆haloalkyl is optionally substituted with one or more OR^{a}; or

R^{b} and R^{c} may, together with the atom to which they are attached, form a 4-, 5-, 6- or 7-membered heterocyclic ring containing one or more heteroatoms selected from N, O or S, wherein said heterocyclic ring is optionally substituted with one or more halo, C₁₋₃alkyl or C₁₋₃haloalkyl, said C₁₋₃alkyl or C₁₋₃haloalkyl is optionally further substituted with one or more C₁₋₃alkoxy;

Rⁱ is C₁₋₃alkyl or C₁₋₃haloalkyl, said C₁₋₃alkyl or C₁₋₃haloalkyl is optionally substituted with one or more OR^{a};

R^{j} is an arylor heteroaryl ring, wherein said aryl or heteroaryl ring is optionally substituted with one or more C₁₋₃alkyl, OR^{a}, halo or cyano; as a free base or a pharmaceutically acceptable salt, solvate or solvate of a salt thereof.

One embodiment of the present invention provides the use of the compound according to formula I or formula Ia wherein R⁶ is selected from CH₃ and C₆alkyl; or

R⁶ is a 6-membered heterocyclic ring containing one or more heteroatoms selected from N, O or S, wherein said heterocyclic ring is optionally substituted with one or more C₁₋₃alkyl or C₁₋₃haloalkyl; as a free base or a pharmaceutically acceptable salt, solvate or solvate of a salt thereof.

Another embodiment of the present invention provides the use of the compound of formula I or formula Ia, wherein R¹ is selected from hydrogen, cyano, C₁₋₃haloalkyl, SO₂NR^{b}R^{c}, C(O)NR^{b}R^{c}, CH₂NR^{b}R^{c}, SO₂Rⁱ and C(O)R^{j};

R² and R⁴ are independently selected from hydrogen, halo, cyano, NO₂, C₁₋₃haloalkyl, OR^{a}, C(O)NR^{b}R^{c} and SO₂Rⁱ;

R³ and R⁵ independently are selected from hydrogen, C₁₋₃alkyl, and OR^{a};

R⁶ is selected from CH₃ and C₆alkyl; or

R⁶ is a 6-membered heterocyclic ring containing one or more heteroatoms selected from N, O or S, wherein said heterocyclic ring is optionally substituted with one or more C₁₋₃alkyl or C₁₋₃haloalkyl;

R⁷ is selected from C₁₋₃alkyl and C₁₋₃haloalkyl;

R¹⁰ is hydrogen;

R⁸ and R⁹ independently are selected from hydrogen and halo;

R^{a} is C₁₋₃alkyl or C₁₋₃haloalkyl;

R¹ and R^{c} are independently selected from hydrogen, C₁₋₆alkyl, said C₁₋₆alkyl optionally substituted with one or more OR^{a} or

R^{b} and R^{c} may, together with the atom to which they are attached, together form a 4-, 5- or 6-membered heterocyclic ring containing one or more heteroatoms selected from N, O or S, wherein said heterocyclic ring is optionally substituted with one or more halo or C₁₋₃alkyl;

Rⁱ is C₁₋₃alkyl;

R^{j} is an aryl or heteroaryl ring, wherein said aryl or heteroaryl ring is optionally substituted with one or more C₁₋₃alkyl, OR^{a}, halo or cyano

as a free base or a pharmaceutically acceptable salt, solvate or solvate of a salt thereof.

Yet another additional embodiment of the present invention provides the use of the compound according to formula I, wherein

R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} and C(O)R^{j};

R² and R⁴ are independently selected from hydrogen, halo, cyano, NO₂, C₁₋₄alkyl, C₁₋₃haloalkyl, OR^{a}, SO₂Rⁱ, C(O)NR^{b}R^{c} and C(O)OR^{a}; or

R¹ and R², together with the atoms to which they are attached join to form a 5- or 6-membered heterocyclic ring containing at least one N, O or S, in which any of the hydrogen sof the CH₂-groups within the said heterocyclic ring can be substituted with oxo, hydroxy or halo and in which any sulphur atom within said heterocyclic ring is optionally oxidised to —SO₂—;

R³ and R⁵ are independently selected from hydrogen, C₁₋₃alkyl, and OR^{a};

R⁶ is selected from CH₃ and C₆alkyl; or

R⁶ is a 6-membered heterocyclic ring containing one or more heteroatoms selected from N or O, wherein said heterocyclic ring is optionally substituted with one or more C₁₋₃alkyl;

R⁷ is selected from C₁₋₃alkyl, cyano, and C₁₋₃haloalkyl;

R⁸ and R⁹ are independently selected from hydrogen and halo;

R^{a} is selected from hydrogen, C₁₋₃alkyl and C₁₋₃haloalkyl, wherein said C₁₋₃alkyl is optionally substituted with one or more C₁₋₃alkoxy;

R^{b} and R^{c} are independently selected from hydrogen, C₁₋₆alkyl and heterocyclyl, wherein said C₁₋₆alkyl, heterocyclyl is optionally substituted with one or more cyano, OR^{a} or NR^{d}R^{e}; or

R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};

R^{d} and R^{e} are independently selected from hydrogen and C₁₋₆alkyl, wherein said C₁₋₆alkyl is optionally substituted with one or more OR^{a}; or

R^{d} and R^{e} may, together with the atom to which they are attached, form a heterocyclic ring wherein said heterocyclic ring is optionally substituted with one or more halo;

Rⁱ is selected from C₁₋₆alkyl and heterocyclyl, wherein said C₁₋₆alkyl or heterocyclyl is optionally substituted with one or more di-(C₁₋₄alkyl)amino-, heterocyclyl or OR^{a};

R^{j} is an aryl or heteroaryl ring, wherein said aryl or heteroaryl ring is optionally substituted with one or more C₁₋₃alkyl; as a free base or a pharmaceutically acceptable salt, solvate or solvate of a salt thereof.

A further embodiment of the present invention relates to the use of a compound according to formula I, wherein R³ and R⁵ are hydrogen.

Yet a further embodiment of the present invention relates to the use of a compound according to formula I, wherein R⁸ is hydrogen and R⁹ is hydrogen or fluoro.

Another embodiment of the present invention relates to the use of a compound according to formula I, wherein R⁶ is C₆alkyl. One additional embodiment of the present invention provides the use of a compound according to formula I, wherein R⁶ is tetrahydropyran.

Yet one additional embodiment of the present invention provides the use of a compound according to formula I, wherein R⁷ is methyl or trifluoromethyl.

One embodiment of the present invention provides the use of a compound according to formula I, wherein R⁴ is selected from hydrogen, halo, NO₂, C₁₋₄alkyl, C₁₋₃haloalkyl, OR^{a}, SO₂Rⁱ, C(O)NR^{b}R^{c} and C(O)OR^{a}. According to one additional embodiment of the present invention, R⁴ is C(O)NR^{b}R^{c} and R^{b} and R^{c} are independently selected from hydrogen and C₁₋₆alkyl, and said C₁₋₆alkyl is optionally substituted with one or more OR^{a} and R^{a} is C₁₋₃alkyl. According to a further embodiment of the present invention, R⁴ is trifluoromethyl. According to yet another embodiment of the present invention R⁴ is chloro. According to a further embodiment of the present invention, R^{a} is trifluoromethyl. Another embodiment of the present invention relates to the use of a compound according to formula I, wherein R² is hydrogen, halo, C₁₋₃alkyl or OR^{a}. According to one additional embodiment of the present invention, R² is chloro.

Yet another embodiment of the present invention provides the use of a compound according to formula I, wherein R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} and C(O)R^{j}. According to one additional embodiment of the present invention, R¹ is C₀₋₂alkylC(O)NR^{b}R^{c} and R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to one additional embodiment of the present invention R^{b} and R^{c}, together with the atom to which they are attached, form a heterocyclic ring, wherein said heterocyclic ring is optionally substituted with one or more halo, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to yet one additional embodiment of the present invention said heterocyclic ring is substituted with methyl.

According to another embodiment of the present invention, R¹ is C₁₋₄alkylNR^{b}R^{c} and R^{b} and R^{c} together with the atom to which they are attached, form a heterocyclic ring.

According to yet another embodiment of the present invention R¹ is SO₂Rⁱ and k is C₁₋₆alkyl, wherein said C₁₋₆alkyl is optionally substituted with one or more OR^{a}. According to one additional embodiment of the present invention Rⁱ is methyl. According to one additional embodiment of the present invention, R¹ is SO₂NR^{b}R^{c} and R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{d}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to a further additional embodiment of the present invention, R^{b} and R^{c}, together with the atom to which they are attached form a heterocyclic ring, wherein said heterocyclic ring is optionally substituted with one or more halo, C₁₋₆alkyl or C₁₋₃haloalkyl. According to a further embodiment of the present invention said heterocyclic ring is substituted with a C₁₋₆alkyl. According to a further embodiment of the present invention said C₁₋₆alkyl is methyl.

One embodiment of the present invention relates to the use of a compound according to formula I, wherein said compound is selected from:
4-(1,2-Dimethyl-1H-imidazol-5-yl)-5-fluoro-N-[3-methoxy-5-(trifluoromethyl)phenyl]pyrimidin-2-amine;
N-(3,5-Dichlorophenyl)-4-(1,2-dimethyl-1H-imidazol-5-yl)-5-fluoropyrimidin-2-amine;
(4-{[4-(1,2-Dimethyl-1H-imidazol-5-yl)-5-fluoropyrimidin-2-yl]amino}phenyl)(phenyl)methanone;
4-(1,2-Dimethyl-1H-imidazol-5-yl)-5-fluoro-N-{2-methyl-4-[(4-methylpiperazin-1-yl)carbonyl]phenyl}pyrimidin-2-amine;
4-(1,2-Dimethyl-1H-imidazol-5-yl)-5-fluoro-N-{4-[(4-methylpiperazin-1-yl)carbonyl]-3-nitrophenyl}pyrimidin-2-amine;
4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride;
5-Fluoro-N-{4-[(4-methylpiperazin-1-yl)sulfonyl]phenyl}-4-[2-methyl-1-(tetrahydro-2H-pyran-4-yl)-1H-imidazol-5-yl]pyrimidin-2-amine hydrochloride;
5-Fluoro-N-{4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-{3-methoxy-4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)sulfonyl]-3-(trifluoromethoxy)phenyl]-4-[2-methyl-1-(tetrahydro-2H-pyran-4-yl)-1H-imidazol-5-yl]pyrimidin-2-amine hydrochloride;
5-Fluoro-4-[2-methyl-1-(tetrahydro-2H-pyran-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(morpholin-4-ylsulfonyl)phenyl]pyrimidin-2-amine hydrochloride;
[4-({5-Fluoro-4-[2-methyl-1-(tetrahydro-2H-pyran-4-yl)-1H-imidazol-5-yl]pyrimidin-2-yl}amino)phenyl](pyridin-2-yl)methanone hydrochloride;
5-Fluoro-4-[2-methyl-1-(tetrahydro-2H-pyran-4-yl)-1H-imidazol-5-yl]-N-[4-(morpholin-4-ylmethyl)phenyl]pyrimidin-2-amine hydrochloride;
5-Fluoro-4-[2-methyl-1-(tetrahydro-2H-pyran-4-yl)-1H-imidazol-5-yl]-N-[4-(piperidin-1-ylcarbonyl)phenyl]pyrimidin-2-amine hydrochloride;
4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride;
4-(1-Cyclohexyl-2-methyl-1H-imidazol-5-yl)-5-fluoro-N-{4-[(4-methylpiperazin-1-yl)sulfonyl]phenyl}pyrimidin-2-amine hydrochloride;
5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride;
5-Fluoro-4-[2-methyl-1-(1-methylpiperidin-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(trifluoromethyl)phenyl]pyrimidin-2-amine hydrochloride;
5-Fluoro-N-[3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)phenyl]-4-[2-methyl-1-(tetrahydro-2H-pyran-4-yl)-1H-imidazol-5-yl]pyrimidin-2-amine hydrochloride;
3-({5-Fluoro-4-[2-methyl-1-(tetrahydro-2H-pyran-4-yl)-1H-imidazol-5-yl]pyrimidin-2-yl}amino)benzonitrile hydrochloride;
4-(1,2-Dimethyl-1H-imidazol-5-yl)-5-fluoro-N-[4-(morpholin-4-ylmethyl)phenyl]pyrimidin-2-amine hydrochloride;
4-(1,2-Dimethyl-1H-imidazol-5-yl)-5-fluoro-N-{4-[(4-methylpiperazin-1-yl)sulfonyl]phenyl}pyrimidin-2-amine;
4-(1,2-Dimethyl-1H-imidazol-5-yl)-5-fluoro-N-[4-(piperidin-1-ylcarbonyl)phenyl]pyrimidin-2-amine hydrochloride;
4-(1,2-Dimethyl-1H-imidazol-5-yl)-5-fluoro-N-{4-[(4-methylpiperazin-1-yl)carbonyl]phenyl}pyrimidin-2-amine hydrochloride;
4-(1,2-Dimethyl-1H-imidazol-5-yl)-5-fluoro-N-{4-[(4-methylpiperazin-1-yl)methyl]phenyl}pyrimidin-2-amine hydrochloride;
4-(1,2-Dimethyl-1H-imidazol-5-yl)-5-fluoro-N-{3-[(4-methylpiperazin-1-yl)carbonyl]phenyl}pyrimidin-2-amine hydrochloride;
(4-{[4-(1,2-Dimethyl-1H-imidazol-5-yl)-5-fluoropyrimidin-2-yl]amino}phenyl)(pyridin-2-yl)methanone hydrochloride;
4-({5-Fluoro-4-[2-methyl-1-(tetrahydro-2H-pyran-4-yl)-1H-imidazol-5-yl]pyrimidin-2-yl}amino)benzonitrile hydrochloride;
5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride;
5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride;
N-{4-[(Dimethylamino)methyl]phenyl}-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(1-morpholin-4-ylethyl)phenyl]pyrimidin-2-amine;
N-[4-(1-Azetidin-1-ylethyl)phenyl]-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(2-morpholin-4-ylethyl)phenyl]pyrimidin-2-amine;
N-[4-(Methylsulfonyl)phenyl]-4-[2-methyl-1-(tetrahydro-2H-pyran-4-yl)-1H-imidazol-5-yl]pyrimidin-2-amine;
N-{4-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{4-[(4-Methylpiperazin-1-yl)carbonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(morpholin-4-ylmethyl)phenyl]pyrimidin-2-amine;
4-[2-methyl-1-(tetrahydro-2H-pyran-4-yl)-1H-imidazol-5-yl]-N-[4-(morpholin-4-ylsulfonyl)phenyl]pyrimidin-2-amine;
N-(4-{[4-(2-Methoxyethyl)piperazin-1-yl]sulfonyl}phenyl)-4-[2-methyl-1-(tetrahydro-2H-pyran-4-yl)-1H-imidazol-5-yl]pyrimidin-2-amine;
N-{4-[(4-Isopropylpiperazin-1-yl)sulfonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(pyrrolidin-1-ylsulfonyl)phenyl]pyrimidin-2-amine;
(N-(1-Methylpiperidin-4-yl)-4-({4-[2-methyl-1-(tetrahydro-2H-pyran-4-yl)-1H-imidazol-5-yl]pyrimidin-2-yl}amino)benzenesulfonamide;
N-{4-[(4-Methyl-1,4-diazepan-1-yl)sulfonyl]phenyl}-4-[2-methyl-1-(tetrahydro-2H-pyran-4-yl)-1H-imidazol-5-yl]pyrimidin-2-amine;
N,N-Diethyl-4-({4-[2-methyl-1-(tetrahydro-2H-pyran-4-yl)-1H-imidazol-5-yl]pyrimidin-2-yl}amino)benzenesulfonamide;
N-[4-(Azetidin-1-ylsulfonyl)phenyl]-4-[2-methyl-1-(tetrahydro-2H-pyran-4-yl)-1H-imidazol-5-yl]pyrimidin-2-amine;
N-{3-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Chloro-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(3R)-3-methylmorpholin-4-yl]sulfonyl}phenyl)-4-[2-methyl-1-(tetrahydro-2H-pyran-4-yl)-1H-imidazol-5-yl]pyrimidin-2-amine;
5-Fluoro-N-{3-methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(1S,4S)-5-methyl-2,5-diazabicyclo[2.2.1]hept-2-yl]sulfonyl}phenyl)-4-[2-methyl-1-(tetrahydro-2H-pyran-4-yl)-1H-imidazol-5-yl]pyrimidin-2-amine;
4-({5-Fluoro-4-[2-methyl-1-(tetrahydro-2H-pyran-4-yl)-1H-imidazol-5-yl]pyrimidin-2-yl}amino)-N,N-dimethylbenzenesulfonamide;
N-[4-(Azetidin-1-ylsulfonyl)phenyl]-5-fluoro-4-[2-methyl-1-(tetrahydro-2H-pyran-4-yl)-1H-imidazol-5-yl]pyrimidin-2-amine;
Methyl 3-{[4-(1,2-dimethyl-1H-imidazol-5-yl)-5-fluoropyrimidin-2-yl]amino}benzoate;
3-[[4-(2,3-Dimethylimidazol-4-yl)-5-fluoro-pyrimidin-2-yl]amino]-N-(3-methoxypropyl)benzamide hydrochloride;
[4-[[4-(2,3-Dimethylimidazol-4-yl)-5-fluoro-pyrimidin-2-yl]amino]-2-(trifluoromethoxy)phenyl]-(4-methylpiperazin-1-yl)methanone hydrochloride;
N-[4-(Azetidin-1-ylcarbonyl)phenyl]-5-fluoro-4-[2-methyl-1-(tetrahydro-2H-pyran-4-yl)-1H-imidazol-5-yl]pyrimidin-2-amine hydrochloride;
N-{4-[(3,3-Difluoroazetidin-1-yl)carbonyl]phenyl}-5-fluoro-4-[2-methyl-1-(tetrahydro-2H-pyran-4-yl)-1H-imidazol-5-yl]pyrimidin-2-amine hydrochloride;
5-Fluoro-N-[3-methyl-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-[4-(morpholin-4-ylmethyl)phenyl]-4-[3-oxan-4-yl-2-(trifluoromethyl)imidazol-4-yl]-pyrimidin-2-amine hydrochloride;
5-Fluoro-N-{4-[(4-fluoropiperidin-1-yl)carbonyl]phenyl}-4-[2-methyl-1-(tetrahydro-2H-pyran-4-yl)-1H-imidazol-5-yl]pyrimidin-2-amine hydrochloride;
Ethyl 4-({5-fluoro-4-[2-methyl-1-(tetrahydro-2H-pyran-4-yl)-1H-imidazol-5-yl]pyrimidin-2-yl}amino)benzoate;
N,N-Diethyl-4-({5-fluoro-4-[2-methyl-1-(tetrahydro-2H-pyran-4-yl)-1H-imidazol-5-yl]pyrimidin-2-yl}amino)benzamide hydrochloride;
4-[5-fluoro-4-(2-methyl-3-tetrahydropyran-4-yl-imidazol-4-yl)-pyrimidin-2-yl]amino-N-(3-methoxypropyl)benzamide hydrochloride;
[4-[5-fluoro-4-(2-methyl-3-tetrahydropyran-4-yl-imidazol-4-yl)-pyrimidin-2-yl]aminophenyl]-(1,4-oxazepan-4-yl)methanone hydrochloride;
(4-ethylpiperazin-1-yl)-[4-[5-fluoro-4-(2-methyl-3-tetrahydropyran-4-yl-imidazol-4-yl)-pyrimidin-2-yl]aminophenyl]-methanone hydrochloride;
(2,6-dimethylmorpholin-4-yl)-[4-[5-fluoro-4-(2-methyl-3-tetahydropyran-4-yl-imdazol-4-yl)-pyrimidin-2-yl]aminophenyl]-methanone hydrochloride;
[4-[5-fluoro-4-(2-methyl-3-tetrahydropyran-4-yl-imidazol-4-yl)-pyrimidin-2-yl]aminophenyl]-(3-fluoropyrrolidin-1-yl)-methanone hydrochloride;
(3,3-difluoropyrrolidin-1-yl)-[4-[5-fluoro-4-(2-methyl-3-tetrahydropyran-4-yl-imidazol-4-yl)-pyrimidin-2-yl]aminophenyl]-methanone hydrochloride;
4-[5-fluoro-4-(2-methyl-3-tetrahydropyran-4-yl-imidazol-4-yl)-pyrimidin-2-yl]amino-N-methyl-benzamide hydrochloride;
4-[5-fluoro-4-(2-methyl-3-tetahydropyran-4-yl-imidazol-4-yl)-pyrimidin-2-yl]amino-N-tetrahydropyran-4-yl-benzamide hydrochloride;
[4-[5-fluoro-4-(2-methyl-3-tetrahydropyran-4-yl-imidazol-4-yl)-pyrimidin-2-yl]aminophenyl]-(3-hydroxypyrrolidin-1-yl)-methanone hydrochloride;
N-(2-cyanoethyl)-4-[5-fluoro-4-(2-methyl-3-tetahydropyran-4-yl-imidazol-4-yl)-pyrimidin-2-yl]amino-N-methyl-benzamide hydrochloride;
N-ethyl-4-[5-fluoro-4-(2-methyl-3-tetrahydropyran-4-yl-imidazol-4-yl)-pyrimidin-2-yl]amino-N-(2-hydroxyethyl)benzamide hydrochloride;
4-[5-fluoro-4-(2-methyl-3-tetrahydropyran-4-yl-imidazol-4-yl)-pyrimidin-2-yl]amino-N-(2-hydroxyethyl)-N-methyl-benzamide hydrochloride;
4-[5-fluoro-4-(2-methyl-3-tetahydropyran-4-yl-imidazol-4-yl)-pyrimidin-2-yl]amino-N-(2-hydroxyethyl)benzamide hydrochloride;
N-(2-dimethylaminoethyl)-4-[5-fluoro-4-(2-methyl-3-tetrahydropyran-4-yl-imidazol-4-yl)-pyrimidin-2-yl]amino-benzamide hydrochloride;
(4-dimethylamino-1-piperidyl)-[4-[5-fluoro-4-(2-methyl-3-tetrahydropyran-4-yl-imidazol-4-yl)-pyrimidin-2-yl]aminophenyl]-methanone hydrochloride;
[4-[5-fluoro-4-(2-methyl-3-tetahydropyran-4-yl-imidazol-4-yl)-pyrimidin-2-yl]aminophenyl]-[4-(2-methoxyethyl)piperazin-1-yl]-methanone hydrochloride;
4-[5-fluoro-4-(2-methyl-3-tetrahydropyran-4-yl-imidazol-4-yl)-pyrimidin-2-yl]amino-N-[2-(1-piperidyl)ethyl]benzamide hydrochloride;
4-[5-fluoro-4-(2-methyl-3-tetrahydropyran-4-yl-imidazol-4-yl)-pyrimidin-2-yl]amino-N-(2-morpholinoethyl)benzamide hydrochloride;
4-[5-fluoro-4-(2-methyl-3-tetrahydropyran-4-yl-imidazol-4-yl)-pyrimidin-2-yl]amino-N-isopropyl-benzamide hydrochloride;
N-[2-(3,3-difluoropyrrolidin-1-yl)ethyl]-4-[5-fluoro-4-(2-methyl-3-tetrahydropyran-4-yl-imidazol-4-yl)-pyrimidin-2-yl]amino-benzamide hydrochloride;
[4-[5-fluoro-4-(2-methyl-3-tetrahydropyran-4-yl-imidazol-4-yl)-pyrimidin-2-yl]aminophenyl]-(4-isopropylpiperazin-1-yl)-methanone hydrochloride;
[4-[5-fluoro-4-(2-methyl-3-tetrahydropyran-4-yl-imidazol-4-yl)-pyrimidin-2-yl]aminophenyl]-(4-methyl-1,4-diazepan-1-yl)-methanone hydrochloride;
4-[5-fluoro-4-(2-methyl-3-tetrahydropyran-4-yl-imidazol-4-yl)-pyrimidin-2-yl]amino-N-tetrahydrofuran-3-yl-benzamide hydrochloride;
5-Fluoro-N-[4-(methylsulfonyl)phenyl]-4-[1-(tetrahydro-2H-pyran-4-yl)-2-(trifluoromethyl)-1H-imidazol-5-yl]pyrimidin-2-amine;
N-[4-(Azetidin-1-ylcarbonyl)phenyl]-5-fluoro-4-[1-(tetrahydro-2H-pyran-4-yl)-2-(trifluoromethyl)-1H-imidazol-5-yl]pyrimidin-2-amine;
N-[4-(Azetidin-1-ylcarbonyl)-3-chlorophenyl]-4-(1,2-dimethyl-1H-imidazol-5-yl)-5-fluoropyrimidin-2-amine;
N-[4-(Azetidin-1-ylcarbonyl)-3-methylphenyl]-4-(1,2-dimethyl-1H-imidazol-5-yl)-5-fluoropyrimidin-2-amine;
N-[3-Chloro-4-(methylsulfonyl)phenyl]-4-(1,2-dimethyl-1H-imidazol-5-yl)-5-fluoropyrimidin-2-amine;
4-(1,2-Dimethyl-1H-imidazol-5-yl)-5-fluoro-N-[4-(methylsulfonyl)phenyl]pyrimidin-2-amine;
N-{3-Chloro-4-[(4-methylpiperazin-1-yl)sulfonyl]phenyl}-4-(1,2-dimethyl-1H-imidazol-5-yl)-5-fluoropyrimidin-2-amine;
4-(1,2-Dimethyl-1H-imidazol-5-yl)-5-fluoro-N-{3-methyl-4-[(4-methylpiperazin-1-yl)sulfonyl]phenyl}pyrimidin-2-amine;
N-[4-(Azetidin-1-ylcarbonyl)-3-(trifluoromethoxy)phenyl]-4-(1,2-dimethyl-1H-imidazol-5-yl)-5-fluoropyrimidin-2-amine;
5-Fluoro-N-[4-(4-methylpiperazin-1-yl)sulfonylphenyl]-4-[3-methyl-2-(trifluoromethyl)imidazol-4-yl]-pyrimidin-2-amine hydrochloride;
5-Fluoro-4-[3-methyl-2-(trifluoromethyl)imidazol-4-yl]-N-[4-(morpholin-4-ylmethyl)phenyl]-pyrimidin-2-amine hydrochloride;
[4-[5-Fluoro-4-[3-methyl-2-(trifluoromethyl)imidazol-4-yl]-pyrimidin-2-yl]aminophenyl]-(4-methylpiperazin-1-yl)-methanone hydrochloride;
[4-[5-Fluoro-4-[3-tetrahydropyran-4-yl-2-(trifluoromethyl)imidazol-4-yl]-pyrimidin-2-yl]aminophenyl]-(4-methylpiperazin-1-yl)-methanone hydrochloride;
5-Fluoro-N-[3-(methylsulfonyl)-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)-3-(trifluoromethyl)phenyl]-4-[2-methyl-1-(tetrahydro-2H-pyran-4-yl)-1H-imidazol-5-yl]pyrimidin-2-amine hydrochloride;
6-({5-Fluoro-4-[2-methyl-1-(tetrahydro-2H-pyran-4-yl)-1H-imidazol-5-yl]pyrimidin-2-yl}amino)-2,3-dihydro-4H-thiochromen-4-one 1,1-dioxide hydrochloride;
6-({5-Fluoro-4-[2-methyl-1-(tetrahydro-2H-pyran-4-yl)-1H-imidazol-5-yl]pyrimidin-2-yl}amino)thiochroman-4-ol 1,1-dioxide hydrochloride;
N-(3-Dimethylaminopropyl)-3-[[4-(2,3-dimethylimidazol-4-yl)-5-fluoro-pyrimidin-2-yl]amino]benzamide;
N-(3-Dimethylaminopropyl)-3-[[4-(2,3-dimethylimidazol-4-yl)-5-fluoro-pyrimidin-2-yl]amino]-N-methyl-benzamide hydrochloride;
[3-[[4-(2,3-Dimethylimidazol-4-yl)-5-fluoro-pyrimidin-2-yl]amino]phenyl]-[3-(hydroxymethyl)-1-piperidyl]methanone;
N-{3-Chloro-4-[(4-methylpiperazin-1-yl)carbonyl]phenyl}-5-fluoro-4-[2-methyl-1-(tetrahydro-2H-pyran-4-yl)-1H-imidazol-5-yl]pyrimidin-2-amine;
5-Fluoro-N-{3-[(4-methylpiperazin-1-yl)carbonyl]phenyl}-4-[2-methyl-1-(tetrahydro-2H-pyran-4-yl)-1H-imidazol-5-yl]pyrimidin-2-amine;
(4-{[4-(1,2-Dimethyl-1H-imidazol-5-yl)-5-fluoropyrimidin-2-yl]amino}phenyl)(pyridin-2-yl)methanol;
5-Fluoro-N-[4-(isopropylsulfonyl)phenyl]-4-[2-methyl-1-(tetrahydro-2H-pyran-4-yl)-1H-imidazol-5-yl]pyrimidin-2-anine;
N-[4-(Ethylsulfonyl)phenyl]-5-fluoro-4-[2-methyl-1-(tetrahydro-2H-pyran-4-yl)-1H-imidazol-5-yl]pyrimidin-2-amine;
5-Fluoro-N-{4-[(2-methoxyethyl)sulfonyl]phenyl}-4-[2-methyl-1-(tetrahydro-2H-pyran-4-yl)-1H-imidazol-5-yl]pyrimidin-2-amine;
N-(4-{[2-(Diethylamino)ethyl]sulfonyl}phenyl)-5-fluoro-4-[2-methyl-1-(tetrahydro-2H-pyran-4-yl)-1H-imidazol-5-yl]pyrimidin-2-amine;
2-{[4-({5-Fluoro-4-[2-methyl-1-(tetrahydro-2H-pyran-4-yl)-1H-imidazol-5-yl]pyrimidin-2-yl}amino)phenyl]sulfonyl}ethanol;
{5-Fluoro-4-[3-(tetrahydro-pyran-4-yl)-3H-imidazol-4-yl]-pyrimidin-2-yl}-[4-(4-methyl-piperazine-1-sulfonyl)-phenyl]-amine;
5-{5-Fluoro-2-[4-(4-methyl-piperazine-1-sulfonyl)-phenylamino]-pyrimidin-4-yl}-1-(tetrahydro-pyran-4-yl)-1H-imidazole-2-carbonitrile; and
{5-Fluoro-4-[2-methyl-3-(tetrahydro-pyran-4-yl)-3H-imidazol-4-yl]-pyrimidin-2-yl}-[4-(tetrahydro-pyran-2-ylmethanesulfonyl)-phenyl]-amine.
   in the manufacture of a medicament for prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

According to one embodiment of the present invention, the disease is Alzheimer's Disease.

The present invention also relates to a compound of the formula I:

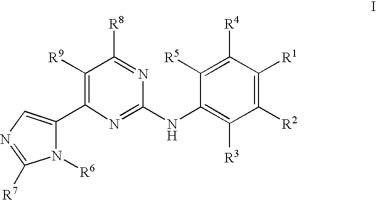

wherein

R¹ is selected from hydrogen, cyano, C₁₋₃haloalkyl, OR^{a}, SO₂NR^{b}R^{c}, C₀₋₂alkylC(O)NR^{b}R^{c}, C₁₋₄alkylNR^{b}R^{c}, CH₂OR^{h}, SO₂Rⁱ, C(O)OR^{a}, CH(OH)R^{j} and C(O)R^{j};

R² and R⁴ are independently selected from hydrogen, halo, cyano, NO₂, C₁₋₄alkyl, C₁₋₃haloalkyl, OR^{a}, C(O)NR^{b}R^{c}, SO₂Rⁱ and C(O)OR^{a}; or

R¹ and R², together with the atoms to which they are attached form a 5- or 6-membered heterocyclic ring containing at least one N, O or S, in which any of the hydrogens of the CH₂-groups within said heterocyclic ring can be substituted with oxo, hydroxy or halo and in which any sulphur atom within said heterocyclic ring is optionally oxidised to —SO₂—;

R³ and R⁵ are independently selected from hydrogen, C₁₋₃alkyl and OR^{a};

R⁶ is selected from CH₃ and C₆alkyl; or

R⁶ is a 6-membered heterocyclic ring containing one or more heteroatoms selected from N, is O or S, wherein said heterocyclic ring is optionally substituted with one or more C₁₋₃alkyl or C₁₋₃haloalkyl, wherein said C₁₋₃alkyl or C₁₋₃haloalkyl is optionally further substituted with one or more C₁₋₃alkoxy;

R⁷ is selected from hydrogen, C₁₋₃alkyl, cyano and C₁₋₃haloalkyl, wherein said C₁₋₃aryl or C₁₋₃haloalkyl is optionally substituted with one or more OR^{a};

R⁸ and R⁹ are independently are selected from hydrogen and halo;

R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;

R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or

R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};

R^{d} and R^{e} are independently selected from hydrogen, C₁₋₆alkyl and C₁₋₆haloalkyl, wherein said C₁₋₆alkyl or C₁₋₆haloalkyl is optionally substituted with one or more OR^{a}; or

R^{d} and R^{e} may, together with the atom to which they are attached, form a heterocyclic ring wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;

R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy;

Rⁱ is selected from C₁₋₆alkyl, heterocyclyl, aryl, heteroaryl and C₁₋₃haloalkyl, wherein said C₁₋₆alkyl, heterocyclyl, aryl, heteroaryl or C₁₋₃haloalkyl is optionally substituted with one or more halo, cyano, di-(C₁₋₄alkyl)amino-, C₁₋₃haloalkyl, C₁₋₃alkyl, heterocyclyl or OR^{a};

R^{j} is an aryl or heteroaryl ring, wherein said aryl or heteroaryl ring is optionally substituted with one or more C₁₋₃alkyl, OR^{a}, halo or cyano;

as a free base or a pharmaceutically acceptable salt, solvate or solvate of a salt thereof.

The present invention also relates to a compound of the formula Ib:

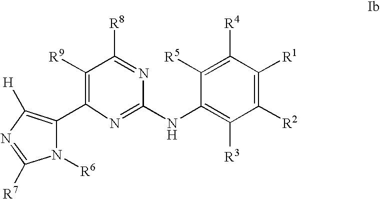

wherein

R¹ is selected from hydrogen, cyano, C₁₋₃haloalkyl, SO₂NR^{b}R^{c}, C(O)NR^{b}R^{c}, CH₂NR^{b}R^{c}, CH₂OR^{h}, SO₂Rⁱ and C(O)R^{j};

R² and R⁴ are independently selected from hydrogen, halo, cyano, NO₂, C₁₋₃haloalkyl, OR^{a}, C(O)NR^{b}R^{c}, and SO₂Rⁱ;

R³ and R⁵ independently are selected from hydrogen, C₁₋₃alkyl, and OR^{a};

R⁶ is selected from CH₃ and C₆alkyl; or

R⁶ is a 6-membered heterocyclic ring containing one or more heteroatoms selected from N, or S, wherein said heterocyclic ring is optionally substituted with one or more C₁₋₃alkyl or C₁₋₃haloalkyl;

R⁷ is selected from C₁₋₃alkyl and C₁₋₃haloalkyl;

R⁸ and R⁹ independently are selected from hydrogen and halo;

R^{a} is C₁₋₃alkyl or C₁₋₃haloalkyl;

R^{b} and R^{c} are independently selected from hydrogen and C₁₋₆alkyl, optionally substituted with one or more OR^{a}; or

R^{b} and R^{c} may, together with the atom to which they are attached, form a 4-, 5- or 6-membered heterocyclic ring containing one or more heteroatoms selected from N or O, wherein said heterocyclic ring is optionally substituted with one or more halo or C₁₋₃alkyl;

R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy;

Rⁱ is C₁₋₃alkyl;

R^{j} is an aryl or heteroaryl ring;

as a free base or a pharmaceutically acceptable salt, solvate or solvate of a salt thereof.

One embodiment of the present invention relates to a compound of formula I, wherein R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} and C(O)R^{j};

R² and R⁴ are independently selected from hydrogen, halo, cyano, NO₂, C₁₋₄alkyl, C₁₋₃haloalkyl, OR^{a}, SO₂Rⁱ, C(O)NR^{b}R^{c} and C(O)OR^{a}; or

R¹ and R², together with the atoms to which they are attached join to form a 5- or 6-membered heterocyclic ring containing at least one N, O or S, in which any of the hydrogens of the CH₂-groups within the said heterocyclic ring can be substituted with oxo, hydroxy or halo and in which any sulphur atom within said heterocyclic ring is optionally oxidised to —SO₂—;

R³ and R⁵ are independently selected from hydrogen, C₁₋₃alkyl, and OR^{a};

R⁶ is selected from CH₃ and C₆alkyl; or

R⁶ is a 6-membered heterocyclic ring containing one or more heteroatoms selected from N or O, wherein said heterocyclic ring is optionally substituted with one or more C₁₋₃alkyl;

R⁷ is selected from C₁₋₃alkyl, cyano, and C₁₋₃haloalkyl;

R⁸ and R⁹ are independently selected from hydrogen and halo;

R^{a} is selected from hydrogen, C₁₋₃alkyl and C₁₋₃haloalkyl, wherein said C₁₋₃alkyl is optionally substituted with one or more C₁₋₃alkoxy;

R^{b} and R^{c} are independently selected from hydrogen, C₁₋₆alkyl and heterocyclyl, wherein said C₁₋₆alkyl, heterocyclyl is optionally substituted with one or more cyano, OR^{a} or NR^{d}R^{e}; or

R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};

R^{d} and R^{e} are independently selected from C₁₋₆alkyl; or

R^{d} and R^{e} may, together with the atom to which they are attached, form a heterocyclic ring wherein said heterocyclic ring is optionally substituted with one or more halo;

Rⁱ is selected from C₁₋₆alkyl and heterocyclyl, wherein said C₁₋₆alkyl or heterocyclyl is optionally substituted with one or more di-(C₁₋₄alkyl)amino-, heterocyclyl or OR^{a};

R^{j} is an aryl or heteroaryl ring, wherein said aryl or heteroaryl ring is optionally substituted with one or more C₁₋₃alkyl; as a free base or a pharmaceutically acceptable salt, solvate or solvate of a salt thereof.

Another embodiment of the present invention relates to a compound of formula I, wherein R³ and R⁵ are hydrogen.

Yet another embodiment of the present invention provides a compound of formula I, wherein R⁸ is hydrogen and R⁹ is hydrogen or fluoro.

A further embodiment of the present invention provides a compound of formula I, wherein R⁶ is C₆alkyl. According to one additional embodiment of the present invention, R⁶ is tetrahydropyran.

Yet another embodiment of the present invention provides a compound of formula I, wherein R⁷ is methyl or trifluoromethyl.

One embodiment of the present invention provides a compound of formula I, wherein R⁴ is selected from hydrogen, halo, NO₂, C₁₋₄alkyl, C₁₋₃haloalkyl, OR^{a}, SO₂Rⁱ, C(O)NR^{b}R^{c} and C(O)OR^{a}. According to another embodiment of the present invention, R^{b} and R^{c} are independently selected from hydrogen and C₁₋₆alkyl, wherein said C₁₋₆alkyl is optionally substituted with one or more OR^{a} and wherein R^{a} is C₁₋₃alkyl. According to yet another embodiment of the present invention, R⁴ is trifluoromethyl. According to one additional embodiment of the present invention, R⁴ is chloro. According to yet one additional embodiment of the present invention, R^{a} is trifluoromethyl.

One embodiment of the present invention provides a compound of formula I, wherein R² is hydrogen, halo, C₁₋₃alkyl or OR^{a}. According to one additional embodiment of the present invention, R² is chloro.

Yet one embodiment of the present invention provides a compound of formula I, wherein R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} and C(O)R^{j}. According to one additional embodiment of the present invention, R¹ is C₀₋₂alkylC(O)NR^{b}R^{c} and R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to yet one additional embodiment of the present invention R^{b} and R^{c}, together with the atom to which they are attached, form a heterocyclic ring, wherein said heterocyclic ring is optionally substituted with one or more halo, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to another embodiment of the present invention, said heterocyclic ring is substituted with methyl.

According to a further embodiment of the present invention, R¹ is C₁₋₄alkylNR^{b}R^{c} and R^{b} and R^{c} together with the atom to which they are attached, form a heterocyclic ring.

According to yet a further embodiment of the present invention, SO₂R¹ and R¹ is C₁₋₆alkyl, wherein said C₁₋₆alkyl is optionally substituted with one or more OR^{a}. According to yet one additional embodiment of the present invention Rⁱ is methyl.

According to another embodiment of the present invention, R¹ is SO₂NR^{b}R^{c} and R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR¹ or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}. According to one additional embodiment of the present invention, R^{b} and R^{c} together with the atom to which they are attached form a heterocyclic ring, wherein said heterocyclic ring is optionally substituted with one or more halo, C₁₋₆alkyl or C₁₋₃haloalkyl. According to yet one additional embodiment of the present invention said heterocyclic ring is substituted with a C₁₋₆alkyl. According to yet a further additional embodiment of the present invention, said C₁₋₆alkyl is methyl.

One embodiment of the present invention provides a compound of formula I selected from:
4-(1,2-Dimethyl-1H-imidazol-5-yl)-5-fluoro-N-[3-methoxy-5-(trifluoromethyl)phenyl]pyrimidin-2-amine;
N-(3,5-Dichlorophenyl)-4-(1,2-dimethyl-1H-imidazol-5-yl)-5-fluoropyrimidin-2-amine;
(4-{[4-(1,2-Dimethyl-1H-imdazol-5-yl)-5-fluoropyrimidin-2-yl]amino}phenyl)(phenyl)methanone;
4-(1,2-Dimethyl-1H-imidazol-5-yl)-5-fluoro-N-{2-methyl-4-[(4-methylpiperazin-1-yl)carbonyl]phenyl}pyrimidin-2-amine;
4-(1,2-Dimethyl-1H-imidazol-5-yl)-5-fluoro-N-{4-[(4-methylpiperazin-1-yl)carbonyl]-3-nitrophenyl}pyrimidin-2-amine;
4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride;
5-Fluoro-N-{4-[(4-methylpiperazin-1-yl)sulfonyl]phenyl}-4-[2-methyl-1-(tetrahydro-2H-pyran-4-yl)-1H-imidazol-5-yl]pyrimidin-2-amine hydrochloride;
5-Fluoro-N-{4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-{3-methoxy-4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)sulfonyl]-3-(trifluoromethoxy)phenyl]-4-[2-methyl-1-(tetrahydro-2H-pyran-4-yl)-1H-imidazol-5-yl]pyrimidin-2-amine hydrochloride;
5-Fluoro-4-[2-methyl-1-(tetrahydro-2H-pyran-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(morpholin-4-ylsulfonyl)phenyl]pyrimidin-2-amine hydrochloride;
[4-({5-Fluoro-4-[2-methyl-1-(tetrahydro-2H-pyran-4-yl)-1H-imidazol-5-yl]pyrimidin-2-yl}amino)phenyl](pyridin-2-yl)methanone hydrochloride;
5-Fluoro-4-[2-methyl-1-(tetrahydro-2H-pyran-4-yl)-1H-imidazol-5-yl]-N-[4-(morpholin-4-ylmethyl)phenyl]pyrimidin-2-amine hydrochloride;
5-Fluoro-4-[2-methyl-1-(tetrahydro-2H-pyran-4-yl)-1H-imidazol-5-yl]-N-[4-(piperidin-1-ylcarbonyl)phenyl]pyrimidin-2-amine hydrochloride;
4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride;
4-(1-Cyclohexyl-2-methyl-1H-imidazol-5-yl)-5-fluoro-N-{4-[(4-methylpiperazin-1-yl)sulfonyl]phenyl}pyrimidin-2-amine hydrochloride;
5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride;
5-Fluoro-4-[2-methyl-1-(1-methylpiperidin-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(trifluoromethyl)phenyl]pyrimidin-2-amine hydrochloride;
5-Fluoro-N-[3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)phenyl]-4-[2-methyl-1-(tetrahydro-2H-pyran-4-yl)-1H-imidazol-5-yl]pyrimidin-2-amine hydrochloride;
3-({5-Fluoro-4-[2-methyl-1-(tetrahydro-2H-pyran-4-yl)-1H-imidazol-5-yl]pyrimidin-2-yl}amino)benzonitrile hydrochloride;
4-(1,2-Dimethyl-1H-imidazol-5-yl)-5-fluoro-N-[4-(morpholin-4-ylmethyl)phenyl]pyrimidin-2-amine hydrochloride;
4-(1,2-Dimethyl-1H-imidazol-5-yl)-5-fluoro-N-{4-[(4-methylpiperazin-1-yl)sulfonyl]phenyl}pyrimidin-2-amine;
4-(1,2-Dimethyl-1H-imidazol-5-yl)-5-fluoro-N-[4-(piperidin-1-ylcarbonyl)phenyl]pyrimidin-2-amine hydrochloride;
4-(1,2-Dimethyl-1H-imidazol-5-yl)-5-fluoro-N-{4-[(4-methylpiperazin-1-yl)carbonyl]phenyl}pyrimidin-2-amine hydrochloride;
4-(1,2-Dimethyl-1H-imidazol-5-yl)-5-fluoro-N-{4-[(4-methylpiperazin-1-yl)methyl]phenyl}pyrimidin-2-amine hydrochloride;
4-(1,2-Dimethyl-1H-imidazol-5-yl)-5-fluoro-N-{3-[(4-methylpiperazin-1-yl)carbonyl]phenyl}pyrimidin-2-amine hydrochloride;
(4-{[4-(1,2-Dimethyl-1H-imidazol-5-yl)-5-fluoropyrimidin-2-yl]amino}phenyl)(pyridin-2-yl)methanone hydrochloride;
4-({5-Fluoro-4-[2-methyl-1-(tetrahydro-2H-pyran-4-yl)-1H-imidazol-5-yl]pyrimidin-2-yl}amino)benzonitrile hydrochloride;
5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride; and
5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride.

One embodiment of the present invention relates to the compounds disclosed above for use is in therapy.

The present invention also relates to a compound selected from:
2-Chloro-4-(1,2-dimethyl-1H-imidazol-5-yl)-5-fluoropyrimidine;
2-Methyl-4-[(4-methylpiperazin-1-yl)carbonyl]aniline;
4-[(4-Methylpiperazin-1-yl)carbonyl]-3-nitroaniline;
4-[(4-Methylpiperazin-1-yl)carbonyl]-2-(trifluoromethoxy)aniline;
4-[N-Acetyl-N-(tetrahydro-2H-pyran-4-yl)]amino-5-methylisoxazole;
5-Acetyl-2-methyl-1-(tetrahydro-2H-pyran-4-yl)-1H-imidazole;
(2E)-3-Dimethylamino-1-[2-methyl-1-(tetrahydro-2H-pyran-4-yl)-1H-imidazol-5-yl]prop-2-en-1-one;
(2Z)-3-Dimethylamino-2-fluoro-1-[2-methyl-1-(tetrahydro-2H-pyran-4-yl)-1H-imidazol-5-yl]prop-2-en-1-one;
5-Fluoro-4-[2-methyl-1-(tetrahydro-2H-pyran-4-yl)-1H-imidazol-5-yl]pyrimidin-2-amine;
1-(4-Chloro-2-methoxybenzoyl)-4-methylpiperazine;
1-[4-Bromo-2-(methylsulfonyl)benzoyl]-4-methylpiperazine;
4-(N-Acetyl-N-cyclohexyl)amino-5-methylisoxazole;
5-Acetyl-1-cyclohexyl-2-methyl-1H-imidazole;
(2E)-3-Dimethylamino-1-(1-cyclohexyl-2-methyl-1H-imidazol-5-yl)prop-2-en-1-one;
(2Z)-3-Dimethylamino-2-fluoro-1-(1-cyclohexyl-2-methyl-1H-imidazol-5-yl)prop-2-en-1-one;
4-(1-Cyclohexyl-2-methyl-1H-imidazol-5-yl)-5-fluoropyrimidin-2-amine;
5-Acetyl-2-methyl-1-(1-methylpiperidin-4-yl)-1H-imidazole;
(2E)-3-Dimethylamino-1-[2-methyl-1-(1-methylpiperidin-4-yl)-1H-imidazol-5-yl]prop-2-en-1-one;
(2Z)-3-Dimethylamino-2-fluoro-1-[2-methyl-1-(1-methylpiperidin-4-yl)-1H-imidazol-5-yl]prop-2-en-1-one;
5-Fluoro-4-[2-methyl-1-(1-methylpiperidin-4-yl)-1H-imidazol-5-yl]pyrimidin-2-amine;
4-(1,2-Dimethyl-1H-imidazol-5-yl)-5-fluoropyrimidin-2-amine;
1-(tert-Butoxycarbonyl)-4-(4-bromo-benzenesulfonyl)-piperazine;
5-Acetyl-1-(tetrahydro-2H-pyran-4-yl)-2-trifluoromethyl-1H-imidazole;
(2E)-3-Dimethylamino-1-[1-(tetrahydro-2H-pyran-4-yl)-2-trifluoromethyl-1H-imidazol-5-yl]prop-2-en-1-one;
(2Z)-3-Dimethylamino-2-fluoro-1-[1-(tetrahydro-2H-pyran-4-yl)-2-trifluoromethyl-1H-imidazol-5-yl]prop-2-en-1-one;
5-Fluoro-4-[1-(tetrahydro-2H-pyran-4-yl)-2-(trifluoromethyl)-1H-imidazol-5-yl]pyrimidin-2-amine;
4-[2-Methyl-1-(tetrahydro-2H-pyran-4-yl)-1H-imidazol-5-yl]pyrimidin-2-amine;
1-[(4-Bromo-2-chlorophenyl)sulfonyl]-4-methylpiperazine;
(3R)-4-[(4-Bromophenyl)sulfonyl]-3-methylmorpholine;
(1S,4S)-2-[(4-Bromophenyl)sulfonyl]-5-methyl-2,5-diazabicyclo[2.2.1]heptane;
Methyl 4-bromo-2-(trifluoromethoxy)benzoate;
4-Bromo-2-(trifluoromethoxy)benzoic acid;
4-(4-Chloro-2-methylbenzyl)morpholine; Lithium 4-({5-fluoro-4-[2-methyl-1-(tetrahydro-2H-pyran-4-yl)-1H-imidazol-5-yl]pyrimidin-2-yl}amino)benzoate;
1-(4-Bromo-2-methylbenzoyl)azetidine;
4-Bromo-2-(trifluoromethoxy)benzoic acid;
1-[4-Bromo-2-(trifluoromethoxy)benzoyl]azetidine;
2,2,2-Trifluoro-N-methyl-N-(5-methylisoxazol-4-yl)acetamide;
1-[1-Methyl-2-(trifluoromethyl)-1H-imidazol-5-yl]ethanone;
(2E)-3-(Dimethylamino)-1-[1-methyl-2-(trifluoromethyl)-1H-imidazol-5-yl]prop-2-en-1-one;
(2Z)-3-(Dimethylamino)-2-fluoro-1-[1-methyl-2-(trifluoromethyl)-1H-imidazol-5-yl]prop-2-en-1-one;
5-Fluoro-4-[1-methyl-2-(trifluoromethyl)-1H-imidazol-5-yl]pyrimidin-2-amine;
4-[4-Bromo-2-(methylsulfonyl)benzyl]morpholine;
2-[(4-Bromophenyl)sulfonyl]ethyl methyl ether;
2-[(4-Bromophenyl)sulfonyl]ethyl diethyl-amine;
N-(5-Methyl-isoxazol-4-yl)-N-(tetrahydro-pyran-4-yl)-formamide;
5-Acetyl-1-(tetrahydro-pyran-4-yl)-1H-imidazole;
(E)-3-Dimethylamino-1-[3-(tetrahydro-pyran-4-yl)-3H-imidazol-4-yl]-propenone;
(Z)-3-Dimethylamino-2-fluoro-1-[3-(tetrahydro-pyran-4-yl)-3H-imidazol-4-yl]-propenone;
5-Fluoro-4-[1-(tetrahydro-2H-pyran-4-yl)-1H-imidazol-5-yl]pyrimidin-2-amine; and
5-{5-Fluoro-2-[4-(4-methyl-piperazine-1-sulfonyl)-phenylamino]-pyrimid in-4-yl}-1-(tetrahydro-pyran-4-yl)-1H-imidazole-2-carbaldehyde.

The present invention also provides the use of the compounds disclosed above for the preparation of a compound of formula I.

Listed below are definitions of various terms used in the specification and claims to describe the present invention.

In this specification the term “alkyl” includes both straight and branched chain as well as cyclic alkyl groups. The term C₁₋₃alkyl having 1 to 3 carbon atoms and may be, but is not limited to, methyl, ethyl, n-propyl, i-propyl, or cyclopropyl. The term C₁₋₆alkyl having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl or cyclohexyl. The term C₆alkyl having 6 carbon atoms and may be, but is not limited to, n-hexyl, i-hexyl or cyclohexyl. The term C₁₋₄alkylNR^{b}R^{c} includes, but is not limited to, —CH₂NR^{b}R^{c}, —CH₂CH₂NR^{b}R^{c} and —CH(CH₃)NR^{b}R^{c}. The term C₀₋₂alkylC(O)NR^{b}R^{c} is intended to include, but is not limiting, C(O)NR^{b}R^{c} —CH₂C(O)NR^{b}R^{c}, —CH₂CH₂C(O)NR^{b}R^{c} and —CH(CH₃)C(O)NR^{b}R^{c}.

The term “alkenyl” refers to a straight or branched chain alkenyl group. The term C₆alkenyl having 6 carbon atoms and one double bond, and may be, but is not limited to, hexenyl or i-hexenyl.

The term “alkynyl” refers to a straight or branched chain alkynyl group. The term C₆alkynyl having 6 carbon atoms and one triple bond, and may be, but is not limited to, hexynyl or i-hexynyl.

The term “C₁₋₃alkoxy” includes both straight and branched chains. The term “C₁₋₃alkoxy” having 1 to 3 carbon atoms and may be, but is not limited to, methoxy, ethoxy, n-propoxy, or i-propoxy.

The term “halogen” refers to fluorine, chlorine, bromine and iodine.

The term “haloalkyl” refers to an alkyl group, defined as above, in which one or more of the hydrogen substituents have been replaced by halogen substituents, in which the term halogen is defined as above. Examples include trifluoromethyl- and difluoromethyl-.

The term “aryl” refers to an optionally substituted monocyclic or bicyclic hydrocarbon ring system containing at least one unsaturated aromatic ring. The “aryl” may be fused with a C₅₋₇cycloalkyl ring to form a bicyclic hydrocarbon ring system. Examples and suitable values of the term “aryl”, but not limiting, are phenyl, naphthyl, indanyl or tetralinyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

The term “heterocyclic ring” refers to a 4-, 5-, 6- or 7-membered ring containing one or more heteroatoms independently selected from N, O, or S, said ring can be a mono- or bicyclic, which may be saturated or partly saturated and which may optionally contain a carbonyl function and which may be, but is not limited to, azetidinyl, imidazolidinyl, imidazolinyl, morpholinyl, piperazinyl, piperidinyl, piperidonyl, pyrazolidinyl, pyrazolinyl, pyrrolidinyl, pyrrolinyl, 1-methyl-1,4-diazepane, tetrahydropyranyl or thiomorpholinyl. In the case where the heterocyclic ring contains a heteroatom selected from S or N, these atoms may optionally be in an oxidised form, such as S this includes optionally SO and SO₂.

The term “hydrochloride” includes monohydrochloride, dihydrochloride, trihydrochloride and tetrahydrochloride salts.

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have stereogenic centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

An object of the invention is to provide compounds of formula I for therapeutic use, especially compounds that are useful for the prevention and/or treatment of conditions associated with glycogen synthase kinase-3 (GSK3) in mammals including man. Particularly, compounds of formula I exhibiting a selective affinity for GSK-3.

### Methods of Preparation

Another aspect of the present invention provides a process for preparing a compound of formula I, or a pharmaceutically acceptable salt or an in vivo hydrolysable ester thereof, which process (wherein R¹, R², R³, R⁴, R⁵, R⁶, R⁷, R⁸ and R⁹ and are, unless otherwise specified, as defined in formula I) comprises of:

### Process a) Reaction of a Pyrimidine of Formula (II):

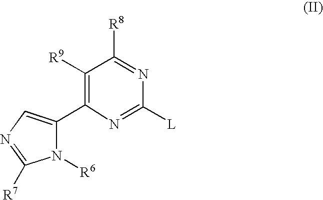

wherein L is a displaceable group; with an aniline of formula (III):

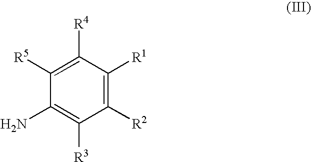

or

### Process B) Reacting a Pyrimidine of Formula (IV):

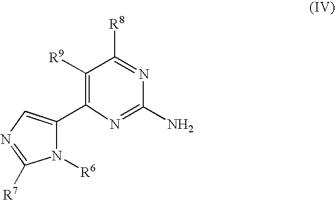

with a compound of formula (V):

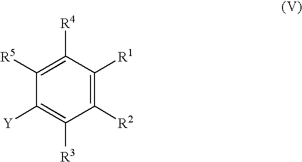

wherein Y is a displaceable group;
and thereafter if necessary:

i) converting a compound of the formula I into another compound of the formula I;

ii) removing any protecting groups; and

iii) forming a pharmaceutically acceptable salt or in vivo hydrolysable ester.

L is a displaceable group, suitable values for L are for example, a halogeno or sulphonyloxy group, for example a chloro, bromo, methanesulphonyloxy or toluene-4-sulphonyloxy group.

Y is a displaceable group, suitable values for Y are for example, a halogeno or sulphonyloxy group, for example a chloro, bromo, iodo or trifluoromethanesulphonyloxy group. Preferably Y is bromo or iodo.

Specific reaction conditions for the above reactions are as follows.

Process a). Pyrimidines of formula (II) and anilines of formula (III) may be reacted together under standard Buchwald-Hartwig conditions (for example see J. Am. Chem. Soc., 118, 7215; J. Am. Chem. Soc., 119, 8451; J. Am. Chem. Soc., 125, 6653; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl or 2-dicyclohexylphosphino-2′,4′,6′-triiso-propyl-1,1′-biphenyl and at a temperature in the range of +25 to +80° C.

Pyrimidines of the formula (II), in which R⁶ is CH₃ and L is chloro, may be prepared according to Scheme 1:

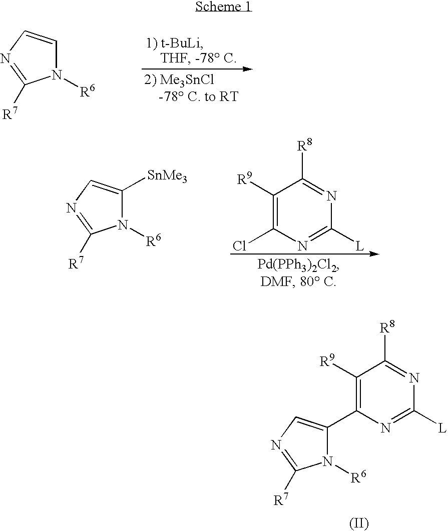

Anilines of formula (III) are commercially available compounds, or they are known in the literature, or they are prepared by standard processes known in the art.

Process b). Compounds of formula (IV) and amines of formula (V) may be reacted together under standard Buchwald conditions as described in Process a.

A synthesis of pyrimidines of formula (IV) is described in Scheme 2(R^{x} may be the same or different and is C₁₋₆alkyl):T should not be there

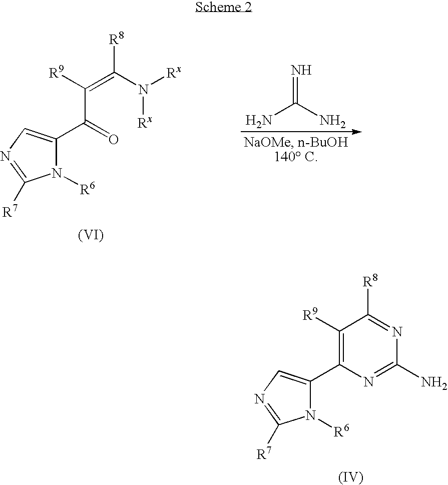

Compounds of formula (V) are commercially available compounds, or they are known in the literature, or they are prepared by standard processes known in the art.

Compounds of formula (VI) in which R⁶ has the general structure R^{a}—CH—R^{b} (wherein R^{a} and R^{b} are as defined in formula I and R^{x} may be the same or different and is C₁₋₆alkyl) and R⁹ is F may be prepared according to Scheme 3

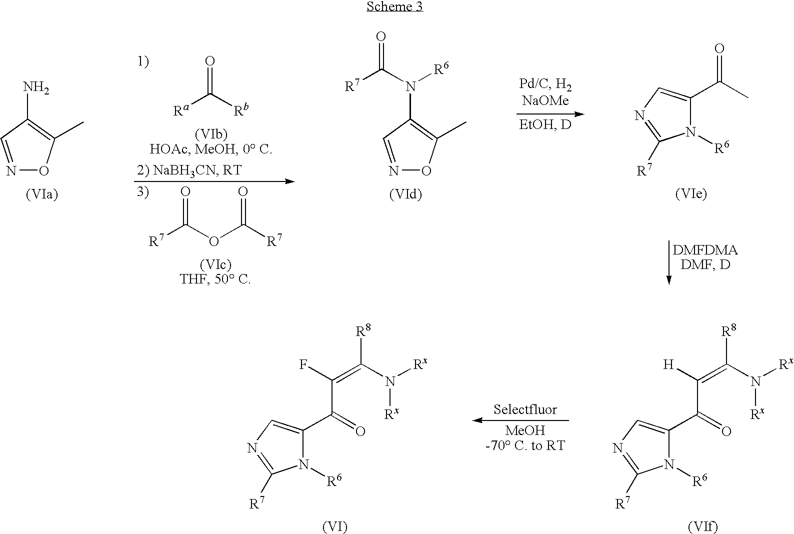

Compounds of formula (VIa), (VIb) and (VIc) are commercially available compounds, or they are known in the literature, or they are prepared by standard processes known in the art.

In one aspect of the invention, there is provided a process for preparing a compound of formula I which is a process selected from Process a) and Process b).

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Greene, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

The present invention also relates to intermediates for the end products of the present invention. These intermediates are useful in the preparation of a compound of formula I as defined above. These intermediates are represented by, but not limited to, the following
2-Chloro-4-(1,2-dimethyl-1H-imidazol-5-yl)-5-fluoropyrimidine;
2-Methyl-4-[(4-methylpiperazin-1-yl)carbonyl]aniline;
4-[(4-Methylpiperazin-1-yl)carbonyl]-3-nitroaniline;
4-[(4-Methylpiperazin-1-yl)carbonyl]-2-(trifluoromethoxy)aniline;
4-[N-Acetyl-N-(tetrahydro-2H-pyran-4-yl)]amino-5-methylisoxazole;
5-Acetyl-2-methyl-1-(tetrahydro-2H-pyran-4-yl)-1H-imidazole;
(2E)-3-Dimethylamino-1-[2-methyl-1-(tetrahydro-2H-pyran-4-yl)-1H-imidazol-5-yl]prop-2-en-1-one;
(2Z)-3-Dimethylamino-2-fluoro-1-[2-methyl-1-(tetrahydro-2H-pyran-4-yl)-1H-imidazol-5-yl]prop-2-en-1-one;
5-Fluoro-4-[2-methyl-1-(tetrahydro-2H-pyran-4-yl)-1H-imidazol-5-yl]pyrimidin-2-amine;
1-(4-Chloro-2-methoxybenzoyl)-4-methylpiperazine;
1-[4-Bromo-2-(methylsulfonyl)benzoyl]-4-methylpiperazine;
4-(N-Acetyl-N-cyclohexyl)amino-5-methylisoxazole;
5-Acetyl-1-cyclohexyl-2-methyl-1H-imidazole;
(2E)-3-Dimethylamino-1-(1-cyclohexyl-2-methyl-1H-imidazol-5-yl)prop-2-en-1-one;
(2Z)-3-Dimethylamino-2-fluoro-1-(1-cyclohexyl-2-methyl-1H-imidazol-5-yl)prop-2-en-1-one;
4-(1-Cyclohexyl-2-methyl-1H-imidazol-5-yl)-5-fluoropyrimidin-2-amine;
5-Acetyl-2-methyl-1-(1-methylpiperidin-4-yl)-1H-imidazole;
(2E)-3-Dimethylamino-1-[2-methyl-1-(1-methylpiperidin-4-yl)-1H-imidazol-5-yl]prop-2-en-1-one;
(2Z)-3-Dimethylamino-2-fluoro-1-[2-methyl-1-(1-methylpiperidin-4-yl)-1H-imidazol-5-yl]prop-2-en-1-one;
5-Fluoro-4-[2-methyl-1-(1-methylpiperidin-4-yl)-1H-imidazol-5-yl]pyrimidin-2-amine;
4-(1,2-Dimethyl-1H-imidazol-5-yl)-5-fluoropyrimidin-2-amine;
1-(tert-Butoxycarbonyl)-4-(4-bromo-benzenesulfonyl)-piperazine;
5-Acetyl-1-(tetrahydro-2H-pyran-4-yl)-2-trifluoromethyl-1H-imidazole;
(2E)-3-Dimethylamino-1-[1-(tetrahydro-2H-pyran-4-yl)-2-trifluoromethyl-1H-imidazol-5-yl]prop-2-en-1-one;
(2Z)-3-Dimethylamino-2-fluoro-1-[1-(tetrahydro-2H-pyran-4-yl)-2-trifluoromethyl-1H-imidazol-5-yl]prop-2-en-1-one;
5-Fluoro-4-[1-(tetrahydro-2H-pyran-4-yl)-2-(trifluoromethyl)-1H-imidazol-5-yl]pyrimidin-2-amine;
4-[2-Methyl-1-(tetrahydro-2H-pyran-4-yl)-1H-imidazol-5-yl]pyrimidin-2-amine;
1-[(4-Bromo-2-chlorophenyl)sulfonyl]-4-methylpiperazine;
(3R)-4-[(4-Bromophenyl)sulfonyl]-3-methylmorpholine;
(1S,4S)-2-[(4-Bromophenyl)sulfonyl]-5-methyl-2,5-diazabicyclo[2.2.1]heptane;
Methyl 4-bromo-2-(trifluoromethoxy)benzoate;
4-Bromo-2-(trifluoromethoxy)benzoic acid;
4-Bromo-2-(trifluoromethoxy)benzoic acid;
4-(4-Chloro-2-methylbenzyl)morpholine;
Lithium 4-({5-fluoro-4-[2-methyl-1-(tetrahydro-2H-pyran-4-yl)-1H-imidazol-5-yl]pyrimidin-2-yl}amino)benzoate;
1-(4-Bromo-2-methylbenzoyl)azetidine;
4-Bromo-2-(trifluoromethoxy)benzoic acid;
1-[4-Bromo-2-(trifluoromethoxy)benzoyl]azetidine;
2,2,2-Trifluoro-N-methyl-N-(5-methylisoxazol-4-yl)acetamide;
1-[1-Methyl-2-(trifluoromethyl)-1H-imidazol-5-yl]ethanone;
(2E)-3-(Dimethylamino)-1-[1-methyl-2-(trifluoromethyl)-1H-imidazol-5-yl]prop-2-en-1-one;
(2Z)-3-(Dimethylamino)-2-fluoro-1-[1-methyl-2-(trifluoromethyl)-1H-imidazol-5-yl]prop-2-en-1-one;
5-Fluoro-4-[1-methyl-2-(trifluoromethyl)-1H-imidazol-5-yl]pyrimidin-2-amine;
4-[4-Bromo-2-(methylsulfonyl)benzyl]morpholine;
2-[(4-Bromophenyl)sulfonyl]ethyl methyl ether;
2-[(4-Bromophenyl)sulfonyl]ethyl diethyl-amine;
N-(5-Methyl-isoxazol-4-yl)-N-(tetrahydro-pyran-4-yl)-formamide;
5-Acetyl-1-(tetrahydro-pyran-4-yl)-1H-imidazole;
(E)-3-Dimethylamino-1-[3-(tetrahydro-pyran-4-yl)-3H-imidazol-4-yl]-propenone;
(Z)-3-Dimethylamino-2-fluoro-1-[3-(tetrahydro-pyran-4-yl)-3H-imidazol-4-yl]-propenone;
5-Fluoro-4-[1-(tetrahydro-2H-pyran-4-yl)-1H-imidazol-5-yl]pyrimidin-2-amine; and
5-{5-Fluoro-2-[4-(4-methyl-piperazine-1-sulfonyl)-phenylamino]-pyrimid in-4-yl}-1-(tetrahydro-pyran-4-yl)-1H-imidazole-2-carbaldehyde.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded on a Varian Unity+400 NMR Spectrometer equipped with a 5 mm BBO probehead with Z-gradients, or a Varian Gemini 300 NMR spectrometer equipped with a 5 mm BBI probehead, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13.

The following reference signals were used: the middle line of DMSO-d₆ δ 2.50 (1H), δ 39.51 (13C); the middle line of CD₃OD δ 3.31 (1H) or δ 49.15 (13C); CDCl₃ δ 7.26 (1H) and the middle line of CDCl₃ δ 77.16 (13C) (unless otherwise indicated).

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to +40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Alternatively, compound identification was performed on a GC-MS system (GC 6890, 5973N MSD) supplied by Agilent Technologies. The column used was a VF-5 MS, ID 0.25 mm×15 m, 0.25 μm (Varian Inc.). A linear temperature gradient was applied starting at 40° C. (hold 1 min) and ending at +300° C. (hold 1 min), +25° C./minute. The mass spectrometer was equipped with a chemial ionisation (CI) ion source and the reactant gas was methane. The mass spectrometer was equipped with an electron impact (EI) ion source and the electron voltage was set to 70 eV. The mass spectrometer scanned between m/z 50-500 and the scan speed was set to 3.25 scan/s.

Microwave heating was performed in a single-mode microwave cavity producing continuous irradiation at 2450 MHz.

HPLC analyses were performed on an Agilent HPI000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to +40° C. and the flow rate to 1.0 ml/min. The Diode Array Detector was scanned from 210-300 nm, step and peak width were set to 2 nm and 0.05 min, respectively. A linear gradient was applied, starting at 100% A (A: 95:5 10 mM NH₄OAc:MeCN) and ending at 100% B (B: MeCN), in 4 min.

Alternatively, HPLC analyses were performed on a Gynkotek P580 HPG consisting of gradient pump with a Gynkotek UVD 170S UV-vis.-detector equipped with a Chromolith Performance RP column (C18, 100 mm×4.6 mm). The column temperature was set to +25° C. A linear gradient was applied using MeCN/0.1 trifluoroacetic acid in MilliQ water, run from 10% to 100% MeCN in 5 minutes. Flow rate: 3 ml/min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was performed on a Combi Flash® Companion™ using RediSep™ normal-phase flash columns or using Merck Silica gel 60 (0.040-0.063 mm). Typical solvents used for flash chromatography were mixtures of chloroform/methanol, dichloromethane/methanol, heptane/ethyl acetate, chloroform/methanol/ammonia (aq.) and dichlorormethane/methanol/NH₃ (aq.). SCX ion exchange columns were performed on Isolute® columns. Chromatography through ion exchange columns were typically performed in solvents such a methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 m/min.

The formation of hydrochloride salts of the final products were typically performed in solvents or solvents mixtures such as diethyl ether, tetrahydrofuran, dichloromethane/toluene, dichloromethane/methanol, followed by addition of 1M hydrogen chloride in diethyl ether.

The following abbreviations have been used:
aq. aqueous;
CHCl₃. chloroform;
CDCl₃ deuterated chloroform;
CD₃OH deuterated methanol;
CH₂Cl₂ dichloromethane;
Cs₂CO₃ caesium carbonate;
DCM dichloromethane;
DIPEA N,N-diisopropylethylaamine;
DMF N—N-dimethylformamide;
DMFDMA dimethylformamide dimethylacetal;
DMSO dimethyl sulphoxide;
DMSO-d₆ deuterated dimethyl sulphoxide;
dppp 1,3-bis(diphenylphosphino)propane;
EDC 1-ethyl-3-(3-dimethylaminopropyl)carbodiimide;
ether diethyl ether;
EtOAc ethyl acetate;
EtOH ethanol;
H₂ hydrogen gas;
HCOOH acetic acid;
HCl hydrochloride;
HOAc acetic acid;
HOBt 1-hydroxybenzotriazole;
(i-Pr)₂NEt N—N-diisopropylethylamine;
MeCN acetonitrile;
MeI methyl iodide;
CD₃OD deuterated methanol;
MeOH methanol;
Me₃SnCl trimethyltin chloride;
MgSO₄ magnesium sulphate;
NaBH₃CN sodium cyanoborohydride;
NaHCO₃ sodium bicarbonate;
NaOMe sodium methoxide;
Na₂SO₄ sodium sulphate;
n-BuOH n-butanol;
NH₃ ammonia;
NH₄OAc ammonium acetate;
NH₄OH ammonium hydroxide;
Pd/C palladium on carbon;
Pd(PPh₃)₂Cl₂ bis(triphenylphosphine)palladium dichloride;
Pd(t-Bu₃P)₂ bis(tri-tert-butylphosphine)palladium;
Pd₂(dba)₃ tris(dibenzylideneacetone)dipalladium;
Pd(OAc)₂ palladium diacetate;
r.t. or RT room temperature;
Selectfluor N-fluoro-N′-chloromethyl-triethylenediamine-bis(tetrafluoroborate);
t-BuLi tert-butyllithium;
THF tetrahydrofuran;
X-Phos 2-dicyclohexylphosphino-2′,4′,6′-triiso-propyl-1,1′-biphenyl.

Starting materials used were either available from commercial sources or prepared according to literature procedures and had experimental data in accordance with those reported. The following is an example of a starting material that was prepared: (4-Bromophenyl)(pyridin-2-yl)methanone: Bruce, R. B. et al., J. Med. Chem. 1968, 5, 1031-1034.

Compounds have been named either using ACD/Name, version 8.08, software from Advanced Chemistry Development, Inc. (ACD/Labs), Toronto ON, Canada, www.acdlabs.com, 2004 or using Openeye lexichem version 1.4 (Copyright© 1997-2006 OpenEye Scientific Software, Santa Fe, N. Mex.) to generate the IUPAC name.

In the following general methods A to I, the groups R¹, R², R³ and R⁴ are used independently to indicate the diversity of substitution within each structure. The identity of R¹, R², R³ and R⁴ will be clear to a person skilled in the art based on the starting materials and intermediates for each specific example. For instance in Example 39, which refers to General method E, E1 is 4-[2-methyl-1-(tetrahydro-2H-pyran-4-yl)-1H-imidazol-5-yl]pyrimidin-2-amine such that R¹ is tetrahydropyranyl, R³ is methyl and R⁴ is hydrogen and E2 is 1-bromo-4-(methylsulfonyl)benzene such that R² is sulphonylmethane para to the halogen.

### General Method A

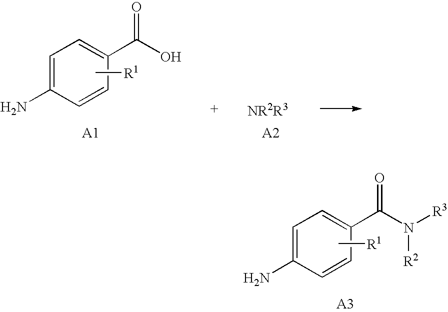

(i-Pr)₂NEt (2.1 equiv.), HOBt (1.05 equiv.), EDC hydrochloride (1.05 equiv.) and the amine A2 (1.05 equiv.) were added to a stirred solution of the benzoic acid A1 (1.0 equiv.) in anhydrous DMF at r.t. After 15 h, the reaction mixture was poured onto water and extracted with EtOAc. The organic layer was washed with water and brine, dried Na₂SO₄), filtered and evaporated in vacuo to afford the crude product, which was used in the next step without further purification.

### General Method B

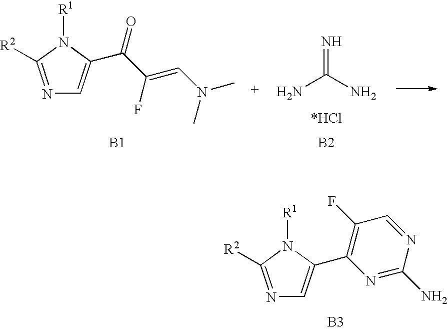

A reaction mixture of B1 (1.0 equiv.), guanidine hydrochloride B2 (4.0 equiv.) and sodium methoxide (4.0 equiv.) in 1-butanol was heated in a microwave reactor for 10 minutes at +140° C. under argon or nitrogen atmosphere. The mixture was filtered and the filter was rinsed with CH₂Cl₂. The solvent was evaporated in vacuo and the crude product was purified using flash column chromatography.

### General Method C

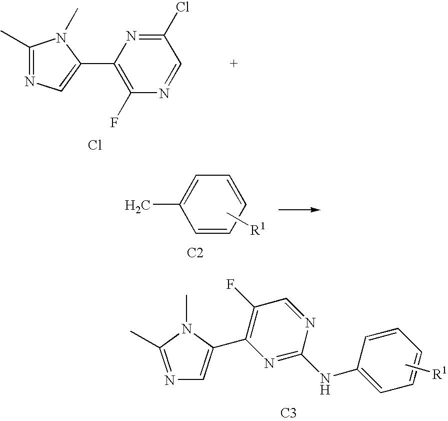

2-Chloro-4-(1,2-dimethyl-1H-imidazol-5-yl)-5-fluoropyrimidine C1 (1.0 equiv.), aniline C2 (1.1 equiv.) and sodium tert-butoxide (1.4 equiv.) were mixed in 1,4-dioxane and the mixture was flushed with argon for 5 minutes. Pd(OAc)₂ (0.05 equiv.) and Pd(t-Bu₃P)₂ (0.05 equiv.) were added and the reaction was stirred for 15 h at +110° C. The solvent was removed in vacuo and the residue was partitioned between EtOAc and water. After extraction the organic layer was dried (MgSO₄), filtered and evaporated in vacuo to afford a crude material which was purified by preparative HPLC.

### General Method D

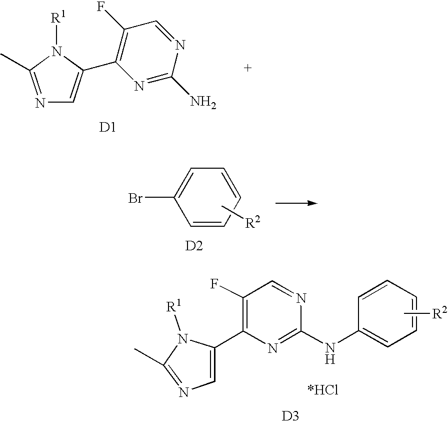

D1 (1.0 equiv.), D2 (0.85-1.24 equiv.) and sodium tert-butoxide (1.34-1.46 equiv.) were mixed in 1,4-dioxane and the mixture was flushed with argon for 5-10 minutes before Pd(OAc)₂ (0.04-0.082 equiv.) and Pd(t-Bu₃P)₂ (0.044-0.06 equiv.) were added. The mixture was flushed with argon then heated in a sealed tube at +110-+120° C. until the reaction was complete (as monitored by TLC or LC-MS). If the reaction was not complete after 24 h more Pd(OAc)₂, Pd(t-Bu₃P)₂ and sodium tert-butoxide were added. The solvent was removed in vacuo and the residue was partitioned between CH₂Cl₂ and water. After extraction the organic layer was dried (Na₂SO₄), filtered and evaporated. The crude of the free base was purified using preparative HPLC. MeCN was evaporated in vacuo and the aqueous phase was extracted with CH₂Cl₂. The organic phase was washed with water at pH 9 (diluted NaHCO₃ solution), dried (Na₂SO₄), filtered and evaporated. The residue was dissolved in CH₂Cl₂ and the HCl-adduct of the product was precipitated from the solution by addition of 0.1M HCl in ether (1-5 equiv. HCl). The solvent was evaporated and the residue was dissolved in water and freeze dried.

### General Method E

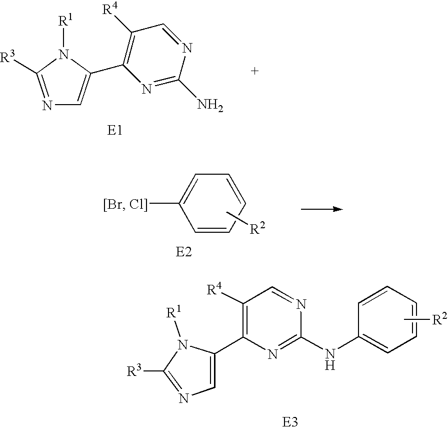

E1 (0.85-1.27 equiv.), E2 (1.0 equiv.) and Cs₂CO₃ (1.29-2.25 equiv.) were mixed in anhydrous 1,4-dioxane and the mixture was flushed with argon for 5-10 minutes before Pd₂(dba)₃ (0.02-0.08 equiv.) and X-Phos (0.04-0.16 equiv.) were added. The mixture was flushed with argon, then heated in a sealed tube at +90-+100° C. until the reaction was complete.

Workup and purification was performed according to either procedure A, B or C as follows. Procedure A) The solvent was removed in vacuo and the residue was taken up in CH₂Cl₂ and washed with diluted NaHCO₃ (aq.) or water. The organic layer was dried (Na₂SO₄), filtered and evaporated. The crude of the base product was purified using preparative HPLC. Procedure B) The reaction mixture was diluted with H₂O or a mixture of H₂O/CHCl₃, the product was extracted with CHCl₃, the combined organic phases was, if needed, dried (Na₂SO₄), filtered, concentrated and purified using flash column chromatography. Procedure C) The reaction mixture was diluted with CH₂Cl₂, filtered and evaporated. The residue was taken up in CH₂Cl₂ and the organic phase was washed with H₂O. Residual water was removed from the organic phase either by treatment with Na₂SO₄ or addition of absolute EtOH before evaporation. The crude of the base product was is purified using preparative HPLC.

Alternatively, the general example above was followed but with a slight modification in the order of addition of reagents. For example Cs₂CO₃ can be added together with E1 and E2 before the first argon flush.

### General Method F

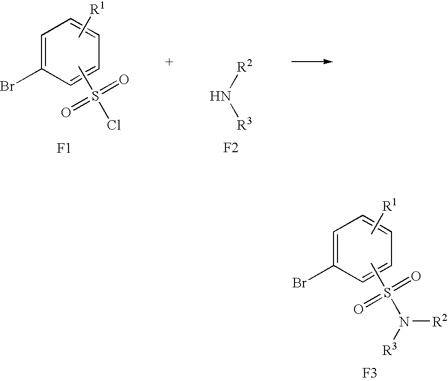

F1 (1 equiv) was dissolved in CH₂Cl₂ (2 mL) and F2 (1.0-1.1 equiv) was added. The reaction mixture was stirred at room temperature for 3 hours whereafter it was washed with saturated NaHCO₃ (2 mL). The organic phase was dried (Na₂SO₄), filtered and concentrated to afford F3.

### General Method G

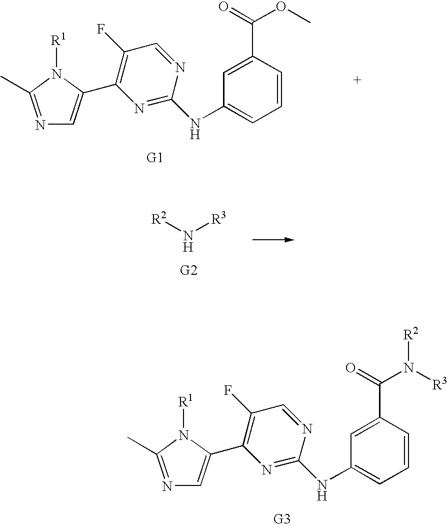

G1 (1.0 equiv.) and G2 (6.0 equiv.) was mixed in toluene (1-4 mL) in a thick walled vial of 10 mL volume and an inert atmosphere (Ar or N₂) was established. The sealed vial was cooled in an oil bath (r.t.) or in a dry-ice/ethanol bath (−70° C.) and Al(CH₃)₃, (2M in toluene) (10 equiv.) was added by a syringe. The reaction mixture was heated in an oil-bath at +90-100° C. for 1-4 h, cooled to r.t., and added dropwise into ice-cold sat. NaHCO₃ (aq) under vigorous stirring. The product was extracted with CH₂Cl₂ and the organic layer was dried, either by treatment with Na₂SO₄ or addition of absolute EtOH before evaporation. The crude base of the product was purified using flash column chromatography or preparative HPLC.

### General Method H

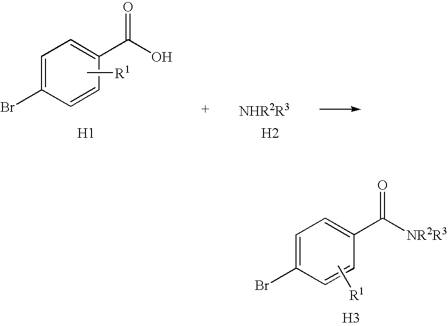

Thionyl chloride (5 mL) was added to H1 (1.0 equiv.). After addition of 2 drops of anhydrous DMF, the reaction mixture was refluxed for 15-30 minutes under an atmosphere of nitrogen. The solvent was evaporated in vacuo and the residue was dissolved in CH₂Cl₂ (until a clear solution was obtained). H2 (1.0 equiv.) was added dropwise followed by addition of triethylamine (1.0 equiv.). The reaction mixture was stirred at r.t. for 15-30 minutes before it was diluted with CH₂Cl₂, washed with saturated NaHCO₃ (aq.), dried (Na₂SO₄) and filtered. The solvent was evaporated in vacuo and the crude product was purified using flash column chromatography.

### General Method I

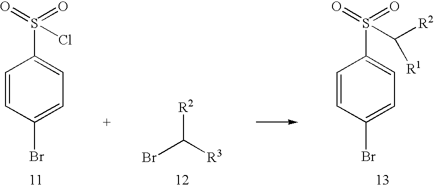

Sulfones of the type I3 were prepared following a modified procedure from Richard W. Brown (J. Org. Chem. 1991, 56, 4974-4976). 4-Bromobenzenesulfonyl chloride (I1, 1 equiv.), Na₂SO₃ (1 equiv.) and NaHCO₃ (3 equiv.) in water (0.2M) were stirred at +90° C. for 1 hour. 12 (1-3 equiv.) was then added and the resulting mixture stirred at +50-100° C. until the formation of the sulfone I3 was completed according to GC-MS analysis. Water was added to the reaction mixture and extracted with DCM. After extraction the organic layer was dried (MgSO₄), filtered and evaporated in vacuo to afford a crude material which was purified by flash chromatography.

### EXAMPLES

Below follows a number of non-limiting examples of compounds of the invention.

### Example 1

### 4-(1,2-Dimethyl-1H-imidazol-5-yl)-5-fluoro-N-[3-methoxy-5-(trifluoromethyl)phenyl]pyrimidin-2-amine

### Example 1(a) 1,2-Dimethyl-5-(trimethylstannyl)-1N-imidazole

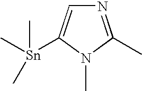

1,2-Dimethylimidazole (0.960 g, 10.0 mmol) was diluted in dry THF (50 mL) under an argon atmosphere and the solution was cooled to −78° C. tert-Butyllithium (1.7M in pentane, 6.47 mL, 11.0 mmol) was added dropwise over 5 minutes. The reaction mixture was stirred for 1 h at −78° C. and then treated with a solution of trimethyltin chloride (2.2 g, 11.0 mmol) in anhydrous THF (10 mL). The mixture was stirred for 60 h from −78° C. to r.t. The solvent was then evaporated in vacuo to give the title compound (1.29 g, 50%). The crude product was used in the next step without further purification.

¹H NMR (CDCl₃) δ ppm 6.87 (s, 1H), 3.56 (s, 3H), 2.41 (s, 3H), 0.45-0.18 (m, 9H); MS (CI) m/z 261 (¹²⁰Sn) (M+1).

### Example 1(b) 2-Chloro-4-(1,2-dimethyl-1H-imidazol-5-yl)-5-fluoropyrimidine

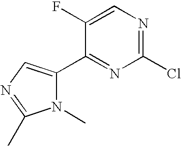

1,2-Dimethyl-5-(trimethylstannyl)-1H-imidazole (0.950 g, 3.68 mmol, obtained from Example 1(a)) and 2,4-dichloro-5-fluoropyrimidine (0.601 g, 3.60 mmol) were diluted in anhydrous DMF (20 mL) and the solution was degassed with argon. Pd(PPh₃)₂Cl₂ (0.126 g, 0.17 mmol) was added and the reaction mixture was stirred at +80° C. for 15 h. The reaction mixture was cooled down to r.t. and concentrated under reduced pressure. Saturated potassium fluoride (aq., 50 mL) was added and the mixture was stirred for 30 minutes before extraction with EtOAc. The organic layer was dried (MgSO₄), filtered and concentrated under reduced pressure. The crude product was purified by flash chromatography (heptane/EtOAc, 7:3) to give the title compound (0.41 g, 50%).

¹H NMR (CDCl₃, 600 MHz) δ ppm 8.40 (d, J=2.9 Hz, 1H), 7.86 (d, J=4.4 Hz, 1H), 3.97 (s, 3H), 2.53 (s, 3H); MS (ESI) m/z 227 (M+1).

### Example 1(c) 4-(1,2-Dimethyl-1H-imidazol-5-yl)-5-fluoro-N-[3-methoxy-5-(trifluoromethyl)phenyl]pyrimidin-2-amine

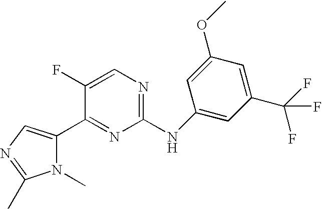

The title compound was prepared in accordance with the general method C using 2-chloro-4-(1,2-dimethyl-1H-imidazol-5-yl)-5-fluoropyrimidine (50 mg, 0.221 mmol, obtained from Example 1(b)) and 3-methoxy-5-(trifluoromethyl)aniline (46 mg, 0.243 mmol) to give the title compound (21 mg, 25%).

¹H NMR (CDCl₃) δ ppm 8.39-8.18 (m, 1H), 7.75 (d, J=4.3 Hz, 1H), 7.46 (s, 1H), 7.30 (s, 1H), 7.14 (s, 1H), 6.81 (s, 1H), 4.00-3.89 (m, 3H), 3.85 (s, 3H), 2.49 (s, 3H);

¹⁹F NMR (376 MHz, CDCl₃) δ ppm −63.16 (s, 3F), −144.69 (t, 1F); MS (ESI) m/z 382 (M+1).

### Example 2

### N-(3,5-Dichlorophenyl)-4-(1,2-dimethyl-1H-imidazol-5-yl)-5-fluoropyrimidin-2-amine

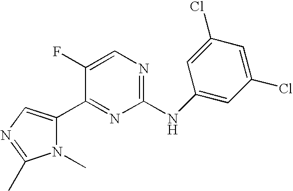

The title compound was prepared in accordance with the general method C using 2-chloro-4-(1,2-dimethyl-1H-imidazol-5-yl)-5-fluoropyrimidine (obtained from Example 1(b)) (50 mg, 0.221 mmol) and 3,5-dichloroaniline (39 mg, 0.243 mmol) to give the title compound (15 mg, 19%).

¹H NMR (DMSO-d₆) δ ppm 10.12 (s, 1H), 8.74 (d, J=2.8 Hz, 1H), 7.96 (d, J=2.8 Hz, 1H), 7.79 (d, J=2.0 Hz, 1H), 7.72-7.55 (m, 1H), 7.16 (t, J=1.8 Hz, 1H), 4.00 (s, 3H), 2.57 (s, 3H); MS (ESI) m/z 352 (M+1).

### Example 3

### (4-{[4-(1,2-Dimethyl-1H-imidazol-5-yl)-5-fluoropyrimidin-2-yl]amino}phenyl)(phenyl)methanone

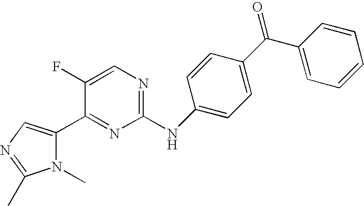

The title compound was prepared in accordance with the general method C using 2-chloro-4-(1,2-dimethyl-1H-imidazol-5-yl)-5-fluoropyrimidine (obtained from Example 1(b)) (80 mg, 0.354 mmol) and (4-aminophenyl)(phenyl)methanone (84 mg, 0.424 mmol), Pd(OAc)₂ (4.7 mg, 0.021 mmol) and Pd(t-Bu₃P)₂ (10.7 mg, 0.021 mmol) to give the title compound (56 mg, 41%).

¹H NMR (CDCl₃) δ ppm 8.32 (s, 1H), 8.00-7.63 (m, 7H), 7.60-7.33 (m, 4H), 3.96 (s, 3H), 2.51 (s, 3H); MS (ESI) m/z 388 (M+1).

### Example 4

### 4-(1,2-Dimethyl-1H-imidazol-5-yl)-5-fluoro-N-{2-methyl-4-[(4-methylpiperazin-1-yl)carbonyl]phenyl}pyrimidin-2-amine

### Example 4(a) 2-Methyl-4-[(4-methylpiperazin-1-yl)carbonyl]aniline

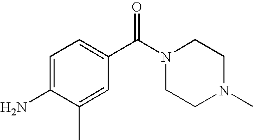

The title compound was prepared in accordance with the general method A using N-methylpiperazine (0.44 mL, 4.0 mmol) and 4-amino-3-methylbenzoic acid (0.692 g, 3.8 mmol) to give the title compound (0.421 g, 47%).

¹H NMR (CDCl₃) δ ppm 7.30 (s, 1H), 7.21 (s, 1H), 7.18-7.10 (m, 1H), 4.50-3.80 (br s, 4H), 3.91 (s, 3H), 3.20-2.50 (br s, 4H), 2.77 (m, 7H).

### Example 4(b) 4-(1,2-Dimethyl-1H-imidazol-5-yl)-5-fluoro-N-{2-methyl-4-[(4-methylpiperazin-1-yl)carbonyl]phenyl}pyrimidin-2-amine

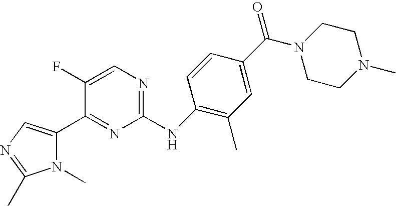

The title compound was prepared in accordance with the general method C using 2-chloro-4-(1,2-dimethyl-1H-imidazol-5-yl)-5-fluoropyrimidine (obtained from Example 11(b)) (50 mg, 0.221 mmol) and 2-methyl-4-[(4-methylpiperazin-1-yl)carbonyl]aniline (57 mg, 0.243 mmol) to give the title compound (15 mg, 16%).

¹H NMR (CD₃OD) δ ppm 8.31 (d, J=3.5 Hz, 1H), 7.70 (d, J=8.3 Hz, 1H), 7.63 (d, J=4.0 Hz, 1H), 7.35 (s, 1H), 7.29 (dd, J=8.2, 1.9 Hz, 1H), 3.83 (s, 3H), 3.79-3.42 (m, 4H), 2.63-2.47 (m, 4H), 2.45 (s, 3H), 2.38 (s, 3H), 2.35 (s, 3H); ¹⁹F NMR (CD₃OD) δ ppm −151.35 (s, 1F); MS (ESI) m/z 424 (M+1).

### Example 5

### 4-(1,2-Dimethyl-1H-imidazol-5-yl)-5-fluoro-N-{4-[(4-methylpiperazin-1-yl)carbonyl]-3-nitrophenyl}pyrimidin-2-amine

### Example 5(a) 4-[(4-Methylpiperazin-1-yl)carbonyl]-3-nitroaniline

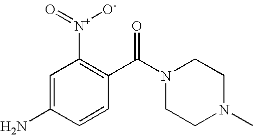

The title compound was prepared in accordance with the general method A using N-methylpiperazine (0.44 mL, 4.0 mmol) and 4-amino-2-nitrobenzoic acid (0.692 g, 3.8 mmol) to give the title compound (0.531 g, 53%).

¹H NMR (CD₃OD) δ ppm 7.39 (d, J=2.3 Hz, 1H), 7.13 (d, J=8.1 Hz, 1H), 6.91 (dd, J=8.1, 2.3 Hz, 1H), 4.16 (s, 2H), 3.96 (s, 2H), 3.41 (s, 2H), 2.69 (s, 2H), 2.59-2.48 (m, 2H), 2.47-2.36 (m, 3H).

### Example 5(b) 4-(1,2-Dimethyl-1H-imidazol-5-yl)-5-fluoro-N-{4-[(4-methylpiperazin-1-yl)carbonyl]-3-nitrophenyl}pyrimidin-2-amine

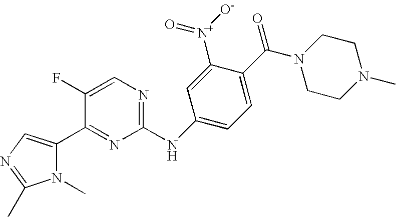

The title compound was prepared in accordance with the general method C using 2-chloro-4-(1,2-dimethyl-1H-imidazol-5-yl)-5-fluoropyrimidine (obtained from Example 1(b)) (50 mg, 0.221 mmol) and 4-[(4-methylpiperazin-1-yl)carbonyl]-3-nitroaniline (64 mg, 0.243 mmol, obtained from Example 5(a)) to give the title compound (21 mg, 21%).

¹H NMR (CD₃OD) δ ppm 8.80 (d, J=2.0 Hz, 1H), 8.45 (d, J=3.3 Hz, 1H), 7.97 (dd, J=8.3, 2.1 Hz, 1H), 7.66 (d, J=4.0 Hz, 1H), 7.40 (d, J=8.3 Hz, 1H), 4.06 (s, 3H), 3.83 (s, 2H), 3.43-3.36 (m, 2H), 2.61 (t, J=5.2 Hz, 2H), 2.55-2.48 (s, 3H), 2.48-2.41 (m, 2H), 2.37 (s, 3H); ¹⁹F NMR (CD₃OD) [symbol] ppm-147.52 (s, 1F); MS (ESI) m/z 455 (M+1).

### Example 6

### 4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride

### Example 6(a) 4-[(4-Methylpiperazin-1-yl)carbonyl]-2-(trifluoromethoxy)aniline

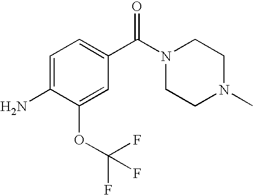

The title compound was prepared in accordance with the general method A using N-methylpiperazine (0.44 mL, 4.0 mmol) and 4-amino-3-(trifluoromethyoxy)benzoic acid (0.840 g, 3.8 mmol) to give the title compound (0.663 g, 57%).

¹H NMR (CD₃OD) δ ppm 7.33 (s, 1H), 7.25 (dd, J=8.2, 1.9 Hz, 1H), 6.83 (d, J=8.2 Hz, 1H), 4.21 (s, 2H), 3.96 (s, 4H), 3.16-2.33 (m, 7H).

### Example 6(b) 4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride

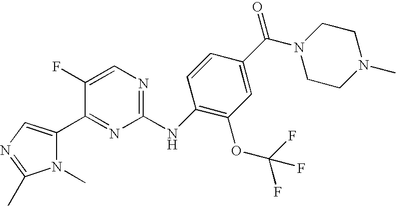

The base of the title compound was prepared in accordance with the general method C using 2-chloro-4-(1,2-dimethyl-1H-imidazol-5-yl)-5-fluoropyrimidine (obtained from Example 1(b)) (50 mg, 0.221 mmol) and 4-[(4-methylpiperazin-1-yl)carbonyl]-2-(trifluoromethoxy)aniline (73 mg, 0.243 mmol, obtained from Example 6(a)). The hydrochloride salt was obtained by dissolving the base product in anhydrous THF (5 mL) and a solution of 1M HCl in ether (1 mL) was added. The solvent was evaporated in vacuo and the residue was dried to give the title compound (21 mg, 19%).

1H NMR (CD₃OD) δ ppm 8.62 (s, 1H), 8.23 (s, 1H), 8.13 (s, 1H), 7.57 (br s, 2H), 4.39 (s, 2H), 4.09 (s, 3H), 3.57 (s, 4H), 3.24 (s, 2H), 2.96 (s, 3H), 2.76 (s, 3H); ¹⁹F NMR (CD₃OD) δ ppm-59.79 (s, 3F), −147.62 (s, 1F); MS (ESI) m/z 494 (M+1).

### Example 7

### 5-Fluoro-N-{4-[(4-methylpiperazin-1-yl)sulfonyl]phenyl}-4-[2-methyl-1-(tetrahydro-2H-pyran-4-yl)-1H-imidazol-5-yl]pyrimidin-2-amine hydrochloride

### Example 7(a) 4-[N-Acetyl-N-(tetrahydro-2H-pyran-4-yl)]amino-5-methylisoxazole

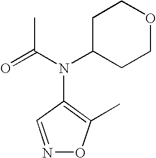

5-Methyl-4-amino-isoxazole (Reiter, L. A., J. Org. Chem. 1987, 52, 2714-2726) (0.68 g, 5.1 mmol) and acetic acid (0.61 g, 10.2 mmol) were dissolved in MeOH (20 mL). Tetrahydro-2H-pyran-4-one (0.76 g, 7.6 mmol) was added and the mixture was cooled to 0-(−5)° C. and stirred for 1 h. Sodium cyanoborohydride (0.32 g, 5.1 mmol) was added to is the reaction mixture at −5° C., causing weak exothermic and gas evolution. The cooling bath was removed and the mixture was stirred at r.t. for 1 h, followed by the addition of a second portion of sodium cyanoborohydride (0.1 g, 1.6 mmol). After stirring for 2 h at r.t., the mixture was filtered and the filtrate was concentrated in vacuo. The residue was dissolved in toluene and re-concentrated. The residue was dissolved in THF (10 mL) and acetic anhydride (1.56 g, 15.3 mmol) was added. The resulting mixture was stirred overnight at r.t. then for 1 h at +50° C. The volatiles were removed in vacuo and the residue was dissolved in toluene and concentrated in vacuo to give the title compound (1.36 g, 78%) as a solid.

¹H NMR (CDCl₃) ppm δ 8.04 (s, 1H), 4.86-4.73 (m, 1H), 4.00-3.89 (m, 2H), 3.52-3.42 (m, 2H), 2.35 (s, 3H), 1.81 (s, 3H), 1.70-1.57 (m, 2H), 1.49-1.23 (m, 2H); MS (ESI) m/z 225 (M+1).

### Example 7(b) 5-Acetyl-2-methyl-1-(tetrahydro-2H-pyran-4-yl)-1H-imidazole

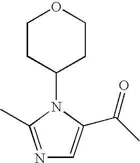

Sodium bicarbonate (0.8 g, 9.52 mmol) was added to a stirred solution of 4-[N-acetyl-N-(tetrahydro-2H-pyran-4-yl)]amino-5-methylisoxazole (4.8 g, 21.4 mmol, obtained from Example 7(a)) in EtOH (30 ml), and the mixture was hydrogenated over Pd/C (10%, wet paste, 0.10 g) at 3 bar. The reaction mixture was stirred at +50° C. for 3 h. An additional amount of Pd/C (10%, wet paste, 0.15 g) was added and the mixture was continued stirring at +50° C. for 3 h. Sodium methoxide (1.70 g, 31.46 mmol) was added and the resulting mixture was heated to reflux for 30 h. Ammonium chloride was added to quench the reaction. The mixture was filtrated through diatomaceous earth and the filtrate was evaporated in vacuo. The residue was diluted with saturated sodium bicarbonate (aq.) and extracted with EtOAc, then with CHCl₃. The combined organic layers were dried (Na₂SO₄) and concentrated in vacuo. The crude product was purified by flash chromatography (EtOAc) to give the title compound (3.7 g, 83%) as a yellow solid.

¹H NMR (CDCl₃) δ 7.70 (s, 1H), 5.40-5.30 (m, 1H), 4.13-4.01 (m, 2H), 3.57-3.44 (m, 2H), 2.57 (s, 3H), 2.44 (s, 3H), 2.43-2.30 (m, 2H), 1.80-1.72 (m, 2H).

### Example 7(c) (2E)-3-Dimethylamino-1-[2-methyl-1-(tetrahydro-2H-pyran-4-yl)-1H-imidazol-5-yl]prop-2-en-1-one

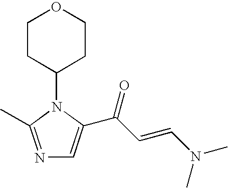

5-Acetyl-2-methyl-1-(tetrahydro-2H-pyran-4-yl)-1H-imidazole (3.7 g, 17.79 mmol, obtained 7(b)) was dissolved in DMFDMA/DMF (1:1, 100 mL) and the mixture was stirred under reflux overnight. After cooling to r.t. the mixture was extracted with CH₂Cl₂. The organic phase was dried (Na₂SO₄), filtered and concentrated in vacuo. The crude product was purified by flash chromatography (CH₂Cl₂/MeOH 15:1) to give the title compound (3.85 g, 82%) as an oil that crystallized in the refrigerator.

¹H NMR (CDCl₃) δ 7.65 (d, J=12.6 Hz, 1H), 7.46 (s, 1H), 5.55-5.42 (m, 2H), 4.08 (dd, J=11 Hz, 4.4 Hz, 2H), 3.52 (t, J=11 Hz, 2H), 2.99 (br s, 6H), 2.56 (s, 3H), 2.45-2.32 (m, 2H), 1.80-1.72 (m, 2H); MS (ESI) m/z 264 (M+1).

### Example 7(d) (2Z)-3-Dimethylamino-2-fluoro-1-[2-methyl-1-(tetrahydro-2H-pyran-4-yl)-1H-imidazol-5-yl]prop-2-en-1-one

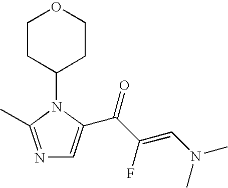

Selectfluor (7.75 g, 21.87 mmol) was added in portions to a stirred solution of (2E)-3-dimethylamino-1-[2-methyl-1-(tetrahydro-2H-pyran-4-yl)-1H-imidazol-5-yl]prop-2-en-1-one (3.85 g, 14.58 mmol, obtained from Example 7(c)) in MeOH (100 mL) at r.t. After stirring at r.t. for 3 h the reaction mixture was cooled in ice/acetone and filtered. The filtrate was evaporated under reduced pressure and the residue was taken into CH₂Cl₂. It was washed with aq. ammonia, brine, dried (Na₂SO₄) and concentrated in vacuo. The crude product was purified by flash chromatography (CH₂Cl₂/MeOH 15:1). The reaction was not run to completion, and the reaction was repeated again with Selectfluor (1.5 equiv.) followed by the same workup. The title compound (1.47 g, 36%) was obtained as a yellow oil.

¹H NMR (CDCl₃, 300 MHz) δ 7.34 (s, 1H), 6.84 (d, J=27.9 Hz, 1H), 5.00-4.88 (m, 1H), 4.04 (dd, J=11.2 Hz, 4.2 Hz, 2H), 3.46 (t, J=11 Hz, 2H), 3.08 (s, 6H), 2.53 (s, 3H), 2.42-2.28 (m, 2H), 1.84-1.75 (m, 2H); MS (ESI) m/z 282 (M⁺+1).

### Example 7(e) 5-Fluoro-4-[2-methyl-1-(tetrahydro-2H-pyran-4-yl)-1H-imidazol-5-yl]pyrimidin-2-amine

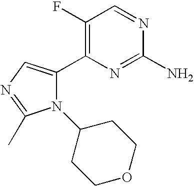

The title compound was prepared in accordance with the general method B with the exception that guanidine carbonate was used. Using (2Z)-3-dimethylamino-2-fluoro-1-[2-methyl-1-(tetrahydro-2H-pyran-4-yl)-1H-imidazol-5-yl]prop-2-en-one (1.47 g, 5.22 mmol, obtained from Example 7(d)) and guanidine carbonate (2.35 g, 13.06 mmol) the title compound (1.21 g, 84%) was obtained as a solid after purification by flash chromatography (CH₂Cl₂/MeOH 20:1).

¹H NMR (CDCl₃, 300 MHz) δ 8.17 (d, J=3.3 Hz, 1H), 7.59 (d, J=3.9 Hz, 1H), 5.27-5.13 (m, 1H), 4.93 (br s, 2H), 4.13 (dd, J=11.5 Hz, 4.3 Hz, 2H), 3.48 (t, J=11 Hz, 2H), 2.62 (s, 3H), 2.58-2.40 (m, 2H), 1.95-1.84 (m, 2H); MS (ESI) m/z 278 (M+1).

### Example 7(f) 5-Fluoro-N-{4-[(4-ethylpiperazin-1-yl)sulfonyl]phenyl}-4-[2-methyl-1-(tetrahydro-2H-pyran-4-yl)-1H-imidazol-5-yl]pyrimidin-2-amine hydrochloride

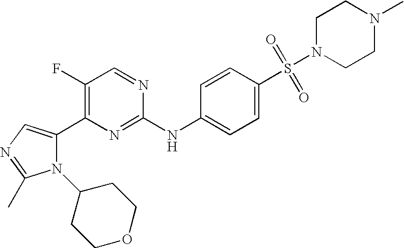

The title compound was prepared in accordance with the general method D, with the exception that the base of the product was purified by flash chromatography (CHCl₃/MeOH/NH₃ aq. 200:10:1) before purification by preparative HPLC. Using 5-fluoro-4-[2-methyl-1-(tetrahydro-2H-pyran-4-yl)-1H-imidazol-5-yl]pyrimidin-2-amine (0.075 g, 0.27 mmol, obtained from Example 7(e)), 1-(4-bromo-benzenesulfonyl)-4-methylpiperazine (described in WO 2003004472) (0.108 g, 0.338 mmol), sodium tert-butoxide (0.036 g, 0.38 mmol), Pd(OAc)₂ (0.012 g, 0.054 mmol) and Pd(t-Bu₃P)₂ (0.14 g, 0.027 mmol), the title compound (0.018 g, 11%) was obtained as a yellow solid.

¹H NMR (DMSO-d₆, 300 MHz) δ 10.64 (br s, 1H), 10.43 (s, 1H), 8.90 (s, 1H), 8.07 (s, 1H), 7.96 (d, J=8.4 Hz, 2H), 7.70 (d, J=8.4 Hz, 2H), 5.03-4.93 (m, 1H), 3.90-3.70 (m, 4H), 3.36-3.15 (m, 4H), 2.81 (s, 3H), 2.74 (s, 3H), 2.25-2.15 (m, 2H), 2.00-1.92 (m, 2H); MS (ESI) m/z 516 (M+1).

### Example 8

### 5-Fluoro-N-{4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride

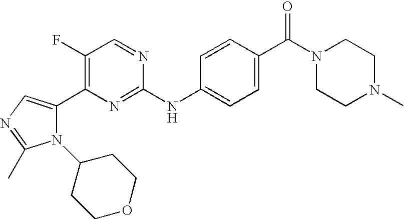

The title compound was prepared in accordance with the general method D, with the exception that the base of the product was purified by flash chromatography (CHCl₃/MeOH/NH₃ aq. 200:10:1) before purification by preparative HPLC. Using 5-fluoro-4-[2-methyl-1-(tetrahydro-2H-pyran-4-yl)-1H-imidazol-5-yl]pyrimidin-2-amine (obtained from Example 7(e)) (0.075 g, 0.27 mmol), 1-(4-bromobenzoyl)-4-methylpiperazine (0.115 g, 0.405 mmol), sodium tert-butoxide (0.036 g, 0.38 mmol), Pd(OAc)₂ (0.012 g, 0.054 mmol) and Pd(t-Bu₃P)₂ (0.14 g, 0.027 mmol), the base of title compound (0.023 g, 18%) was obtained. The hydrochloride of the title compound was prepared in accordance with the general method D.

¹H NMR (DMSO-d₆, 300 MHz) δ 11.02 (br s, 1H), 10.14 (s, 1H), 8.86 (s, 1H), 8.12 (s, 1H), 7.72 (d, J=8.1 Hz, 2H), 7.41 (d, J=8.1 Hz, 2H), 5.03-4.93 (m, 1H), 4.25-4.05 (m, 4H), 3.45-3.35 (m, 4H), 3.23-3.00 (m, 4H), 2.83 (s, 3H), 2.78 (s, 3H), 2.20-2.10 (m, 2H), 1.98-1.90 (m, 2H); MS (ESI) m/z 480 (M+1).

### Example 9

### 5-Fluoro-N-{3-methoxy-4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride

### Example 9(a) 1-(4-Chloro-2-methoxybenzoyl)-4-methylpiperazine

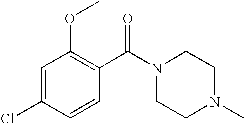

Thionyl chloride (5 mL) was added to 4-chloro-2-methoxybenzoic acid (0.501 g, 2.68 mmol). After addition of 1 drop of anhydrous DMF, the reaction mixture was refluxed for 30 minutes under an atmosphere of nitrogen. The solvent was evaporated in vacuo and the residue was dissolved in CH₂Cl₂ (until a clear solution was obtained). N-Methylpiperazine (0.31 nm, 2.81 mmol)) was added dropwise followed by addition of triethylamine (0.39 mL, 2.81 mmol). The reaction mixture was stirred at r.t. for 15 minutes before it was diluted with CH₂Cl₂, washed with saturated NaHCO₃ (aq.), water, dried (Na₂SO₄), filtered and concentrated in vacuo to give the title compound in quantitative yield. The isolated material was used in the next step without further purification.

¹H NMR (DMSO-d₆) δ ppm 7.18 (d, J=8.0 Hz, 1H), 7.17 (d, J=2.0 Hz, 1H), 7.05 (dd, J=8.0, 1.8 Hz, 1H), 3.81 (s, 3H), 3.67-3.51 (m, 2H), 3.14-3.04 (m, 2H), 2.38-2.27 (m, 2 H), 2.27-2.19 (m, 2H), 2.18 (s, 3H).

### Example 9(b) 5-Fluoro-N-(3-methoxy-4-[(4-methylpiperazin-1-yl)carbonyl]phenyl)-4-[2-methyl-1-(tetrahydro-2H-pyran-4-yl)-1H-imidazol-5-yl]pyrimidin-2-amine hydrochloride

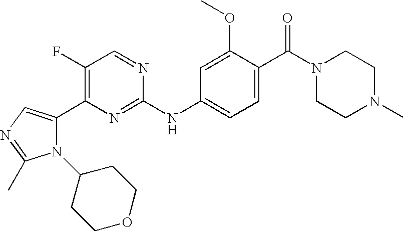

The title compound was prepared in accordance with the general method E, with the exception that the base of the product was purified by flash chromatography (CH₂Cl₂/MeOH/NH₃ aq. 200:10:1). Using 5-fluoro-4-[2-methyl-1-(tetrahydro-2H-pyran-4-yl)-1H-imidazol-5-yl]pyrimidin-2-amine (obtained from Example 7(e)) (0.075 g, 0.27 mmol), 1-(4-chloro-2-methoxybenzoyl)-4-methylpiperazine (0.065 g, 0.24 mmol, obtained from Example 9(a)), Cs₂CO₃ (176 mg, 0.54 mmol), Pd₂(dba)₃ (12 mg, 0.013 mmol) and X-Phos (13 mg, 0.027 mmol), the base of the title compound (105 mg, 67%) was obtained as a white solid. The hydrochloride of the title compound was prepared in accordance with the general method D.

¹H NMR (DMSO-d₆, 300 MHz) δ 11.45 (br s, 1H), 10.08 (s, 1H), 8.85 (s, 1H), 8.13 (s, 1 H), 7.43 (d, J=7.8 Hz, 1H), 7.35 (s, 1H), 7.18 (d, J=7.8 Hz, 1H), 5.03-4.93 (m, 1H), 4.75-4.50 (m, 3H), 3.90-3.78 (m, 4H), 3.52-3.39 (m, 4H), 3.27-3.12 (m, 4H), 2.85 (s, 3H), 2.77 (s, 3H), 2.20-2.10 (m, 2H), 1.98-1.90 (m, 2H); MS (ESI) m/z 510 (M+1).

### Example 10

### 5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride

### Example 10(a) 1-[4-Bromo-2-(methylsulfonyl)benzoyl]-4-methylpiperazine

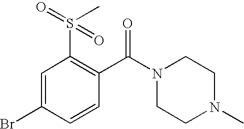

Thionyl chloride (5 mL) was added to 4-bromo-2-(methylsulfonyl)benzoic acid (0.50 g, 1.67 mmol). After addition of 1 drop of anhydrous DMF, the reaction mixture was refluxed for 30 minutes under an atmosphere of nitrogen. The solvent was evaporated in vacuo and the residue was dissolved in CH₂Cl₂ (until a clear solution was obtained). N-Methylpiperazine (0.195 mL, 1.75 mmol) was added dropwise followed by addition of triethylamine (0.243 mL, 1.75 mmol). The reaction mixture was stirred at r.t. for 15 minutes before it was diluted with CH₂Cl₂, washed with saturated NaHCO₃ (aq.), water, dried (Na₂SO₄), filtered and concentrated in vacuo to give the title compound in quantitative yield. The isolated material was used in the next step without further purification.

¹H NMR (DMSO-d₆, Signals corresponding to 3 protons were overlapping with the solvents) 5 ppm 8.06 (d, J=2.0 Hz, 1H), 8.01 (dd, J=8.3, 2.0 Hz, 1H), 7.46 (d, J=8.0 Hz, 1H), 3.65-3.53 (m, 2H), 3.19-3.00 (m, 2H), 2.43-2.33 (m, 2H), 2.33-2.21 (m, 2H), 2.19 (s, 3H); MS (ESI) m/z 361, 363 (M+1).

### Example 10(b) 5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride

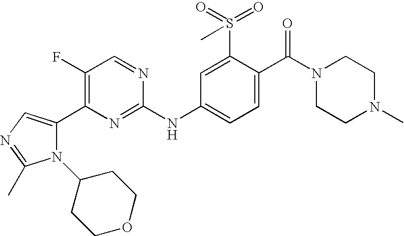

The title compound was prepared in accordance with the general method E, with the exception that the base of the product was purified by flash chromatography (CHCl₃/MeOH/NH₃ aq. 200:10:1). Using 5-fluoro-4-[2-methyl-1-(tetrahydro-2H-pyran-4-yl)-1H-imidazol-5-yl]pyrimidin-2-amine (obtained from Example 7(e)) (67 mg, 0.242 mmol), 1-[4-bromo-2-(methylsulfonyl)benzoyl]-4-methylpiperazine (70 mg, 0.194 mmol, obtained from 10(a)), Cs₂CO₃ (71 mg, 0.22 mmol), Pd₂(dba)₃ (11 mg, 0.012 mmol) and X-Phos (10 mg, 0.021 mmol), the base of the title compound (0.100 g, 92%) was obtained as a solid. The hydrochloride of the title compound was prepared in accordance with the general method D.

¹H NMR (DMSO-d₆, 300 MHz) δ 11.15 (br s, 1H), 10.41 (s, 1H), 8.91 (s, 1H), 8.30-8.25 (m, 1H), 8.20-8.11 (m, 2H), 7.55-7.49 (m, 1H), 5.01-4.88 (m, 1H), 4.70-4.50 (m, 1H), 3.95-3.62 (m, 6H), 3.59-3.44 (m, 2H), 3.40-3.08 (m, 7H), 2.84 (s, 3H), 2.81-2.75 (m, 3H), 2.23-2.11 (m, 2H), 2.01-1.94 (m, 2H); MS (ESI) m/z 558 (M+1).

### Example 11

### 5-Fluoro-N-[4-[(4-methylpiperazin-1-yl)sulfonyl]-3-(trifluoromethoxy)phenyl]-4-[2-methyl-1-(tetrahydro-2H-pyran-4-yl)-1H-imidazol-5-yl]pyrimidin-2-amine hydrochloride

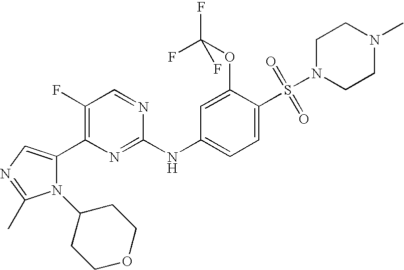

The title compound was prepared in accordance with the general method E, with the exception that the base of the title product was purified by flash chromatography (CH₂Cl₂/MeOH 20:1). Using 5-fluoro-4-[2-methyl-1-(tetrahydro-2H-pyran-4-yl)-1H-imidazol-5-yl]pyrimidin-2-amine (obtained from Example 7(e)) (32 mg, 0.116 mmol), 1-(4-bromo-2-trifluoromethoxy-benzenesulfonyl)-4-methyl-piperazine (described in WO 2003004472) (0.042 g, 0.104 mmol), Cs₂CO₃ (38 mg, 0.12 mmol), Pd₂(dba)₃ (6 mg, 0.006 mmol) and X-Phos (5 mg, 0.011 mmol), the base of the title compound (38 mg, 61%) was obtained as a solid. The hydrochloride of the title compound was prepared in accordance with the general method D.

¹H NMR (DMSO-d₆, 300 MHz) δ 11.31 (br s, 1H), 10.70 (s, 1H), 8.96 (s, 1H), 8.10 (s, 2H), 7.96 (d, J=9.0 Hz, 1H), 7.84 (d, J=9.0 Hz, 1H), 5.00-4.85 (m, 1H), 3.93-3.83 (m, 2H), 3.79-3.69 (m, 2H), 3.48-3.39 (m, 2H), 3.36-3.25 (m, 2H), 3.18-2.98 (m, 4H), 2.84 (s, 3H), 2.74 (s, 3H), 2.20-2.10 (m, 2H), 2.00-1.92 (m, 2H); MS (ESI) m/z 600 (M+1).

### Example 12

### 5-Fluoro-4-[2-methyl-1-(tetrahydro-2H-pyran-4-yl)-1H-imidazol-5-yl]-N-[4-(pyrrolidin-1-ylsulfonyl)phenyl]pyrimidin-2-amine hydrochloride

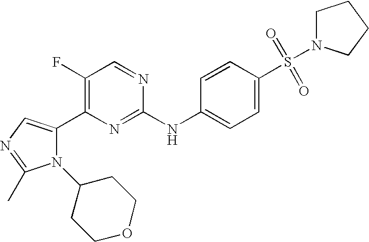

The title compound was prepared in accordance with the general method E, with the exception that the base of the title product was purified by flash chromatography (CH₂Cl₂/MeOH 30:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 1-(4-bromo-benzenesulfonyl)-pyrrolidine (33 mg, 0.113 mmol), Cs₂CO₃ (41 mg, 0.13 mmol), Pd₂(dba)₃ (6 mg, 0.007 mmol) and X-Phos (6 mg, 0.012 mmol), the base of the title compound (54 mg, 98%) was obtained as a solid. The hydrochloride of the title compound was prepared in accordance with the general method D.

¹H NMR (DMSO-d₆, 300 MHz) δ 10.37 (s, 1H), 8.89 (s, 1H), 8.32 (s, 1H), 8.10 (s, 1H), 7.89 (d, J=8.4 Hz, 2H), 7.72 (d, J=8.4 Hz, 2H), 5.03-4.93 (m, 1H), 3.87-3.78 (m, 2H), 3.29-3.07 (m, 6H), 2.83 (s, 3H), 2.22-2.12 (m, 2H), 2.00-1.92 (m, 2H), 2.64 (s, 4H); MS (ESI) m/z 487 (M+1).

### Example 13

### 5-Fluoro-4-[2-methyl-1-(tetrahydro-2H-pyran-4-yl)-1H-imidazol-5-yl]-N-[4-(morpholin-4-ylsulfonyl)phenyl]pyrimidin-2-amine hydrochloride

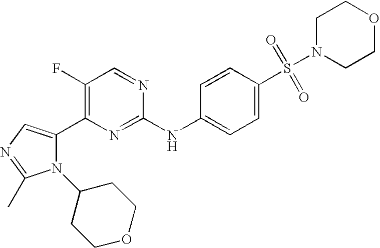

The title compound was prepared in accordance with the general method E, with the exception that the base of the title product was purified by flash chromatography (CH₂Cl₂/MeOH 30:1). Using 5-fluoro-4-[2-methyl-1-(tetrahydro-2H-pyran-4-yl)-1H-imidazol-5-yl]pyrimidin-2-amine (obtained from Example 7(e)) (30 mg, 0.108 mmol), 4-(4-bromo-benzenesulfonyl)-morpholine (0.032 g, 0.103 mmol), Cs₂CO₃ (38 mg, 0.116 mmol), Pd₂(dba)₃ (6 mg, 0.006 mmol) and X-Phos (5 mg, 0.011 mmol), the base of the title compound (52 mg, quantitative yield) was obtained as a solid. The hydrochloride of the title compound was prepared in accordance with the general method D.

¹H NMR (DMSO-d₆, 300 MHz) δ 10.41 (s, 1H), 8.90 (s, 1H), 8.10 (s, 1H), 7.92 (d, J=8.4 Hz, 2H), 7.65 (d, J=8.4 Hz, 2H), 5.03-4.91 (m, 1H), 3.88-3.78 (m, 2H), 3.62 (s, 4H), 3.29-3.14 (m, 2H), 2.85-2.81 (m, 7H), 2.23-2.12 (m, 2H), 2.00-1.92 (m, 2H); MS (ESI) m/z 503 (M+1).

### Example 14

### [4-({5-Fluoro-4-[2-methyl-1-(tetrahydro-2H-pyran-4-yl)-1H-imidazol-5-yl]pyrimidin-2-yl}amino)phenyl](pyridin-2-yl)methanone hydrochloride

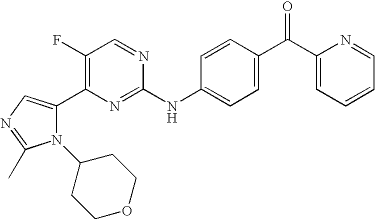

The title compound was prepared in accordance with the general method E, with the exception that the base of the title product was purified by flash chromatography (CH₂Cl₂/MeOH 30:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), (4-bromophenyl)(pyridin-2-yl)methanone (Bruce R. B. et al., J. Med. Chem. 1968, 11, 1031-1034) (32 mg, 0.103 mmol), Cs₂CO₃ (44 mg, 0.13 mmol), Pd₂(dba)₃ (6 mg, 0.007 mmol) and X-Phos (6 mg, 0.013 mmol), the base of the title compound (53 mg, 96%) was given as a solid. The hydrochloride of the title compound was prepared in accordance with the general method D.

¹H NMR (DMSO-d₆, 300 MHz) δ 10.40 (s, 1H), 8.90 (s, 1H), 8.73-8.68 (m, 1H), 8.14 (s, 1H), 8.10-7.89 (m, 4H), 7.82 (d, J=8.4 Hz, 2H), 7.69-7.63 (m, 1H), 5.07-4.94 (m, 1H), 3.88-3.80 (m, 2H), 3.26-3.14 (m, 2H), 2.85 (s, 3H), 2.22-2.11 (m, 2H), 2.01-1.93 (m, 2H); MS (ESI) m/z 459 (M+1).

### Example 15

### 5-Fluoro-4-[2-methyl-1-(tetrahydro-2H-pyran-4-yl)-1H-imidazol-5-yl]-N-[4-(morpholin-4-ylmethyl)phenyl]pyrimidin-2-amine hydrochloride

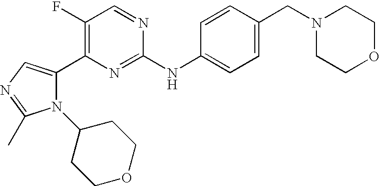

The title compound was prepared in accordance with the general method E, with the exception that the reaction was heated to +100° C. for an additional 20 h, and the base of the product was purified by flash chromatography (CH₂Cl₂/MeOH 15:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 4-(4-bromobenzyl)-morpholine (0.031 g, 0.120 mmol), Cs₂CO₃ (44 mg, 0.13 mmol), Pd₂(dba)₃ (6 mg, 0.007 mmol) and X-Phos (6 mg, 0.013 mmol), the base of the title compound (28 mg, 49%) was obtained as a solid. The hydrochloride of the title compound was prepared in accordance with the general method D.

¹H NMR (DMSO-d₆, 300 MHz) δ 11.48 (br s, 1H), 10.07 (s, 1H), 8.83 (s, 1H), 8.09 (s, 1H), 7.69 (d, J=8.3 Hz, 2H), 7.54 (d, J=8.3 Hz, 2H), 5.03-4.91 (m, 1H), 4.25 (s, 2H), 3.96-3.76 (m, 6H), 3.25-3.12 (m, 4H), 3.10-2.97 (m, 2H), 2.83 (s, 3H), 2.22-2.11 (m, 2H), 2.00-1.90 (m, 2H); MS (ESI) m/z 453 (M+1).

### Example 16

### 5-Fluoro-4-[2-methyl-1-(tetrahydro-2H-pyran-4-yl)-1H-imidazol-5-yl]-N-[4-(piperidin-1-ylcarbonyl)phenyl]pyrimidin-2-amine hydrochloride

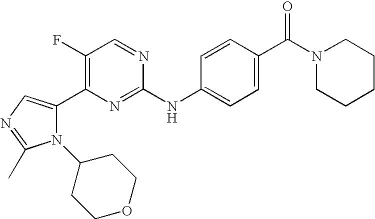

The title compound was prepared in accordance with the general method E, with the exception that the base of the title compound was purified by flash chromatography (CH₂Cl₂/MeOH 25:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 1-(4-bromobenzoyl)piperidine (0.042 g, 0.157 mmol), Cs₂CO₃ (46 mg, 0.14 mmol), Pd₂(dba)₃ (7 mg, 0.008 mmol) and X-Phos (7 mg, 0.014 mmol), the base of the title compound (52 mg, 89%) was obtained as a solid. The hydrochloride of the title compound was prepared in accordance with the general method D.

¹H NMR (DMSO-d₆, 300 MHz) δ 10.05 (s, 1H), 8.83 (s, 1H), 8.08 (s, 1H), 7.66 (d, J=8.4 Hz, 2H), 7.31 (d, J=8.4 Hz, 2H), 5.05-4.94 (m, 1H), 3.85-3.77 (m, 2H), 3.50-3.32 (m, 4H), 3.21-3.09 (m, 2H), 2.82 (s, 3H), 2.19-2.10 (m, 2H), 1.96-1.88 (m, 2H), 1.63-1.58 (m, 2H), 1.54-1.42 (m, 4H); MS (ESI) m/z 465 (M+1).

### Example 17

### 4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride

### Example 17(a) 4-(N-Acetyl-N-cyclohexyl)amino-5-methylisoxazole

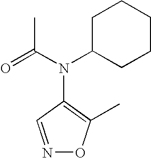

The title compound was prepared in accordance with the general method of Example 7 (a), with the exception that the product was purified by flash chromatography (heptane/EtOAc 1:1). Using 5-methyl-4-amino-isoxazole (Reiter, L. A, J. Org. Chem. 1987, 52, 2714-2726) (2.5 g, 25.48 mmol) and cyclohexanone (2.74 g, 28 mmol), the title compound was obtained (4.35 g, 77%) as a solid.

MS (ESI) m/z 223 (M+1).

### Example 17(b) 5-Acetyl-1-cyclohexyl-2-methyl-1H-imidazole

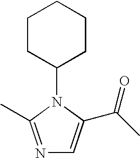

The title compound was prepared in accordance with the general method of Example 7(b), with the exception that the product was purified by flash chromatography (CH₂Cl₂/MeOH, 20:1). Using 4-(N-acetyl-N-cyclohexyl)amino-5-methylisoxazole (4.35 g, 19.6 mmol, is obtained from Example 17(a)) the title compound was obtained (1.2 g, 30%) as a yellow oil.

MS (ESI) m/z 207 (M+1).

### Example 17(c) (2E)-3-Dimethylamino-1-(1-cyclohexyl-2-methyl-1H-imidazol-5-yl)prop-2-en-1-one

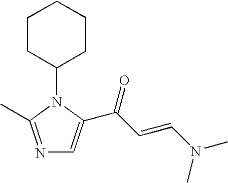

The title compound was prepared in accordance with the general method of Example 7 (c), with the exception that the product was purified by flash chromatography (CH₂Cl₂/MeOH, 25:1). Using 5-acetyl-1-cyclohexyl-2-methyl-1H-imidazole (1.2 g, 5.80 mmol, obtained from 17(b)) the title compound was obtained (1.4 g, 93%) as an oil that solidified upon standing.

¹H NMR (CDCl₃, 300 MHz) δ 7.62 (d, J=12.6 Hz, 1H), 7.43 (s, 1H), 5.50 (d, J=12.6 Hz, 1H), 5.04-4.90 (m, 1H), 2.97 (br. s, 6H), 2.50 (s, 3H), 2.17-2.02 (m, 2H), 1.89-1.81 (m, 4H), 1.72-1.64 (m, 1H), 1.48-1.19 (m, 3H); MS (ESI) m/z 262 (M+1).

### Example 17(d) (2Z)-3-Dimethylamino-2-fluoro-1-(1-cyclohexyl-2-methyl-1H-imidazol-5-yl)prop-2-en-1-one

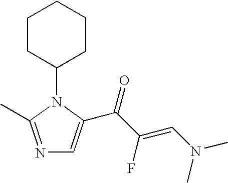

The title compound was prepared according to the general method of Example 7 (d) with the following modification. The reaction was repeated two times (first with 1.5 equiv. of Selectfluor and then with 0.7 equiv.) in order to get full conversion of the starting material. The product was purified by flash chromatography (CH₂Cl₂/MeOH, 30:1 then 20:1) after every treatment with Selectfluor. Starting from (2E)-3-dimethylamino-1-(1-cyclohexyl-2-methyl-1H-imidazol-5-yl)prop-2-en-1-one (1.39 g, 5.32 mmol, obtained from Example 17(c)) the title compound was obtained (0.42 g, 28%).

MS (ESI) m/z 280 (M+1).

### Example 17(e) 4-(1-Cyclohexyl-2-methyl-1H-imidazol-5-yl)-5-fluoropyrimidin-2-amine

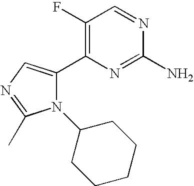

The title compound was prepared in accordance with the general method B with the exception that guanidine carbonate was used. Using (2Z)-3-dimethylamino-2-fluoro-1-(1-cyclohexyl-2-methyl-1H-imidazol-5-yl)prop-2-en-one (0.42 g, 1.50 mmol, obtained from Example 17(d)) and guanidine carbonate (0.68 g, 3.76 mmol) the title compound (0.35 g, 85%) was obtained as a white solid.

¹H NMR (CDCl₃, 300 MHz) δ 8.15 (d, J=3.3 Hz, 1H), 7.53 (d, J=3.9 Hz, 1H), 4.97-4.81 (m, 3H), 2.60 (s, 3H), 2.10-1.91 (m, 6H), 1.79-1.71 (m, 1H), 1.43-1.19 (m, 3H); MS (ESI) m/z 276 (M+1).

### Example 17(f) 4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride

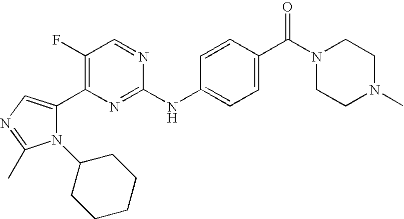

The title compound was prepared in accordance with the general method D, with the exception that the base of the product was purified by flash chromatography (CH₂Cl₂/MeOH gradient; 20:1 to 10:1) before purification by preparative HPLC. Using 4-(1-cyclohexyl-2-methyl-1H-imidazol-5-yl)-5-fluoropyrimidin-2-amine (0.075 g, 0.270 mmol, obtained from Example 17(e)), 1-(4-bromobenzoyl)-4-methylpiperazine (0.115 g, 0.405 mmol), sodium tert-butoxide (0.036 g, 0.38 mmol), Pd(OAc)₂ (0.012 g, 0.054 mmol) and Pd(t-Bu₃P)₂ (0.14 g, 0.027 mmol), the base of the title compound (0.040 g, 31%) was obtained. The hydrochloride of the title compound was prepared in accordance with the general method D.

¹H NMR (DMSO-d₆, 300 MHz) δ 11.34 (br s, 1H), 10.18 (s, 1H), 8.85 (s, 1H), 8.11 (s, 1 H), 7.73 (d, J=8.4 Hz, 2H), 7.41 (d, J=8.4 Hz, 2H), 4.68-4.58 (m, 1H), 4.20-3.90 (m, 4H), 3.13-2.97 (m, 2H), 2.83 (s, 3H), 2.76 (s, 3H), 2.00-1.90 (m, 4H), 1.69-1.57 (m, 2H), 1.55-1.45 (m, 1H), 1.20-1.00 (m, 3H); MS (ESI) m/z 478 (M+1).

### Example 18

### 4-(1-Cyclohexyl-2-methyl-1H-imidazol-5-yl)-5-fluoro-N-{4-[(4-methylpiperazin-1-yl)sulfonyl]phenyl}pyrimidin-2-amine hydrochloride

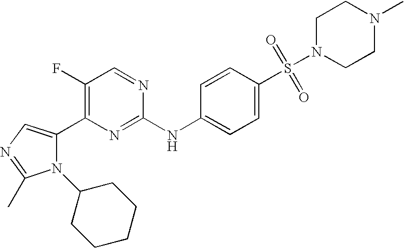

The title compound was prepared in accordance with the general method D, with the exception that the base of the product was purified by flash chromatography (CH₂Cl₂/MeOH/NH₃ aq. 200:10:1) before purification by preparative HPLC. Using 4-(1-cyclohexyl-2-methyl-1H-imidazol-5-yl)-5-fluoropyrimidin-2-amine (obtained from Example 17(e)) (0.075 g, 0.270 mmol), 1-(4-bromo-benzenesulfonyl)-4-methylpiperazine (described in WO 2003004472) (0.105 g, 0.320 mmol), sodium tert-butoxide (0.036 g, 0.38 mmol), Pd(OAc)₂ (0.012 g, 0.054 mmol) and Pd(t-Bu₃P)₂ (0.14 g, 0.027 mmol), the base of the title compound (0.018 g, 14%) was obtained. The hydrochloride of the title compound was prepared in accordance with the general method D.

¹H NMR (DMSO-d₆, 300 MHz) δ 10.88 (br s, 1H), 10.48 (s, 1H), 8.90 (s, 1H), 8.10 (s, 1H), 7.96 (d, J=8.1 Hz, 2H), 7.68 (d, J=8.1 Hz, 2H), 4.69-4.59 (m, 1H), 3.76-3.66 (m, 2H), 3.19-3.05 (m, 2H), 2.82 (s, 3H), 2.73 (s, 3H), 2.02-1.85 (m, 4H), 1.70-1.60 (m, 2H), 1.55-1.48 (m, 1H), 1.25-1.02 (m, 3H); MS (ESI) m/z 514 (M+1).

### Example 19

### 5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride

### Example 19(a) 5-Acetyl-2-methyl-1-(1-methylpiperidin-4-yl)-1H-imidazole

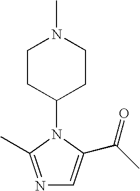

The intermediate acetamide (N-(1-aminopiperidin-4-yl)-N-isoxazol-4-ylacetamide) was prepared in accordance with the general method of Example 7(a) starting from 5-methyl-4-amino-isoxazole (Reiter, L. A, J. Org. Chem., 1987, 52, 2714-2726) (0.61 g, 10.2 mmol), N-methylpiperidine-4-one (0.63 g, 5.6 mmol) and sodium cyanoborohydride (0.32 g, 5.1 mmol+0.1 g, 1.6 mmol). The title compound was prepared in accordance with the general method of Example 7(b) using the crude intermediate acetamide (N-(1-aminopiperidin-4-yl)-N-isoxazol-4-ylacetamide) to give the title compound (0.40 g, 45%) after purification by flash chromatography (CH₂Cl₂/MeOH/NH₃ aq. 150:10:1).

¹H NMR (CDCl₃, 300 MHz) δ 7.85 (s, 1H), 5.28-5.16 (m, 1H), 3.02-2.92 (m, 2H), 2.56 (s, 3H), 2.43 (s, 3H), 2.40-2.24 (m, 5H), 2.19-2.06 (m, 2H), 1.86-1.76 (m, 2H);

MS (ESI) m/z 222 (M+1).

### Example 19(b) (2E)-3-Dimethylamino-1-[2-methyl-1-(1-methylpiperidin-4-yl)-1H-imidazol-5-yl]prop-2-en-1-one

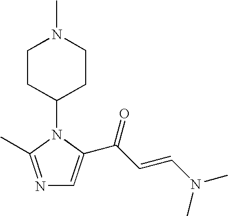

The title compound was prepared in accordance with the general method of Example 7(c), using 5-acetyl-2-methyl-1-(1-methylpiperidin-4-yl)-1H-imidazole (2.1 g, 9.50 mmol, is obtained from Example 19(a)). After purification by flash chromatography (CH₂Cl₂/MeOH/NH₃ aq. gradient; 200:10:1 to 200:15:1.5) the title compound was obtained (1.92 g, 73%) as a yellow oil that solidified upon standing.

¹H NMR (CDCl₃, 300 MHz) δ 7.63 (d, J=12.3 Hz, 1H), 7.43 (s, 1H), 5.48 (d, J=12.3 Hz, 1H), 5.30-5.18 (m, 1H), 3.12-2.85 (m, 8H), 2.54 (s, 3H), 2.40-2.30 (m, 2H), 2.29 (s, 3H), 2.17-2.07 (m, 2H), 1.88-1.80 (m, 2H); MS (ESI) m/z 277 (M+1).

### Example 19(c) (2Z)-3-Dimethylamino-2-fluoro-1-[2-methyl-1-(1-methylpiperidin-4-yl)-1H-imidazol-5-yl]prop-2-en-1-one

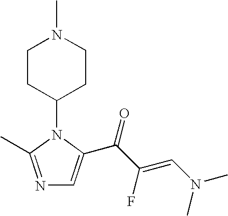

Selectfluor (2.50 g, 6.95 mmol) was added in portions over 5 minutes to a stirred solution of (2E)-3-dimethylamino-1-[2-methyl-1-(1-methylpiperidin-4-yl)-1H-imidazol-5-yl]prop-2-en-1-one (1.92 g, 6.95 mmol) in MeOH (30 mL) at 0° C. After stirring for 90 minutes at r.t. more Selectfluor (1.25 g, 3.5 mmol) was added and the mixture was stirred for 2 h.

When the reaction was complete it was concentrated in vacuo, diluted with NH₃ (3%, aq., 50 mL) and extracted with CHCl₃ (3×50 mL, contained 5% MeOH). The combined organic phases were dried (Na₂SO₄), filtered and concentrated in vacuo. The crude product was purified by flash chromatography (CH₂Cl₂/MeOH/NH₃ aq. 200:10:1), to give the title compound (0.68 g, 33%) as an oil.

¹H NMR (CDCl₃, 300 MHz) δ 7.35 (s, 1H), 6.85 (d, J=27.3 Hz, 1H), 4.82-4.70 (m, 1H), 3.09 (s, 6H), 3.02-2.92 (m, 2H), 2.55 (s, 3H), 2.40-2.25 (m, 5H), 2.16-2.05 (m, 2H), 1.91-1.83 (m, 2H); MS (ESI) m/z 295 (M+1).

### Example 19(d) 5-Fluoro-4-[2-methyl-1-(1-methylpiperidin-4-yl)-1H-imidazol-5-yl]pyrimidin-2-amine

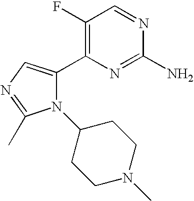

The title compound was prepared in accordance with the general method B with the exception that guanidine carbonate was used. Using (2Z)-3-dimethylamino-2-fluoro-1-[2-methyl-1-(1-methylpiperidin-4-yl)-1H-imidazol-5-yl]prop-2-en-1-one (0.68 g, 2.31 mmol, obtained from Example 19(c)) and guanidine carbonate (1.05 g, 5.78 mmol) the title compound was obtained (0.372 g, 55%) as a white solid after purification by flash chromatography (CH₂Cl₂/MeOH/NH₃ aq. gradient; 200:10:1 to 100:10:1).

¹H NMR (CDCl₃, 300 MHz) δ 8.15 (d, J=3 Hz, 1H), 7.56 (d, J=3.6 Hz, 1H), 5.03-2.90 (m, 3H), 3.05-2.95 (m, 2H), 2.62 (s, 3H), 2.52-2.35 (m, 2H), 2.32 (s, 3H), 2.12-2.00 (m, 2H), 1.99-1.90 (m, 2H); MS (ESI) m/z 291 (M+1).

### Example 19(e) 5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride

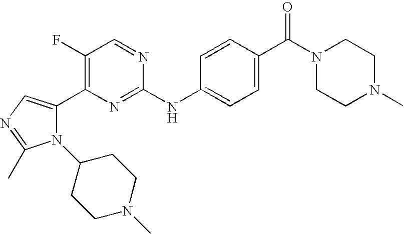

The title compound was prepared in accordance with the general method D, with the exception that the base of the product was purified by flash chromatography (CH₂Cl₂/MeOH/NH₃ aq., 500:30:3) before purification by preparative HPLC. Using 5-fluoro-4-[2-methyl-1-(1-methylpiperidin-4-yl)-1H-imidazol-5-yl]pyrimidin-2-amine (0.075 g, 0.256 mmol, obtained from Example 19(d)), 1-(4-bromobenzoyl)-4-methylpiperazine (0.087 g, 0.307 mmol), sodium tert-butoxide (0.036 g, 0.38 mmol), Pd(OAc)₂ (0.012 g, 0.054 mmol) and Pd(t-Bu₃P)₂ (0.14 g, 0.027 mmol), the base of the title compound was obtained (0.027 g, 21%). The hydrochloride of the title compound was prepared in accordance with the general method D.

¹H NMR (DMSO-d₆, 300 MHz) δ 11.31 (br s, 1H), 10.22 (s, 1H), 8.85 (s, 1H), 8.16 (s, 1H), 7.81 (d, J=8.1 Hz, 2H), 7.43 (d, J=8.1 Hz, 2H), 5.14-5.02 (m, 1H), 3.50-3.35 (m, 4H), 3.11-2.97 (m, 2H), 2.91 (s, 3H), 2.77 (s, 3H), 2.68 (s, 3H), 2.35-2.20 (m, 4H);

MS (ESI) m/z 493 (M+1).

### Example 20

### 5-Fluoro-4-[2-methyl-1-(1-methylpiperidin-4-yl)-1H-imidazol-5-yl]-N-[4-(pyrrolidin-1-ylsulfonyl)phenyl]pyrimidin-2-amine hydrochloride

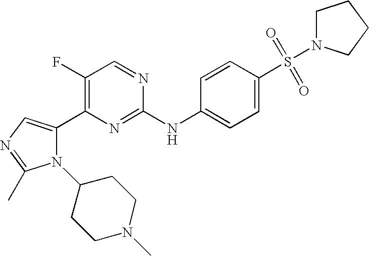

The title compound was prepared in accordance with the general method E, with the exception that the base of the product was purified by flash chromatography (CH₂Cl₂/MeOH/NH₃ aq. 200:10:1). Using 5-fluoro-4-[2-methyl-1-(tetrahydro-2H-pyran-4-yl)-1H-imidazol-5-yl]pyrimidin-2-amine (obtained from Example 19(d)) (40 mg, 0.136 mmol), 1-(4-bromo-benzenesulfonyl)-pyrrolidine (43 mg, 0.149 mmol), Cs₂CO₃ (50 mg, 0.15 mmol), Pd₂(dba)₃ (7 mg, 0.008 mmol) and X-Phos (7 mg, 0.015 mmol), the base of the title compound (58 mg, 85%) was obtained as a solid. The hydrochloride of the title compound was prepared in accordance with the general method D.

¹H NMR (DMSO-d₆, 300 MHz) δ 11.32 (br s, 1H), 10.48 (s, 1H), 8.89 (s, 1H), 8.16 (s, 1H), 8.00 (d, J=8.1 Hz, 2H), 7.73 (d, J=8.1 Hz, 2H), 5.16-5.03 (m, 1H), 3.45-3.35 (m, 2H), 3.18-3.03 (m, 5H), 2.91 (s, 3H), 2.75-2.64 (m, 4H), 2.35-2.22 (m, 2H), 2.64 (s, 4H). MS (ESI) m/z 500 (M+1).

### Example 21

### 5-Fluoro-4-[2-methyl-1-(tetrahydro-2H-pyran-4-yl)-1H-imidazol-5-yl]-N-[4-(trifluoromethyl)phenyl]pyrimidin-2-amine hydrochloride

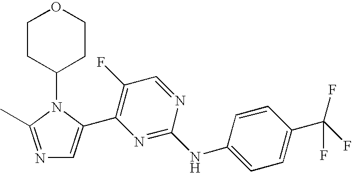

The title compound was prepared in accordance with the general method E, with the exception that the base of the product was purified by flash chromatography (CH₂Cl₂/MeOH 40:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 1-bromo-4-(trifluoromethyl)benzene (0.031 g, 0.139 mmol), Cs₂CO₃ (92 mg, 0.28 mmol), Pd₂(dba)₃ (7 mg, 0.008 mmol) and X-Phos (7 mg, 0.014 mmol), the base of the title compound (50 mg, 92%) was obtained as a solid. The hydrochloride of the title compound was prepared in accordance with the general method D.

¹H NMR (DMSO-d₆, 300 MHz) δ 10.28 (s, 1H), 8.87 (s, 1H), 8.10 (s, 1H), 7.84 (d, J=8.1 Hz, 2H), 7.64 (d, J=8.1 Hz, 2H), 5.05-4.93 (m, 1H), 3.84-3.77 (m, 2H), 3.20-3.10 (m, 2H), 2.83 (s, 3H), 2.20-2.11 (m, 2H), 1.98-1.90 (m, 2H); MS (ESI) m/z 422 (M+1).

### Example 22

### 5-Fluoro-N-[3-(methylsulfonyl)phenyl]-4-[2-methyl-1-(tetrahydro-2H-pyran-4-yl)-1H-imidazol-5-yl]pyrimidin-2-amine hydrochloride

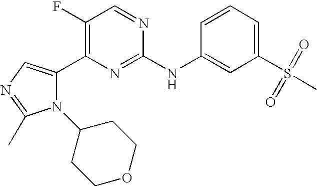

The title compound was prepared in accordance with the general method E, with the exception that the base of the product was purified by flash chromatography (CH₂Cl₂/MeOH 25:1). Using 5-fluoro-4-[2-methyl-1-(tetrahydro-2H-pyran-4-yl)-1H-imidazol-5-yl]pyrimidin-2-amine (obtained from Example 7 (e)) (35 mg, 0.126 mmol), 1-bromo-3-(methylsulfonyl)benzene (0.030 g, 0.126 mmol), Cs₂CO₃ (92 mg, 0.28 mmol), Pd₂(dba)₃ (7 mg, 0.008 mmol) and X-Phos (7 mg, 0.014 mmol), the base of the title compound (44 mg, 81%) was obtained as a solid. The hydrochloride of the title compound was prepared in accordance with the general method D.

¹H NMR (DMSO-d₆, 300 MHz) δ 10.19 (s, 1H), 8.86 (s, 1H), 8.17 (s, 1H), 8.09 (s, 1H), 8.01 (d, J=6.8 Hz, 1H), 7.62-7.52 (m, 2H), 5.03-4.91 (m, 1H), 3.87-3.79 (m, 2H), 3.24-3.10 (m, 5H), 2.82 (s, 3H), 2.20-2.10 (m, 2H), 1.97-1.90 (m, 2H). MS (ESI) m/z 432 (M+1).

### Example 23

### 5-Fluoro-N-[4-(methylsulfonyl)phenyl]-4-[2-methyl-1-(tetrahydro-2H-pyran-4-yl)-1H-imidazol-5-yl]pyrimidin-2-amine hydrochloride

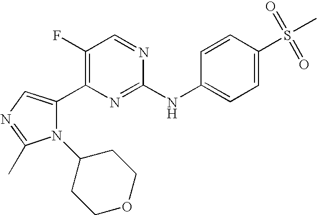

The title compound was prepared in accordance with the general method E, with the exception that the base of the product was purified by flash chromatography (CH₂Cl₂/MeOH 20:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 1-chloro-4-(methylsulfonyl)benzene (0.0215 g, 0.113 mmol), Cs₂CO₃ (92 mg, 0.28 mmol), Pd₂(dba)₃ (7 mg, 0.008 mmol) and X-Phos (7 mg, 0.014 mmol), the base of the title compound (45 mg, 94%) was obtained as a solid. The hydrochloride of the title compound was prepared in accordance with the general method D.

¹H NMR (DMSO-d₆, 300 MHz) δ 10.39 (s, 1H), 8.89 (s, 1H), 8.10 (s, 1H), 7.88 (d, J=8.6 Hz, 2H), 7.81 (d, J=8.6 Hz, 2H), 5.03-4.92 (m, 1H), 3.87-3.79 (m, 2H), 3.25-3.10 (m, 5H), 2.83 (s, 3H), 2.21-2.10 (m, 2H), 1.98-1.91 (m, 2H). MS (ESI) m/z 432 (M+1).

### Example 24

### 3-({5-Fluoro-4-[2-methyl-1-(tetrahydro-2H-pyran-4-yl)-1H-imidazol-5-yl]pyrimidin-2-yl}amino)benzonitrile hydrochloride

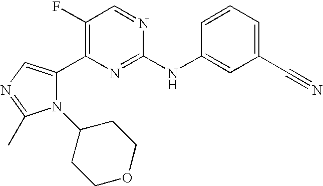

The title compound was prepared in accordance with the general method E, with the exception that the base of the product was purified by flash chromatography (CH₂Cl₂/MeOH 40:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 3-bromobenzonitrile (0.023 g, 0.126 ml-ol), Cs₂CO₃ (92 mg, 0.28 mmol), Pd₂(dba)₃ (7 mg, 0.008 mmol) and X-Phos (7 mg, 0.014 mmol), the base of the title compound (44 mg, 92%) was obtained as a solid. The hydrochloride of the title compound was prepared in accordance with the general method D.

¹H NMR (DMSO-d₆, 300 MHz) δ 10.22 (s, 1H), 8.87 (s, 1H), 8.18-8.05 (m, 2H), 7.90 (s, 1H), 7.55-7.25 (m, 2H), 5.03-4.91 (m, 1H), 3.85-3.77 (m, 2H), 3.22-3.10 (m, 2H), 2.83 (s, 3H), 2.21-2.11 (m, 2H), 1.97-1.90 (m, 2H). MS (ESI) m/z 379 (M+1).

### Example 25

### 4-(1,2-Dimethyl-1H-imidazol-5-yl)-5-fluoro-N-[4-(morpholin-4-ylmethyl)phenyl]pyrimidin-2-amine hydrochloride

### Example 25(a) 4-(1,2-Dimethyl-1H-imidazol-5-yl)-5-fluoropyrimidin-2-amine

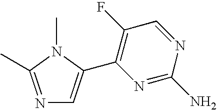

2-Chloro-4-(1,2-dimethyl-1H-imidazol-5-yl)-5-fluoropyrimidine (0.295 g, 1.30 mmol) (obtained from Example 1(b)) was dissolved in 1-propanol (3.0 mL) in a microwave vial. Ammonium hydroxide (28%, 1.0 mL) was added, the vial was sealed and the mixture heated in a microwave oven (+140° C., 4 h). The reaction mixture was cooled to r.t. and the solvent was evaporated. The residue was partitioned between CH₂Cl₂ and 1M aqueous HCl. The aqueous phase, containing the product, was neutralized with saturated aqueous NaHCO₃ and the product extracted with CH₂Cl₂. The organic phase was co-evaporated with ethanol and the residue was purified by flash chromatography using (CH₂Cl₂/MeOH gradient; 100:1 to 94:6) to give the title compound (0.210 g, 78%) as a solid.

¹H NMR (CDCl₃) δ ppm 8.15 (d, J=3.5 Hz, 1H), 7.71 (d, J=4.3 Hz, 1H), 4.87 (br s, 2H), 3.97 (s, 3H), 2.49 (s, 3H); MS (ESI) m/z 208 (M+1).

### Example 25(b) 4-(1,2-Dimethyl-1H-imidazol-5-yl)-5-fluoro-N-[4-(morpholin-4-ylmethyl)phenyl]pyrimidin-2-amine hydrochloride

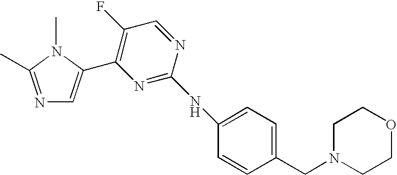

The title compound was prepared in accordance with the general method E. Using 4-(1,2-dimethyl-1H-imidazol-5-yl)-5-fluoropyrimidin-2-amine (21 mg, 0.10 mmol, obtained from Example 25(a)), 4-(4-bromobenzyl)morpholine (28 mg, 0.11 mmol), Cs₂CO₃ (58 mg, 0.18 mmol), Pd₂(dba)₃ (7 mg, 0.007 mmol) and X-Phos (8 mg, 0.017 mmol), the base of the title compound was obtained as a solid. The hydrochloride of the title compound was prepared in accordance with the general method D, with the exceptions that dilute ammonium hydroxide was used instead of dilute NaHCO₃ in the washing of the organic phase. The organic phase was co-evaporated with absolute ethanol instead of drying with Na₂SO₄ before evaporation, and the precipitated salt was collected by filtration and re-dissolved in water before freeze drying, giving the title compound (28 mg, 61%) as a yellow solid.

¹H NMR (DMSO-d₆) δ ppm 10.84 (br s, 1H), 10.02 (s, 1H), 8.75 (d, J=2.5 Hz, 1H), 8.15 (d, J=2.5 Hz, 1H), 7.75 (d, J=8.5 Hz, 2H), 7.51 (d, J=8.5 Hz, 2H), 4.26 (s, 2H), 4.01 (s, 3H), 3.99-3.86 (m, 2H), 3.76 (t, J=11.8 Hz, 2H), 3.25-3.14 (m, 2H), 3.13-2.96 (m, 2H), 2.66 (s, 3H); MS (ESI) m/z 381 (M+1).

### Example 26

### 4-(1,2-Dimethyl-If-imidazol-5-yl)-5-fluoro-N-{4-[(4-methylpiperazin-1-yl)sulfonyl]phenyl}pyrimidin-2-amine

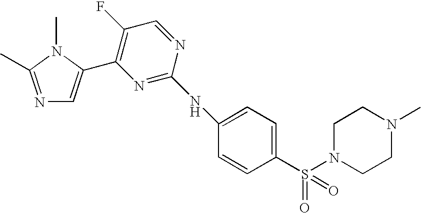

The title compound was prepared in accordance with the general method E. Using 4-(1,2-dimethyl-1H-imidazol-5-yl)-5-fluoropyrimidin-2-amine (obtained from Example 25(a)) (23 mg, 0.11 mmol), 1-[(4-bromophenyl)sulfonyl]-4-methylpiperazine (37 mg, 0.11 mmol), Cs₂CO₃ (59 mg, 0.18 mmol), Pd₂(dba)₃ (6 mg, 0.006 mmol) and X-Phos (6 mg, 0.013 mmol), the base of the title compound was obtained as a solid. The hydrochloride of the title compound was prepared in accordance with the procedure described in Example 25 (b), giving the title compound (33 mg, 57%) as a yellow solid.

¹H NMR (DMSO-d₆) δ ppm 10.44 (br s, 1H), 10.42 (s, 1H), 8.82 (d, J=2.5 Hz, 1H), 8.13 (br s, 1H), 7.99 (d, J=8.8 Hz, 2H), 7.73 (d, J=8.8 Hz, 2H), 4.03 (s, 3H), 3.83-3.63 (m, 2H), 3.23-3.02 (m, 4H), 2.74 (s, 3H), 2.65 (s, 3H), 2.70-2.56 (m, 2H); MS (ESI) m/z 446 (M+1).

### Example 27

### 4-(1,2-Dimethyl-1H-imidazol-5-yl)-5-fluoro-N-[4-(piperidin-1-ylcarbonyl)phenyl]pyrimidin-2-amine hydrochloride

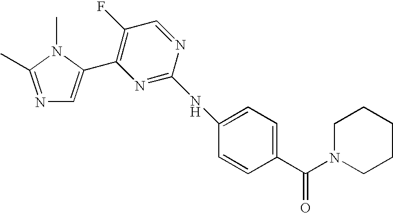

The title compound was prepared in accordance with the general method E. Using 4-(1,2-dimethyl-1H-imidazol-5-yl)-5-fluoropyrimidin-2-amine (obtained from Example 25 (a)) (25 mg, 0.12 mmol), 1-(4-bromobenzoyl)piperidine (33 mg, 0.12 mmol), Cs₂CO₃ (66 mg, 0.20 mmol), Pd₂(dba)₃ (6 mg, 0.007 mmol) and X-Phos (6 mg, 0.013 mmol), the base of the title compound was obtained as a solid. The hydrochloride of the title compound was prepared in accordance with the procedure described in Example 25(b), giving the title compound (35 mg, 61%) as a white solid.

¹H NMR (DMSO-d₆) δ ppm 10.04 (s, 1H), 8.77 (d, J=2.8 Hz, 1H), 8.17 (d, J=2.8 Hz, 1H), 7.73 (d, J=8.5 Hz, 2H), 7.35 (d, J=8.8 Hz, 2H), 4.02 (s, 3H), 3.50-3.41 (m, 4H), 2.66 (s, 3H), 1.67-1.57 (m, 2H), 1.57-1.42 (m, 4H); MS (ESI) m/z 395 (M+1).

### Example 28

### 4-(1,2-Dimethyl-1H-imidazol-5-yl)-5-fluoro-N-{4-[(4-methylpiperazin-1-yl)carbonyl]phenyl}pyrimidin-2-amine hydrochloride

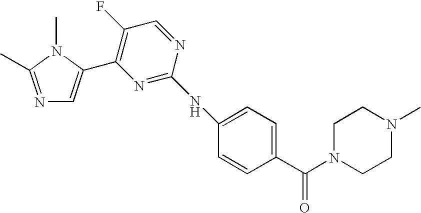

The title compound was prepared in accordance with the general method E. Using 4-(1,2-dimethyl-1H-imidazol-5-yl)-5-fluoropyrimidin-2-amine (obtained from Example 25(a)) (18 mg, 0.089 mmol), 1-(4-bromobenzoyl)-4-methylpiperazine (25 mg, 0.089 mmol), Cs₂CO₃ (42 mg, 0.13 mmol), Pd₂(dba)₃ (4 mg, 0.004 mmol) and X-Phos (4 mg, 0.009 mmol), the base of the title compound was obtained as a solid. The hydrochloride of the title compound was prepared in accordance with the procedure described in Example 25 (b), giving the title compound (28 mg, 64%) as a yellow solid.

¹H NMR (DMSO-d₆) δ ppm 10.71 (br s, 1H), 10.13 (s, 1H), 8.78 (d, J=2.8 Hz, 1H), 8.18 (d, J=2.5 Hz, 1H), 7.78 (d, J=8.5 Hz, 2H), 7.45 (d, J=8.5 Hz, 2H), 4.20 (br s, 2H), 4.03 (s, 3H), 3.07 (br s, 2H), 2.79 (s, 3H), 2.67 (s, 3H); MS (ESI) m/z 410 (M+1).

### Example 29

### 4-(1,2-Dimethyl-1H-imidazol-5-yl)-5-fluoro-N-{4-[(4-methylpiperazin-1-yl)methyl]phenyl}pyrimidin-2-amine hydrochloride

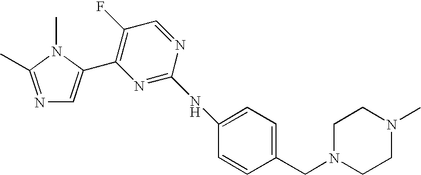

The title compound was prepared in accordance with the general method E. Using 4-(1,2-dimethyl-1H-imidazol-5-yl)-5-fluoropyrimidin-2-amine (obtained from Example 25 (a)) (19 mg, 0.091 mmol), 1-(4-bromobenzyl)-4-methylpiperazine (Organ, M. G. et al, J. Comb. Chem. 2001, 3, 473-476) (28 mg, 0.10 mmol), Cs₂CO₃ (42 mg, 0.13 mmol), Pd₂(dba)₃ (5 mg, 0.005 mmol) and X-Phos (4 mg, 0.009 mmol), the base of the title compound was obtained as a solid. The hydrochloride of the title compound was prepared in accordance with the procedure described in Example 25 (b), giving the title compound (28 mg, 61%) as a yellow solid.

¹H NMR (DMSO-d₆) δ ppm 10.00 (br s, 1H), 8.76 (d, J=2.5 Hz, 1H), 8.20 (d, J=2.8 Hz, 1H), 7.72 (d, J=8.3 Hz, 2H), 7.55-7.38 (m, 2H), 4.02 (s, 3H), 4.02 (br s, 1H), 2.79 (s, 3 H), 2.68 (s, 3H); MS (ESI) m/z 396 (M+1).

### Example 30

### 4-(1,2-Dimethyl-1H-imidazol-5-yl)-5-fluoro-N-{3-[(4-methylpiperazin-1-yl)carbonyl]phenyl}pyrimidin-2-amine hydrochloride

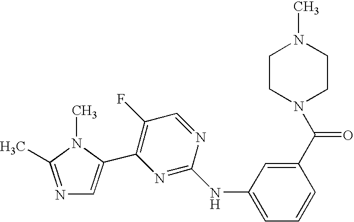

The title compound was prepared in accordance with the general method E. Using 4-(1,2-dimethyl-1H-imidazol-5-yl)-5-fluoropyrimidin-2-amine (obtained from Example 25 (a)) (18 mg, 0.084 mmol), 1-(3-chlorobenzoyl)-4-methylpiperazine (21 mg, 0.87 mmol), Cs₂CO₃ (43 mg, 0.13 mmol), Pd₂(dba)₃ (4 mg, 0.004 mmol) and X-Phos (4 mg, 0.009 mmol), the base of the title compound was obtained as a solid. The hydrochloride of the title compound was prepared in accordance with the procedure described in Example 25 (b), giving the title compound (13 mg, 32%) as a yellow solid.

¹H NMR (DMSO-d₆) δ ppm 10.69 (br s, 1H), 10.00 (s, 1H), 8.74 (d, J=2.5 Hz, 1H), 8.12 (br s, 1H), 7.83-7.72 (m, 2H), 7.42 (t, J=7.9 Hz, 1H), 7.09 (d, J=7.5 Hz, 1H), 4.50 (br s, 1H), 4.01 (s, 3H), 2.80 (s, 3H), 2.65 (s, 3H); MS (ESI) m/z 410 (M+1).

### Example 31

### (4-{[4-(1,2-Dimethyl-1H-imidazol-5-yl)-5-fluoropyrimidin-2-yl]amino}phenyl)(pyridin-2-yl)methanone hydrochloride

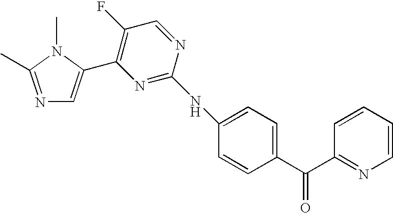

The title compound was prepared in accordance with the general method E. Using 4-(1,2-dimethyl-1H-imidazol-5-yl)-5-fluoropyrimidin-2-amine (obtained from Example 25(a)) (23 mg, 0.11 mmol), (4-bromophenyl)(pyridin-2-yl)methanone (29 mg, 0.11 mmol), Cs₂CO₃ (72 mg, 0.22 mmol), Pd₂(dba)₃ (6 mg, 0.007 mmol) and X-Phos (7 mg, 0.016 mmol), the base of the title compound was obtained as a solid. The hydrochloride of the title compound was prepared in accordance with the procedure described in Example 25(b), giving the title compound (28 mg, this compound appeared to be a non-stoichiometric salt, gives an estimated yield of 55%) as a yellow solid.

¹H NMR (DMSO-d₆) δ ppm 10.36 (s, 1H), 8.84 (d, J=2.5 Hz, 1H), 8.75-8.69 (m, 1H), 8.20 (d, J=2.8 Hz, 1H), 8.09-8.00 (m, 3H), 7.92 (d, J=7.8 Hz, 1H), 7.87 (d, J=8.8 Hz, 2H), 7.68-7.62 (m, 1H), 4.04 (s, 3H), 2.67 (s, 3H); MS (ESI) m/z 389 (M+1).

### Example 32

### 4-({5-Fluoro-4-[2-methyl-1-(tetrahydro-2H-pyran-4-yl)-1H-imidazol-5-yl]pyrimidin-2-yl}amino)benzonitrile hydrochloride

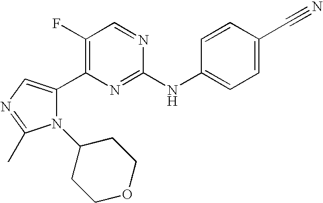

The title compound was prepared in accordance with the general method E, with the exception that the base of the product was purified by flash chromatography (CH₂Cl₂/MeOH 30:1). Using 5-fluoro-4-[2-methyl-1-(tetrahydro-2H-pyran-4-yl)-1H-imidazol-5-yl]pyrimidin-2-amine (obtained from Example 7(e)) (35 mg, 0.126 mmol), 4-chlorobenzonitrile (15.5 g, 0.113 mmol), Cs₂CO₃ (92 mg, 0.28 mmol), Pd₂(dba)₃ (7 mg, 0.008 mmol) and X-Phos (7 mg, 0.014 mmol), the base of the title compound (38 mg, 88%) was obtained as a solid. The hydrochloride of the title compound was prepared in accordance with the general method D.

¹H NMR (DMSO-d₆, 300 MHz) δ 10.42 (s, 1H), 8.90 (s, 1H), 8.09 (s, 1H), 7.86 (d, J=8.5 Hz, 2H), 7.74 (d, J=8.5 Hz, 2H), 5.04-4.92 (m, 1H), 3.89-3.81 (m, 2H), 3.26-3.16 (m, 2H), 2.83 (s, 3H), 2.20-2.12 (m, 2H), 1.97-1.90 (m, 2H); MS (ESI) m/z 379 (M+1).

### Example 33

### 5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride

### Example 33(a) 1-(tert-Butoxycarbonyl)-4-(4-bromo-benzenesulfonyl)-piperazine

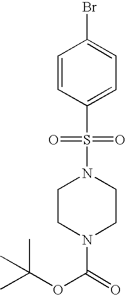

N-boc-piperazine (0.5 g, 2.68 mmol) and diisopropyethylamine (0.69 g, 5.36 mmol) were dissolved in CH₂Cl₂ (50 mL) and cooled to 0° C. 4-Bromo-phenylsulphonyl chloride (0.68 g, 2.68 mmol) in CH₂Cl₂ (10 mL) was added dropwise under vigorous stirring. After stirring for 15 h at r.t. the reaction mixture was washed with saturated aqueous NaHCO₃ (2×20 mL), brine, then dried (Na₂SO₄) and concentrated to dryness. The solid residue was recrystallized from heptane/EtOAc mixture (2:1), filtered and washed with cold heptane. The title compound was obtained (0.8 g, 74%) as a white solid.

¹H NMR (CDCl₃, 300 MHz) δ 7.68 (d, J=8.4 Hz, 2H), 7.61 (d, J=8.4 Hz, 2H), 3.54-3.47 (m, 4H), 3.01-2.94 (m, 4H), 1.41 (s, 9H).

### Example 33(b) 5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride

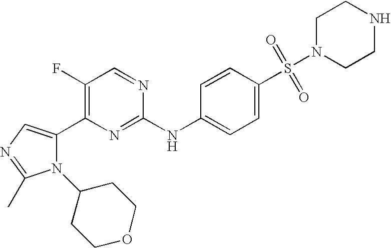

5-Fluoro-4-[2-methyl-1-(tetrahydro-2H-pyran-4-yl)-1H-imidazol-5-yl]pyrimidin-2-amine (obtained from Example 7(e)) (45 mg, 0.162 mmol) and 1-(tert-butoxycarbonyl)-4-(4-bromo-benzenesulfonyl)-piperazine (62 mg, 0.154 mmol) were dissolved in dioxane (3 mL). Cesium carbonate (105 mg, 0.324 mmol) was added and nitrogen gas was bubbled throw the stirred suspension for 2-3 min followed by the addition of Pd₂(dba)₃ (7.5 mg, 0.0081 mmol) and X-Phos (7.7 g, 0.0162 mmol). The vessel was closed and heated to +90° C. and stirred at this temperature for 20 h. The cooled mixture was diluted with chloroform (15 ml) and water (20 mL), the aqueous layer was separated and extracted with chloroform (20 mL). The combined organic layers were dried (Na₂SO₄) and the solvent was removed in vacuo. The residue was purified by flash chromatography using CH₂Cl₂/MeOH (40:1) as eluent. Product-containing fractions were concentrated, dissolved in MeOH (20 mL) and treated with HCl (37% aqueous, 0.5 mL). The mixture was stirred overnight at r.t. and concentrated to dryness. Diluted with chloroform (20 mL) and water (20 mL) and the aqueous layer was separated and extracted with chloroform (3×20 mL). The combined organic layers were dried (Na₂SO₄), the solvents were removed in vacuo and the residue was purified by flash chromatography using CH₂Cl₂/MeOH/aqueous NH₃ (150:10:1 to 100:10:1) as eluent, obtaining the base (22 mg, 28%) as an oil. The base of the title compound was dissolved in chloroform/ether (1:1) and treated with 1M HCl in ether. The resulting precipitate was collected by filtration and washed with ether to obtain of the title compound (13 mg) as a solid.

¹H NMR (DMSO-d₆, 300 MHz) δ 10.45 (s, 1H), 9.12 (br s, 1H), 8.90 (s, 1H), 8.07 (s, 1H), 7.96 (d, J=8.7 Hz, 2H), 7.70 (d, J=8.7 Hz, 2H), 5.03-4.93 (m, 1H), 3.89-3.81 (m, 2H), 3.65-3.40 (m, 2H), 3.28-3.09 (m, 8H), 2.82 (s, 3H), 2.22-2.10 (m, 2H), 2.00-1.92 (m, 2H); MS (ES) m/z 502 (M+1).

### Example 34

### 5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride

### Example 34(a) 5-Acetyl-1-(tetrahydro-2H-pyran-4-yl)-2-trifluoromethyl-1H-imidazole

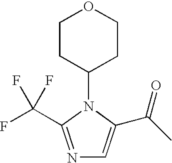

5-Methyl-4-amino-isoxazole (1.7 g, 17.25 mmol) and acetic acid (1.1 g, 19 mmol) were dissolved in methanol (50 mL). Tetrahydro-2H-pyran-4-one (1.9 g, 19 mmol) was added and the mixture was cooled to 0-(−5)° C. and stirred for 1 h. Sodium cyanoborohydride (0.812 g, 12.9 mmol) was added in portions to the reaction mixture at −5° C., causing weak exothermic and gas evolution. The cooling bath was removed and the mixture was stirred at r.t. for 2 h followed by addition of water (20 mL). The methanol was removed from the reaction mixture by vacuum distillation, and the intermediate amine was extracted with ethyl acetate (3×80 mL). The combined organic layers were dried (Na₂SO₄), concentrated to dryness, dissolved in toluene and re-concentrated. The crude intermediate amine, was dissolved in CH₂Cl₂ (20 mL) and pyridine (2 mL, 26 mmol) was added. The mixture was cooled to 0° C. and trifluoroacetic anhydride (4.35 g, 20.7 mmol) was added dropwise. The mixture was continued stirring for 2 h at r.t. then washed with water and saturated NaHCO₃. The aqueous layer was extracted with CH₂Cl₂ (2×30 mL), the organic extracts were dried (Na₂SO₄) and concentrated to dryness to give a second crude intermediate, 4-[N-(tetrahydro-2H-pyran-4-yl)]-N-trifluoroacetyl-amino-5-methylisoxazole. MS (ES) m/z 279 (M⁺+1). The title compound was prepared in accordance with the general method of Example 7 (b) using the intermediate 4-[N-(tetrahydro-2H-pyran-4-yl)]-N-trifluoroacetyl-amino-5-methylisoxazole (max 17.25 mmol), with the exception that the product was purified by flash chromatography (heptane/EtOAc 3:2), giving the title compound (3.03 g, 67%) as an oil.

¹H NMR (CDCl₃, 300 MHz) δ 7.85 (s, 1H), 4.89-4.75 (m, 1H), 4.17-4.07 (m, 2H), 3.54-3.44 (m, 2H), 2.75-2.60 (m, 2H), 2.56 (s, 3H), 1.72-1.63 (m, 2H);

MS (ES) m/z 263 (M+1).

### (b) (2E)-3-Dimethylamino-1-[1-(tetrahydro-2H-pyran-4-yl)-2-trifluoromethyl-1H-imidazol-5-yl]prop-2-en-1-one

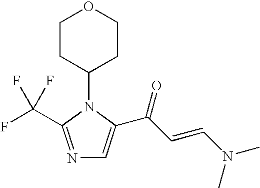

The title compound was prepared in accordance with the general method of Example 7 (c), with the exception that the product was purified by flash chromatography (EtOAc). Using 5-acetyl-1-(tetrahydro-2H-pyran-4-yl)-2-trifluoromethyl-1H-imidazole (3.03 g, 11.55 mmol, obtained from Example 34(a)) the title compound was obtained (3.2 g, 87%) as an oil.

¹H NMR (CDCl₃, 300 MHz) δ 7.72 (d, J=12.3 Hz, 1H), 7.49 (s, 1H), 5.50 (d, J=12.3 Hz, 1H), 4.89-4.75 (m, 1H), 4.14-4.05 (m, 2H), 3.54-3.44 (m, 2H), 3.16 (br. s, 3H), 2.93 (br. s, 3H), 2.86-2.72 (m, 2H), 1.80-1.72 (m, 2H); MS (ES) m/z 318 (M+1).

### Example 34(c) (2Z)-3-Dimethylamino-2-fluoro-1-[1-(tetrahydro-2H-pyran-4-yl)-2-trifluoromethyl-1H-imidazol-5-yl]prop-2-en-1-one

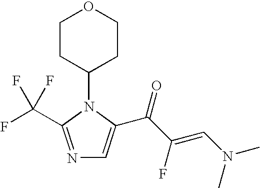

Selectfluor (0.370 g, 1.04 mmol) was added in portions to a stirred solution of (2E)-3-dimethylamino-1-[1-(tetrahydro-2H-pyran-4-yl)-2-trifluoromethyl-1H-imidazol-5-yl]prop-2-en-1-one (0.300 g, 0.946 mmol, obtained from Example 34(b)) in MeCN (20 mL) at 0° C. After stirring for 0.5 h at r.t. more Selectfluor (0.050 g, 0.14 mmol) was added, and the mixture was stirred for 0.5 h. The solvent was evaporated in vacuo, diluted with 3% aqueous NH₃ (20 mL) and extracted with CHCl₃ (3×20 mL). The organic extracts were dried (Na₂SO₄), evaporated in vacuo and the crude product was purified by flash chromatography (heptane/EtOAc 1:2), followed by neat EtOAc) to obtain the title compound (0.170 g, 53%) as an oil.

¹H NMR (CDCl₃, 300 MHz) δ 7.34 (s, 1H), 6.85 (d, J=26.7 Hz, 1H), 4.67-4.54 (m, 1H), 4.11-4.03 (m, 2H), 3.50-3.38 (m, 2H), 3.14 (s, 6H), 2.72-2.56 (m, 2H), 1.83-1.74 (m, 2H);

MS (ES) m/z 336 (M+1).

### Example 34(d) 5-Fluoro-4-[1-(tetrahydro-2H-pyran-4-yl)-2-(trifluoromethyl)-1H-imidazol-5-yl]pyrimidin-2-amine

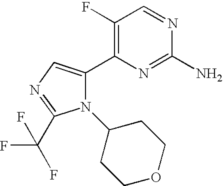

The title compound was prepared in accordance with the general method B with the exception that guanidine carbonate was used. Using (2Z)-3-dimethylamino-2-fluoro-1-[1-(tetrahydro-2H-pyran-4-yl)-2-trifluoromethyl-1H-imidazol-5-yl]prop-2-en-1-one (0.330 g, 1.0 mmol, obtained from Example 34(c)) and guanidine carbonate (0.45 g, 2.50 mmol). After purification by flash chromatography (heptane/EtOAc 1:2), the title compound was obtained (0.170 g, 51%) as a white solid.

¹H NMR (CDCl₃, 300 MHz) δ 8.29 (s, 1H), 7.63 (d, J=2.7 Hz, 1H), 5.10 (br.s., 2H), 4.88-4.76 (m, 1H), 4.16-4.07 (m, 2H), 3.53-3.42 (m, 2H), 2.80-2.65 (m, 2H), 1.89-1.81 (m, 2H); MS (ES) m/z 332 (M+1).

### Example 34(e) 5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride

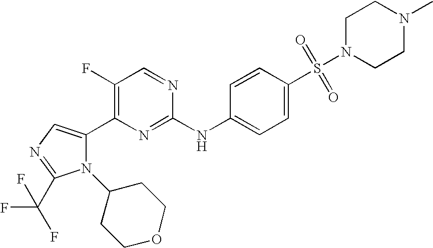

The title compound was prepared in accordance with the general method of Example 33(b). Using 5-fluoro-4-[1-(tetrahydro-2H-pyran-4-yl)-2-(trifluoromethyl)-1H-imidazol-5-yl]pyrimidin-2-amine (33 mg, 0.100 mmol, obtained from Example 34(d)) and 1-(4-bromo-benzenesulfonyl)-4-methylpiperazine (described in WO 2003004472) (29 mg, 0.090 mmol), the base of the title compound was obtained (48 mg, 94%) after purification by flash chromatography (CH₂Cl₂/MeOH 30:1 to 15:1). The hydrochloride of the title compound was prepared in accordance with the general method D.

¹H NMR (DMSO-d₆, 300 MHz) δ 10.68 (br s, 1H), 10.41 (s, 1H), 8.87 (s, 1H), 7.97 (d, J=8.4 Hz, 2H), 7.71 (d, J=8.4 Hz, 2H), 7.57 (s, 1H), 4.84-4.75 (m, 1H), 3.89-3.80 (m, 2H), 3.77-3.68 (m, 2H), 3.47-3.39 (m, 2H), 3.33-3.23 (m, 2H), 3.16-3.08 (m, 2H), 2.73 (s, 3H), 2.68-2.59 (m, 2H), 2.20-2.10 (m, 2H), 1.97-1.90 (m, 2H); MS (ES) m/z 570 (M+1).

### Example 35

### N-{4-[(Dimethylamino)methyl]phenyl}-5-fluoro-4-[2-methyl-1-(tetrahydro-2H-pyran-4-yl)-1H-imidazol-5-yl]pyrimidin-2-amine

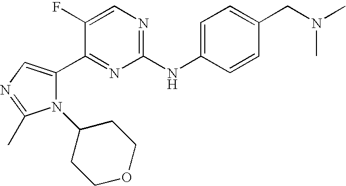

5-Fluoro-4-[2-methyl-1-(tetrahydro-2H-pyran-4-yl)-1H-imidazol-5-yl]pyrimidin-2-amine (50 mg, 180 μmol, obtained from 7(e)) and 1-(4-bromophenyl)-N,N-dimethylmethanamine (38.6 mg, 180 μmol) in dry dioxane (2.3 mL) were purged with Ar (gas) for 10 min. Pd₂(dba)₃ (8.3 mg, 9 μmol), X-Phos (8.6 mg, 18 μmol) and Cs₂CO₃ (102 mg, 289 μmol) were added and Ar (gas) was bubbled through the mixture for 5 min prior to heating at +90° C. for 47 h. The mixture was allowed to cool, diluted with CH₂Cl₂ and filtered through diatomaceous earth. The organics were washed with water, dried (Na₂SO₄), filtered and concentrated. The crude was purified by flash silica gel chromatography CHCl₃/MeOH 9:1-4:1 to give 50 mg (68%).

¹H NMR (400 MHz, DMSO-d₆) δ 9.52 (s, 1H), 8.56 (d, 1H), 7.53 (d, 2H), 7.34 (d, 1H), 7.18 (d, 2H), 5.04 (m, 1H), 3.79 (m, 2H), 3.35 (s, 2H), 3.06 (t, 2H), 2.53 (s, 3H), 2.21-2.11 (m, 8H), 1.78 (m, 2H); MS (ES) m/z 409 (M−1).

### Example 36

### 5-Fluoro-4-[2-methyl-1-(tetrahydro-2H-pyran-4-yl)-1H-imidazol-5-yl]-N-[4-(1-morpholin-4-ylethyl)phenyl]pyrimidin-2-amine

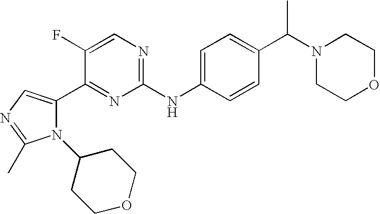

5-Fluoro-4-[2-methyl-1-(tetrahydro-2H-pyran-4-yl)-1H-imidazol-5-yl]pyrimidin-2-amine (50 mg, 180 μmol, obtained from Example 7(e)) and 4-[1-(4-bromophenyl)ethyl]morpholine (obtained from Example 36(a)) (48.7 mg, 180 μmol) in dry dioxane (2.3 mL) were purged with Ar (gas) for 10 min. Pd₂(dba)₃ (8.3 mg, 9 μmol), X-Phos (8.6 mg, 18 μmol) and Cs₂CO₃ (102 mg, 289 μmol) were added and Ar (g) was bubbled through the mixture for 5 min prior to heating at +90° C. for 45 h. The mixture was allowed to cool, diluted with CH₂Cl₂ and filtered through diatomaceous earth. The organics were washed with water, dried (Na₂SO₄), filtered and concentrated. The crude was purified twice by flash silica gel chromatography CHCl₃/MeOH 20:1 and EtOAc/MeOH 40:1-20:1 to give 53 mg of the title compound (63%).

¹H NMR (400 MHz, DMSO-d₆) δ 9.51 (s, 1H), 8.55 (d, 1H), 7.52 (d, 2H), 7.33 (d, 1H), 7.18 (d, 2H), 5.05 (m, 1H), 3.80 (m, 2H), 3.54 (t, 4H), 3.28 (q, 1H), 3.08 (t, 2H), 2.53 (s, 3H), 2.37 (m, 2H), 2.28-2.11 (m, 4H), 1.78 (m, 2H), 1.26 (d, 3H); MS (ES) m/z 465 (M−1).

### Example 36(a) 4-[1-(4-Bromophenyl)ethyl]morpholine

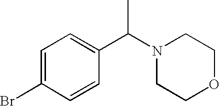

DIPEA (1.98 mL, 11.4 mmol) and morpholine (398 μL, 4.56 mmol) were added to 1-bromo-4-(1-chloroethyl)benzene (Woolman et al. J. Chem. Soc. 1943, 99-101) (500 mg, 2.28 mmol) in dry MeCN (2.5 mL) and the solution was heated at +60° C. for 72 h. The mixture was allowed to cool, concentrated and diluted in CH₂Cl₂. The organics were washed by water, brine and water, dried (Na₂SO₄), filtered and concentrated to give 616 mg (100%) of the desired product.

¹H NMR (400 MHz, DMSO-d₆) δ 7.50 (m, 2H), 7.26 (m, 2H), 3.54 (m, 4H), 3.33 (q, 1H), 2.38 (m, 2H), 2.24 (m, 2H), 1.24 (d, 3H); MS (ES) m/z 270/272 (M/M+2).

### Example 37

### N-[4-(1-Azetidin-1-ylethyl)phenyl]-5-fluoro-4-[2-methyl-1-(tetrahydro-2H-pyran-4-yl)-1H-4-imidazol-5-yl]pyrimidin-2-amine

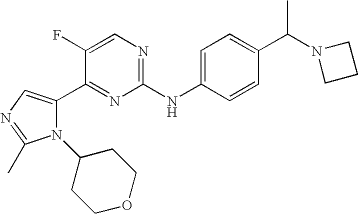

5-Fluoro-4-[2-methyl-1-(tetrahydro-2H-pyran-4-yl)-1H-imidazol-5-yl]pyrimidin-2-amine (50 mg, 180 μmol, obtained from Example 7(e)) and 1-[1-(4-bromophenyl)ethyl]azetidine (obtained from Example 37(a)) (43.3 mg, 180 μmol) in dry dioxane (2.3 mL) were purged with Ar (gas) for 10 min. Pd₂(dba)₃ (8.3 mg, 9 μmol), X-Phos (8.6 mg, 18 μmol) and Cs₂CO₃ (102 mg, 289 μmol) were added and Ar (g) was bubbled through the mixture for 5 min prior to heating at +90° C. for 51 h. The mixture was allowed to cool, diluted with CH₂Cl₂ and filtered through diatomaceous earth. The organics were washed with water, dried (Na₂SO₄), filtered and concentrated. The crude was purified by flash silica gel chromatography CHCl₃/MeOH 9:1-3:1 to give 40 mg (51%).

¹H NMR (600 MHz, CD₃OD) δ 8.39 (d, 1H), 7.55 (d, 2H), 7.42 (d, 1H), 7.26 (d, 2H), 5.21 (m, 1H), 3.91 (m 2H), 3.52 (m, 1H), 3.41 (m, 2H), 3.25 (m, 2H), 3.19 (t, 2H), 2.61 (s, 3H), 2.34 (m, 2H), 2.11 (quintet, 2H), 1.86 (m, 2H), 1.27 (d, 3H); MS (ES) m/z 435 (M−1).

### Example 37(a) 1-[1-(4-Bromophenyl)ethyl]azetidine

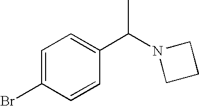

DIPEA (1.98 mL, 11.4 mmol) and azetidine (307 μL, 4.56 mmol) were added to 1-bromo-4-(1-chloroethyl)benzene (Woolman et al. J. Chem. Soc. 1943, 99-101) (500 mg, 2.28 mmol) in dry MeCN (2.5 mL) and the solution was heated at +60° C. for 74 h. More azetidine (100 μL, 1.48 mmol) was added, and after heating for 23 more hours. even more azetidine (100 μL, 1.48 mmol) and DIPEA (500 μL, 2.87 mmol) were added and the mixture was heated for a further 27 h. After cooling and concentration the crude was diluted with CH₂Cl₂. The organics were washed with water, brine and water, dried Na₂SO₄), filtered and concentrated to give 481 mg (87%) of the desired product.

¹H NMR (400 MHz, DMSO-d₆) δ 7.47 (m, 2H), 7.24 (m, 2H), 3.22 (q, 1H), 3.04 (m, 2H), 2.96 (m, 2H), 1.88 (quintet, 2H), 1.05 (d, 3H); MS (ES) m/z 240/242 (M/M+2).

### Example 38

### 5-Fluoro-4-[2-methyl-1-(tetrahydro-2H-pyran-4-yl)-1H-imidazol-5-yl]-N-[4-(2-morpholin-4-ylethyl)phenyl]pyrimidin-2-amine

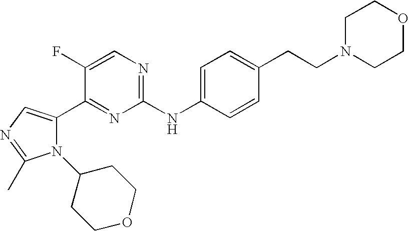

5-Fluoro-4-[2-methyl-1-(tetrahydro-2H-pyran-4-yl)-1H-imidazol-5-yl]pyrimidin-2-amine (50 mg, 180 μmol, obtained from Example 7(e)) and 4-[2-(4-bromophenyl)ethyl]morpholine (King et al. Tet. Lett. 2005, 46, 1471-1474) (48.7 mg, 180 μmol) in dry dioxane (2.3 mL) were purged with Ar (gas) for 10 min. Pd₂(dba)₃ (8.3 mg, 9 μmol), X-Phos (8.6 mg, 18 μmol) and Cs₂CO₃ (102 mg, 289 μmol) were added and Ar (g) was bubbled through the mixture for 5 min prior to heating at +90° C. for 72 h. The mixture was allowed to cool, diluted with CH₂Cl₂ and filtered through diatomaceous earth. The organics were washed with water, dried (Na₂SO₄), filtered and concentrated. The crude was purified by flash silica gel chromatography EtOAc/MeOH 20:1-5:1, the residue was dissolved in CHCl₃ and filtered through tightly packed glass wool and concentrated to give 41 mg of the title compound (49%).

¹H NMR (400 MHz, DMSO-d₆) δ 9.42 (s, 1H), 8.53 (d, 1H), 7.46 (d, 2H), 7.33 (d, 1H), 7.12 (d, 2H), 5.03 (m, 1H), 3.80 (m, 2H), 3.57 (t, 4H), 3.03 (t, 2H), 2.68 (t, 2H), 2.53 (s, 3H), 2.47 (m, 2H), 2.41 (m, 4H), 2.15 (m, 2H), 1.76 (m, 2H); MS (ES) m/z 467 (M+1).

### Example 39

### N-[4-(Methylsulfonyl)phenyl]-4-[2-methyl-1-(tetrahydro-2H-pyran-4-yl)-1H-imidazol-5-yl]pyrimidin-2-amine

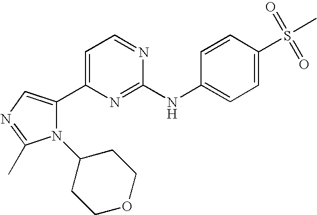

The title compound was prepared in accordance with the general method E using 4-[2-methyl-1-(tetrahydro-2H-pyran-4-yl)-1H-imidazol-5-yl]pyrimidin-2-amine (obtained from Example 39(a)) (60.4 mg, 0.233 mmol), 1-bromo-4-(methylsulfonyl)benzene (54.8 mg, 0.233 mmol), Cs₂CO₃ (152 mg, 0.466 mmol), Pd₂(dba)₃ (5 mg, 0.006 mmol) and X-Phos (7 mg, 0.012 mmol) to give the title compound (48 mg, 50%).

¹H NMR (400 MHz, CDCl₃) δ ppm 8.43 (d, J=5.3 Hz, 1H) 7.96 (s, 1H) 7.78-7.88 (m, 4H) 7.43 (s, 1H) 7.02 (d, J=5.3 Hz, 1H) 5.12-5.24 (m, 1H) 4.04 (dd, J=1.5, 4.4 Hz, 2H) 3.24-3.34 (m, 2H) 3.04 (s, 3H) 2.61 (s, 3H) 2.39-2.53 (m, 2H) 1.85 (dd, J=12.4, 2.8 Hz, 2H); MS (ESI) m/z 414 (M+1).

### Example 39(a) 4-[2-Methyl-1-(tetrahydro-2H-pyran-4-yl)-1H-imidazol-5-yl]pyrimidin-2-amine

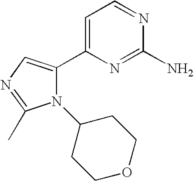

(2E)-3-Dimethylamino-1-[2-methyl-1-(tetrahydro-2H-pyran-4-yl)-1H-imidazol-5-yl]prop-2-en-1-one (described within WO 2005101997) (1.824 g, 6.925 mmol) and guanidine carbonate (3.12 g, 17.31 mmol) were dissolved in n-BuOH (31 mL) and MeONa (1.50 g, 27.70 mmol) was added. The mixture was heated at +125° C. (oil bath temperature) for 20 hours. Saturated NH₄Cl (20 mL) was added and the mixture was extracted with CH₂Cl₂ (3×30 mL). Drying (Na₂SO₄), filtration and concentration gave a crude product which was purified by flash chromatography (CH₂Cl₂/MeOH 25:1→20:1→15:1) to afford a solid (1.3 g, 72%).

¹H NMR (400 MHz, CDCl₃) δ ppm 8.24 (d, J=5.3 Hz, 1H) 7.37 (s, 1H) 6.83 (d, J=5.3 Hz, 1H) 5.23-5.34 (m, 1H) 5.04 (s, 2H) 4.13 (dd, J=11.6, 4.5 Hz, 2H) 3.44-3.54 (m, 2H) 2.60 (s, 3H) 2.43-2.56 (m, 2H) 1.85-1.93 (m, 2H); MS (ESI) m/z 260 (M+1).

### Example 40

### N-{4-[(4-Methylpiperazin-1-yl)sulfonyl]phenyl}-4-[2-methyl-1-(tetrahydro-2H-pyran-4-yl)-1H-imidazol-5-yl]pyrimidin-2-amine

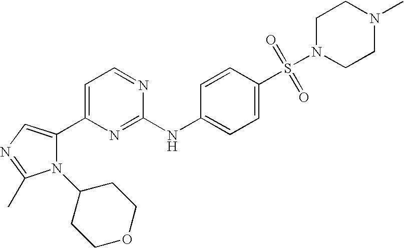

The title compound was prepared in accordance with the general method E using 4-[2-methyl-1-(tetrahydro-2H-pyran-4-yl)-1H-imidazol-5-yl]pyrimidin-2-amine (64.6 mg, 0.249 mmol, obtained Example 39(a)), 1-[(4-bromophenyl)sulfonyl]-4-methylpiperazine (described in WO 2003004472) (79.5 mg, 0.249 mmol), Cs₂CO₃ (162 mg, 0.498 mmol), Pd₂(dba)₃ (6 mg, 0.006 mmol) and X-Phos (7 mg, 0.013 mmol) to give the title compound (54 mg, 43%).

¹H NMR (400 MHz, CDCl₃) δ ppm 8.42 (d, J=5.3 Hz, 1H) 7.89 (s, 1H) 7.75-7.81 (m, 2H) 7.65 (d, J=8.8 Hz, 2H) 7.41 (s, 1H) 7.00 (d, J=5.3 Hz, 1H) 5.12-5.26 (m, 1H) 4.05 (dd, J=1.6, 4.3 Hz, 2H) 3.24-3.36 (m, 2H) 3.02 (s, 4H) 2.61 (s, 3H) 2.37-2.53 (m, 6H) 2.24 (s, 3H) 1.85 (dd, J=12.1, 2.8 Hz, 2H); MS (ESI) m/z 498 (M+1).

### Example 41

### N-{4-[(4-Methylpiperazin-1-yl)carbonyl]phenyl}-4-[2-methyl-1-(tetrahydro-2H-pyran-4-yl)-1H-imidazol-5-yl]pyrimidin-2-amine

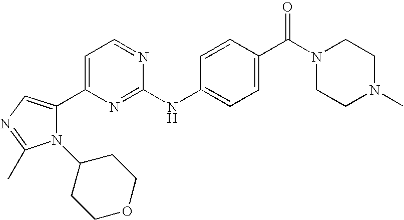

The title compound was prepared in accordance with the general method E using 4-[2-methyl-1-(tetrahydro-2H-pyran-4-yl)-1H-imidazol-5-yl]pyrimidin-2-amine (64.5 mg, 0.249 mmol, obtained Example 39(a)), 1-(4-bromobenzoyl)-4-methylpiperazine (described in WO 2003004472) (70.4 mg, 0.249 mmol), Cs₂CO₃ (162 mg, 0.497 mmol), Pd₂(dba)₃ (6 mg, 0.006 mmol) and X-Phos (7 mg, 0.012 mmol) to give the title compound (42 mg, 37%).

¹H NMR (400 MHz, CDCl₃) δ ppm 8.38 (d, J=5.3 Hz, 1H) 7.69 (s, 1H) 7.62 (d, J=8.6 Hz, 2H) 7.33-7.42 (m, 3H) 6.94 (d, J=5.3 Hz, 1H) 5.17-5.27 (m, 1H) 4.01 (dd, J=11.5, 4.4 Hz, 2H) 3.64 (s, 4H) 3.16-3.32 (m, 2H) 2.60 (s, 3H) 2.33-2.53 (m, 6H) 2.31 (s, 3H) 1.84 (dd, J=12.3, 2.7 Hz, 2H); MS (ESI) m/z 462 (M+1).

### Example 42

### 4-[2-Methyl-1-(tetrahydro-2H-pyran-4-yl)-1H-imidazol-5-yl]-N-[4-(morpholin-4-ylmethyl)phenyl]pyrimidin-2-amine

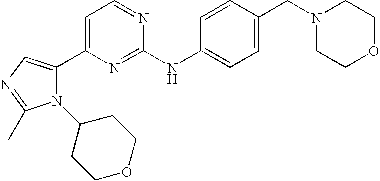

The title compound was prepared in accordance with the general method E using 4-[2-methyl-1-(tetrahydro-2H-pyran-4-yl)-1H-imidazol-5-yl]pyrimidin-2-amine (65.8 mg, 0.254 mmol, obtained Example 39(a)), 4-(4-bromobenzyl)morpholine (65.0 mg, 0.254 mmol), Cs₂CO₃ (165 mg, 0.508 mmol), Pd₂(dba)₃ (6 mg, 0.006 mmol) and X-Phos (7 mg, 0.013 mmol) to give the title compound (24 mg, 21%).

¹H NMR (400 MHz, CDCl₃) δ ppm 8.29 (d, J=5.3 Hz, 1H) 7.42 (d, J=8.3 Hz, 2H) 7.31 (s, 2H) 7.19 (d, J=7.8 Hz, 2H) 6.83 (d, J=5.3 Hz, 1H) 5.16-5.28 (m, 1H) 3.88 (dd, J=1.6, 4.3 Hz, 2H) 3.59-3.68 (m, 4H) 3.39 (s, 2H) 3.03-3.14 (m, 2H) 2.54 (s, 3H) 2.35-2.42 (m, 4H) 2.22-2.35 (m, 2H) 1.76 (dd, J=12.3, 2.4 Hz, 2H); MS (ESI) m/z 435 (M+1).

### Example 43

### 4-[2-methyl-1-(tetrahydro-2H-pyran-4-yl)-1H-imidazol-5-yl]-N-[4-(morpholin-4-ylsulfonyl)phenyl]pyrimidin-2-amine

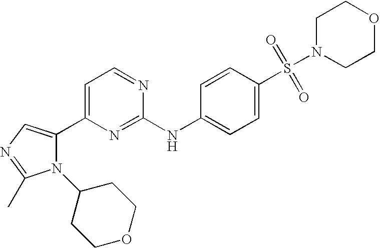

The title compound was prepared in accordance with the general method E using 4-[2-methyl-1-(tetrahydro-2H-pyran-4-yl)-1H-imidazol-5-yl]pyrimidin-2-amine (66.5 mg, 0.256 mmol, obtained Example 39(a)), 4-[(4-bromophenyl)sulfonyl]morpholine (78.5 mg, 0.256 mmol), Cs₂CO₃ (167 mg, 0.513 mmol), Pd₂(dba)₃ (6 mg, 0.006 mmol) and X-Phos (7 mg, 0.013 mmol) to give the title compound (30 mg, 24%).

¹H NMR (400 MHz, CDCl₃) δ ppm 8.44 (d, J=5.3 Hz, 1H) 7.83 (d, J=8.8 Hz, 2H) 7.69 (d, J=8.8 Hz, 2H) 7.64 (s, 1H) 7.45 (s, 1H) 7.03 (d, J=5.3 Hz, 1H) 5.10-5.24 (m, 1H) 4.09 (dd, J=11.5, 4.4 Hz, 2H) 3.69-3.80 (m, 4H) 3.30-3.41 (m, 2H) 2.94-3.07 (m, 4H) 2.63 (s, 3H) 2.44-2.59 (m, 2H) 1.87 (dd, J=12.4, 2.8 Hz, 2H); MS (ESI) m/z 485 (+1).

### Example 44

### N-(4-{[4-(2-Methoxyethyl)piperazin-1-yl]sulfonyl}phenyl)-4-[2-methyl-1-(tetrahydro-2H-pyran-4-yl)-1H-imidazol-5-yl]pyrimidin-2-amine

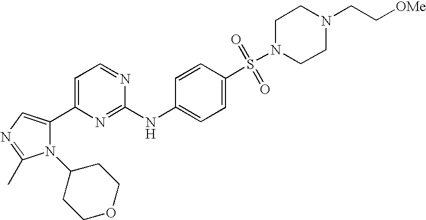

The title compound was prepared in accordance with the general method E using 4-[2-methyl-1-(tetrahydro-2H-pyran-4-yl)-1H-imidazol-5-yl]pyrimidin-2-amine (76.0 mg, 0.293 mmol, obtained from Example 39(a)), 1-[(4-bromophenyl)sulfonyl]-4-(2-methoxyethyl)piperazine (106.5 mg, 0.293 mmol, obtained from Example 44(a)), Cs₂CO₃ (191 mg, 0.586 mmol), Pd₂(dba)₃ (7 mg, 0.007 mmol) and X-Phos (9 mg, 0.015 mmol) to give the title compound (41 mg, 26%).

¹H NMR (400 MHz, CDCl₃) δ ppm 8.43 (d, J=5.3 Hz, 1H) 7.78 (d, J=9.1 Hz, 3H) 7.66 (d, J=8.8 Hz, 2H) 7.42 (s, 1H) 7.01 (d, J=5.3 Hz, 1H) 5.14-5.25 (m, 1H) 4.06 (dd, J=11.5, 4.4 Hz, 2H) 3.43 (t, J=5.3 Hz, 2H) 3.26-3.37 (m, 5H) 3.05 (s, 4H) 2.62 (s, 3H) 2.55 (q, J=5.5 Hz, 6H) 2.40-2.53 (m, 2H) 1.86 (dd, J=12.3, 2.7 Hz, 2H); MS (ESI) m/z 542 (M+1).

### Example 44(a) 1-[(4-Bromophenyl)sulfonyl]-4-(2-methoxyethyl)piperazine

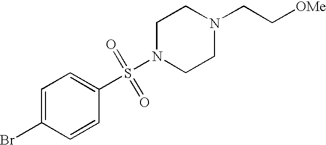

The title compound was prepared in accordance with the general method F using 4-bromobenzenesulfonyl chloride (201.7 mg, 0.789 mmol) and 1-(2-methoxyethyl)piperazine (113.8 mg, 0.789 mmol) to give the title compound (277 mg, 97%).

¹H NMR (400 MHz, CDCl₃) δ ppm 7.64-7.69 (m, 2H) 7.57-7.62 (m, 2H) 3.47 (t, J=5.2 Hz, 2H) 3.30 (s, 3H) 3.08 (s, 4H) 2.62 (d, J=4.8 Hz, 6H); MS (ESI) m/z 364 (M+1).

### Example 45

### N-{4-[(4-Isopropylpiperazin-1-yl)sulfonyl]phenyl}-4-[2-methyl-1-(tetrahydro-2H-pyran-4-yl)-1H-imidazol-5-yl]pyrimidin-2-amine

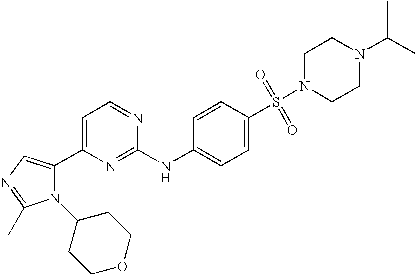

The title compound was prepared in accordance with the general method E using 4-[2-methyl-1-(tetrahydro-2H-pyran-4-yl)-1H-imidazol-5-yl]pyrimidin-2-amine (72.4 mg, 0.279 mmol, obtained from Example 39(a)), 1-[(4-bromophenyl)sulfonyl]-4-isopropylpiperazine (prepared as described in Example 45a) (97.0 mg, 0.279 mmol), Cs₂CO₃ (182.0 mg, 0.559 mmol), Pd₂(dba)₃ (6 mg, 0.007 mmol) and X-Phos (8 mg, 0.014 mmol) to give the title compound (35 mg, 24%).

¹H NMR (400 MHz, CDCl₃) δ ppm 8.43 (d, J=5.1 Hz, 1H) 7.75-7.81 (m, 2H) 7.72 (s, 1H) 7.67 (d, J=8.6 Hz, 2H) 7.43 (s, 1H) 7.02 (d, J=5.1 Hz, 1H) 5.14-5.25 (m, 1H) 4.07 (dd, J=1.5, 4.4 Hz, 2H) 3.27-3.38 (m, 2H) 3.02 (s, 4H) 2.64-2.71 (m, 1H) 2.62 (s, 3H) 2.55-2.61 (m, 4H) 2.41-2.54 (m, 2H) 1.86 (dd, J=12.4, 2.8 Hz, 2H) 0.99 (d, J=6.6 Hz, 6H); MS (ESI) m/z 526 (M+1).

### Example 45(a) 1-[(4-Bromophenyl)sulfonyl]-4-isopropylpiperazine

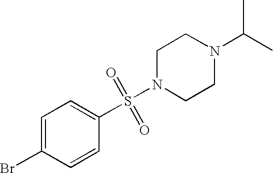

The title compound was prepared in accordance with the general method F using 4-bromobenzenesulfonyl chloride (272.5 mg, 1.067 mmol) and 1-isopropylpiperazine (136.7 mg, 1.067 mmol) to give the title compound (360 mg, 97%).

¹H NMR (400 MHz, CDCl₃) δ ppm 7.64-7.70 (m, 2H) 7.59-7.64 (m, 2H) 3.02 (s, 4H) 2.64-2.72 (m, 1H) 2.55-2.63 (m, 4H) 1.00 (d, J=6.6 Hz, 6H); MS (ESI) m/z 348 (M+1).

### Example 46

### 4-[2-Methyl-1-(tetrahydro-2H-pyran-4-yl)-1H-imidazol-5-yl]-N-[4-(pyrrolidin-1-ylsulfonyl)phenyl]pyrimidin-2-amine

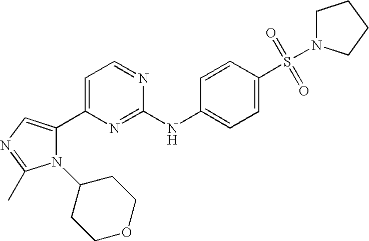

The title compound was prepared in accordance with the general method E using 4-[2-methyl-1-(tetrahydro-2H-pyran-4-yl)-1H-imidazol-5-yl]pyrimidin-2-amine (62.4 mg, 0.241 mmol, obtained from Example 39(a)), 1-[(4-bromophenyl)sulfonyl]pyrrolidine (69.8 mg, 0.241 mmol), Cs₂CO₃ (156.8 mg, 0.481 mmol), Pd₂(dba)₃ (6 mg, 0.006 mmol) and X-Phos (7 mg, 0.012 mmol) to give the title compound (58 mg, 51%).

¹H NMR (400 MHz, CDCl₃) 5 ppm 8.43 (d, J=5.3 Hz, 1H) 7.86 (s, 1H) 7.72-7.81 (m, 4H) 7.42 (s, 1H) 7.00 (d, J=5.3 Hz, 1H) 5.13-5.24 (m, 1H) 4.05 (dd, J=11.5, 4.4 Hz, 2H) 3.26-3.36 (m, 2H) 3.18-3.26 (m, 4H) 2.61 (s, 3H) 2.40-2.53 (m, 2H) 1.85 (dd, J=12.3, 2.7 Hz, 2H) 1.70-1.78 (m, 4H); MS (ESI) m/z 469 (M+1).

### Example 47

### (N-(1-Methylpiperidin-4-yl)-4-({4-[2-methyl-1-(tetrahydro-2H-pyran-4-yl)-1H-imidazol-5-yl]pyrimidin-2-yl}amino)benzenesulfonamide

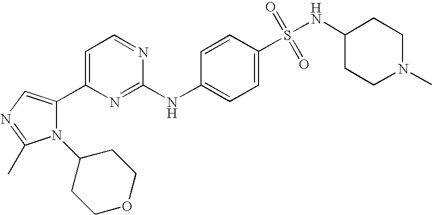

The title compound was prepared in accordance with the general method E using 4-[2-methyl-1-(tetrahydro-2H-pyran-4-yl-1H-imidazol-5-yl]pyrimidin-2-amine (71.5 mg, 0.276 mmol, obtained from Example 39(a)), 4-bromo-N-(1-methylpiperidin-4-yl)benzenesulfonamide (obtained from Example 47(a)) (91.9 mg, 0.276 mmol), Cs₂CO₃ (179.7 mg, 0.552 mmol), Pd₂(dba)₃ (6 mg, 0.007 mmol) and X-Phos (8 mg, 0.014 mmol) to give the title compound (61 mg, 43%).

¹H NMR (400 MHz, CDCl₃) δ ppm 8.44 (d, J=5.3 Hz, 1H) 7.81 (q, J=9.1 Hz, 4H) 7.45 (s, 1H) 7.04 (d, J==5.3 Hz, 1H) 5.12-5.24 (m, 1H) 4.10 (dd, J=11.6, 4.5 Hz, 2H) 3.34 (t, J=1.1 Hz, 2H) 3.00 (s, 2H) 2.62-2.68 (m, 4H) 2.53 (dd, J=12.6, 4.5 Hz, 4H) 2.45 (s, 3H) 2.02 (s, 3H) 1.88 (dd, J=12.9, 3.3 Hz, 2H) some overlap of protons; MS (ESI) m/z 512 (M+1).

### Example 47(a) 4-Bromo-N-(1-methylpiperidin-4-yl)benzenesulfonamide

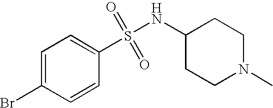

The title compound was prepared in accordance with the general method F using 4-bromobenzenesulfonyl chloride (200.5 mg, 0.785 mmol) and 1-methylpiperidin-4-amine (89.6 mg, 0.785 mmol) to give the title compound (245 mg, 94%).

¹H NMR (400 MHz, CDCl₃) δ ppm 7.72-7.78 (m, 2H) 7.63-7.68 (m, 2H) 3.11-3.22 (m, 1H) 2.70 (d, J=11.6 Hz, 2H) 2.24 (s, 3H) 2.03 (t, J=10.9 Hz, 2H) 1.73-1.83 (m, 2H) 1.44-1.57 (m, 2H); MS (ESI) m/z 334 (M+1).

### Example 48

### N-{4-[(4-Methyl-1,4-diazepan-1-yl)sulfonyl]phenyl}-4-[2-methyl-1-(tetrahydro-2H-pyran-4-yl)-1H-imidazol-5-yl]pyrimidin-2-amine

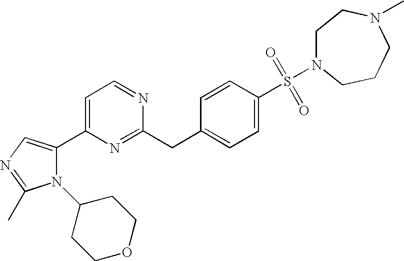

The title compound was prepared in accordance with the general method E using 4-[2-methyl-1-(tetrahydro-2H-pyran-4-yl)-1H-imidazol-5-yl]pyrimidin-2-amine (53.0 mg, 0.204 mmol, obtained from Example 39(a)), 1-[(4-bromophenyl)sulfonyl]-4-methyl-1,4-diazepane (as described in WO 2003004472) (75.6 mg, 0.204 mmol), Cs₂CO₃ (199.8 mg, 0.613 mmol), Pd₂(dba)₃ (5 mg, 0.005 mmol) and X-Phos (6 mg, 0.010 mmol) to give the title compound (15 mg, 15%).

¹H NMR (400 MHz, CDCl₃) δ ppm 8.43 (d, J=5.3 Hz, 1H) 7.69-7.81 (m, 4H) 7.61 (s, 1H) 7.44 (s, 1H) 7.02 (d, J=5.3 Hz, 1H) 5.12-5.24 (m, 1H) 4.08 (dd, J=11.6, 4.5 Hz, 2H) 3.27-3.46 (m, 6H) 2.64-2.73 (m, 4H) 2.63 (s, 3H) 2.44-2.57 (m, 2H) 2.38 (s, 3H) 1.82-1.94 (m, 4H); MS (ESI) m/z 512 (M+1).

### Example 49

### N,N-Diethyl-4-({4-[2-methyl-1-(tetrahydro-2H-pyran-4-yl)-1H-imidazol-5-yl]pyrimidin-2-yl}amino)benzenesulfonamide

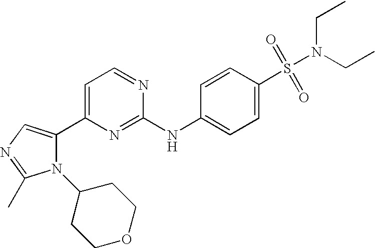

The title compound was prepared in accordance with the general method E using 4-[2-methyl-1-(tetrahydro-2H-pyran-4-yl)-1H-imidazol-5-yl]pyrimidin-2-amine (54.9 mg, 0.212 mmol, obtained from Example 39(a)), 4-bromo-N,N-diethylbenzenesulfonamide (as described in J. Med. Chem., 2000, 43, 3878) (61.9 mg, 0.212 mmol), Cs₂CO₃ (138.0 mg, 0.423 mmol), Pd₂(dba)₃ (5 mg, 0.005 mmol) and X-Phos (6 mg, 0.011 mmol) to give the title compound (69 mg, 69%).

¹H NMR (400 MHz, CDCl₃) 5 ppm 8.41 (d, J=5.3 Hz, 1H) 7.96 (s, 1H) 7.67-7.77 (m, 4H) 7.40 (s, 1H) 6.99 (d, J=5.1 Hz, 1H) 5.13-5.24 (m, 1H) 4.02 (dd, J=11.5, 4.4 Hz, 2H) 3.24-3.33 (m, 2H) 3.21 (q, J=7.2 Hz, 4H) 2.60 (s, 3H) 2.36-2.51 (m, 2H) 1.84 (dd, J=12.4, 2.8 Hz, 2H) 1.11 (t, J=7.2 Hz, 6H); MS (ESI) m/z 471 (M+1).

### Example 50

### N-[4-(Azetidin-1-ylsulfonyl)phenyl]-4-[2-methyl-1-(tetrahydro-2H-pyran-4-yl)-1H-imidazol-5-yl]pyrimidin-2-amine

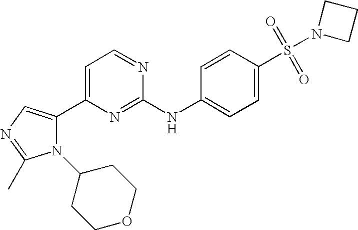

The title compound was prepared in accordance with the general method E using 4-[2-methyl-1-(tetrahydro-2H-pyran-4-yl)-1H-imidazol-5-yl]pyrimidin-2-amine (59.2 mg, 0.228 mmol, obtained from Example 39(a)), 1-[(4-bromophenyl)sulfonyl]azetidine (obtained from Example 50(a)) (63.0 mg, 0.228 mmol), Cs₂CO₃ (148.8 mg, 0.457 mmol), Pd₂(dba)₃ (5 mg, 0.006 mmol) and X-Phos (7 mg, 0.011 mmol) to give the title compound (28 mg, 27%).

¹H NMR (400 MHz, CDCl₃) δ ppm 8.45 (d, J=5.3 Hz, 1H) 7.81-7.88 (m, 2H) 7.74-7.81 (m, 3H) 7.44 (s, 1H) 7.03 (d, J=5.3 Hz, 1H) 5.13-5.25 (m, 1H) 4.08 (dd, J=11.6, 4.5 Hz, 2H) 3.77 (t, J=7.7 Hz, 4H) 3.28-3.39 (m, 2H) 2.63 (s, 3H) 2.43-2.57 (m, 2H) 2.02-2.11 (m, 2H) 1.87 (dd, J=12.4, 2.8 Hz, 2H); MS (ESI) m/z 455 (+1).

### Example 50(a) 1-[(4-Bromophenyl)sulfonyl]azetidine

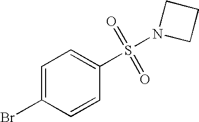

The title compound was prepared in accordance with the general method F using 4-bromobenzenesulfonyl chloride (314.4 mg, 1.230 mmol) and azetidine (70.3 mg, 1.230 mmol) to give the title compound (315 mg, 93%).

¹H NMR (400 MHz, CDCl₃) δ ppm 7.72 (d, J=1.5 Hz, 4H) 3.80 (t, 4H) 2.06-2.17 (m, 2H); MS (ESI) m/z 277 (M+1).

### Example 51

### N-{3-[(4-Methylpiperazin-1-yl)sulfonyl]phenyl}-4-[2-methyl-1-(tetrahydro-2H-pyran-4-yl)-1H-imidazol-5-yl]pyrimidin-2-amine

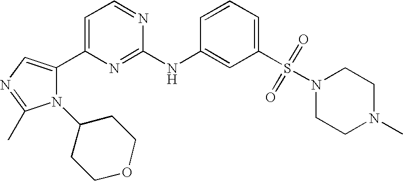

The title compound was prepared in accordance with the general method E using 4-[2-methyl-1-(tetrahydro-2H-pyran-4-yl)-1H-imidazol-5-yl]pyrimidin-2-amine (60.1 mg, 0.232 mmol, obtained from Example 39(a)), 1-[(3-bromophenyl)sulfonyl]-4-methylpiperazine (obtained from Example 51a) (74.0 mg, 0.232 mmol), Cs₂CO₃ (151.0 mg, 0.464 mmol), Pd₂(dba)₃ (5 mg, 0.006 mmol) and X-Phos (7 mg, 0.012 mmol) to give the title compound (65 mg, 57%).

¹H NMR (400 MHz, CDCl₃) δ ppm 8.38 (d, J=5.3 Hz, 1H) 8.00 (s, 1H) 7.96 (s, 1H) 7.86 (d, J=8.6 Hz, 1H) 7.34-7.46 (m, 3H) 6.95 (d, J=5.3 Hz, 1H) 5.12-5.24 (m, 1H) 3.99 (dd, J=11.5, 4.2 Hz, 2H) 3.20 (t, J=11.2 Hz, 2H) 3.03 (s, 4H) 2.59 (s, 3H) 2.30-2.48 (m, 6H) 2.22 (s, 3H) 1.84 (dd, J=12.1, 2.5 Hz, 2H); MS (ESI) m/z 498 (M+1).

### Example 51 (a) 1-[(3-Bromophenyl)sulfonyl]-4-methylpiperazine

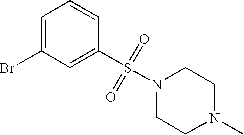

The title compound was prepared in accordance with the general method F using 3-bromobenzenesulfonyl chloride (357.1 mg, 1.398 mmol) and 1-methylpiperazine (153.9 mg, 1.537 mmol) to give the title compound (393 mg, 100%).

¹H NMR (400 MHz, CDCl₃) 5 ppm 7.90 (t, J=1.8 Hz, 1H) 7.71-7.75 (m, 1H) 7.66-7.70 (m, 1H) 7.41 (t, J=8.0 Hz, 1H) 3.06 (s, 4H) 2.45-2.53 (m, 4H) 2.28 (s, 3H); MS (ESI) m/z 320 (M+1).

### Example 52

### N-{3-Chloro-4-[(4-methylpiperazin-1-yl)sulfonyl]phenyl}-4-[2-methyl-1-(tetrahydro-2H-pyran-4-yl)-1H-imidazol-5-yl]pyrimidin-2-amine

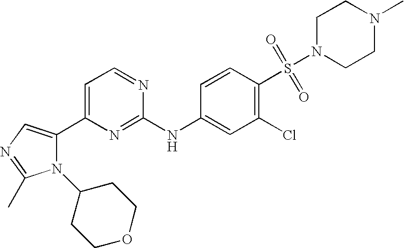

The title compound was prepared in accordance with the general method E using 4-[2-methyl-1-(tetrahydro-2H-pyran-4-yl)-1H-imidazol-5-yl]pyrimidin-2-amine (59.0 mg, 0.228 mmol, obtained from Example 39(a)), 1-[(4-bromo-2-chlorophenyl)sulfonyl]-4-methylpiperazine (obtained from Example 52(a)) (80.5 mg, 0.228 mmol), Cs₂CO₃ (148.3 mg, 0.455 mmol), Pd₂(dba)₃ (5 mg, 0.006 mmol) and X-Phos (7 mg, 0.011 mmol) to give the title compound (59 mg, 49%).

¹H NMR (400 MHz, CDCl₃) δ ppm 8.43 (d, J=5.3 Hz, 1H) 8.05 (s, 1H) 8.00 (d, J=2.3 Hz, 1H) 7.90 (d, J=8.8 Hz, 1H) 7.50 (dd, J=8.7, 2.1 Hz, 1H) 7.42 (s, 1H) 7.03 (d, J=5.3 Hz, 1H) 5.12-5.24 (m, 1H) 4.05 (dd, J=11.5, 4.4 Hz, 2H) 3.22-3.35 (m, 6H) 2.61 (s, 3H) 2.37-2.52 (m, 6H) 2.26 (s, 3H) 1.87 (dd, J=12.4, 2.8 Hz, 2H); MS (ESI) m/z 533 (M+1).

### Example 52(a) 1-[(4-Bromo-2-chlorophenyl)sulfonyl]-4-methylpiperazine

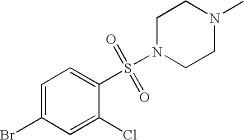

The title compound was prepared in accordance with the general method F using 4-bromo-2-chlorobenzenesulfonyl chloride (326.0 mg, 1.124 mmol) and 1-methylpiperazine (123.9 mg, 1.234 mmol) to give the title compound (406 mg, 100%).

¹H NMR (400 MHz, CDCl₃) δ ppm 7.86 (d, J=8.6 Hz, 1H) 7.67 (d, J=1.8 Hz, 1H) 7.51 (dd, J=8.6, 2.0 Hz, 1H) 3.26-3.34 (m, 4H) 2.42-2.49 (m, 4H) 2.29 (s, 3H); MS (ESI) m/z 354 (M+1).

### Example 53

### N-{3-Methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine

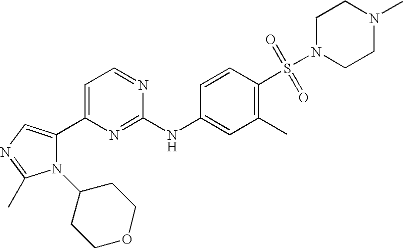

The title compound was prepared in accordance with the general method E using 4-[2-methyl-1-(tetrahydro-2H-pyran-4-yl)-1H-imidazol-5-yl]pyrimidin-2-amine (60.1 mg, 0.232 mmol, obtained from Example 39(a)), 1-[(4-bromo-2-methylphenyl)sulfonyl]-4-methylpiperazine (obtained from Example 53(a)) (77.2 mg, 0.232 mmol), Cs₂CO₃ (151.0 mg, 0.463 mmol), Pd₂(dba)₃ (5 mg, 0.006 mmol) and X-Phos (7 mg, 0.012 mmol) to give the title compound (49 mg, 41%).

¹H NMR (400 MHz, CDCl₃) δ ppm 8.42 (d, J=5.3 Hz, 1H) 7.81 (d, J=8.6 Hz, 1H) 7.63-7.70 (m, 2H) 7.46 (d, J=2.0 Hz, 1H) 7.42 (s, 1H) 6.99 (d, J=5.3 Hz, 1H) 5.13-5.25 (m, 1H) 4.05 (dd, J=11.6, 4.3 Hz, 2H) 3.24-3.35 (m, 2H) 3.11-3.22 (m, 4H) 2.60 (d, J=7.6 Hz, 6H) 2.38-2.53 (m, 6H) 2.27 (s, 3H) 1.84 (dd, J=12.3, 2.7 Hz, 2H); MS (ESI) m/z 512 (M+1).

### Example 53(a) 1-[(4-Bromo-2-methylphenyl)sulfonyl]-4-methylpiperazine

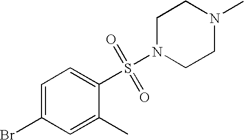

The title compound was prepared in accordance with the general method F using 4-bromo-2-methylbenzenesulfonyl chloride (332.6 mg, 1.234 mmol) and 1-methylpiperazine (135.9 mg, 1.357 mmol) to give the title compound (411 mg, 100%).

¹H NMR (400 MHz, CDCl₃) δ ppm 7.75 (d, J=8.3 Hz, 1H) 7.44-7.52 (m, 2H) 3.23 (s, 4H) 2.61 (s, 3H) 2.49 (s, 4H) 2.32 (s, 3H); MS (ESI) m/z 334 (M+1).

### Example 54

### 5-Fluoro-N-(4-{[(3R)-3-methylmorpholin-4-yl]sulfonyl}phenyl)-4-[2-methyl-1-(tetrahydro-2H-pyran-4-yl)-1H-imidazol-5-yl]pyrimidin-2-amine

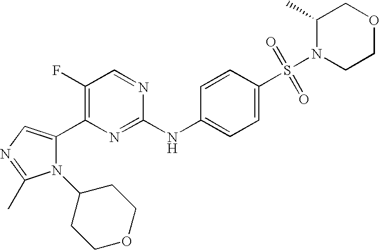

The title compound was prepared in accordance with the general method E using 5-Fluoro-4-[2-methyl-1-(tetrahydro-2H-pyran-4-yl)-1H-imidazol-5-yl]pyrimidin-2-amine (52.0 mg, 0.188 mmol, obtained from Example 7(e)), (3R)-4-[(4-bromophenyl)sulfonyl]-3-methylmorpholine (obtained from Example 54(a)) (60.0 mg, 0.188 mmol), Cs₂CO₃ (122.2 mg, 0.375 mmol), Pd₂(dba)₃ (4 mg, 0.005 mmol) and X-Phos (7 mg, 0.009 mmol) to give the title compound (24 mg, 25%).

¹H NMR (400 MHz, CDCl₃) δ ppm 8.36 (d, J=3.0 Hz, 1H) 7.70-7.77 (m, 5H) 7.68 (d, J=3.8 Hz, 1H) 4.99-5.12 (m, 1H) 4.10 (dd, J=11.7, 4.7 Hz, 2H) 3.92 (q, J=6.7 Hz, 1H) 3.78-3.85 (m, 1H) 3.54-3.63 (m, 2H) 3.40-3.52 (m, 2H) 3.34 (t, J=11.6 Hz, 2H) 3.21-3.30 (m, 1H) 2.65 (s, 3H) 2.48-2.62 (m, 2H) 1.87 (dd, J=12.8, 3.7 Hz, 2H) 1.17 (d, J=6.8 Hz, 3H); MS (ESI) m/z 517 (M+1).

### Example 54(a) (3R)-4-[(4-Bromophenyl)sulfonyl]-3-methylmorpholine

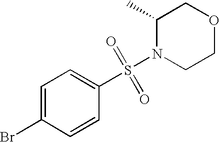

The title compound was prepared in accordance with the general method F using 4-bromobenzenesulfonyl chloride (294.9 mg, 1.154 mmol) and (3R)-3-methylmorpholine (128.4 mg, 1.269 mmol) to give the title compound (368 mg, 99%).

¹H NMR (400 MHz, CDCl₃) δ ppm 7.67 (d, J=2.3 Hz, 4H) 3.80-3.86 (m, 1H) 3.58 (d, J=2.3 Hz, 2H) 3.42-3.50 (m, 2H) 3.21-3.30 (m, 1H) 1.45 (d, J=6.6 Hz, 1H) 1.15 (d, J=6.8 Hz, 3H); MS (ESI) m/z 321 (M+1).

### Example 55

### 5-Fluoro-N-{3-methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine

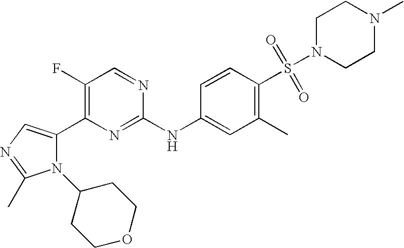

The title compound was prepared in accordance with the general method E using 5-Fluoro-4-[2-methyl-1-(tetrahydro-2H-pyran-4-yl)-1H-imidazol-5-yl]pyrimidin-2-amine (54.6 mg, 0.202 mmol, obtained from Example 7(e)), 1-[(4-bromo-2-methylphenyl)sulfonyl]-4-methylpiperazine (obtained from Example 53(a)) (67.3 mg, 0.202 mmol), Cs₂CO₃ (131.6 mg, 0.404 mmol), Pd₂(dba)₃ (5 mg, 0.005 mmol) and X-Phos (6 mg, 0.010 mmol) to give the title compound (46 mg, 43%).

¹H NMR (400 MHz, CDCl₃) δ ppm 8.34 (d, J=2.8 Hz, 1H) 7.81 (d, J=8.6 Hz, 1H) 7.65 (d, J=3.8 Hz, 1H) 7.57-7.63 (m, 2H) 7.42 (d, J=2.0 Hz, 1H) 5.03-5.14 (m, 1H) 4.07 (dd, J=11.6, 4.5 Hz, 2H) 3.26-3.37 (m, 2H) 3.11-3.23 (m, 4H) 2.63 (s, 3H) 2.59 (s, 3 H) 2.39-2.57 (m, 6H) 2.27 (s, 3H) 1.85 (dd, J=12.1, 3.0 Hz, 2H); MS (ESI) m/z 530 (M+1).

### Example 56

### 5-Fluoro-N-(4-{[(1S,4S)-2,5-methyl-2,5-diazabicyclo[2.2.1]hept-2-yl]sulfonyl}phenyl)-4-[2-methyl-1-(tetrahydro-2H-pyran-4-yl)-1H-imidazol-5-yl]pyrimidin-2-amine

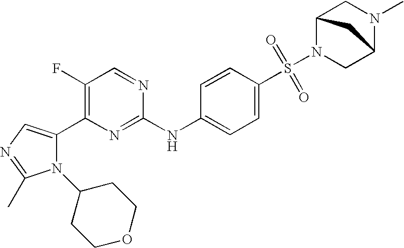

The title compound was prepared in accordance with the general method E using 5-Fluoro-4-[2-methyl-1-(tetrahydro-2H-pyran-4-yl)-1H-imidazol-5-yl]pyrimidin-2-amine (53.3 mg, 0.197 mmol, obtained from Example 7(e)), (1S,4S)-2-[(4-bromophenyl)sulfonyl]-5-methyl-2,5-diazabicyclo[2.2.1]heptane (obtained from Example 56(a)) (65.3 mg, 0.197 mmol), Cs₂CO₃ (128.5 mg, 0.394 mmol), Pd₂(dba)₃ (5 mg, 0.005 mmol) and X-Phos (6 mg, 0.010 mmol) to give the title compound (60 mg, 57%).

¹H NMR (400 MHz, CDCl₃) 5 ppm 8.34 (d, J=2.8 Hz, 1H) 7.88 (s, 1H) 7.73 (s, 4H) 7.64 (d, J=3.8 Hz, 1H) 5.00-5.12 (m, 1H) 4.22 (s, 1H) 4.06 (dd, J=11.6, 4.5 Hz, 2H) 3.53 (d, J=9.9 Hz, 1H) 3.27-3.37 (m, 3H) 3.00 (dd, J=9.6, 2.3 Hz, 1H) 2.83 (dd, J=9.9, 2.5 Hz, 1H) 2.65 (s, 1H) 2.62 (s, 3H) 2.43-2.57 (m, 2H) 2.33 (s, 3H) 1.85 (dd, J=12.5, 3.4 Hz, 2H) 1.67 (d, J=9.9 Hz, 1H) 1.12 (d, J=10.1 Hz, 1H); MS (ESI) m/z 528 (M+1).

### Example 56(a) (1S,4S)-2-[(4-Bromophenyl)sulfonyl]-5-methyl-2,5-diazabicyclo[2.2.1]heptane

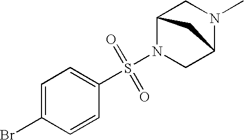

The title compound was prepared in accordance with the general method F using 4-bromobenzenesulfonyl chloride (309.0 mg, 1.209 mmol), (1S,4S)-2-methyl-2,5-diazabicyclo[2.2.1]heptane hydrobromide (364.5 mg, 1.330 mmol) and also adding Et₃N (367.1 mg, 3.628 mmol) to give the title compound (400 mg, 100%).

¹H NMR (400 MHz, CDCl₃) δ ppm 7.65-7.73 (m, 4H) 4.27 (s, 1H) 3.56 (dd, J=9.6, 1.3 Hz, 1H) 3.36 (s, 1H) 3.02 (dd, J=9.6, 2.3 Hz, 1H) 2.86 (dd, J=9.9, 2.5 Hz, 1H) 2.65 (dd, J=10.0, 1.1 Hz, 1H) 2.36 (s, 3H) 1.74 (d, J=9.9 Hz, 1H) 1.17 (d, J=9.9 Hz, 1H); MS (ESI) m/z 332 (M+1).

### Example 57

### 4-({5-Fluoro-4-[2-methyl-1-(tetrahydro-2H-pyran-4-yl)-1H-imidazol-5-yl]pyrimidin-2-yl}amino)-N,N-dimethylbenzenesulfonamide

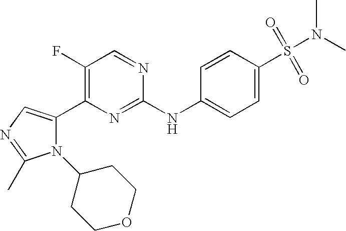

The title compound was prepared in accordance with the general method E using 5-Fluoro-4-[2-methyl-1-(tetrahydro-2H-pyran-4-yl)-1H-imidazol-5-yl]pyrimidin-2-amine (54.1 mg, 0.200 mmol, obtained from Example 7(e)), 4-bromo-N,N-dimethylbenzenesulfonamide (52.9 mg, 0.200 mmol), Cs₂CO₃ (130.4 mg, 0.400 mmol), Pd₂(dba)₃ (5 mg, 0.005 mmol) and X-Phos (6 mg, 0.010 mmol) to give the title compound (44 mg, 47%).

¹H NMR (400 MHz, CDCl₃) δ ppm 8.35 (d, J=2.8 Hz, 1H) 7.82 (s, 1H) 7.73-7.78 (m, 2H) 7.67-7.71 (m, 2H) 7.65 (d, J=3.8 Hz, 1H) 5.03-5.13 (m, 1H) 4.07 (dd, J=11.6, 4.5 Hz, 2H) 3.28-3.38 (m, 2H) 2.69 (s, 6H) 2.64 (s, 3H) 2.44-2.58 (m, 2H) 1.86 (dd, J=12.1, 3.0 Hz, 2H); MS (ESI) m/z 461 (M+1).

### Example 58

### N-[4-(Azetidin-1-ylsulfonyl)phenyl]-5-fluoro-4-[2-methyl-1-(tetrahydro-2H-pyran-4-yl)-1H-imidazol-5-yl]pyrimidin-2-amine

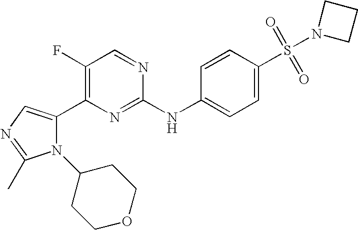

The title compound was prepared in accordance with the general method E using 5-Fluoro-4-[2-methyl-1-(tetrahydro-2H-pyran-4-yl)-1H-imidazol-5-yl]pyrimidin-2-amine (54.3 mg, 0.201 mmol, obtained from Example 7(e)), 1-[(4-bromophenyl)sulfonyl]azetidine (55.5 mg, 0.201 mmol, obtained from Example 50(a)), Cs₂CO₃ (130.9 mg, 0.402 mmol), Pd₂(dba)₃ (5 mg, 0.005 mmol) and X-Phos (6 mg, 0.010 mmol) to give the title compound (38 mg, 40%).

¹H NMR (400 MHz, CDCl₃) δ ppm 8.36 (d, J=3.0 Hz, 1H) 7.85 (s, 1H) 7.73-7.83 (m, 4H) 7.66 (d, J=3.8 Hz, 1H) 5.00-5.17 (m, 1H) 4.08 (dd, J=1.6, 4.8 Hz, 2H) 3.76 (t, J=7.7 Hz, 4H) 3.28-3.40 (m, 2H) 2.64 (s, 3H) 2.44-2.59 (m, 2H) 2.01-2.13 (m, 2H) 1.87 (dd, J=12.3, 3.2 Hz, 2H); MS (ESI) m/z 473 (M+1).

### Example 59

### Methyl 3-{[4-(1,2-dimethyl-1H-imidazol-5-yl)-5-fluoropyrimidin-2-yl]amino}benzoate

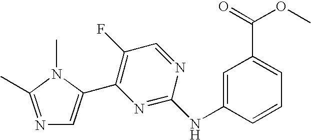

2-Chloro-4-(1,2-dimethyl-1H-imidazol-5-yl)-5-fluoropyrimidine (49 mg, 0.22 mmol, 1.0 equiv., obtained from Example 1(b)), methyl-3-aminobenzoate (38 mg, 0.25 mmol, 1.15 equiv.) and Cs₂CO₃ (0.11 g, 0.33 mmol, 1.5 equiv.) were mixed in 1,4-dioxane (2 mL) and the mixture was flushed with argon for 10 minutes. Pd₂(dba)₃ (11 mg, 0.012. mmol, 054 equiv.) and X-Phos (11 mg, 0.022 mmol, 0.10 equiv.) were added and the reaction mixture was flushed with argon for another 10 minutes before the reaction was stirred for 16 h at +90° C. under an atmosphere of Argon. The reaction mixture was diluted with CH₂Cl₂, filtered and evaporated. The residue was taken up in CH₂Cl₂ and the organic phase was washed with H₂O. Residual water was removed from the organic phase by addition of absolute EtOH before evaporation. The crude of the product was purified by flash chromatography (gradient from 100% CH₂Cl₂ to 5% MeOH in CH₂Cl₂) to yield a solid (48 mg, 60%).

¹H NMR (400 MHz, DMSO-d₆) δ ppm 9.81 (s, 1H) 8.54 (d, 1H) 8.34 (t, 1H) 7.92 (dd, 1H) 7.59-7.52 (m, 2H) 7.43 (t, 1H) 3.93 (s, 3H) 3.85 (s, 3H) 2.41 (s, 3H); MS (ESI) m/z 340 (M−1).

### Example 60

### 3-[[4-(2,3-Dimethylimidazol-4-yl)-5-fluoro-pyrimidin-2-yl]amino]-N-(3-methoxypropyl)benzamide hydrochloride

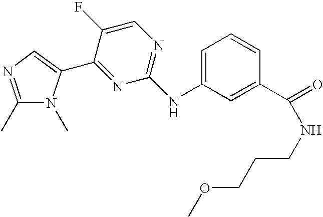

The title compound was prepared in accordance with the general method G using flash chromatography (gradient from 100% EtOAc to 5% MeOH in EtOAc) for purification. Using methyl 3-{[4-(1,2-dimethyl-1H-imidazol-5-yl)-5-fluoropyrimidin-2-yl]amino}benzoate (44.5 mg, 0.13 mmol, obtained from Example 59), Al(CH₃)₃ (94 mg, 1.3 mmol, 2.0 M in toluene) and 3-methoxypropan-1-amine (68.9 mg, 0.78 mmol), the base of the title compound (26 mg, 46%) was obtained as a solid. The hydrochloride was prepared in accordance with the method described within general method D.

¹H NMR (400 MHz, DMSO-d₆) δ ppm 9.97 (s, 1H) 8.76 (d, 1H) 8.45-8.38 (m, 1H) 8.19 (d, 1H) 8.13 (t, 1H) 7.83-7.76 (m, 1H) 7.47 (d, 1H) 7.39 (t, 1H) 4.02 (s, 3H) 3.39-3.29 (m, 4H) 3.24 (s, 3H) 2.67 (s, 3H) 1.80-1.71 (m, 2H); MS (ESI) m/z 399 (M+1).

### Example 61

### [4-[[4-(2,3-Dimethylimidazol-4-yl)-5-fluoro-pyrimidin-2-yl]amino]-2-(trifluoromethoxy)phenyl]-(4-methylpiperazin-1-yl)methanone hydrochloride

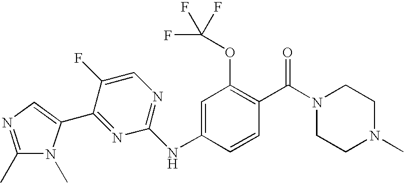

The title compound was prepared in accordance with the general method G using flash chromatography (gradient from 100% dichloromethane to 10% MeOH in dichloromethane) for purification. Using methyl 4-{[4-(1,2-dimethyl-1H-imidazol-5-yl)-5-fluoropyrimidin-2-yl]amino}-2-(trifluoromethoxy)benzoate (obtained from Example 61(b)) (33 mg, 0.078 mmol), Al(CH₃)₃ (56 mg, 0.78 mmol, 2.0 M in toluene) and 1-methylpiperazine (47 mg, 0.47 mmol), the base of the title compound (18 mg, 40%) was obtained as a solid. The hydrochloride was prepared in accordance with th method described within general method D.

¹H NMR (400 MHz, DMSO-d₆) δ ppm 10.34 (s, 1H) 8.82 (d, 1H) 8.16 (d, 1H) 7.91 (s, 1 E11) 7.83 (dd, 1H) 7.49 (d, 1H) 4.03 (s, 3H) 3.09-2.87 (m, 4H) 2.80 (s, 3H) 2.66 (s, 3H); MS (ESI) m/z 493 (M+1).

### Example 61(a) Methyl 4-bromo-2-(trifluoromethoxy)benzoate

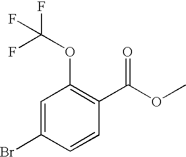

4-Bromo-1-iodo-2-(trifluoromethoxy)benzene (0.340 g, 0.93 mmol), Pd(OAc)₂ (0.011 g, 0.049 mmol), dppp (0.020 g, 0.048 mmol) and triethylamine (0.218 g, 2.15 mmol) were suspended in MeOH (10 mL) in a 300 mL glass vessel. The vessel was evacuated and filled with nitrogen gas (repeated 3 times) followed by evacuation and filling with CO gas (repeated 2 times) to establish a homogenous CO gas atmosphere at 3.5 bar. Heating in an oil bath at +65° C. for 90 minutes resulted in 50% conversion of the start material as juged by GCMS. After addition of more Pd(OAc)₂ (0.009 g, 0.040 mmol), dppp (0.018 g, 0.044 mmol) and triethylamine (0.58 g, 0.57 mmol) CO gas atmosphere was established as described above and the reaction was continued at +65° C. for another 130 minutes. When the mixture was cool (r.t) it was filtered through diatomaceous earth and the solvent was evaporated. The crude product was purified by flash chromatography (gradient from 100% heptane to 20% EtOAc in heptane) to give the title compound as a clear liquid (0.068 g, 24.5%).

¹H NMR (400 MHz, DMSO-d₆) δ ppm 7.92-7.85 (m, 1H) 7.84-7.76 (m, 2H) 3.85 (s, 3H); MS (CI) m/z 299 (79Br) (M+1).

### Example 61 (b) Methyl 4-{[4-(1,2-dimethyl-1H-imidazol-5-yl)-5-fluoropyrimidin-2-yl]amino}-2-(trifluoromethoxy)benzoate

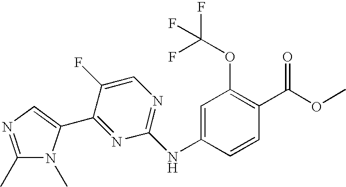

The title compound was prepared in accordance with the general method E (workup procedure A) with the exception that purification of the crude product was done by flash chromatography (gradient from 100% heptane to 100% EtOAc). Using 4-(1,2-Dimethyl-1H-imidazol-5-yl)-5-fluoropyrimidin-2-amine (obtained from Example 25(a)) (38 mg, 0.18 mmol), methyl 4-bromo-2-(trifluoromethoxy)benzoate (obtained from Example 61(a)) (64 mg, 0.21 mmol), Cs₂CO₃ (90 mg, 0.28 mmol), Pd₂(dba)₃ (8 mg, 0.009 mmol) and X-Phos (8 mg, 0.017 mmol), the title compound (33 mg, 42%) was obtained as a solid.

¹H NMR (400 MHz, DMSO-d₆) δ ppm 10.25 (s, 1H) 8.63 (d, 1H) 7.98-7.82 (m, 3H) 7.58 (d, 1H) 3.95 (s, 3H) 3.81 (s, 3H) 2.42 (s, 3H); MS (ESI) m/z 426 (M+1).

### Example 62

### N-[4-(Azetidin-1-ylcarbonyl)phenyl]-5-fluoro-4-[2-methyl-1-(tetrahydro-2H-pyran-4-yl)-1H-imidazol-5-yl]pyrimidin-2-amine hydrochloride

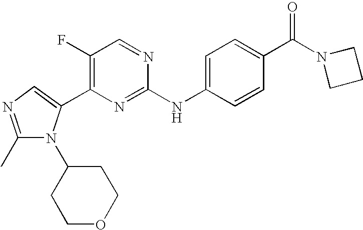

The title compound was prepared in accordance with the general method E (Workup procedure C), with the exception that the base of the product was purified by flash chromatography (gradient from 100% DCM to 5% MeOH in DCM). Using 5-fluoro-4-[2-methyl-1-(tetrahydro-2H-pyran-4-yl)-1H-imidazol-5-yl]pyrimidin-2-amine (obtained from Example 7(e)) (53 mg, 0.19 mmol), 1-(4-chlorobenzoyl)azetidine (J. Org. Chem., 1974, 39(13), 1973) (39 mg, 0.20 mmol), Cs₂CO₃ (95 mg, 0.29 mmol), Pd₂(dba)₃ (8 mg, 0.009 mmol) and X-Phos (10 mg, 0.02 mmol), the base of the title compound (52 mg, 62%) was obtained as a solid. The hydrochloride was prepared in accordance with the method described in general method D.

¹H NMR (400 MHz, DMSO-d₆) δ ppm 10.09 (s, 1H) 8.81 (d, 1H) 8.02 (s, 1H) 7.69 (d, 2H) 7.56 (d, 2H) 5.05-4.90 (m, 1H) 4.40-4.18 (m, 2H) 4.12-3.91 (m, 2H) 3.81 (dd, 2H) 3.16 (t, 2H) 2.78 (s, 3H) 2.30-2.07 (m, 4H) 1.92 (dd, 2H); MS (ESI) m/z 437 (M+1).

### Example 63

### N-{4-[(3,3-Difluoroazetidin-1-yl)carbonyl]phenyl}-5-fluoro-4-[2-methyl-1-(tetrahydro-2H-pyran-4-yl)-1H-imidazol-5-yl]pyrimidin-2-amine hydrochloride

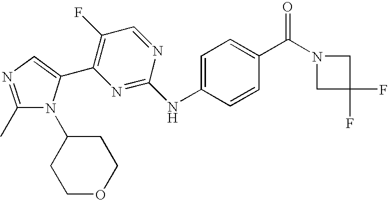

The title compound was prepared in accordance with the general method E (Workup procedure C), with the exception that the base of the product was purified by flash chromatography (gradient from 100% CH₂Cl₂ to 5% MeOH in CH₂Cl₂). Using 5-fluoro-4-[2-methyl-1-(tetrahydro-2H-pyran-4-yl)-1H-imidazol-5-yl]pyrimidin-2-amine (obtained from Example 7(e)) (49 mg, 0.18 mmol), 1-(4-chlorobenzoyl)-3,3-difluoroazetidine (44 mg, 0.19 mmol), Cs₂CO₃ (104 mg, 0.32 mmol), Pd₂(dba)₃ (9 mg, 0.010 mmol) and X-Phos (10 mg, 0.02 mmol), the base of the title compound (68 mg, 64%) was obtained as a solid. The hydrochloride was prepared in accordance with the general method D.

¹H NMR (400 MHz, DMSO-d₆) δ ppm 10.15 (s, 1H) 8.83 (d, 1H) 8.02 (s, 1H) 7.74 (d, 2H) 7.63 (d, 2H) 5.03-4.91 (m, 1H) 5.0-4.2 (m, 4H) 3.82 (dd, 2H) 3.18 (t, 2H) 2.78 (s, 3H) 2.24-2.08 (m, 2H) 1.99-1.85 (m, 2H); MS (ESI) m/z 473 (M+1).

### Example 63(a) 4-Bromo-2-(trifluoromethoxy)benzoic acid

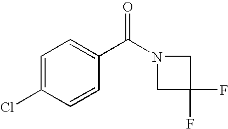

Thionyl chloride (5 mL) was added to 4-chloro-benzoic acid (0.49 g, 3.1 mmol). After addition of 2 drops of anhydrous DMF, the reaction mixture was refluxed for 15 minutes under an atmosphere of nitrogen. The solvent was evaporated in vacuo and the residue was dissolved in CH₂Cl₂ (5 mL). 3,3-difluoroazetidine hydrochloride (0.42 g, 3.3 mmol)) was added followed by addition of triethylamine (0.91 mL, 6.6 mmol). The reaction mixture was stirred at r.t. for 15 minutes before it was diluted with CH₂Cl₂, washed with i) saturated NaHCO₃ (aq.) and ii) water. To the organic phase Abs. (absolute) EtOH was added (until a clear solution was obtained) and the solvents were evaporated i/7 vacuo to give the title compound as a solid in 94% yield. The isolated material was used in the next step without further purification.

¹H NMR (400 MHz, DMSO-d₆) δ ppm 7.75-7.66 (m, 2H) 7.58-7.48 (m, 2H) 5.06-4.15 (m, 4H); MS (ESI) m/z 232 (M+1).

### Example 64

### 5-Fluoro-N-[3-methyl-4-(morpholin-4-ylmethyl)phenyl]-4-[2-methyl-1-(tetrahydro-2H-pyran-4-yl)-1H-imidazol-5-yl]pyrimidin-2-amine hydrochloride

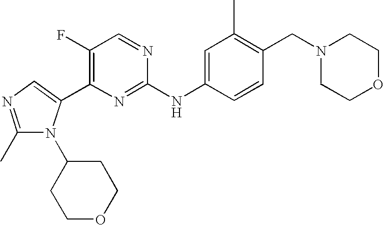

The title compound was prepared in accordance with the general method E (Workup procedure C), with the exception that the base of the product was purified by flash chromatography (gradient from 100% CH₂Cl₂ to 6% MeOH in CH₂Cl₂) before final purification by preparative HPLC. Using 5-fluoro-4-[2-methyl-1-(tetrahydro-2H-pyran-4-yl)-1H-imidazol-5-yl]pyrimidin-2-amine (obtained from Example 7(e)) (70 mg, 0.25 mmol), 4-(4-chloro-2-methylbenzyl)morpholine (obtained from Example 64(a)) (60 mg, 0.27 mmol), Cs₂CO₃ (136 mg, 0.42 mmol), Pd₂(dba)₃ (13 mg, 0.014 mmol) and X-Phos (15 mg, 0.031 mmol), the base of the title compound was prepared and transformed into the hydrochloride in accordance with the method described within general method D to yield (67 mg, 53%).

¹H NMR (400 MHz, DMSO-d₆) δ ppm 10.54 (br s, 1H) 9.90 (s, 1H) 8.79 (d, 1H) 8.00 (s, 1H) 7.63-7.50 (m, 2H) 7.48 (d, 1H) 5.05-4.90 (m, 1H) 4.27 (br. s., 2H) 3.98-3.75 (m, 6H) 3.11-3.24 (m, 6H, partly obscured by HDO signal) 2.78 (s, 3H) 2.38 (s, 3H) 2.24-2.07 (m, 2H) 1.92 (dd, 2H); MS (ESI) m/z 465 (M−1).

### Example 64(a) 4-(4-Chloro-2-methylbenzyl)morpholine

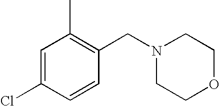

To a stirred, cooled (0° C.) solution of 4-chloro-2-methylbenzaldehyde (0.23 g, 1.5 mmol) in MeOH (5 mL) was added morpholine (0.15 g, 1.7 mmol), NaCNBH₃ (0.49 g, 7.8 mmol) and HOAc (0.063 g, 1.0 mmol) and the reaction was stirred at r.t. over night. The solvent was removed in vacuo and the crude product was partitioned between EtOAc/1M NaHCO₃ (aq.). The organic phase was dried (Na₂SO₄), filtered, concentrated and purified twice by flash chromatography (gradient from 100% pentane to 10% EtOAc in pentane) to give the title compound as a clear liquid (0.120 g, 35%).

¹H NMR (400 MHz, DMSO-d₆) δ ppm 7.27-7.21 (m, 2H) 7.20-7.14 (m, 1H) 3.57-3.49 (m, 4H) 3.38 (s, 2H) 2.36-2.31 (m, 4H) 2.31 (s, 3H); MS (ESI) m/z 226 (M+1).

### Example 65

### 5-Fluoro-N-[4-(morpholin-4-ylmethyl)phenyl]-4-[3-oxan-4-yl-2-(trifluoromethyl)imidazol-4-yl]-pyrimidin-2-amine hydrochloride

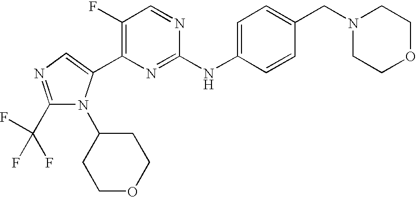

The title compound was prepared in accordance with the general method E and work-up procedure B. The product was purified by flash chromatography (CH₂Cl₂/MeOH 20:1). Using 5-Fluoro-4-[1-(tetrahydro-2H-pyran-4-yl)-2-(trifluoromethyl)-1H-imidazol-5-yl]pyrimidin-2-amine (obtained from Example 34(d)) (38 mg, 0.115 mmol), 4-(4-bromobenzyl)-morpholine (0.028 g, 0.11 mmol), Cs₂CO₃ (75 mg, 0.23 mmol), Pd₂(dba)₃ (8 mg, 0.0086 mmol) and X-Phos (8.2 mg, 0.017 mmol), the base of the title compound (42 mg, 76%) was obtained as a solid. The hydrochloride was prepared in accordance with the method described in general method D.

¹H NMR (400 MHz, DMSO-d₆) δ 9.74 (s, 1H), 8.73 (d, 1H), 7.55-7.53 (m, 3H), 7.21 (d, 2H), 4.81 (m, 1H), 3.78 (m, 2H), 3.55 (t, 4H), 3.39 (s, 2H), 3.18 (t, 2H), 2.32 (m, 4H), 2.13 (m, 2H), 1.86 (m, 2H); MS (ES) m/z 505 (M−1).

### Example 66

### 5-Fluoro-N-{4-[(4-fluoropiperidin-1-yl)carbonyl]phenyl}-4-[2-methyl-1-(tetrahydro-2H-pyran-4-yl)-1H-imidazol-5-yl]pyrimidin-2-amine hydrochloride

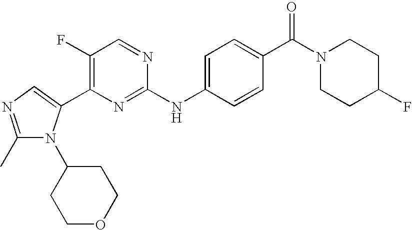

To a solution of lithium 4-({5-fluoro-4-[2-methyl-1-(tetrahydro-2H-pyran-4-yl)-1H-imidazol-5-yl]pyrimidin-2-yl}amino)benzoate (obtained from Example 66(a)) (50 mg, 0.12 mmol) in anhydrous DMF (0.5 mL) was added a solution of HBTU (56 mg, 0.15 mmol) in DMF (0.5 mL) and the mixture was shaken for 1 h at r.t. 4-Fluoropiperidine hydrochloride (22 mg, 0.16 mmol) was then added followed by the addition of DIPEA (65 mg, 0.50 mmol) and the reaction mixture was shaken o.n. (over night) at r.t. The crude of the base product was purified using preparative HPLC and was transferred into the hydrochloride in accordance with the method described within general method D to yield the title compound (37 mg, 54%) as a solid.

¹H NMR (400 MHz, DMSO-d₆) δ ppm 10.02 (s, 1H) 8.80 (d, 1H) 8.02 (s, 1H) 7.66 (d, 2H) 7.35 (d, 2H) 4.79-5.04 (m, 2H) 3.81 (dd, 2H) 3.57 (br.s., 2H) 3.14 (t, 2H) 2.78 (s, 3H) 2.23-2.07 (m, 2H) 1.99-1.79 (m, 4H) 1.71 (br.s., 2H); MS (ESI) m/z 483 (M+1).

### Example 66(a) Lithium 4-({5-fluoro-4-[2-methyl-1-(tetrahydro-2H-pyran-4-yl)-1H-imidazol-5-yl]pyrimidin-2-yl}amino)benzoate

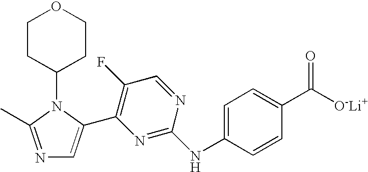

Ethyl 4-({5-fluoro-4-[2-methyl-1-(tetrahydro-2H-pyran-4-yl)-1H-imidazol-5-yl]pyrimidin-2-yl}amino)benzoate (obtained from Example 67) (1.16 g, 2.73 mmol) and LiOH x H₂O (115 mg, 2.74 mmol) were slurried in EtOH (15 mL) and H₂O (4.4 mL). The slurry was heated at +50-60° C. under an atmosphere of Argon for 20 h then the reaction mixture was allowed to stand for 6 days at r.t. The solvents were then evaporated and the residue was slurried in THF/H₂O 9:1 and heated at +60° C. for 24 h. No more conversion of the ester was seen (LCMS). LiOH x H₂O (59 mg, 1.16 mmol) was added in two portions and the slurry was heated at +60° C. for ˜20 h. Removal of the solvents in vacuo gave the title compound as a solid (1.17 g). The isolated material was used in amidation reactions without further purification.

¹H NMR (400 MHz, DMSO-d₆) δ ppm 9.49 (s, 1H) 8.54 (d, 1H) 7.75 (d, 2H) 7.45 (d, 2H) 7.32 (d, 1H) 5.11-4.98 (m, 1H) 3.78 (dd, 2H) 3.05 (t, 2H) 2.53 (s, 3H) 2.24-2.08 (m, 2H) 1.78 (dd, 2H); MS (ESI) m/z 398 (M+1).

### Example 67

### Ethyl 4-({5-fluoro-4-[2-methyl-1-(tetrahydro-2H-pyran-4-yl)-1H-imidazol-5-yl]pyrimidin-2-yl}amino)benzoate

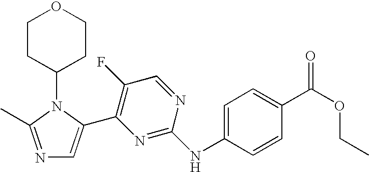

The title compound was prepared in accordance with the general method E (Workup procedure C), with the exception that the base of the product was purified by flash chromatography (gradient from 100% CH₂Cl₂ to 5% MeOH in CH₂Cl₂). Using 5-fluoro-4-[2-methyl-1-(tetrahydro-2H-pyran-4-yl)-1H-imidazol-5-yl]pyrimidin-2-amine (obtained from Example 7(e)) (794 mg, 2.86 mmol), ethyl 4-iodobenzoate (820 mg, 2.97 mmol), Cs₂CO₃ (1.48 g, 4.54 mmol), Pd₂(dba)₃ (59 mg, 0.064 mmol) and X-Phos (63 mg, 0.13 mmol), the title compound (1.16 g, 95%) was obtained as a solid.

¹H NMR (400 MHz, DMSO-d₆) δ ppm 9.97 (s, 1H) 8.64 (d, 1H) 7.86 (d, 2H) 7.76 (d, 2H) 7.35 (d, 1H) 5.08-4.96 (m, 1H) 4.27 (q, 2H) 3.81 (dd, 2H) 3.12 (t, 2H) 2.54 (s, 3H) 2.27-2.11 (m, 2H) 1.82 (dd, 2H) 1.30 (t, 3H); MS (ESI) m/z 426 (M+1).

### Example 68

### N,N-Diethyl-4-({5-fluoro-4-[2-methyl-1-(tetrahydro-2H-pyran-4-yl)-1H-imidazol-5-yl]pyrimidin-2-yl}amino)benzamide hydrochloride

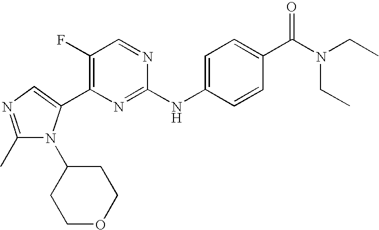

To a solution of lithium 4-({5-fluoro-4-[2-methyl-1-(tetrahydro-2H-pyran-4-yl)-1H-imidazol-5-yl]pyrimidin-2-yl}amino)benzoate (obtained from Example 66(a)) (50 mg, 0.12 mmol) in anhydrous DMF (0.5 mL) was added a solution of HBTU (56 mg, 0.15 mmol) in DMF (0.5 mL) and the mixture was shaken for 1 h at r.t. Diethyl amine (13 mg, 0.18 mmol) was then added followed by the addition of DIPEA (48 mg, 0.37 mmol) and the reaction mixture was shaken over night. at r.t. The crude of the base product was purified using preparative HPLC and was transferred into the hydrochloride in accordance with the method described in the general method D to yield the title compound (34 mg, 56%) as a solid.

¹H NMR (400 MHz, DMSO-d₆) δ ppm 9.99 (s, 1H) 8.81 (d, 1H) 8.03 (s, 1H) 7.66 (d, 2H) 7.29 (d, 2H) 5.05-4.92 (m, 1H) 3.82 (dd, 2H) 3.16 (t, 3H) 2.78 (s, 3H) 2.24-2.08 (m, 2H) 1.97-1.86 (m, 2H) 1.10 (t, 6H); MS (ESI) m/z 453 (M+1).

### Examples 69-91

The following Examples were prepared according to the procedure described in Examples 66 and 68 except that the quantity of DIPEA used in each case was adjusted depending on whether the starting amine was a free base, mono- or higher salt. 3 equivalents of DIPEA were used for amines that were freebases and one additional equivalent was added for every additional salt. The group R is an amine connected via the nitrogen to form an amide.

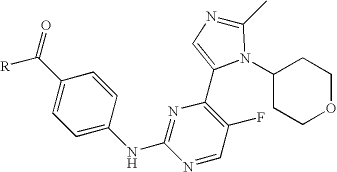

| Ex | R | NMR | Yield | M/z |
|---|---|---|---|---|
| 69 | 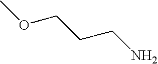 | ¹H NMR (400 MHz, DMSO-d₆)δ ppm 9.78 (s, 1 H) 8.61 (d, 1 H)8.26 (t, 1 H) 7.80-7.72 (m, 2 H) 7.70-7.63 (m, 2 H) 7.34 (d, 1H) 5.08-4.97 (m, 1 H) 3.81 (dd,2 H) 3.23 (s, 3 H) 3.08 (t, 2 H)2.54 (s, 3 H) 2.25-2.11 (m, 2 H)1.80 (dd, 2 H) 1.78-1.69 (m, 2 H) | 67% | 469 |
| 70 | 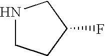 | 9.80 (s, 1 H) 8.61 (d, 1 H) 7.68(d, 2 H) 7.55-7.43 (m, 2 H)7.34 (d, 1 H) 5.33 (dd, 1 H)5.08-4.94 (m, 1 H) 3.90-3.50(m, 6-7 H uncertain integral)3.17-3.04 (m, 2 H) 2.54 (s, 3 H)2.25-1.95 (m, 4 H) 1.86-1.75 (m, 2 H) | 67% | 469 |
| 71 | 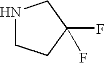 | 9.82 (s, 1 H) 8.61 (d, 1 H) 7.70(d, 2 H) 7.50 (d, 2 H) 7.34 (d, 1H) 5.07-4.94 (m, 1 H) 3.89 (t, 2H) 3.82 (dd, 2 H) 3.71 (t, 2 H)3.11 (t, 2 H)2.54 (s, 3H) 2.5-2.37(m, 2 H) 2.24-2.11 (m, 2 H) 1.81(dd, 2 H) | 73% | 487 |
| 72 | 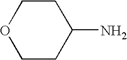 | 9.78 (s, 1 H) 8.61 (d, 1 H) 8.08(d, 1 H) 7.81-7.75 (m, 2 H)7.71-7.64 (m, 2 H) 7.34 (d, 1H) 5.07-4.95 (m, 1 H) 4.04-3.92(m, 1 H) 3.91-3.84 (m, 2 H)3.81 (dd, 2 H) 3.10 (t, 2 H)2.54 (s, 3 H) 2.25-2.11 (m, 2 H)1.86-1.69 (m, 4 H) 1.63-1.50(m, 2 H). 27 of 29 signalsreported, signals obscured by HDO | 48% | 481 |
| 73 | 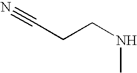 | 9.78 (s, 1 H) 8.60 (d, 1 H) 7.68(d, 2 H) 7.38-7.30 (m, 3 H)5.07-4.95 (m, 1 H) 3.82 (dd, 2H) 3.63 (br. s., 2 H) 3.11 (t, 2 H)2.84 (t, 2 H) 2.53 (s, 3 H) 2.24-2.11(m, 2 H) 1.80 (dd, 2 H) | 55% | 464 |
| 74 | 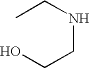 | 9.72 (s, 1 H) 8.59 (d, 1 H) 7.64(d, 2 H) 7.33 (d, 1 H) 7.30 (d, 2H) 5.06-4.94 (m, 1 H) 4.75 (t, 1H) 3.83 (dd, 2 H) 3.53 (br. s., 2H) 3.11 (t, 2 H) 2.53 (s, 3 H)2.24-2.10 (m, 2 H) 1.80 (dd, 2H) 1.15-1.00 (m,3 H) | 33% | 469 |
| 75 | 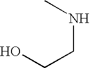 | 9.74 (s, 1 H) 8.60 (d, 1 H) 7.64(d, 2 H)7.39-7.28 (m, 3 H)5.07-4.94 (m, 1 H) 4.81-4.71(m, 1 H) 3.82 (dd, 2 H) 3.54 (br.s., 2 H) 3.11 (t, 2 H) 2.96 (s, 3H) 2.54 (s, 3 H) 2.24-2.10 (m, 2H) 1.80 (dd, 2 H) | 35% | 455 |
| 76 | 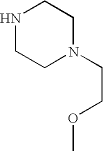 | 9.76 (s, 1 H) 8.59 (d, 1 H) 7.69-7.63(m, 2 H) 7.36-7.28 (m, 3H) 5.06-4.94 (m, 1 H) 3.81 (dd,2 H) 3.55-3.40 (m, 6 Huncertain integral) 3.22 (s, 3 H)3.09 (t, 2 H) 2.53 (s, 3 H) 2.42(br. s., 3 H uncertain integral)2.24-2.10 (m, 2 H) 1.79 (dd, 2 H) | 64% | 524 |
| 77 | 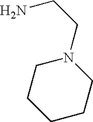 | 9.79 (s, 1 H) 8.61 (d, 1 H) 8.23-8.12(m, 1 H) 7.79-7.71 (m, 2H) 7.70-7.64 (m, 2 H) 7.34 (d,1 H) 5.08-4.97 (m, 1 H) 3.81(dd, 2 H) 3.08 (t, 2 H) 2.54 (s, 3H) 2.38 (br s, 4 H uncertainintegral) 2.24-2.11 (m, 2 H)1.80 (dd, 2 H) 1.55-1.43 (m, 4H) 1.42-1.31 (m, 2 H) | 73% | 508 |
| 78 | 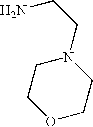 | 9.80 (s, 1 H) 8.61 (d, 1 H) 8.24-8.14(m, 1 H) 7.79-7.71 (m, 2H) 7.70-7.63 (m, 2 H) 7.34 (d,1 H) 5.08-4.97 (m, 1 H) 3.81(dd, 2 H) 3.62-3.51 (m, 4 H)3.07 (t, 2 H) 2.54 (s, 3 H)2.5-2.34 (m, 6 H uncertain integral)2.24-2.11 (m, 2 H) 1.80 (dd, 2 H) | 56% | 510 |
| 79 | 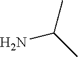 | 9.76 (s, 1 H) 8.61 (d, 1 H) 7.99(d, 1 H) 7.77 (d, 2 H) 7.66( d, 2H) 7.35 (d, 1 H) 5.07-4.95 (m,1 H) 4.14-4.01 (m, 1 H) 3.81(dd, 2 H) 3.10 (t, 2 H) 2.54 (s, 3H) 2.25-2.10 (m, 2 H) 1.80 (dd,2 H) 1.15 (d, 6 H) | 51% | 439 |
| 80 | 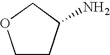 | 9.79 (s, 1 H) 8.61 (d, 1 H) 8.30(d, 1 H) 7.79 (d, 2 H) 7.68 (d, 2H) 7.34 (d, 1 H) 5.07-4.95 (m,1 H) 4.48-4.39 (m, 1 H)3.89-3.76 (m, 4 H) 3.74-3.66 (m, 1H) 3.56 (dd, 1 H) 3.10 (t, 2 H)2.54 (s, 3 H) 2.25-2.08 (m, 3 H)1.95-1.85 (m, 1 H)1.80 (dd, 2 H) | 51% | 467 |
| 81 | 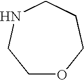 | 9.74 (s, 1 H) 8.60 (d, 1 H) 7.66(d, 2 H) 7.37-7.26 (m, 3 H)5.07-4.94 (m, 1 H) 3.82 (dd, 2H) 3.78-3.55 (m, 6 H) 3.50(br.s., 2 H) 3.10 (t, 2 H) 2.53 (s,3 H) 2.24-2.10 (m, 2 H) 1.80(dd, 2H) 1.91-1.67 (m, 2 H) | 68% | 481 |
| 82 | 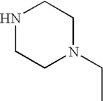 | 9.76 (s, 1 H) 8.59 (d, 1 H) 7.66(d, 2 H) 7.36-7.27 (m, 3 H5.06-4.94 (m, 1 H) 3.81 (dd, 2H) 3.47 (m, 4 H) 3.09 (t, 2 H)2.53 (s, 3 H) 2.43-2.26 (m, 6 H)2.24-2.10 (m, 2 H) 1.79 (dd, 2H) 0.99 (t, 3 H) | 68% | 494 |
| 83 | 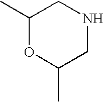 | 9.78 (s, 1 H) 8.60 (d, 1 H) 7.67(d, 2 H) 7.38-7.27 (m, 3 H)5.07-4.96 (m, 1 H) 3.81 (dd, 2H) 3.57-3.47 (m, 2 H) 3.08 (t, 2H) 2.53 (s, 3 H) 2.24-2.10 (m, 2H) 1.79 (dd, 2 H) 1.06 (br. s., 6 H) | 44% | 495 |
| 84 | 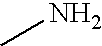 | 9.79 (s, 1 H) 8.61 (d, 1 H) 8.23(q, 1 H) 7.78-7.72 (m, 2 H)7.69-7.64 (m, 2 H) 7.34 (d, 1H) 5.08-4.96 (m, 1 H) 3.81 (dd,2 H) 3.08 (t, 2 H) [2.76 (s, 1.5 H)2.75 (s, 1.5 H)] rot. 2.54 (s, 3 H)2.24-2.11 (m, 2 H) 1.80 (dd, 2 H | 23% | 411 |
| 85 | 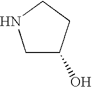 | 9.77 (s, 1 H) 8.60 (d, 1 H) 7.66(d, 2 H) 7.50-7.40 (m, 2 H)7.33 (d, 1 H) 5.07-4.95 (m, 1H) 4.95 (dd, 1 H) 4.35-4.18 (m,1 H) 3.88-3.76 (m, 2 H) 3.65-3.20(multiplets partly obscuredby HDO-signal, no reliableintegral) 3.11 (t, 2 H) 2.54 (s, 3 H)2.25-2.10 (m, 2 H) 1.99-1.72 (m, 4 H) | 36% | 467 |
| 86 | 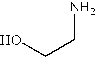 | 9.79 (s, 1 H) 8.61 (d, 1 H) 8.24(t, 1 H) 7.81-7.75 (m, 2 H)7.71-7.65 (m, 2 H) 7.35 (d, 1 H)5.07-4.96 (m, 1 H) 4.70 (t, 1 H)3.82 (dd, 2 H) 3.53-3.45 (m, 3H) 3.10 (t, 2 H) 2.54 (s, 3 H)2.25-2.11 (m, 2 H) 1.81 (dd, 2 H) | 53% | 441 |
| 87 | 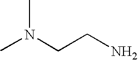 | 9.79 (s, 1 H) 8.61 (d, 1 H) 8.19(t, 1 H) 7.79-7.72 (m, 2 H)7.70-7.64 (m, 2 H) 7.34 (d, 1 H)5.08-4.96 (m, 1 H) 3.81 (dd, 2H) 3.09 (t, 2 H) 2.54 (s, 3 H)2.47-2.38 (m, 2 H) 2.26-2.11(m, 8 H) 1.80 (dd, 2 H) | 60% | 468 |
| 88 | 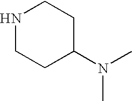 | 9.75 (s, 1 H) 8.59 (d, 1 H) 7.65(d, 2 H) 7.38-7.25 (m, 3 H)5.07-4.94 (m, 1 H) 3.81 (dd, 2H) 3.09 (t, 2 H) 2.90 (br. s., 2 H)2.53 (s, 3 H) 2.30-2.09 (m, 8 H)1.86-1.67 (m, 4 H) 1.43-1.26(m, 2 H) | 67% | 508 |
| 89 | 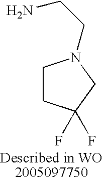 | 9.80 (s, 1 H) 8.61 (d, 1 H) 8.27(t, 1 H) 7.80-7.72 (m, 2 H)7.71-7.64 (m, 2 H) 7.35 (d, 1 H)5.07-4.97 (m, 1 H) 3.81 (dd, 2H) 3.08 (t, 2 H) 2.93 (t, 2 H)2.73 (t, 2 H) 2.59 (t, 2 H) 2.54 (s,H) 2.27-2.10 (m, 4 H) 1.80(dd, 2 H) | 71% | 530 |
| 90 | 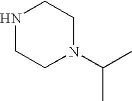 | 9.76 (s, 1 H) 8.60 (d, 1 H) 7.66(d, 2 H) 7.36-7.27 (m, 3 H)5.06-4.94 (m, 1 H) 3.81 (dd, 2H) 3.45 (br s not possible tointegrate) 3.09 (t, 2 H) 2.53 (s, 3H) 2.42 (br. s., 4 H) 2.24-2.10(m, 2 H) 1.79 (dd, 2 H) 0.97 (s, 3H) 0.96 (s, 3 H). Two broadoverlapping signals could not beintegrated accurately | 65% | 508 |
| 91 | 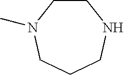 | 9.73 (s, 1 H) 8.59 (d, 1 H) 7.65(d, 2 H) 7.33 (d, 1 H) 7.3 (d, 2H) 5.06-4.95 (m, 1 H) 3.81 (dd,2 H) 3.58 (br.s., 2 H) 3.43 (br.s., 2 H uncertain integral) 3.09 (t,2 H) 2.63 (br. s., 1 H uncertainintegral) 2.53 (s, 3 H) 2.33-2.10(m, 5 H) 1.88- 1.69 (m, 4 H) | 66% | 494 |

### Example 92

### 5-Fluoro-N-[4-(methylsulfonyl)phenyl]-4-[1-(tetrahydro-2H-pyran-4-yl)-2-(trifluoromethyl)-1H-imidazol-5-yl]pyrimidin-2-amine

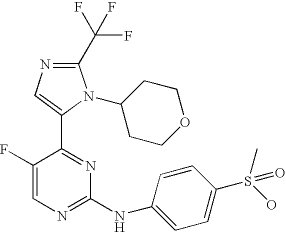

The title compound was prepared in accordance with the general method E using 5-fluoro-4-[1-(tetrahydro-2H-pyran-4-yl)-2-(trifluoromethyl)-1H-imidazol-5-yl]pyrimidin-2-anine (66 mg, 0.2 mmol, obtained from Example 34(d)) and 4-bromophenyl methyl sulfone (47 mg, 0.2 mmol) to give the title compound (43 mg, 44%).

¹H NMR (400 MHz, CDCl₃) δ ppm 8.46 (d, J=2.27 Hz, 1H) 7.76-7.87 (m, 4H) 7.63 (d, J=3.03 Hz, 1H) 4.72-4.85 (m, 1H) 4.04 (dd, J=11.87, 4.80 Hz, 2H) 3.37-3.49 (m, 2H) 3.02 (s, 3H) 2.56-2.73 (m, 2H); MS (ESI) m/z 486 (M+1).

### Example 93

### N-[4-(Azetidin-1-ylcarbonyl)phenyl]-5-fluoro-4-[1-(tetrahydro-2H-pyran-4-yl)-2-(trifluoromethyl)-1H-imidazol-5-yl]pyrimidin-2-amine

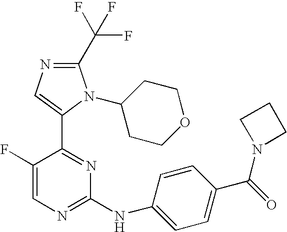

The title compound was prepared in accordance with the general method E using 5-fluoro-4-[1-(tetrahydro-2H-pyran-4-yl)-2-(trifluoromethyl)-1H-imidazol-5-yl]pyrimidin-2-amine (66 mg, 0.2 mmol, obtained from Example 34(d)) and 1-(4-bromobenzoyl)azetidine (obtained from 93(a)) (48 mg, 0.2 mmol) to give the title compound (48 mg, 49%).

¹H NMR (400 MHz, CDCl₃) δ ppm 8.46 (d, J=2.53 Hz, 1H) 7.71 (d, J=3.03 Hz, 1H) 7.57-7.69 (m, 4H) 4.80-4.92 (m, 1H) 4.30 (d, J=44.21 Hz, 4H) 4.08 (dd, J=11.62, 4.80 Hz, 2H) 2.64-2.79 (m, 2H) 2.30-2.44 (m, 2H) 1.86 (d, J=8.84 Hz, 2H); MS (ESI) m/z 491 (M+1).

### Example 93 (a) 1-(4-Bromobenzoyl)azetidine

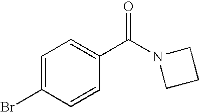

Azetidine (275 mg, 4.82 mmol) followed by Et₃N (0.66 mL, 4.8 mmol) was added dropwise to 4-bromobenzoyl chloride (1.0 g, 4.56 mmol) in DCM (10 mL). The mixture was stirred 30 min before it was diluted with CH₂Cl₂, washed with saturated NaHCO₃ (aq.), water, dried (Na₂SO₄), filtered and concentrated in vacuo. The crude product was purified by flash chromatography (heptan to Heptan:EtOAc 1:4) to give the title compound (765 mg, 70%) as a solid.

¹H NMR (400 MHz, CDCl₃) δ ppm 7.47-7.58 (m, 4H) 4.26 (t, J=7.83 Hz, 4H) 2.29-2.43 (m, 2H); MS (ESI) m/z 240 (M+1).

### Example 94

### N-[4-(Azetidin-1-ylcarbonyl)-3-chlorophenyl]-4-(1,2-dimethyl-1H-imidazol-5-yl)-5-fluoropyrimidin-2-amine

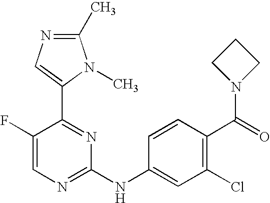

The title compound was prepared in accordance with the general method E using 4-(1,2-dimethyl-1H-imidazol-5-yl)-5-fluoropyrimidin-2-amine (26 mg, 0.125 mmol, obtained from Example 25(a)) and 1-(4-bromo-2-chlorobenzoyl)azetidine (obtained from Example 94(a)) (35 mg, 0.127 mmol) to give the title compound (17 mg, 34%).

¹H NMR (400 MHz, CDCl₃) δ ppm 8.46 (d, J=2.53 Hz, 1H) 7.71 (d, J=3.03 Hz, 1H) 7.57-7.69 (m, 4H) 4.80-4.92 (m, 1H) 4.30 (d, J=44.21 Hz, 4H) 4.08 (dd, J=111.62, 4.80 Hz, 2H) 3.42 (t, J=12.00 Hz, 2H) 2.64-2.79 (m, 2H) 2.30-2.44 (m, 2H) 1.86 (d, J=8.84 Hz, 2H); MS (ESI) m/z 491 (M+1).

### Example 94(a) 1-(4-Bromo-2-chlorobenzoyl)azetidine

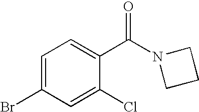

The title compound was prepared in accordance with the general method H using 4-bromo-2-chlorobenzoic acid (0.75 g, 3.19 mmol) and azetidine (192 mg, 3.36 mmol) to give the title compound (800 mg, 91%).

¹H NMR (400 MHz, CDCl₃) δ ppm 7.58 (d, J=2.02 Hz, 1H) 7.45 (dd, J=8.08, 1.77 Hz, 1H) 7.22 (d, J=8.08 Hz, 1H) 4.22 (t, J=7.83 Hz, 2H) 3.97 (t, 2H) 2.28-2.41 (m, 2H); MS (ESI) m/z 274 (M+1).

### Example 95

### N-[4-(Azetidin-1-ylcarbonyl)-3-methylphenyl]-4-(1,2-dimethyl-1H-imidazol-5-yl)-5-fluoropyrimidin-2-amine

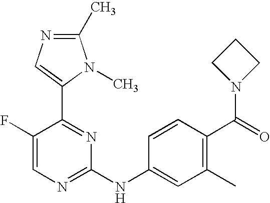

The title compound was prepared in accordance with the general method E using 4-(1,2-dimethyl-1H-imidazol-5-yl)-5-fluoropyrimidin-2-amine (30 mg, 0.145 mmol, obtained from Example 25(a)) and 1-(4-bromo-2-methylbenzoyl)azetidine (obtained from Example 95(a)) (37 mg, 0.145 mmol) to give the title compound (32 mg, 58%).

¹H NMR (400 MHz, CDCl₃) δ ppm 8.27 (d, J=3.03 Hz, 1H) 7.75 (d, J=4.29 Hz, 1H) 7.36-7.45 (m, 2H) 7.18-7.26 (m, 2H) 4.21 (t, J=7.58 Hz, 2H) 3.98 (t, J=7.58 Hz, 2H) 3.93 (s, 3H) 2.48 (s, 3H) 2.25-2.36 (m, 2H); MS (ESI) m/z 381 (M+1).

### Example 95(a) 1-(4-Bromo-2-methylbenzoyl)azetidine

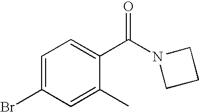

The title compound was prepared in accordance with the general method H using 4-bromo-2-methylbenzoic acid (1.0 g, 3.93 mmol) and azetidine (225 mg, 3.94 mmol) to give the title compound (670 mg, 67%).

¹H NMR (400 MHz, CDCl₃) δ ppm 7.38-7.41 (m, 1H) 7.33 (dd, J=8.08, 1.52 Hz, 1H) 7.11 (d, J=8.08 Hz, 1H) 4.06 (d, J=97.01 Hz, 4H) 2.38 (s, 3H) 2.27-2.36 (m, 2H); MS (ESI) m/z 254 (M+1).

### Example 96

### 4-(1,2-Dimethyl-1H-imidazol-5-yl)-5-fluoro-N-[4-(methylsulfonyl)phenyl]pyrimidin-2-amine

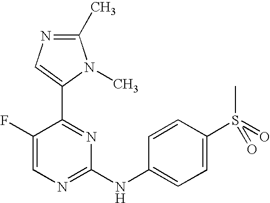

The title compound was prepared in accordance with the general method E using 4-(1,2-dimethyl-1H-imidazol-5-yl)-5-fluoropyrimidin-2-amine (40 mg, 0.193 mmol, obtained from Example 25(a)) and 4-bromophenyl methyl sulfone (47 mg, 0.20 mmol) to give the title compound (21 mg, 30%).

¹H NMR (400 MHz, CDCl₃) δ ppm 8.32 (d, J=3.03 Hz, 1H) 7.84-7.91 (m, 2H) 7.75-7.82 (m, 3H) 7.70 (s, 1H) 3.95 (s, 3H) 3.06 (s, 3H) 2.50 (s, 3H); MS (ESI) m/z 362 (M+1).

### Example 97

### N-[3-Chloro-4-(methylsulfonyl)phenyl]-4-(1,2-dimethyl-1H-imidazol-5-yl)-5-fluoropyrimidin-2-amine

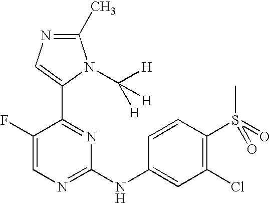

The title compound was prepared in accordance with the general method E using 4-(1,2-dimethyl-1H-imidazol-5-yl)-5-fluoropyrimidin-2-amine (40 mg, 0.193 mmol, obtained from Example 25(a)) and 4-bromo-2-chlorophenyl methyl sulfone (obtained from 97(a)) (54 mg, 0.20 mmol) to give the title compound (21 mg, 27%).

¹H NMR (400 MHz, CDCl₃) δ ppm 8.34 (d, J=3.03 Hz, 1H) 8.10 (d, J=2.02 Hz, 1H) 8.03 (d, J=8.84 Hz, 1H) 7.85 (s, 1H) 7.79 (d, J=4.55 Hz, 1H) 7.49 (dd, J=8.72, 2.15 Hz, 1H) 3.98 (s, 3H) 3.27 (s, 3H) 2.51 (s, 3H); MS (ESI) m/z 396 (M+1).

### Example 97(a) 4-Bromo-2-chlorophenyl methyl sulfone

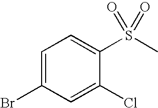

4-Bromo-2-chlorobenzenesulfonyl chloride (960 mg, 3.3 mmol) was added in portion to a solution of Na₂SO₃ (460 mg, 3.6 mmol) and NaHCO₃ (555 mg, 6.6 mmol) in H₂O (5 mL) at +75° C. After 2 h. at +75° C. the reaction mixture was allowed to reach r.t. and MeI (1 mL, 16 mmol) was added. The mixture was heated in a microwave oven (+100° C., 2 min).

The reaction mixture was cooled to r.t. and diluted with CH₂Cl₂. The organic phase was washed with H₂O, dried (Na₂SO₄), filtered and concentrated in vacuo to give the title compound (450 mg, 50%)

¹H NMR (400 MHz, CDCl₃) δ ppm 8.02 (d, J=8.59 Hz, 1H) 7.75 (d, J=1.77 Hz, 1H) 7.64 (dd, J=8.59, 1.77 Hz, 1H) 3.27 (s, 3H);

MS (ESI) m/z 268 (M⁺).

### Example 98

### N-{3-Chloro-4-[(4-methylpiperazin-1-yl)sulfonyl]phenyl}-4-(1,2-dimethyl-1H-imidazol-5-yl)-5-fluoropyrimidin-2-amine

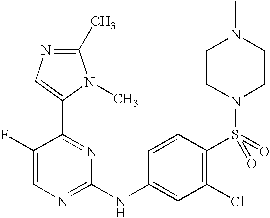

The title compound was prepared in accordance with the general method E using 4-(1,2-dimethyl-1H-imidazol-5-yl)-5-fluoropyrimidin-2-amine (40 mg, 0.193 mmol, obtained from Example 25(a)) and 1-[(4-bromo-2-chlorophenyl)sulfonyl]-4-methylpiperazine (obtained from example 52(a)) (70 mg, 0.198 mmol) to give the title compound (29 mg, 31%).

¹H NMR (400 MHz, CDCl₃) δ ppm 8.34 (d, J=3.03 Hz, 1H) 8.05 (d, J=2.02 Hz, 1H) 7.95 (d, J=8.84 Hz, 1H) 7.80 (d, J=4.55 Hz, 1H) 7.49 (s, 1H) 7.43 (dd, J=8.84, 2.27 Hz, 1H) 3.99 (s, 3H) 3.26-3.35 (m, 4H) 2.52 (s, 3H) 2.44-2.49 (m, 4H) 2.30 (s, 3H); MS (ESI) m/z 480 (M+1).

### Example 99

### 4-(1,2-Dimethyl-1H-imidazol-5-yl)-5-fluoro-N-{3-methyl-4-[(4-methylpiperazin-1-yl)sulfonyl]phenyl}pyrimidin-2-amine

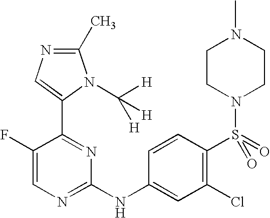

The title compound was prepared in accordance with the general method E using 4-(1,2-dimethyl-1H-imidazol-5-yl)-5-fluoropyrimidin-2-amine (40 mg, 0.193 mmol, obtained from Example 25(a)) and 1-[(4-bromo-2-methylphenyl)sulfonyl]-4-methylpiperazine (obtained from Example 53(a)) (66 mg, 0.198 mmol) to give the title compound (32 mg, 36%).

¹H NMR (400 MHz, CDCl₃) δ ppm 8.34 (d, J=3.03 Hz, 1H) 8.05 (d, J=2.02 Hz, 1H) 7.95 (d, J=8.84 Hz, 1H) 7.80 (d, J=4.55 Hz, 1H) 7.49 (s, 1H) 7.43 (dd, J=8.84, 2.27 Hz, 1H) 3.99 (s, 3H) 3.26-3.37 (m, 4H) 2.52 (s, 3H) 2.44-2.50 (m, 4H) 2.30 (s, 3H); MS (ESI) n7/z 460 (M+1).

### Example 100

### N-[4-(Azetidin-1-ylcarbonyl)-3-(trifluoromethoxy)phenyl]-4-(1,2-dimethyl-1H-imidazol-5-yl)-5-fluoropyrimidin-2-amine

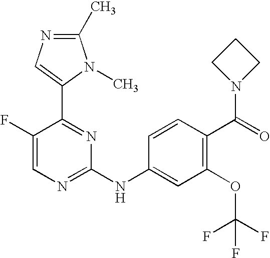

The title compound was prepared in accordance with the general method E using 4-(1,2-dimethyl-1H-imidazol-5-yl)-5-fluoropyrimidin-2-amine (40 mg, 0.193 mmol, obtained from Example 25(a)) and 1-[4-bromo-2-(trifluoromethoxy)benzoyl]azetidine (obtained from Example 100(c)) (64 mg, 0.197 mmol) to give the title compound (29 mg, 34%).

¹H NMR (400 MHz, CDCl₃) δ ppm 8.31 (d, J=3.03 Hz, 1H) 7.72-7.79 (m, 2H) 7.65 (s, 1H) 7.45 (d, J=1.01 Hz, 2H) 4.21 (t, J=7.71 Hz, 2H) 4.05 (t, J=7.71 Hz, 2H) 2.49 (s, 3H) 2.27-2.37 (m, 2H); MS (ESI) m/z 451 (M+1).

### Example 100(a) Methyl 4-bromo-2-(trifluoromethoxy)benzoate

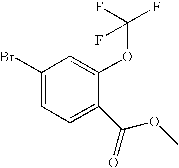

To 4-bromo-1-iodo-2-(trifluoromethoxy)benzene (2.0 g, 5.45 mmol), Pd(OAc)₂ (121 mg, 0.54 mmol), dppp (222 mg, 0.54 mmol) and Et₃N (2.3 mL, 16.3 mmol) in MeOH (50 mL) was introduced CO (g) to a pressure of 2.5 bar. The mixture was stirred at 2.5 bar and at +65° C. for 4 h. The mixture was filtered through diatomaceous earth and the residue was concentrated in vacuo. The crude product was purified by flash chromatography (Heptan to Heptane:EtOAc 4:1) to give the title compound (900 mg, 55%) as a liquid.

¹H NMR (400 MHz, CDCl₃) δ ppm 7.86 (d, J=8.34 Hz, 1H) 7.55 (dd, J=8.46, 1.89 Hz, I H) 7.50-7.53 (m, 1H) 3.94 (s, 3H); MS (ESI) m/z 298 (M)

### Example 100(b) 4-Bromo-2-(trifluoromethoxy)benzoic acid

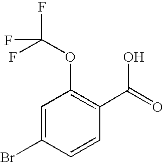

LiOH monohydrate (75 mg, 1.79 mmol) was added to methyl 4-bromo-2-(trifluoromethoxy)benzoate (400 mg, 1.34 mmol, obtained from 100(a)) in THF: H₂O (9:1, 5 mL). The mixture was heated in a microwave oven (+120° C., 10 min). The reaction mixture was cooled to r.t. and diluted with CH₂Cl₂ and H₂O. 2 M HCl was added until pH 1. The mixture was extracted and the water phase was re-extracted with CH₂Cl₂. The organic phases were combined, dried (Na₂SO₄), filtered and concentrated in vacuo to give the title compound (300 mg, 79%) as a solid.

¹H NMR (400 MHz, CDCl₃) δ ppm 7.99 (d, J=8.34 Hz, 1H) 7.59 (dd, J=8.34, 1.77 Hz, 1H) 7.55-7.57 (m, J=1.26 Hz, 1H); MS (ESI) m/z 283 (M−1).

### Example 100(c) 1-[4-Bromo-2-(trifluoromethoxy)benzoyl]azetidine

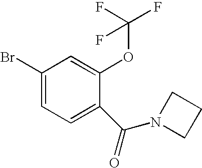

The title compound was prepared in accordance with the general method H using 4-bromo-2-(trifluoromethoxy)benzoic acid (300 mg, 1.05 mmol, obtained from Example 100(b)) and azetidine (70 mg, 1.22 mmol) to give the title compound (200 mg, 59%).

¹H NMR (400 MHz, CDCl₃) δ ppm 7.50 (dd, J=8.21, 1.64 Hz, 1H) 7.45-7.48 (m, 1H) 7.38 (d, J=8.08 Hz, 1H) 4.21 (t, J=7.71 Hz, 2H) 4.00 (t, J=7.58 Hz, 2H) 2.34 (dd, 18H);

MS (ESI) m/z 324 (M+1).

### Example 101

### 5-Fluoro-N-[4-(4-methylpiperazin-1-yl)sulfonylphenyl]-4-[3-methyl-2-(trifluoromethyl)imidazol-4-yl]-pyrimidin-2-amine hydrochloride

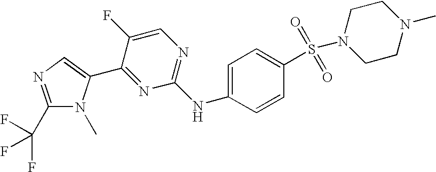

The title compound was prepared in accordance with the general method E using 5-Fluoro-4-[1-methyl-2-(trifluoromethyl)-1H-imidazol-5-yl]pyrimidin-2-amine (obtained from Example 101(e)) (35 mg, 0.135 mmol) and 1-[(4-bromophenyl)sulfonyl]-4-methylpiperazine (described in WO 2003004472) (38 mg, 0.12 mmol) to give (47 mg, 78%). The hydrochloride was prepared in accordance with that described in general method D.

¹H NMR (DMSO-d₆, 300 MHz) δ 10.6-10.3 (m, 2H), 8.82 (s, 1H), 7.99 (s, J=8.4 Hz, 2H), 7.9-7.7 (m, 3H), 4.13 (s, 3H), 3.8-3.6 (m, 2H), 3.5-3.3 (m, 2H), 3.3-3.0 (m, 2H), 2.73 (s, 3H), 2.7-2.5 (m, 2H); MS (ES) m/z 500 (M+1).

### Example 101(a) 2,2,2-Trifluoro-N-methyl-N-(5-methylisoxazol-4-yl)acetamide

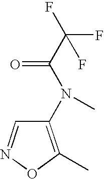

Trifluoroacetic anhydride (10 mL, 71 mmol) in CH₂Cl₂ (100 mL) was added to N,5-dimethylisoxazol-4-amine (Reiter, L. A., J. Org. Chem. 1987, 52, 2714-2726) (6.68 g, 59.6 mmol) in DCM (200 mL) and pyridine (6 mL, 74 mmol) at 0° C. The mixture was stirred at 0° C. for 30 min and at r.t. for 2 h. The reaction mixture was diluted with CH₂Cl₂ (100 mL) and washed with H₂O and saturated NaHCO₃ (aq). The organic layer was dried (Na₂SO₄), concentrated in vacuo to give the title compound (12.4 g, 100%) as a solid.

MS (ESI) m/z 208 (M⁺).

### Example 101(b) 1-[1-Methyl-2-(trifluoromethyl)-1H-imidazol-5-yl]ethanone

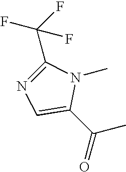

2,2,2-trifluoro-N-methyl-N-(5-methylisoxazol-4-yl)acetamide (12.4 g, 59.6 mmol, obtained from Example 1(a)) in EtOH (30 ml) was hydrogenated over Pd/C (10%, 1.0 g) at 50 psi. The reaction mixture was stirred at +50° C. overnight. Sodium methoxide (5.0 g, 87.7 mmol) was added and the resulting mixture was heated to reflux overnight. The mixture was filtered through diatomaceous earth and the residue was diluted with saturated NaHCO₃ (aq.) and extracted with EtOAc. The combined organic layers were dried (Na₂SO₄) and concentrated in vacuo. The crude product was purified by flash chromatography (Heptane:EtOAc 2:1) to give the title compound (6.1 g, 52%) as an oil.

¹H NMR (400 MHz, CDCl₃) δ ppm 7.77 (s, 1H), 4.07 (s, 3H), 2.54 (s, 3H);

MS (ESI) m/z 192 (M⁺).

### Example 101(c) (2E)-3-(Dimethylamino)-1-[1-methyl-2-(trifluoromethyl)-1H-imidazol-5-yl]prop-2-en-1-one

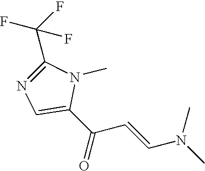

1-[1-methyl-2-(trifluoromethyl)-1H-imidazol-5-yl]ethanone (6.0 g, 31 mmol, obtained from Example 101(b)) was dissolved in DMFDMA/DMF (1:1, 46 mL) and the mixture was stirred at +100° C. overnight. After cooling to r.t. the mixture was diluted with H₂O and extracted with CH₂Cl₂ (three times). The organic phases were combined, dried (Na₂SO₄), filtered and concentrated in vacuo to give the title compound (7.11 g, 93%) as a solid.

MS (ESI) m/z 247 (M⁺); MS (ESI) m/z 248 (M+1).

### Example 101(d) (2Z)-3-(Dimethylamino)-2-fluoro-1-[1-methyl-2-(trifluoromethyl)-1H-imidazol-5-yl]prop-2-en-1-one

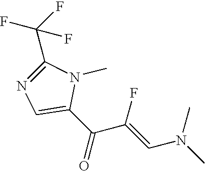

Selectfluor (10.9 g, 30.8 mmol) was added in portions to a stirred solution of (2E)-3-(dimethylamino)-1-[1-methyl-2-(trifluoromethyl)-1H-imidazol-5-yl]prop-2-en-1-one (7.0 g, 28.3 mmol, obtained from Example 101 (c)) in CH₃CN (250 mL) at 0° C. After stirring at 0° C. for 1.5 h the reaction mixture was diluted with H₂O and extracted with CH₂Cl₂ (three times). The organic phases were combined, dried (Na₂SO₄), filtered and concentrated in vacuo to give the crude title compound that was used in the next step without any father purification.

MS (ESI) m/z 265 (M⁺); MS (ESI) m/z 266 (M+1).

### Example 101 (e) 5-Fluoro-4-[1-methyl-2-(trifluoromethyl)-1H-imidazol-5-yl]pyrimidin-2-amine

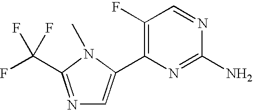

A reaction mixture of (2Z)-3-(dimethylamino)-2-fluoro-1-[1-methyl-2-(trifluoromethyl)-1H-imidazol-5-yl]prop-2-en-1-one (28 3 mmol, crude from Example 101(d)), guanidine carbonate (13.5 g, 75 mmol) and NaOMe (6.5 g, 120 mmol) in 1-butanol (250 mL) was heated to reflux under argon atmosphere for 2.5 h. The mixture was diluted with H₂O and extracted with CH₂Cl₂. The organic phases were combined, dried (Na₂SO₄), filtered and concentrated in vacuo. The crude product was purified by flash chromatography (Heptane:EtOAc 1:1 to Heptane:EtOAc 1:2) to give the title compound (1.76 g, 21%) as a solid.

¹H NMR (400 MHz, CDCl₃) δ ppm 8.27 (d, J=3.03 Hz, 1H) 7.74 (d, J=4.04 Hz, 1H) 5.02 (br. s., 2H) 4.14 (s, 3H); MS (ESI) m/z 261 (M⁺).

### Example 102

### 5-Fluoro-4-[3-methyl-2-(trifluoromethyl)imidazol-4-yl]-N-[4-(morpholin-4-ylmethyl)phenyl]-pyrimidin-2-amine hydrochloride

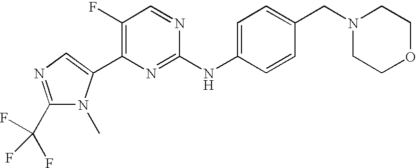

The title compound was prepared in accordance with the general method E using 5-fluoro-4-[1-methyl-2-(trifluoromethyl)-1H-imidazol-5-yl]pyrimidin-2-amine (obtained from Example 101(e)) (35 mg, 0.135 mmol) and 4-(4-bromobenzyl)-morpholine (34 mg, 0.134 mmol) to give (48 mg, 83%). The hydrochloride was prepared in accordance to that described in general method D.

¹H NMR (DMSO-d₆, 300 MHz) δ 10.95 (br s, 1H), 10.00 (s, 1H), 8.73 (s, 1H), 7.9-7.7 (m, 3H), 7.52 (d, J=8.4 Hz, 2H), 4.26 (d, J=4 Hz, 2H), 4.10 (s, 3H), 3.93 (d, J=12 Hz, 2H), 3.77 (d, J=12 Hz, 2H), 3.23 (d, J=12 Hz, 2H), 3.1-3.0 (m, 2H); MS (ESI) m/z 437 (M+1)

### Example 103

### [4-[5-Fluoro-4-[3-methyl-2-(trifluoromethyl)imidazol-4-yl]-pyrimidin-2-yl]aminophenyl]-(4-methylpiperazin-1-yl)-methanone hydrochloride

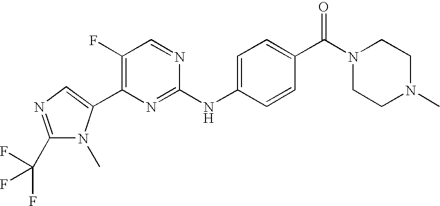

The title compound was prepared in accordance with the general method E using 5-fluoro-4-[1-methyl-2-(trifluoromethyl)-1H-imidazol-5-yl]pyrimidin-2-amine (obtained from Example 101(e)) (35 mg, 0.135 mmol) and 1-(4-bromobenzoyl)-4-methylpiperazine (36 mg, 0.127 mmol) to give (30 mg, 51% yield). The hydrochloride was prepared in accordance to that described in general method D.

¹H NMR (DMSO-d₆, 300 MHz) δ 10.84 (br s, 1H), 10.10 (s, 1H), 8.76 (s, 1H), 7.9-7.7 (m, 3H), 7.45 (d, J=8.4 Hz, 2H), 4.2-4.0 (m, 5H), 3.6-3.2 (m), 3.2-3.0 (m, 2H), 2.77 (s, 3H); hydrogens in the region 3.6-3.2 ppm were not integrated due to the overlap with the water peak; MS (ESI) m/z 464 (M+1)

### Example 104

### [4-[5-Fluoro-4-[3-tetrahydropyran-4-yl-2-(trifluoromethyl)imidazol-4-yl]-pyrimidin-2-yl]aminophenyl]-(4-methylpiperazin-1-yl)-methanone hydrochloride

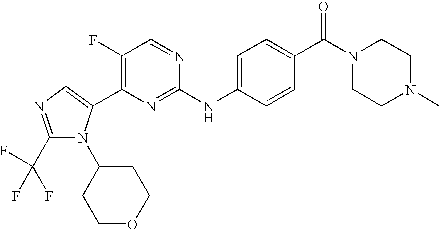

The title compound was prepared in accordance with the general method E and work-up procedure B. The product was purified by flash chromatography (CH₂Cl₂/MeOH 30:1, 20:1 then 15:1). Using 5-fluoro-4-[1-(tetrahydro-2H-pyran-4-yl)-2-(trifluoromethyl)-1H-imidazol-5-yl]pyrimidin-2-amine (obtained from Example 34(d)) (33 mg, 0.1 mmol), 1-(4-bromobenzoyl)-4-methylpiperazine (0.027 g, 0.095 mmol), Cs₂CO₃ (65 mg, 0.2 mmol), Pd₂(dba)₃ (6.8 mg, 0.0075 mmol) and X-Phos (7 mg, 0.015 mmol), the base of the title compound (35 mg, 70%) was obtained as a solid. The hydrochloride was prepared in accordance with the method described in general method D.

¹H NMR (DMSO-d₆, 300 MHz) δ 10.60 (br s, 1H), 10.11 (s, 1H), 8.82 (s, 1H), 7.74 (d, J=8.4 Hz, 2H), 7.56 (s, 1H), 7.42 (d, J=8.4 Hz, 2H), 4.80 (t, 1H), 3.80 (d, J=8.4 Hz, 2H), 3.22 (t, J=11.5 Hz, 2H), 3.2-3.0 (m, 2H), 2.78 (s, 3H), 2.2-2.1 (m, 2H), 2.0-1.8 (m, 2H); 6 Hydrogens were not assigned in the region 3.6-2.2 ppm due to the presence of the water and DMSO peaks in this region; MS (ESI) m/z 534.5 (M+1); MS (ESI) m/z 532.5 (M−1).

### Example 105

### 5-Fluoro-N-[3-(methylsulfonyl)-4-(morpholin-4-ylmethyl)phenyl]-4-[2-methyl-1-(tetrahydro-2H-pyran-4-yl)-1H-imidazol-5-yl]pyrimidin-2-amine hydrochloride

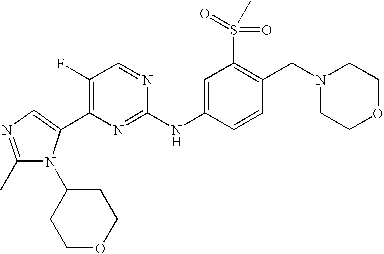

The title compound was prepared in accordance with the general method E (Workup procedure C), with the exception that the base of the product was purified by flash chromatography (gradient from 100% CH₂Cl₂ to 6% MeOH in CH₂Cl₂) before final purification by preparative HPLC. Using 5-fluoro-4-[2-methyl-1-(tetrahydro-2H-pyran-4-yl)-1H-imidazol-5-yl]pyrimidin-2-amine (obtained from Example 7(e)) (35 mg, 0.13 mmol), 4-[4-bromo-2-(methylsulfonyl)benzyl]morpholine (obtained from Example 105(a)) (46 mg, 0.14 mmol), Cs₂CO₃ (66 mg, 0.20 mmol), Pd₂(dba)₃ (6 mg, 0.006 mmol) and X-Phos (8 mg, 0.017 mmol), the base of the title compound (23 mg, 32%) was obtained as a solid. The hydrochloride was prepared in accordance with the method described in general method D.

¹H NMR (400 MHz, DMSO-d₆) δ ppm 10.47 (br.s., 1H) 10.40 (s, 1H) 8.88 (d, 1H) 8.33 (s, 1H) 8.22 (d, 1H) 8.07 (s, 1H) 7.98 (br.s., 1H) 5.00-4.87 (m, 1H) 4.62 (br.s., 2H) 4.03-3.72 (m, 6H) 2.82 (s, 3H) 2.24-2.09 (m, 2H) 2.02-1.90 (m, 2H) additional protons obscured by the HDO signal; MS (ESI) m/z 531 (M+1).

### Example 105(a) 4-[4-Bromo-2-(methylsulfonyl)benzyl]morpholine

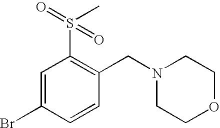

To a stirred solution of 4-bromo-2-(methylsulfonyl)benzaldehyde (0.20 g, 0.76 mmol) in MeOH (1.5 mL) was added morpholine (0.073 g, 0.84 mmol), NaCNBH₃ (0.072 g, 1.1 mmol) and HOAc (0.091 g, 1.5 mmol) and the reaction was stirred at r.t. over night. The solvent was removed in vacuo and the crude product was partitioned between EtOAc/1M NaHCO₃ (aq.). The organic phase was dried (Na₂SO₄), filtered concentrated and purified by flash chromatography (gradient from 100% heptane to 40% EtOAc in heptane) to give the title compound as a solid (0.088 g, 35%).

¹H NMR (400 MHz, DMSO-d₆) δ ppm 8.03 (d, 1H) 7.90 (dd, 1H) 7.56 (d, 1H) 3.82 (s, 2H) 3.59-3.51 (m, 4H) 3.48 (s, 3H) 2.44-2.35 (m, 4H); MS (ESI) m/z 336 (+1).

### Example 106

### 5-Fluoro-N-[4-(methylsulfonyl)-3-(trifluoromethyl)phenyl]-4-[2-methyl-1-(tetrahydro-2H-pyran-4-yl)-1H-imidazol-5-yl]pyrimidin-2-amine hydrochloride

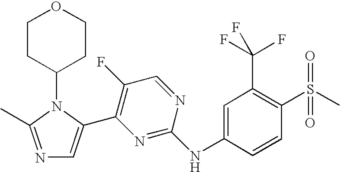

The title compound was prepared in accordance with the general method E (Workup procedure C), with the exception that the base of the product was purified by flash chromatography (gradient from 100% EtOAc to 10% MeOH in EtOAc). Using 5-fluoro-4-[2-methyl-1-(tetrahydro-2H-pyran-4-yl)-1H-imidazol-5-yl]pyrimidin-2-amine (obtained from Example 7(e)) (40 mg, 0.14 mmol), 4-bromo-1-(methylsulfonyl)-2-(trifluoromethyl)benzene (43 mg, 0.14 mmol), Cs₂CO₃ (85 mg, 0.26 mmol), Pd₂(dba)₃ (8 mg, 0.009 mmol) and X-Phos (9 mg, 0.018 mmol), the base of the title compound (64 mg, 83%) was obtained as a solid. The hydrochloride was prepared in accordance with the method described within general method D.

¹H NMR (400 MHz, DMSO-d₆) δ ppm 10.64 (s, 1H) 8.94 (d, 1H) 8.30-8.21 (m, 2H) 8.16-8.01 (m, 2H) 4.99-4.87 (m, 1H) 3.84 (dd, 2H) 3.23 (s, 3H) 3.28-3.20 (m, 2H signal partly obscured by the HDO signal) 2.80 (s, 3H) 2.24-2.09 (m, 2H) 1.94 (dd, 2H); MS (ESI) m/z 500 (M+1).

### Example 107

### 6-({5-Fluoro-4-[2-methyl-1-(tetrahydro-2H-pyran-4-yl)-1H-imidazol-5-yl]pyrimidin-2-yl}amino)-2,3-dihydro-4H-thiochromen-4-one 1,1-dioxide hydrochloride

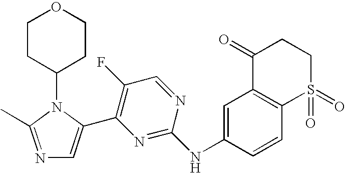

The title compound was prepared in accordance with the general method E (Workup procedure C), with the exception that the base of the product was purified by flash chromatography twice [(i) gradient from 100% EtOAc to 10% MeOH in EtOAc, (ii) gradient from heptane/CH₂Cl₂ 7:3 to 3% MeOH in heptane/CH₂Cl₂ 7:3] followed by precipitation from a solution in MeOH/CH₂Cl₂ 1:3 by addition of toluene. Using 5-fluoro-4-[2-methyl-1-(tetrahydro-2H-pyran-4-yl)-1H-imidazol-5-yl]pyrimidin-2-amine (obtained from Example 7(e)) (51 mg, 0.18 mmol), 6-chloro-2,3-dihydro-4H-thiochromen-4-one 1,1-dioxide (46 mg, 0.20 mmol), Cs₂CO₃ (95 mg, 0.29 mmol), Pd₂(dba)₃ (9 mg, 0.010 mmol) and X-Phos (10 mg, 0.021 mmol), the base of the title compound (20 mg, 23%) was obtained as a solid. The hydrochloride was prepared in accordance with the method described in general method D with the exception that the salt was precipitated from a CH₃CN/CH₂Cl₂ solution.

¹H NMR (400 MHz, DMSO-d₆) δ ppm 10.45 (s, 1H) 8.89 (d, 1H) 8.26 (d, 1H) 8.21 (dd, 1H) 8.03 (br. s., 1H) 7.89 (d, 1H) 5.03-4.90 (m, 1H) 3.95 (t, 2H) 3.82 (dd, 2H) 3.26-3.21 (m, 2H signal partly obscured by the HDO signal) 3.16 (t, 2H) 2.79 (s, 3H) 2.23-2.08 (m, 2H) 1.93 (dd, 2H); MS (ESI) m/z 470 (M−1).

### Example 108

### 6-({5-Fluoro-4-[2-methyl-1-(tetrahydro-2H-pyran-4-yl)-1H-imidazol-5-yl]pyrimidin-2-yl}amino)thiochroman-4-ol 1,1-dioxide hydrochloride

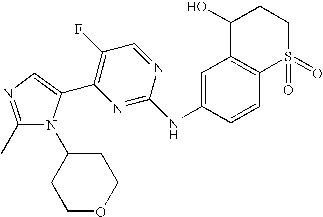

The title compound was prepared in accordance with the general method E (Workup procedure C), with the exception that the base of the product was purified by flash chromatography (gradient from 100% CH₂Cl₂ to 5% MeOH in CH₂Cl₂). Using 5-fluoro-4-[2-methyl-1-(tetrahydro-2H-pyran-4-yl)-1H-imidazol-5-yl]pyrimidin-2-amine (obtained from Example 7(e)) (52 mg, 0.18 mmol), 6-chlorothiochroman-4-ol 1,1-dioxide (Boissier, Jacques R.; Ratouis, Roger, Fr. M. (1970), FR 7499) (46 mg, 0.20 mmol), Cs₂CO₃ (95 mg, 0.29 mmol), Pd₂(dba)₃ (8 mg, 0.009 mmol) and X-Phos (9 mg, 0.019 mmol), the base of the title compound (10 mg, 12%) was obtained as a solid. The hydrochloride was prepared in accordance with the general method D.

¹H NMR (400 MHz, DMSO-d₆) δ ppm 10.19 (s, 1H) 8.83 (d, 1H) 7.98 (br. s., 1H) 7.87 (dd, 1H) 7.79 (d, 1H) 7.66 (d, 1H) 5.81 (br s, 1H) 5.05-4.90 (m, 1H) 4.76-4.66 (m, 1H) 3.83 (dd, 2H) 3.60-3.45 (m, 2H) 3.14 (q, 2H) 2.76 (s, 3H) 2.5-2.40 (m, 1H) 2.36-2.23 (m, 1H) 2.23-2.07 (m, 2H) 1.98-1.85 (m, 2H); MS (ESI) m/z 474 (M+1).

### Example 109

### N-(3-Dimethylaminopropyl)-3-[[4-(2,3-dimethylimidazol-4-yl)-5-fluoro-pyrimidin-2-yl]amino]benzamide

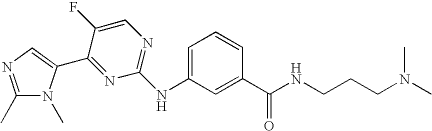

The title compound was prepared in accordance with the general method G using preparative HPLC (gradient from 0% to 40% acetonitrile in ammonium acetate buffer) for purification. Using methyl 3-{[4-(1,2-dimethyl-1H-imidazol-5-yl)-5-fluoropyrimidin-2-yl]amino}benzoate (44 mg, 0.129 mmol, obtained from Example 59), Al(CH₃)₃ (54 mg, 0.75 mmol, 2.0 M in toluene) and N,N-dimethylpropane-1,3-diamine (0.89 mg, 0.87 mmol), the title compound (45 mg, 84%) was obtained as a solid.

¹H NMR (400 MHz, DMSO-d₆) δ ppm 9.70 (s, 1H) 8.53 (d, 1H) 8.42 (t, 1H) 8.12 (s, 1H) 7.82-7.75 (m, 1H) 7.56 (d, 1H) 7.43-7.32 (m, 2H) 3.92 (s, 3H) 2.40 (s, 3H) 2.25 (t, 2H) 2.13 (s, 6H) 1.71-1.58 (m, 2H); MS (ESI) m/z 412 (M+1).

### Example 110

### N-(3-Dimethylaminopropyl)-3-[[4-(2,3-dimethylimidazol-4-yl)-5-fluoro-pyrimidin-2-yl]amino]-N-methyl-benzamide hydrochloride

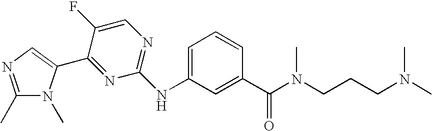

The title compound was prepared in accordance with the general method G using preparative HPLC (gradient from 5% to 45% acetonitrile in ammonium acetate buffer) for purification. Using methyl 3-{[4-(1,2-dimethyl-1H-imidazol-5-yl)-5-fluoropyrimidin-2-yl]amino}benzoate (51 mg, 0.149 mmol, obtained from Example 59), Al(CH₃)₃ (107 mg, 1.49 mmol, 2.0 M in toluene) and N,N′,N′-trimethylpropane-1,3-diamine (0.89 mg, 0.87 mmol), the base of the title compound (38 mg, 51%) was obtained as a solid. The hydrochloride was prepared in accordance with the method described in general method D.

¹H NMR (400 MHz, DMSO-d₆) δ ppm 10.32 (br. s., 1H) 9.99 (br. s., 1H) 8.76 (d, 1H) 8.17 (d, 1H) 7.79-7.68 (m, 2H) 7.39 (t, 1H) 7.06 (br. s., 1H) 4.02 (s, 3H) 3.56-3.47 (m, 2H) 3.13-3.02 (m, 2H) 2.94 (br. s., 3H) 2.77 (br. s., 6H) 2.67 (s, 6H) 2.05-1.93 (m, 2H); MS (ESI) m/z 426 (M+1).

### Example 111

### [3-[[4-(2,3-Dimethylimidazol-4-yl)-5-fluoro-pyrimidin-2-yl]amino]phenyl]-[3-(hydroxymethyl)-1-piperidyl]methanone

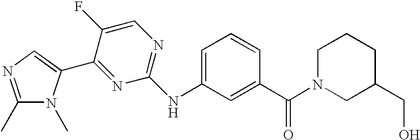

The title compound was prepared in accordance with the general method G using preparative HPLC (gradient from 10% to 50% acetonitrile in ammonium acetate buffer) for purification. Using methyl 3-{[4-(1,2-dimethyl-1H-imidazol-5-yl)-5-fluoropyrimidin-2-yl]amino}benzoate (53 mg, 0.155 mmol, obtained from Example 59), Al(CH₃)₃ (108 mg, 1.50 mmol, 2.0 M in toluene) and 3-piperidylmethanol (0.102 mg, 0.89 mmol), the title compound (56 mg, 85%) was obtained as a solid.

¹H NMR (400 MHz, DMSO-d₆) δ ppm 9.96 (s, 1H) 8.75 (d, 1H) 8.16 (d, 1H) 7.70 (s, 1H) 7.71 (d, 1H) 7.37 (t, 1H) 6.98 (d, 1H) 4.01 (s, 3H) 3.72 (br. s., 1H) 3.02-2.90 (m, 1H) 2.83-2.69 (m, 1H) 2.66 (s, 3H) 1.78-1.68 (m, 2H) 1.58 (br. s., 2H) 1.41 (br. s., 1H) 1.27-1.12 (m, 1H); MS (ESI) m/z 425 (M+1).

### Example 112

### N-{3-Chloro-4-[(4-methylpiperazin-1-yl)carbonyl]phenyl}-5-fluoro-4-[2-methyl-1-(tetrahydro-2H-pyran-4-yl)-1H-imidazol-5-yl]pyrimidin-2-amine

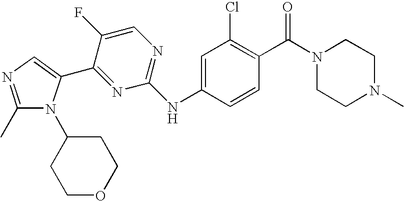

The title compound was prepared in accordance with the general method E (Workup procedure C). Using 5-fluoro-4-[2-methyl-1-(tetrahydro-2H-pyran-4-yl)-1H-imidazol-5-yl]pyrimidin-2-amine (obtained from Example 7(e)) (33.1 mg, 0.119 mmol), 1-(2,4-dichlorobenzoyl)-4-methylpiperazine (33.0 mg, 0.120 mmol), Cs₂CO₃ (62.0 mg, 0.190 mmol), Pd₂(dba)₃ (6.0 mg, 0.0065 mmol) and X-Phos (7.0 mg, 0.015 mmol), the title compound was obtained (34.1 mg, 56%).

¹H NMR (400 MHz, DMSO-d₆) δ ppm 9.89 (s, 1H) 8.65 (d, 1H) 7.85 (d, 1H) 7.61 (dd, 1H) 7.35 (d, 1H) 7.25 (d, 1H) 4.92-5.08 (m, 1H) 3.57-3.67 (m, 2H) 3.13-3.18 (m, 2H) 3.08-3.13 (m, 2H) 2.55 (s, 3H) 2.30-2.41 (m, 4H) 2.22-2.28 (m, 4H) 2.19 (s, 3H) 1.83 (d, 2H); MS (ESI) m/z 514 (M+1).

### Example 113

### 5-Fluoro-N-{3-[(4-methylpiperazin-1-yl)carbonyl]phenyl}-4-[2-methyl-1-(tetrahydro-2H-pyran-4-yl)-1H-imidazol-5-yl]pyrimidin-2-amine

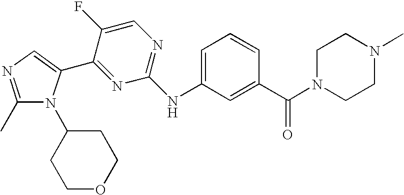

The title compound was prepared in accordance with the general method E (Workup procedure C). Using 5-fluoro-4-[2-methyl-1-(tetrahydro-2H-pyran-4-yl)-1H-imidazol-5-yl]pyrimidin-2-amine (obtained from Example 7(e)) (32.3 mg, 0.116 mmol), 1-(3-chlorobenzoyl)-4-methylpiperazine (28.0 mg, 0.117 mmol), Cs₂CO₃ (60.0 mg, 0.184 mmol), Pd₂(dba)₃ (6.0 mg, 0.0065 mmol) and X-Phos (7.0 mg, 0.015 mmol), the title compound was obtained (31.0 mg, 56%).

¹H NMR (400 MHz, DMSO-d₆) δ ppm 9.67 (s, 1H) 8.59 (d, 1H) 7.69 (d, 1H) 7.66-7.63 (m, 11H) 7.34-7.30 (m, 21H) 6.97 (d, 1H) 3.86-3.78 (m, 2H) 3.12 (t, 21H) 2.53 (s, 3H) 2.36-2.20 (m, 41H) 2.16 (s, 3H) 2.20-2.08 (m, 4H) 1.83-1.74 (m, 2H); MS (ESI) m/z 480 (M+1).

### Example 114

### (4-{[4-(1,2-Dimethyl-1H-imidazol-5-yl)-5-fluoropyrimidin-2-yl]amino}phenyl)(pyridin-2-yl)methanol

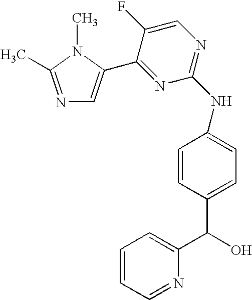

{4-[4-(2,3-Dimethyl-3H-imidazol-4-yl)-pyrimidin-2-ylamino]-phenyl}-pyridin-2-yl-methanone (obtained from Example 31) (10 mg, 0.025 mmol) was treated with NaBH₄ (10 mg, 0.264 mmol) in EtOH (2 mL) at 0° C. The crude material was directly purified by preparative HPLC to give the title compound (2 mg, 20%).

¹H NMR (400 MHz, CDCl₃) δ ppm 8.59 (d, J=4.80 Hz, 1H) 8.25 (d, J=3.28 Hz, 1H) 7.73 (d, J=4.29 Hz, 1H) 7.60-7.68 (m, 1H) 7.52 (d, J=8.34 Hz, 2H) 7.35 (d, J=8.59 Hz, 2H) 7.20-7.25 (m, 1H) 7.17 (d, J=7.83 Hz, 1H) 7.05 (br. s., 1H) 5.75 (s, 1H) 5.30 (br. s., 1H) 3.90 (s, 3H) 2.48 (s, 3H); MS (ES) m/z 391 (M+1).

### Example 115

### 5-Fluoro-N-[4-(isopropylsulfonyl)phenyl]-4-[2-methyl-1-(tetrahydro-2H-pyran-4-yl)-1H-imidazol-5-yl]pyrimidin-2-amine

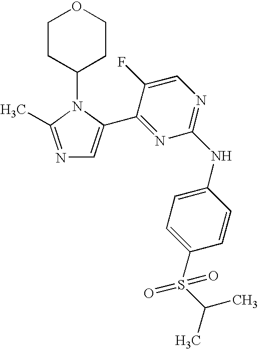

The title compound was prepared in accordance with the general method E. Using 5-fluoro-4-[2-methyl-1-(tetrahydro-2H-pyran-4-yl)-1H-imidazol-5-yl]pyrimidin-2-anine (obtained from Example 7(e)) (0.05 g, 0.18 mmol), 1-bromo-4-(propane-2-sulfonyl)-benzene (0.048 g, 0.18 mmol), Cs₂CO₃ (176 mg, 0.54 mmol), Pd₂(dba)₃ (4 mg, 0.005 mmol) and X-Phos (4 mg, 0.009 mmol), the title compound (16 mg, 20%) was obtained as a solid.

¹H NMR (400 MHz, CDCl₃) δ ppm 8.38 (d, J=3.03 Hz, 1H) 7.77-7.82 (m, 4H) 7.68-7.69 (m, 1H) 7.66 (s, 1H) 5.04-5.12 (m, 1H) 4.11 (dd, J=11.62, 4.55 Hz, 2H) 3.32-3.39 (m, 2H) 3.13-3.23 (m, 1H) 2.66 (s, 3H) 2.50-2.62 (m, 2H) 1.88 (m, 2H) 1.31 (d, J=6.82 Hz, 6H); MS (ES) m/z 458 (M−1)

### Example 116

### N-[4-(Ethylsulfonyl)phenyl]-5-fluoro-4-[2-methyl-1-(tetrahydro-2H-pyran-4-yl)-1H-imidazol-5-yl]pyrimidin-2-amine

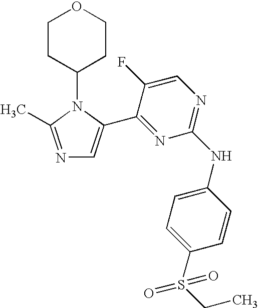

The title compound was prepared in accordance with the general method E. Using 5-fluoro-4-[2-methyl-1-(tetrahydro-2H-pyran-4-yl)-1H-imidazol-5-yl]pyrimidin-2-amine (obtained from Example 7(e)) (0.05 g, 0.18 mmol), 1-bromo-4-ethanesulfonyl-benzene (0.039 g, 0.18 mmol), Cs₂CO₃ (176 mg, 0.54 μmol), Pd₂(dba)₃ (4 mg, 0.005 mmol) and X-Phos (4 mg, 0.009 mmol), the title compound (21 mg, 26%) was obtained as a solid.

¹H NMR (400 MHz, CDCl₃) δ ppm 8.37 (m, 1H) 7.76-7.84 (m, 4H) 7.74 (br. s., 1H) 7.68 (d, J=3.79 Hz, 1H) 5.03-5.13 (m, 1H) 4.10 (dd, J=11.62, 4.55 Hz, 2H) 3.29-3.38 (m, 2H) 3.11 (q, J=7.58 Hz, 2H) 2.66 (s, 3H) 2.60-2.50 (m, 2H) 1.89-1.86 (m, 1H) 1.28 (t, J=7.41 Hz, 3H); MS (ES) m/z 444 (M−1).

### Example 117

### 5-Fluoro-N-{4-[(2-methoxyethyl)sulfonyl]phenyl}-4-[2-methyl-1-(tetrahydro-2H-pyran-4-yl)-1H-imidazol-5-yl]pyrimidin-2-amine

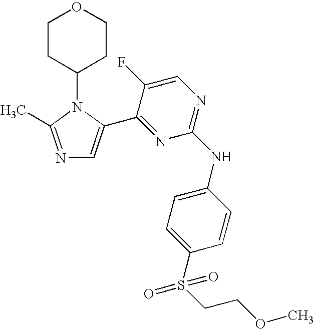

The title compound was prepared in accordance with the general method E. Using 5-fluoro-4-[2-methyl-1-(tetrahydro-2H-pyran-4-yl)-1H-imidazol-5-yl]pyrimidin-2-amine (obtained from Example 7(e)) (0.05 g, 0.18 mmol), 2-(4-bromophenyl)sulfonyl ethyl ether (obtained from Example 117(a)) (0.05 g, 0.18 mmol), Cs₂CO₃ (176 mg, 0.54 mmol), Pd₂(dba)₃ (4 mg, 0.005 mmol) and X-Phos (4 mg, 0.009 mmol), the title compound (21 mg, 26%) was obtained as a solid.

¹H NMR (400 MHz, CDCl₃) δ ppm 8.38 (d, J=2.78 Hz, 1H) 7.75-7.88 (m, 4H) 7.70 (br. s., 1H) 7.67 (br. s., 1H) 5.02-5.13 (m, 1H) 4.11 (dd, J=11.62, 4.55 Hz, 2H) 3.75 (t, J=6.32 Hz, 2H) 3.29-3.42 (m, 4H) 3.27 (s, 3H) 2.66 (s, 3H) 2.50-2.63 (m, 2H) 1.88 (dd, J=12.24, 3.03 Hz, 2H); MS (ES) m/z 474 (M−1)

### Example 117(a) 2-[(4-Bromophenyl)sulfonyl]ethyl methyl ether

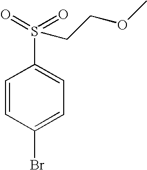

The title compound was prepared in accordance with the general method I. Using 4-bromobenzenesulfonyl chloride (0.256 g, 1 mmol), Na₂SO₃ (0.126 g, 1 mmol), NaHCO₃ (0. 252 g, 3 mmol.) and 2-bromoethyl methyl ether (0.28 mL, 3 mmol) to give the title compound (0.132 g, 50%) as an oil.

¹H NMR (400 MHz, CDCl₃) 5 ppm 7.67-7.83 (m, 4H) 3.73 (t, J=6.06 Hz, 2H) 3.37 (t, J=6.06 Hz, 2H) 3.21 (s, 3H); MS (ES) m/z 280 (M+1).

### Example 118

### N-(4-{[2-(Diethylamino)ethyl]sulfonyl}phenyl)-5-fluoro-4-[2-methyl-1-(tetrahydro-2H-pyran-4-yl)-1H-imidazol-5-yl]pyrimidin-2-amine

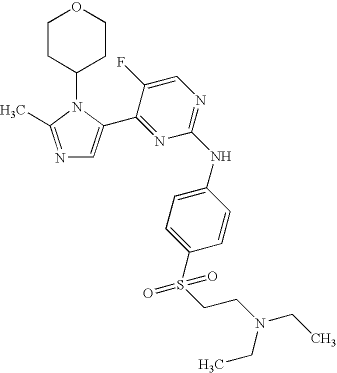

The title compound was prepared in accordance with the general method E. Using 5-fluoro-4-[2-methyl-1-(tetrahydro-2H-pyran-4-yl)-1H-imidazol-5-yl]pyrimidin-2-amine (obtained from Example 7(e)) (0.05 g, 0.18 mmol), [2-(4-bromo-benzenesulfonyl)-ethyl]-diethyl-amine (obtained from Example 118(a)) (0.058 g, 0.18 mmol), Cs₂CO₃ (176 mg, 0.54 mmol), Pd₂(dba)₃ (4 mg, 0.005 mmol) and X-Phos (4 mg, 0.009 mmol), the title compound (50 mg, 54%) was obtained as a solid.

¹H NMR (400 MHz, CDCl₃) δ ppm 8.36 (d, J=2.78 Hz, 1H) 7.88 (br. s., 1H) 7.74-7.84 (m, 4H) 7.62-7.70 (m, 1H) 5.00-5.15 (m, 1H) 4.08 (dd, J=11.62, 4.55 Hz, 2H) 3.27-3.40 (m, 2H) 3.19-3.27 (m, 2H) 2.85-2.95 (m, 2H) 2.63 (s, 3H) 2.48-2.60 (m, 2H) 2.45 (q, J=7.33 Hz, 4H) 1.81-1.91 (m, 2H) 0.94 (t, J=7.33 Hz, 6H); MS (ES) m/z 515 (M−1).

### Example 118(a) 2-[(4-Bromophenyl)sulfonyl]ethyl diethyl-amine

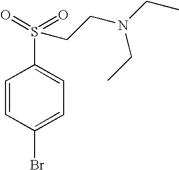

The title compound was prepared in accordance with the general method I. Using 4-bromobenzenesulfonyl chloride (0.256 g, 1 mmol), Na₂SO₃ (0.126 g, 1 mmol), NaHCO₃ (0. 252 g, 3 mmol.) and 2-bromoethyl diethyl amine hydrobromide (0.52 g, 2 mmol) to give the title compound (0.06 g, 19%) as an oil.

¹H NMR (400 MHz, CDCl₃) δ ppm 7.66-7.84 (m, 4H) 3.19-3.29 (m, 2H) 2.86-2.94 (m, 2H) 2.43 (q, J=7.07 Hz, 4H) 0.93 (t, J=7.03 Hz, 6H); MS (ES) m/z 322 (M+2).

### Example 119

### 2-{[4-({5-Fluoro-4-[2-methyl-1-(tetrahydro-2H-pyran-4-yl)-1H-imidazol-5-yl]pyrimidin-2-yl}amino)phenyl]sulfonyl}ethanol

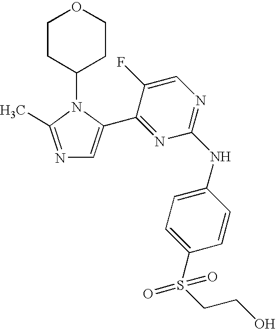

The title compound was prepared in accordance with the general method E. Using 5-fluoro-4-[2-methyl-1-(tetrahydro-2H-pyran-4-yl)-1H-imidazol-5-yl]pyrimidin-2-amine (obtained from Example 7(e)) (0.05 g, 0.18 mmol), 2-[(4-bromo-phenyl)sulfonyl]ethanol (described in DE3530710) (0.048 g, 0.18 mmol), Cs₂CO₃ (176 mg, 0.54 mmol), Pd₂(dba)₃ (4 mg, 0.005 mmol) and X-Phos (4 mg, 0.009 mmol), the title compound (40 mg, 44%) was obtained as a solid.

¹H NMR (400 MHz, CDCl₃) δ ppm 8.37 (d, J=2.78 Hz, 1H) 7.89 (s, 1H) 7.77-7.86 (m, 4H) 7.66 (d, J=4.04 Hz, 1H) 5.00-5.12 (m, 1H) 4.09 (dd, J=11.62, 4.55 Hz, 2H) 3.96-4.03 (m, 2H) 3.28-3.39 (m, 41H) 2.64 (s, 3H) 2.47-2.60 (m, 2H) 1.81-1.92 (m, 2H);

MS (ES) m/z 462 (M+1)

### Example 120

### {5-Fluoro-4-[3-(tetrahydro-pyran-4-yl)-3H-imidazol-4-yl]-pyrimidin-2-yl}-[4-(4-methyl-piperazine-1-sulfonyl)-phenyl]-amine

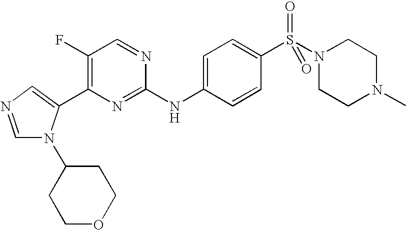

The title compound was prepared in accordance with the general method E. Using 5-fluoro-4-[1-(tetrahydro-2H-pyran-4-yl)-1H-imidazol-5-yl]pyrimidin-2-amine (obtained from Example 120(e)) (0.1 g, 0.38 mmol), 1-[(4-bromophenyl)sulfonyl]-4-methylpiperazine (0.111 g, 0.349 mmol), Cs₂CO₃ (247 mg, 0.76 mmol), Pd₂(dba)₃ (17 mg, 0.019 mmol) and X-Phos (18 mg, 0.038 mmol), the title compound (155 mg, 88%) was obtained as an oil.

¹H NMR (DMSO-d₆, 300 MHz) δ 8.38 (m, 1H), 7.88 (m, 2H), 7.71 (m, 4H), 7.38 (s, 1H), 5.35 (m, 1H), 4.06 (m, 2H) 3.34 (m, 2H), 3.05 (m, 4H), 2.27 (s, 3H), 2.12-1.98 (m, 4H); MS (ES) m/z 502 (M+1).

### Example 120(a) N-(5-Methyl-isoxazol-4-yl)-N-(tetrahydro-pyran-4-yl)-formamide

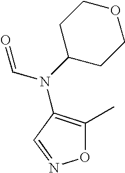

The title compound was prepared following the procedure described in Example 7(a), with the exception that the product was purified by flash chromatography (EtOAc). Using 5-methyl-4-amino-isoxazole (Reiter, L. A, J. Org. Chem. 1987, 52, 2714-2726) (2.5 g, 25.48 mmol), tetrahydro-pyran-4-one (0.26 ml, 28.03 mmol) and formic acid (3.2 g, 15.3 mmol) the title compound was obtained (3.8 g, 71%).

¹H NMR (400 MHz, CDCl₃) δ ppm 8.12 (s, 1H), 8.01 (s, 1H), 4.67 (m, 1H), 3.99 (m, 2H), 3.49 (m, 1H), 2.40 (s, 3H), 1.72 (m, 2H), 1.50 (m, 2H).

### Example 120(b) 5-Acetyl-1-(tetrahydro-pyran-4-yl)-1H-imidazole

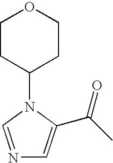

The title compound was prepared in accordance with the general method of Example 7(b), with the exception that the product was purified by flash chromatography (CH₂Cl₂/MeOH, 20:1). Using N-(5-Methyl-isoxazol-4-yl)-N-(tetrahydro-pyran-4-yl)-formamide (3.8 g, 18.1 mmol, obtained from Example 120(a)) the title compound was obtained (2.7 g, 77%).

¹H NMR (400 MHz, CDCl₃) δ ppm 7.80 (m, 2H) 5.15 (m, 1H) 4.09 (m, 2H) 3.57 (m, 2H) 2.48 (s, 3H) 2.06 (m, 2H) 1.92 (m, 21H).

### Example 120(c) (E)-3-Dimethylamino-1-[3-(tetrahydro-pyran-4-yl)-3H-imidazol-4-yl]-propenone

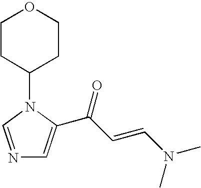

The title compound was prepared in accordance with the general method of Example 7(c), with the exception that the product was purified by flash chromatography (CH₂Cl₂/MeOH, 25:1). Using 5-acetyl-1-(tetrahydro-pyran-4-yl)-1H-imidazole (2.7 g, 13.9 mmol, obtained from Example 120(b)) the title compound was obtained (3.2 g, 92%).

¹H NMR (400 MHz, CDCl₃) δ ppm 7.71-7.61 (m, 3H) 5.53 (m, 1H) 5.37 (m, 1H) 4.07 (m, 2H) 3.57 (m, 2H) 3.10 (br. s., 3H) 2.93 (br.s., 3H) 2.11 (m, 2H) 1.92 (m, 2H).

### Example 120(d) (Z)-3-Dimethylamino-2-fluoro-1-[3-(tetrahydro-pyran-4-yl)-3H-imidazol-4-yl]-propenone

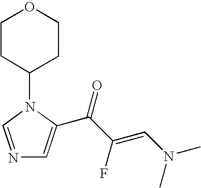

The title compound was prepared in accordance with the general method of Example 7(d), with the exception that the product was purified by flash chromatography (EtOAc/MeOH). Using (E)-3-dimethylamino-1-[3-(tetrahydro-pyran-4-yl)-3H-imidazol-4-yl]-propenone (3.2 g, 12.85 mmol, obtained from Example 120(d)) the title compound was obtained (0.68 g, 20%) as an oil.

¹H NMR (400 MHz, CDCl₃) δ ppm 7.74 (s, 1H) 7.60 (s, 1H) 6.89 (m, 1H) 5.10 (m, 1H) 4.05 (m, 2H) 3.53 (m, 2H) 3.11 (s, 6H) 2.08 (m, 2H) 1.89 (m, 2H).

### Example 120(e) 5-Fluoro-4-[1-(tetrahydro-2H-pyran-4-yl)-1H-imidazol-5-yl]pyrimidin-2-amine

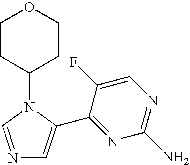

The title compound was prepared in accordance with the general method B with the exception that guanidine carbonate was used. Using (Z)-3-dimethylamino-2-fluoro-1-[3-(tetrahydro-pyran-4-yl)-3H-imidazol-4-yl]-propenone (0.67 g, 2.51 mmol, obtained from Example 120(d)) and guanidine carbonate (1.13 g, 6.27 mmol) the title compound (0.48 g, 73%) was obtained as a solid after purification by flash chromatography (CH₂Cl₂/MeOH 20:1).

¹H NMR (400 MHz, CDCl₃) 5 ppm 8.19 (m, 1H) 7.83 (m, 2H) 5.40 (m, 1H) 4.89 (m, 2H) 4.15 (m, 2H) 3.54 (m, 2H) 2.16 (m, 2H) 2.01 (m, 2H); MS (ES) m/z 264 (M+1).

### Example 121

### 5-{5-Fluoro-2-[4-(4-methyl-piperazine-1-sulfonyl)-phenylamino]-pyrimidin-4-yl}-1-(tetrahydro-pyran-4-yl)-1H-4H-imidazole-2-carbonitrile

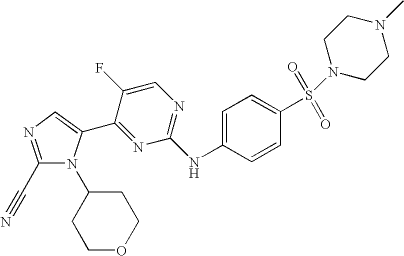

5-{5-Fluoro-2-[4-(4-methyl-piperazine-1-sulfonyl)-phenylamino]-pyrimid in-4-yl}-1-(tetrahydro-pyran-4-yl)-1H-imidazole-2-carbaldehyde (obtained from Example 121(a)) (34 mg, 0.064 mmol) was mixed with NH₂OH.HCl (5 mg, 0.077 mmol) in formic acid (1 mL). The mixture was heated to reflux and monitored by LC until full conversion was achieved. Then, the mixtured was extracted and purified by flash chromatography (CH₂Cl₂/MeOH) to give the title compound (21 mg, 64%).

MS (ES) m/z 527 (M+1).

### Example 121 (a) 5-{5-Fluoro-2-[4-(4-methyl-piperazine-1-sulfonyl)-phenylamino]-pyrimidin-4-yl}-1-(tetrahydro-pyran-4-yl)-1H-imidazole-2-carbaldehyde

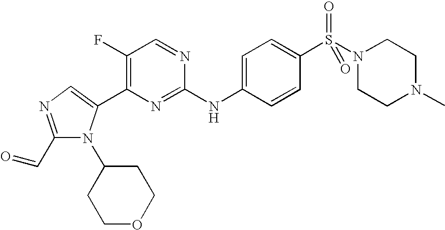

{5-Fluoro-4-[3-(tetrahydro-pyran-4-yl)-3H-imidazol-4-yl]-pyrimidin-2-yl}-[4-(4-methyl-piperazine-1-sulfonyl)-phenyl]-amine (obtained from Example 120) (0.315 g, 0.629 mmol) was treated with n-BuLi (1.8 mL, 1.6M solution in hexane, 2.83 mmol) in THF (10 mL) at low temperature (−60 to −30° C.) for 30 min. Then, DMF (0.137 mg, 1.89 mmol) was added to the mixture at −70° C. and the cooling bath was removed. The resulting mixture was quenched with NH₄Cl (saturated solution) and extracted with CH₂Cl₂. The title compound (0.1 g, 30%) was obtained after purification by flash chromatography (CH₂Cl₂/MeOH 20:1).

¹H NMR (CDCl₃, 300 MHz) δ 9.94 (s, 1H), 8.51 (m, 1H), 7.79-7.68 (m, 6H), 5.33 (m, 1H), 4.07 (m, 2H) 3.38 (m, 2H), 3.03 (m, 4H), 2.71 (m, 2H), 2.48 (m, 4H), 2.26 (s, 3H), 1.80 (m, 4H).

### Example 122

### {5-Fluoro-4-[2-methyl-3-(tetrahydro-pyran-4-yl)-3H-imidazol-4-yl]-pyrimidin-2-yl}-[4-(tetrahydro-pyran-2-ylmethanesulfonyl)-phenyl]-amine

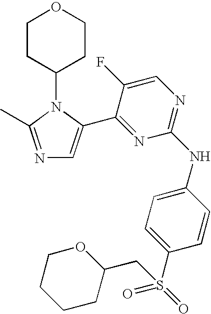

The title compound was prepared in accordance with the general method E. Using 5-fluoro-4-[2-methyl-1-(tetrahydro-2H-pyran-4-yl)-1H-imidazol-5-yl]pyrimidin-2-amine (obtained from Example 7(e)) (0.05 g, 0.18 mmol), 2-(4-bromo-benzenesulfonylmethyl)-tetrahydro-pyran (obtained from Example 122(a)) (0.057 g, 0.18 mmol), Cs₂CO₃ (176 mg, 0.54 mmol), Pd₂(dba)₃ (4 mg, 0.005 mmol) and X-Phos (4 mg, 0.009 mmol), the title compound (21 mg, 23%) was obtained as a solid.

¹H NMR (400 MHz, CDCl₃) δ ppm 8.37 (d, J=3.03 Hz, 1H) 7.80-7.88 (m, 2H) 7.71-7.78 (m, 2H) 7.63-7.71 (m, 2H) 4.92-5.19 (m, 1H) 4.10 (dd, J=11.75, 4.42 Hz, 2H) 3.76-3.92 (m, 2H) 3.28-3.41 (m, 5H) 3.12 (dd, J=14.40, 3.79 Hz, 1H) 2.66 (s, 3H) 2.47-2.61 (m, 2H) 1.23-1.93 (3m, 7H); MS (ES) m/z 516 (M+1).

### Example 122(a) 2-(4-Bromo-benzenesulfonylmethyl)-tetrahydro-pyran

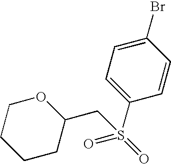

The title compound was prepared in accordance with the general method I. Using 4-bromobenzenesulfonyl chloride (0.256 g, 1 mmol), Na₂SO₃ (0.126 g, 1 mmol), NaHCO₃ (0. 252 g, 3 mmol.) and 2-bromomethyl-tetrahydro-pyran (0.128 mL, 1 mmol) to give the title compound (0.06 g, 19%) as an oil.

MS (ES) m/z 321 (M+1).

### Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with glycogen synthase kinase-3.

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt/solvate (active ingredient) is in association with a pharmaceutically acceptable excipient, diluent or carrier. Dependent on the mode of administration, the pharmaceutical composition may comprise from 0.05 to 99% w (percent by weight), for example from 0.10 to 50% w, of active ingredient, all percentages by weight being based on total composition.

An excipient, diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt, solvate or solvate of salt thereof, as hereinbefore defined, with a pharmaceutically acceptable excipient, diluent or carrier.

An example of a pharmaceutical composition of the invention is an injectable solution containing a compound of the invention, or a a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

### Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man, in need of such prevention and/or treatment.

GSK3 is highly expressed in the central and peripheral nervous system and in other tissues. Thus, it is expected that compounds of the invention are well suited for the prevention and/or treatment of conditions associated with glycogen synthase kinase-3 in the central and peripheral nervous system. In particular, the compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica.

Other conditions are selected from the group consisting of amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication.

Further conditions are selected from the group consisting of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders.

One embodiment of the invention relates to the prevention and/or treatment of dementia and Alzheimer's Disease.

Another embodiment of the invention relates to the prevention and/or treatment of bone-related disorders.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with glycogen synthase kinase-3.

In the context of the present specification, the term “therapy” also includes “prevention” unless there are specific indications to the contrary. The terms “therapeutic” and “therapeutically” should be construed accordingly.

The invention also provides for a method of treatment and/or prevention of conditions associated with glycogen synthase kinase-3 comprising administering to a mammal, is including man in need of such treatment and/or prevention a therapeutically effective amount of a compound of formula I, as hereinbefore defined.

### Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

Determination of ATP competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK30, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

TABLE 1
Specimen results from assay.

| Example no | Kᵢ (nM) | n |
|---|---|---|
| 1 | 10 | 3 |
| 17 | 14 | 4 |
| 22 | 22 | 3 |
| 29 | 126 | 2 |

1-30. (canceled)

31. A compound of formula I: 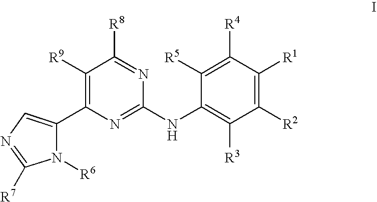
wherein
R¹ is selected from hydrogen, cyano, C₁₋₃haloalkyl, OR^{a}, SO₂NR^{b}R^{c}, C₀₋₂alkylC(O)NR^{b}R^{c}, C₁₋₄alkylNR^{b}R^{c}, CH₂OR^{h}, SO₂Rⁱ, C(O)OR^{a}, CH(OH)R^{j} and C(O)R^{j};
R² and R⁴ are independently selected from hydrogen, halo, cyano, NO₂, C₁₋₄alkyl, C₁₋₃haloalkyl, OR^{a}, C(O)NR^{b}R^{c}, SO₂Rⁱ and C(O)OR^{a}; or
R¹ and R², together with the atoms to which they are attached form a 5- or 6-membered heterocyclic ring containing at least one N, O or S, in which any of the hydrogens of the CH₂-groups within said heterocyclic ring can be substituted with oxo, hydroxy or halo and in which any sulphur atom within said heterocyclic ring is optionally oxidised to —SO₂—;
R³ and R⁵ are hydrogen;
R⁶ is tetrahydropyran;
R⁷ is selected from hydrogen, C₁₋₃alkyl, cyano and C₁₋₃haloalkyl, wherein said C₁₋₃alkyl or C₁₋₃haloalkyl is optionally substituted with one or more OR^{a};
R⁸ is hydrogen;
R⁹ is hydrogen or fluoro;
R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;
R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};
R^{d} and R^{e} are independently selected from hydrogen, C₁₋₆alkyl and C₁₋₆haloalkyl, wherein said C₁₋₆alkyl or C₁₋₆haloalkyl is optionally substituted with one or more OR^{a}; or R^{d} and R^{e} may, together with the atom to which they are attached, form a heterocyclic ring wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy;
Rⁱ is selected from C₁₋₆alkyl, heterocyclyl, aryl, heteroaryl and C₁₋₃haloalkyl, wherein said C₁₋₆alkyl, heterocyclyl, aryl, heteroaryl or C₁₋₃haloalkyl is optionally substituted with one or more halo, cyano, di-(C₁₋₄alkyl)amino-, C₁₋₃haloalkyl, C₁₋₃alkyl, heterocyclyl or OR^{a};
R^{j} is an aryl or heteroaryl ring, wherein said aryl or heteroaryl ring is optionally substituted with one or more C₁₋₃alkyl, OR^{a}, halo or cyano;
as a base or a pharmaceutically acceptable salt, solvate or solvate of a salt thereof.

32. (canceled)

33. A compound according to claim 31, wherein
R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR³, CH(OH)R^{j} and C(O)R^{j};
R² and R⁴ are independently selected from hydrogen, halo, cyano, NO₂, C₁₋₄alkyl, C₁₋₃haloalkyl, OR^{a}, SO₂Rⁱ, C(O)NR^{b}R^{c} and C(O)OR^{a}; or
R¹ and R², together with the atoms to which they are attached join to form a 5- or 6-membered heterocyclic ring containing at least one N, O or S, in which any of the hydrogens of the CH₂-groups within the said heterocyclic ring can be substituted with oxo, hydroxy or halo and in which any sulphur atom within said heterocyclic ring is optionally oxidised to —SO₂—;
R³ and R⁵ are hydrogen;
R⁶ is tetrahydropyran;
R⁷ is selected from C₁₋₃alkyl, cyano, and C₁₋₃haloalkyl;
R⁸ is hydrogen;
R⁹ is hydrogen or fluoro;
R^{a} is selected from hydrogen, C₁₋₃alkyl and C₁₋₃haloalkyl, wherein said C₁₋₃alkyl is optionally substituted with one or more C₁₋₃alkoxy;
R^{b} and R^{c} are independently selected from hydrogen, C₁₋₆alkyl and heterocyclyl, wherein said C₁₋₆alkyl, heterocyclyl is optionally substituted with one or more cyano, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};
R^{d} and R^{e} are independently selected from C₁₋₆alkyl; or R^{d} and R^{e} may, together with the atom to which they are attached, form a heterocyclic ring wherein said heterocyclic ring is optionally substituted with one or more halo;
Rⁱ is selected from C₁₋₆alkyl and heterocyclyl, wherein said C₁₋₆alkyl or heterocyclyl is optionally substituted with one or more di-(C₁₋₄alkyl)amino-, heterocyclyl or OR^{a};
R^{j} is an aryl or heteroaryl ring, wherein said aryl or heteroaryl ring is optionally substituted with one or more C₁₋₃alkyl;
as a base or a pharmaceutically acceptable salt, solvate or solvate of a salt thereof.

34-37. (canceled)

38. A compound according to claim 31 or 33, wherein R⁷ is methyl or trifluoromethyl.

39. A compound according to claim 31 or 33, wherein R⁴ is selected from hydrogen, halo, NO₂, C₁₋₄alkyl, C₁₋₃haloalkyl, OR^{a}, SO₂Rⁱ, C(O)NR^{b}R^{c} or C(O)OR³.

40. A compound according to claim 39, wherein R⁴ is C(O)NR^{b}R^{c} and wherein R^{b} and R^{c} are independently selected from hydrogen and C₁₋₆alkyl, wherein said C₁₋₆alkyl is optionally substituted with one or more OR^{a} and wherein R^{a} is C₁₋₃alkyl.

41. A compound according to claim 39, wherein R⁴ is trifluoromethyl.

42. A compound according to claim 39, wherein R⁴ is chloro.

43. A compound according to claim 39, wherein R^{a} is trifluoromethyl.

44. A compound according to claim 38, wherein R² is hydrogen, halo, C₁₋₃alkyl or OR^{a}.

45. A compound according to claim 44, wherein R² is chloro.

46. A compound according to claim 44, wherein R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} or C(O)R^{j}.

47. A compound according to claim 46, wherein
R¹ is C₀₋₂alkylC(O)NR^{b}R^{c} and
R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}.

48. A compound according to claim 47, wherein R^{b} and R^{c} together with the atom to which they are attached, form a heterocyclic ring, wherein said heterocyclic ring is optionally substituted with one or more halo, C₁₋₆alkyl or C₁₋₃haloalkyl, wherein said C₁₋₆alkyl or C₁₋₃haloalkyl is optionally further substituted with one or more C₁₋₃alkoxy or OR^{a}.

49. A compound according to claim 48, wherein said a heterocyclic ring is substituted with methyl.

50. A compound according to claim 46, wherein R¹ is C₁₋₄alkylNR^{b}R^{c} and R^{b} and R^{c} together with the atom to which they are attached, form a heterocyclic ring.

51. A compound according to claim 46, wherein R¹ is SO₂Rⁱ and Rⁱ is C₁₋₆alkyl, wherein said C₁₋₆alkyl is optionally substituted with one or more OR^{a}.

52. A compound according to claim 51, wherein Rⁱ is methyl.

53. A compound according to claim 46, wherein
R¹ is SO₂NR^{b}R^{c} and
R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}.

54. A compound according to claim 53, wherein R^{b} and R^{c} together with the atom to which they are attached form a heterocyclic ring, wherein said heterocyclic ring is optionally substituted with one or more halo, C₁₋₆alkyl or C₁₋₃haloalkyl.

55. A compound according to claim 54, wherein said heterocyclic ring is substituted with a C₁₋₆alkyl.

56. A compound according to claim 55, wherein said C₁₋₆alkyl is methyl.

57. A compound according to claim 31, wherein said compound is selected from:
5-Fluoro-N-{4-[(4-methylpiperazin-1-yl)sulfonyl]phenyl}-4-[2-methyl-1-(tetrahydro-2H-pyran-4-yl)-1H-imidazol-5-yl]pyrimidin-2-amine hydrochloride;
5-Fluoro-N-{4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-{3-methoxy-4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)sulfonyl]-3-(trifluoromethoxy)phenyl]-4-[2-methyl-1-(tetrahydro-2H-pyran-4-yl)-1H-imidazol-5-yl]pyrimidin-2-amine hydrochloride;
5-Fluoro-4-[2-methyl-1-(tetrahydro-2H-pyran-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(morpholin-4-ylsulfonyl)phenyl]pyrimidin-2-amine hydrochloride;
[4-({5-Fluoro-4-[2-methyl-1-(tetrahydro-2H-pyran-4-yl)-1H-imidazol-5-yl]pyrimidin-2-yl}amino)phenyl] (pyridin-2-yl)methanone hydrochloride;
5-Fluoro-4-[2-methyl-1-(tetrahydro-2H-pyran-4-yl)-1H-imidazol-5-yl]-N-[4-(morpholin-4-ylmethyl)phenyl]pyrimidin-2-amine hydrochloride;
5-Fluoro-4-[2-methyl-1-(tetrahydro-2H-pyran-4-yl)-1H-imidazol-5-yl]-N-[4-(piperidin-1-ylcarbonyl)phenyl]pyrimidin-2-amine hydrochloride;
5-Fluoro-4-[2-methyl-1-(tetrahydro-2H-pyran-4-yl)-1H-imidazol-5-yl]-N-[4-(trifluoromethyl)phenyl]pyrimidin-2-amine hydrochloride;
5-Fluoro-N-[3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)phenyl]-4-[2-methyl-1-(tetrahydro-2H-pyran-4-yl)-1H-imidazol-5-yl]pyrimidin-2-amine hydrochloride;
3-({5-Fluoro-4-[2-methyl-1-(tetrahydro-2H-pyran-4-yl)-1H-imidazol-5-yl]pyrimidin-2-yl}amino)benzonitrile hydrochloride;
4-({5-Fluoro-4-[2-methyl-1-(tetrahydro-2H-pyran-4-yl)-1H-imidazol-5-yl]pyrimidin-2-yl}amino)benzonitrile hydrochloride;
5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride; or
5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride
as a base or an alternative salt thereof.

58. A compound according to claim 31, said compound being selected from:
N-{4-[(Dimethylamino)methyl]phenyl}-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(1-morpholin-4-ylethyl)phenyl]pyrimidin-2-amine;
N-[4-(1-Azetidin-1-ylethyl)phenyl]-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(2-morpholin-4-ylethyl)phenyl]pyrimidin-2-amine;
N-[4-(Methylsulfonyl)phenyl]-4-[2-methyl-1-(tetrahydro-2H-pyran-4-yl)-1H-imidazol-5-yl]pyrimidin-2-amine;
N-{4-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{4-[(4-Methylpiperazin-1-yl)carbonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(morpholin-4-ylmethyl)phenyl]pyrimidin-2-amine;
4-[2-methyl-1-(tetrahydro-2H-pyran-4-yl)-1H-imidazol-5-yl]-N-[4-(morpholin-4-ylsulfonyl)phenyl]pyrimidin-2-amine;
N-(4-{[4-(2-Methoxyethyl)piperazin-1-yl]sulfonyl}phenyl)-4-[2-methyl-1-(tetrahydro-2H-pyran-4-yl)-1H-imidazol-5-yl]pyrimidin-2-amine;
N-{4-[(4-Isopropylpiperazin-1-yl)sulfonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(pyrrolidin-1-ylsulfonyl)phenyl]pyrimidin-2-amine;
(N-(1-Methylpiperidin-4-yl)-4-({4-[2-methyl-1-(tetrahydro-2H-pyran-4-yl)-1H-imidazol-5-yl]pyrimidin-2-yl}amino)benzenesulfonamide;
N-{4-[(4-Methyl-1,4-diazepan-1-yl)sulfonyl]phenyl}-4-[2-methyl-1-(tetrahydro-2H-pyran-4-yl)-1H-imidazol-5-yl]pyrimidin-2-amine;
N,N-Diethyl-4-({4-[2-methyl-1-(tetrahydro-2H-pyran-4-yl)-1H-imidazol-5-yl]pyrimidin-2-yl}amino)benzenesulfonamide;
N-[4-(Azetidin-1-ylsulfonyl)phenyl]-4-[2-methyl-1-(tetrahydro-2H-pyran-4-yl)-1H-imidazol-5-yl]pyrimidin-2-amine;
N-{3-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Chloro-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(3R)-3-methylmorpholin-4-yl]sulfonyl}phenyl)-4-[2-methyl-1-(tetrahydro-2H-pyran-4-yl)-1H-imidazol-5-yl]pyrimidin-2-amine;
5-Fluoro-N-{3-methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(1S,4S)-5-methyl-2,5-diazabicyclo[2.2.1]hept-2-yl]sulfonyl}phenyl)-4-[2-methyl-1-(tetrahydro-2H-pyran-4-yl)-1H-imidazol-5-yl]pyrimidin-2-amine;
4-({5-Fluoro-4-[2-methyl-1-(tetrahydro-2H-pyran-4-yl)-1H-imidazol-5-yl]pyrimidin-2-yl}amino)-N,N-dimethylbenzenesulfonamide;
N-[4-(Azetidin-1-ylsulfonyl)phenyl]-5-fluoro-4-[2-methyl-1-(tetrahydro-2H-pyran-4-yl)-1H-imidazol-5-yl]pyrimidin-2-amine;
Ethyl 4-({5-fluoro-4-[2-methyl-1-(tetrahydro-2H-pyran-4-yl)-1H-imidazol-5-yl]pyrimidin-2-yl}amino)benzoate;
5-Fluoro-N-[4-(methylsulfonyl)phenyl]-4-[1-(tetrahydro-2H-pyran-4-yl)-2-(trifluoromethyl)-1H-imidazol-5-yl]pyrimidin-2-amine;
N-[4-(Azetidin-1-ylcarbonyl)phenyl]-5-fluoro-4-[1-(tetrahydro-2H-pyran-4-yl)-2-(trifluoromethyl)-1H-imidazol-5-yl]pyrimidin-2-amine;
N-{3-Chloro-4-[(4-methylpiperazin-1-yl)carbonyl]phenyl}-5-fluoro-4-[2-methyl-1-(tetrahydro-2H-pyran-4-yl)-1H-imidazol-5-yl]pyrimidin-2-amine;
5-Fluoro-N-{3-[(4-methylpiperazin-1-yl)carbonyl]phenyl}-4-[2-methyl-1-(tetrahydro-2H-pyran-4-yl)-1H-imidazol-5-yl]pyrimidin-2-amine;
5-Fluoro-N-[4-(isopropylsulfonyl)phenyl]-4-[2-methyl-1-(tetrahydro-2H-pyran-4-yl)-1H-imidazol-5-yl]pyrimidin-2-amine;
N-[4-(Ethylsulfonyl)phenyl]-5-fluoro-4-[2-methyl-1-(tetrahydro-2H-pyran-4-yl)-1H-imidazol-5-yl]pyrimidin-2-amine;
5-Fluoro-N-{4-[(2-methoxyethyl)sulfonyl]phenyl}-4-[2-methyl-1-(tetrahydro-2H-pyran-4-yl)-1H-imidazol-5-yl]pyrimidin-2-amine;
N-(4-{[2-(Diethylamino)ethyl]sulfonyl}phenyl)-5-fluoro-4-[2-methyl-1-(tetrahydro-2H-pyran-4-yl)-1H-imidazol-5-yl]pyrimidin-2-amine;
2-{[4-({5-Fluoro-4-[2-methyl-1-(tetrahydro-2H-pyran-4-yl)-1H-imidazol-5-yl]pyrimidin-2-yl}amino)phenyl]sulfonyl}ethanol;
{5-Fluoro-4-[3-(tetrahydro-pyran-4-yl)-3H-imidazol-4-yl]-pyrimidin-2-yl}-[4-(4-methyl-piperazine-1-sulfonyl)-phenyl]-amine;
5-{5-Fluoro-2-[4-(4-methyl-piperazine-1-sulfonyl)-phenylamino]-pyrimidin-4-yl}-1-(tetrahydro-pyran-4-yl)-1H-imidazole-2-carbonitrile; or
{5-Fluoro-4-[2-methyl-3-(tetrahydro-pyran-4-yl)-3H-imidazol-4-yl]-pyrimidin-2-yl}-[4-(tetrahydro-pyran-2-ylmethanesulfonyl)-phenyl]-amine;
or a pharmaceutically acceptable salt thereof.

59. A compound according to claim 31, said compound being selected from:
N-[4-(Azetidin-1-ylcarbonyl)phenyl]-5-fluoro-4-[2-methyl-1-(tetrahydro-2H-pyran-4-yl)-1H-imidazol-5-yl]pyrimidin-2-amine hydrochloride;
N-{4-[(3,3-Difluoroazetidin-1-yl)carbonyl]phenyl}-5-fluoro-4-[2-methyl-1-(tetrahydro-2H-pyran-4-yl)-1H-imidazol-5-yl]pyrimidin-2-amine hydrochloride;
5-Fluoro-N-[3-methyl-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-{4-[(4-fluoropiperidin-1-yl)carbonyl]phenyl}-4-[2-methyl-1-(tetrahydro-2H-pyran-4-yl)-1H-imidazol-5-yl]pyrimidin-2-amine hydrochloride;
N,N-Diethyl-4-({5-fluoro-4-[2-methyl-1-(tetrahydro-2H-pyran-4-yl)-1H-imidazol-5-yl]pyrimidin-2-yl}amino)benzamide hydrochloride;
4-[5-fluoro-4-(2-methyl-3-tetrahydropyran-4-yl-imidazol-4-yl)-pyrimidin-2-yl]amino-N-(3-methoxypropyl)benzamide hydrochloride;
[4-[5-fluoro-4-(2-methyl-3-tetrahydropyran-4-yl-imidazol-4-yl)-pyrimidin-2-yl]aminophenyl]-(1,4-oxazepan-4-yl)methanone hydrochloride;
(4-ethylpiperazin-1-yl)-[4-[5-fluoro-4-(2-methyl-3-tetrahydropyran-4-yl-imidazol-4-yl)-pyrimidin-2-yl]aminophenyl]-methanone hydrochloride;
(2,6-dimethylmorpholin-4-yl)-[4-[5-fluoro-4-(2-methyl-3-tetrahydropyran-4-yl-imidazol-4-yl)-pyrimidin-2-yl]aminophenyl]-methanone hydrochloride;
[4-[5-fluoro-4-(2-methyl-3-tetrahydropyran-4-yl-imidazol-4-yl)-pyrimidin-2-yl]aminophenyl]-(3-fluoropyrrolidin-1-yl)-methanone hydrochloride;
(3,3-difluoropyrrolidin-1-yl)-[4-[5-fluoro-4-(2-methyl-3-tetrahydropyran-4-yl-imidazol-4-yl)-pyrimidin-2-yl]aminophenyl]-methanone hydrochloride;
4-[5-fluoro-4-(2-methyl-3-tetrahydropyran-4-yl-imidazol-4-yl)-pyrimidin-2-yl]amino-N-methyl-benzamide hydrochloride;
4-[5-fluoro-4-(2-methyl-3-tetrahydropyran-4-yl-imidazol-4-yl)-pyrimidin-2-yl]amino-N-tetrahydropyran-4-yl-benzamide hydrochloride;
[4-[5-fluoro-4-(2-methyl-3-tetrahydropyran-4-yl-imidazol-4-yl)-pyrimidin-2-yl]aminophenyl]-(3-hydroxypyrrolidin-1-yl)-methanone hydrochloride;
N-(2-cyanoethyl)-4-[5-fluoro-4-(2-methyl-3-tetrahydropyran-4-yl-imidazol-4-yl)-pyrimidin-2-yl]amino-N-methyl-benzamide hydrochloride;
N-ethyl-4-[5-fluoro-4-(2-methyl-3-tetrahydropyran-4-yl-imidazol-4-yl)-pyrimidin-2-yl]amino-N-(2-hydroxyethyl)benzamide hydrochloride;
4-[5-fluoro-4-(2-methyl-3-tetrahydropyran-4-yl-imidazol-4-yl)-pyrimidin-2-yl]amino-N-(2-hydroxyethyl)-N-methyl-benzamide hydrochloride;
4-[5-fluoro-4-(2-methyl-3-tetrahydropyran-4-yl-imidazol-4-yl)-pyrimidin-2-yl]amino-N-(2-hydroxyethyl)benzamide hydrochloride;
N-(2-dimethylaminoethyl)-4-[5-fluoro-4-(2-methyl-3-tetrahydropyran-4-yl-imidazol-4-yl)-pyrimidin-2-yl]amino-benzamide hydrochloride;
(4-dimethylamino-1-piperidyl)-[4-[5-fluoro-4-(2-methyl-3-tetrahydropyran-4-yl-imidazol-4-yl)-pyrimidin-2-yl]aminophenyl]-methanone hydrochloride;
[4-[5-fluoro-4-(2-methyl-3-tetrahydropyran-4-yl-imidazol-4-yl)-pyrimidin-2-yl]aminophenyl]-[4-(2-methoxyethyl)piperazin-1-yl]-methanone hydrochloride;
4-[5-fluoro-4-(2-methyl-3-tetrahydropyran-4-yl-imidazol-4-yl)-pyrimidin-2-yl]amino-N-[2-(1-piperidyl)ethyl]benzamide hydrochloride;
4-[5-fluoro-4-(2-methyl-3-tetrahydropyran-4-yl-imidazol-4-yl)-pyrimidin-2-yl]amino-N-(2-morpholinoethyl)benzamide hydrochloride;
4-[5-fluoro-4-(2-methyl-3-tetrahydropyran-4-yl-imidazol-4-yl)-pyrimidin-2-yl]amino-N-isopropyl-benzamide hydrochloride;
N-[2-(3,3-difluoropyrrolidin-1-yl)ethyl]-4-[5-fluoro-4-(2-methyl-3-tetrahydropyran-4-yl-imidazol-4-yl)-pyrimidin-2-yl]amino-benzamide hydrochloride;
[4-[5-fluoro-4-(2-methyl-3-tetrahydropyran-4-yl-imidazol-4-yl)-pyrimidin-2-yl]aminophenyl]-(4-isopropylpiperazin-1-yl)-methanone hydrochloride;
[4-[5-fluoro-4-(2-methyl-3-tetrahydropyran-4-yl-imidazol-4-yl)-pyrimidin-2-yl]aminophenyl]-(4-methyl-1,4-diazepan-1-yl)-methanone hydrochloride;
4-[5-fluoro-4-(2-methyl-3-tetrahydropyran-4-yl-imidazol-4-yl)-pyrimidin-2-yl]amino-N-tetrahydrofuran-3-yl-benzamide hydrochloride;
[4-[5-Fluoro-4-[3-tetrahydropyran-4-yl-2-(trifluoromethyl)imidazol-4-yl]-pyrimidin-2-yl]aminophenyl]-(4-methylpiperazin-1-yl)-methanone hydrochloride;
5-Fluoro-N-[3-(methylsulfonyl)-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)-3-(trifluoromethyl)phenyl]-4-[2-methyl-1-(tetrahydro-2H-pyran-4-yl)-1H-imidazol-5-yl]pyrimidin-2-amine hydrochloride;
6-({5-Fluoro-4-[2-methyl-1-(tetrahydro-2H-pyran-4-yl)-1H-imidazol-5-yl]pyrimidin-2-yl}amino)-2,3-dihydro-4H-thiochromen-4-one 1,1-dioxide hydrochloride; or
6-({5-Fluoro-4-[2-methyl-1-(tetrahydro-2H-pyran-4-yl)-1H-imidazol-5-yl]pyrimidin-2-yl}amino)thiochroman-4-ol 1,1-dioxide hydrochloride; ad
as a base or alternative salt thereof.

60-61. (canceled)

62. A compound selected from:
2-Chloro-4-(1,2-dimethyl-1H-imidazol-5-yl)-5-fluoropyrimidine;
2-Methyl-4-[(4-methylpiperazin-1-yl)carbonyl]aniline;
4-[(4-Methylpiperazin-1-yl)carbonyl]-3-nitroaniline;
4-[(4-Methylpiperazin-1-yl)carbonyl]-2-(trifluoromethoxy)aniline;
4-[N-Acetyl-N-(tetrahydro-2H-pyran-4-yl)]amino-5-methylisoxazole;
5-Acetyl-2-methyl-1-(tetrahydro-2H-pyran-4-yl)-1H-imidazole;
(2E)-3-Dimethylamino-1-[2-methyl-1-(tetrahydro-2H-pyran-4-yl)-1H-imidazol-5-yl]prop-2-en-1-one;
(2Z)-3-Dimethylamino-2-fluoro-1-[2-methyl-1-(tetrahydro-2H-pyran-4-yl)-1H-imidazol-5-yl]prop-2-en-1-one;
5-Fluoro-4-[2-methyl-1-(tetrahydro-2H-pyran-4-yl)-1H-imidazol-5-yl]pyrimidin-2-amine;
1-(4-Chloro-2-methoxybenzoyl)-4-methylpiperazine;
1-[4-Bromo-2-(methylsulfonyl)benzoyl]-4-methylpiperazine;
4-(N-Acetyl-N-cyclohexyl)amino-5-methylisoxazole;
5-Acetyl-1-cyclohexyl-2-methyl-1H-imidazole;
(2E)-3-Dimethylamino-1-(1-cyclohexyl-2-methyl-1H-imidazol-5-yl)prop-2-en-1-one;
(2Z)-3-Dimethylamino-2-fluoro-1-(1-cyclohexyl-2-methyl-1H-imidazol-5-yl)prop-2-en-1-one;
4-(1-Cyclohexyl-2-methyl-1H-imidazol-5-yl)-5-fluoropyrimidin-2-amine;
5-Acetyl-2-methyl-1-(1-methylpiperidin-4-yl)-1H-imidazole;
(2E)-3-Dimethylamino-1-[2-methyl-1-(1-methylpiperidin-4-yl)-1H-imidazol-5-yl]prop-2-en-1-one;
(2Z)-3-Dimethylamino-2-fluoro-1-[2-methyl-1-(1-methylpiperidin-4-yl)-1H-imidazol-5-yl]prop-2-en-1-one;
5-Fluoro-4-[2-methyl-1-(1-methylpiperidin-4-yl)-1H-imidazol-5-yl]pyrimidin-2-amine;
4-(1,2-Dimethyl-1H-imidazol-5-yl)-5-fluoropyrimidin-2-amine;
1-(tert-Butoxycarbonyl)-4-(4-bromo-benzenesulfonyl)-piperazine;
5-Acetyl-1-(tetrahydro-2H-pyran-4-yl)-2-trifluoromethyl-1H-imidazole;
(2E)-3-Dimethylamino-1-[1-(tetrahydro-2H-pyran-4-yl)-2-trifluoromethyl-1H-imidazol-5-yl]prop-2-en-1-one;
(2Z)-3-Dimethylamino-2-fluoro-1-[1-(tetrahydro-2H-pyran-4-yl)-2-trifluoromethyl-1H-imidazol-5-yl]prop-2-en-1-one;
5-Fluoro-4-[1-(tetrahydro-2H-pyran-4-yl)-2-(trifluoromethyl)-1H-imidazol-5-yl]pyrimidin-2-amine;
4-[2-Methyl-1-(tetrahydro-2H-pyran-4-yl)-1H-imidazol-5-yl]pyrimidin-2-amine;
1-[(4-Bromo-2-chlorophenyl)sulfonyl]-4-methylpiperazine;
(3R)-4-[(4-Bromophenyl)sulfonyl]-3-methylmorpholine;
(1S,4S)-2-[(4-Bromophenyl)sulfonyl]-5-methyl-2,5-diazabicyclo [2.2.1]heptane;
Methyl 4-bromo-2-(trifluoromethoxy)benzoate;
4-Bromo-2-(trifluoromethoxy)benzoic acid;
4-(4-Chloro-2-methylbenzyl)morpholine;
Lithium 4-({5-fluoro-4-[2-methyl-1-(tetrahydro-2H-pyran-4-yl)-1H-imidazol-5-yl]pyrimidin-2-yl}amino)benzoate;
1-(4-Bromo-2-methylbenzoyl)azetidine;
4-Bromo-2-(trifluoromethoxy)benzoic acid;
1-[4-Bromo-2-(trifluoromethoxy)benzoyl]azetidine;
2,2,2-Trifluoro-N-methyl-N-(5-methylisoxazol-4-yl)acetamide;
1-[1-Methyl-2-(trifluoromethyl)-1H-imidazol-5-yl]ethanone;
(2E)-3-(Dimethylamino)-1-[1-methyl-2-(trifluoromethyl)-1H-imidazol-5-yl]prop-2-en-1-one;
(2Z)-3-(Dimethylamino)-2-fluoro-1-[1-methyl-2-(trifluoromethyl)-1H-imidazol-5-yl]prop-2-en-1-one;
5-Fluoro-4-[1-methyl-2-(trifluoromethyl)-1H-imidazol-5-yl]pyrimidin-2-amine;
4-[4-Bromo-2-(methylsulfonyl)benzyl]morpholine;
2-[(4-Bromophenyl)sulfonyl]ethyl methyl ether;
2-[(4-Bromophenyl)sulfonyl]ethyl diethyl-amine;
N-(5-Methyl-isoxazol-4-yl)-N-(tetrahydro-pyran-4-yl)-formamide;
5-Acetyl-1-(tetrahydro-pyran-4-yl)-1H-imidazole;
(E)-3-Dimethylamino-1-[3-(tetrahydro-pyran-4-yl)-3H-imidazol-4-yl]-propenone;
(Z)-3-Dimethylamino-2-fluoro-1-[3-(tetrahydro-pyran-4-yl)-3H-imidazol-4-yl]-propenone;
5-Fluoro-4-[1-(tetrahydro-2H-pyran-4-yl)-1H-imidazol-5-yl]pyrimidin-2-amine; and
5-{5-Fluoro-2-[4-(4-methyl-piperazine-1-sulfonyl)-phenylamino]-pyrimidin-4-yl}-1-(tetrahydro-pyran-4-yl)-1H-imidazole-2-carbaldehyde.

63. (canceled)

64. A pharmaceutical formulation comprising as active ingredient a therapeutically effective amount of a compound according to claim 31, in association with pharmaceutically acceptable excipients, carriers or diluents.

65. (canceled)

66. A method of prevention and/or treatment of dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, comprising administering to a mammal, including man in need of such prevention and/or treatment, a therapeutically effective amount of a compound of formula I as defined in claim 31.

67. The method according to claim 66, wherein the disease is Alzheimer's Disease.

68. A method of prevention and/or treatment of predemented states, Mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia and Type I and Type II diabetes, diabetic neuropathy and diabetes related disorders, comprising administering to a mammal, including man in need of such prevention and/or treatment, a therapeutically effective amount of a compound of formula I as defined in claim 31.

69. (canceled)

70. A method pr preventing or treating a condition selected from dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, bipolar disorder, affective disorders, depression, schizophrenia or cognitive disorders, comprising administering to a subject in need of such prevention or treatment, a therapeutically effective amount of a compound of formula I: 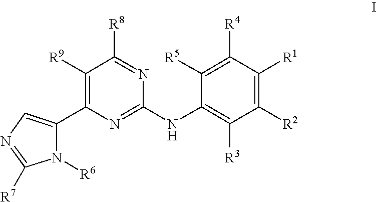
wherein
R¹ is selected from hydrogen, halo, cyano, NO₂, C₁₋₃alkyl, C₁₋₃haloalkyl, OR^{a}, SO₂NR^{b}R^{c}, C₀₋₂alkylC(O)NR^{b}R^{c}, C₁₋₄alkylNR^{b}R^{c}, CH₂OR^{h}, SO₂Rⁱ, C(O)OR^{a}, CH(OH)R^{j} and C(O)R^{j};
R² and R⁴ are independently selected from hydrogen, halo, cyano, NO₂, C₁₋₄alkyl, C₁₋₃haloalkyl, OR^{a}, SO₂NR^{b}R^{c}, C(O)NR^{b}R^{c}, CH₂NR^{b}R^{c}, CH₂OR^{h}, SO₂Rⁱ, C(O)OR^{a} and C(O)R^{j}; or R¹ and R², together with the atoms to which they are attached join to form a 5- or 6-membered heterocyclic ring containing at least one N, O or S, in which any —CH₂-group of said heterocyclic ring can be substituted with oxo, hydroxy or halo and in which any sulphur atom within said heterocyclic ring is optionally oxidised to —SO₂—;
R³ and R⁵ are independently selected from hydrogen, halo, cyano, C₁₋₃alkyl, C₁₋₃haloalkyl and OR^{a};
R⁶ is selected from CH₃ and C₆alkyl, C₆alkenyl, C₆alkynyl and C₆haloalkyl; or R⁶ is a 6-membered heterocyclic ring containing one or more heteroatoms selected from N, O or S, wherein said heterocyclic ring is optionally substituted with one or more C₁₋₃alkyl or C₁₋₃haloalkyl, wherein said C₁₋₃alkyl or C₁₋₃haloalkyl is optionally further substituted with one or more C₁₋₃alkoxy;
R⁷ is selected from hydrogen, C₁₋₃alkyl, cyano, and C₁₋₃haloalkyl, wherein said C₁₋₃alkyl or C₁₋₃haloalkyl is optionally substituted with one or more OR^{a};
R³ and R⁹ are independently selected from hydrogen, cyano and halo;
R^{a} is selected from hydrogen, C₁₋₃alkyl and C₁₋₃haloalkyl, wherein said C₁₋₃alkyl or C₁₋₃haloalkyl is optionally substituted with one or more C₁₋₃alkoxy;
R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a};
R^{d} and R^{e} are independently selected from hydrogen, C₁₋₆alkyl and C₁₋₆haloalkyl, wherein said C₁₋₆alkyl or C₁₋₆haloalkyl is optionally substituted with one or more OR^{a}; or R^{d} and R^{e} may, together with the atom to which they are attached, form a heterocyclic ring wherein said heterocyclic ring is optionally substituted with one or more halo, C₁₋₃alkyl or C₁₋₃haloalkyl, wherein said C₁₋₃alkyl or C₁₋₃haloalkyl is optionally further substituted with one or more C₁₋₃alkoxy;
R^{h} is hydrogen, C₁₋₃alkyl or C₁₋₃haloalkyl, wherein said C₁₋₃alkyl or C₁₋₃haloalkyl is optionally substituted with one or more C₁₋₃alkoxy;
Rⁱ is C₁₋₆alkyl, heterocyclyl, aryl, heteroaryl or C₁₋₃haloalkyl, wherein said C₁₋₆alkyl, heterocyclyl, aryl, heteroaryl or C₁₋₃haloalkyl is optionally substituted with one or more halo, cyano, di-(C₁₋₄alkyl)amino-, C₁₋₃haloalkyl, C₁₋₃alkyl, heterocyclyl or OR^{a};
R^{j} is an aryl or heteroaryl ring, wherein said aryl or heteroaryl ring is optionally substituted with one or more C₁₋₃alkyl, OR^{a}, halo or cyano;
as a base or a pharmaceutically acceptable salt, solvate or solvate of a salt thereof.

71. A method according to claim 70, wherein said compound is a compound in accord with formula I or formula Ia, wherein;
R⁶ is selected from CH₃ and C₆alkyl; or R⁶ is a 6-membered heterocyclic ring containing one or more heteroatoms selected from N, O or S, wherein said heterocyclic ring is optionally substituted with one or more C₁₋₃alkyl or C₁₋₃haloalkyl;
as a base or a pharmaceutically acceptable salt, solvate or solvate of a salt thereof.

72. A method according to claim 70, wherein said compound is a compound in accord with formula I and R³ and R⁵ are hydrogen.

73. A method according to claim 72, wherein R⁸ is hydrogen and R⁹ is hydrogen or fluoro.

74. A method according to claim 73, wherein R⁶ is tetrahydropyran.

75. A method according to claim 72 or 73, wherein R⁷ is methyl or trifluoromethyl.

76. A method according to claim 72, wherein R⁴ is selected from hydrogen, halo, NO₂, C₁₋₄alkyl, C₁₋₃haloalkyl, OR^{a}, SO₂Rⁱ, C(O)NR^{b}R^{c} and C(O)OR^{a}.

77. A method according to claim 72, wherein R⁴ is C(O)NR^{b}R^{c} and wherein R^{b} and R^{c} are independently selected from hydrogen and C₁₋₆alkyl, wherein said C₁₋₆alkyl is optionally substituted with one or more OR^{a} and wherein R^{a} is C₁₋₃alkyl.

78. A method according to claim 72, wherein R² is hydrogen, halo, C₁₋₃alkyl or OR^{a}.

79. A method according to claim 78, wherein R¹ is selected from hydrogen, cyano, C₁₋₃haloalkyl, SO₂NR^{b}R^{c}, C₀₋₂alkylC(O)NR^{b}R^{c}, C₁₋₄alkylNR^{b}R^{c}, SO₂Rⁱ, C(O)OR^{a}, CH(OH)R^{j} and C(O)R^{j}.

80. A method according to claim 79, wherein:
R¹ is C₀₋₂alkylC(O)NR^{b}R¹ and
R^{b} and R^{c} are independently selected from hydrogen, C₁₋₆alkyl, heterocyclyl, aryl, heteroaryl and C₁₋₆haloalkyl, wherein said C₁₋₆alkyl, heterocyclyl, aryl, heteroaryl or C₁₋₆haloalkyl is optionally substituted with one or more C₁₋₄alkyl, C₁₋₄haloalkyl, halo, cyano, methanesulphonyl-, OR^{a} or NR^{d}R^{e}; or R^{b} and R^{c} may, together with the atom to which they are attached, form a heterocyclic ring wherein said heterocyclic ring is optionally substituted with one or more halo, hydroxy, cyano, di-(C₁₋₄alkyl)amino-, C₁₋₆alkyl or C₁₋₃haloalkyl, wherein said C₁₋₆alkyl or C₁₋₃haloalkyl is optionally further substituted with one or more C₁₋₃alkoxy or OR^{a}.

81. A method according to claim 80, wherein R^{b} and R^{c} together with the atom to which they are attached, form a heterocyclic ring, wherein said heterocyclic ring is optionally substituted with one or more halo, C₁₋₆alkyl or C₁₋₃haloalkyl, wherein said C₁₋₆alkyl or C₁₋₃haloalkyl is optionally further substituted with one or more C₁₋₃alkoxy or OR^{a}.

82. A method according to claim 81, wherein said heterocyclic ring is substituted with methyl.

83. A method according to claim 79, wherein R¹ is C₁₋₄alkylNR^{b}R^{c} and R^{b} and R^{c} together with the atom to which they are attached, form a heterocyclic ring.

84. A method according to claim 70, wherein the condition is bipolar disorder.

85. A method pr preventing or treating a subject afflicted with a condition selected from dementia, Alzheimer's Disease, Parkinson's Disease, Frontotemporal dementia Parkinson's Type, Parkinson dementia complex of Guam, HIV dementia, diseases with associated neurofibrillar tangle pathologies and dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, bipolar disorder, affective disorders, depression, schizophrenia or cognitive disorders, comprising administering to a subject in need of such prevention or treatment, a therapeutically effective amount of a compound selected from:
4-(1,2-Dimethyl-1H-imidazol-5-yl)-5-fluoro-N-[3-methoxy-5-(trifluoromethyl)phenyl]pyrimidin-2-amine;
N-(3,5-Dichlorophenyl)-4-(1,2-dimethyl-1H-imidazol-5-yl)-5-fluoropyrimidin-2-amine;
(4-{[4-(1,2-Dimethyl-1H-imidazol-5-yl)-5-fluoropyrimidin-2-yl]amino}phenyl)(phenyl)methanone;
4-(1,2-Dimethyl-1H-imidazol-5-yl)-5-fluoro-N-{2-methyl-4-[(4-methylpiperazin-1-yl)carbonyl]phenyl}pyrimidin-2-amine;
4-(1,2-Dimethyl-1H-imidazol-5-yl)-5-fluoro-N-{4-[(4-methylpiperazin-1-yl)carbonyl]-3-nitrophenyl}pyrimidin-2-amine;
4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride;
5-Fluoro-N-{4-[(4-methylpiperazin-1-yl)sulfonyl]phenyl}-4-[2-methyl-1-(tetrahydro-2H-pyran-4-yl)-1H-imidazol-5-yl]pyrimidin-2-amine hydrochloride;
5-Fluoro-N-{4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-{3-methoxy-4-[(4-methylpiperazin-1-yl)carbonyl]phenyl}-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)carbonyl]-3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-[(4-methylpiperazin-1-yl)sulfonyl]-3-(trifluoromethoxy)phenyl]-4-[2-methyl-1-(tetrahydro-2H-pyran-4-yl)-1H-imidazol-5-yl]pyrimidin-2-amine hydrochloride;
5-Fluoro-4-[2-methyl-1-(tetrahydro-2H-pyran-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(morpholin-4-ylsulfonyl)phenyl]pyrimidin-2-amine hydrochloride;
[4-({5-Fluoro-4-[2-methyl-1-(tetrahydro-2H-pyran-4-yl)-1H-imidazol-5-yl]pyrimidin-2-yl}amino)phenyl] (pyridin-2-yl)methanone hydrochloride;
5-Fluoro-4-[2-methyl-1-(tetrahydro-2H-pyran-4-yl)-1H-imidazol-5-yl]-N-[4-(morpholin-4-ylmethyl)phenyl]pyrimidin-2-amine hydrochloride;
5-Fluoro-4-[2-methyl-1-(tetrahydro-2H-pyran-4-yl)-1H-imidazol-5-yl]-N-[4-(piperidin-1-ylcarbonyl)phenyl]pyrimidin-2-amine hydrochloride;
4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride;
4-(1-Cyclohexyl-2-methyl-1H-imidazol-5-yl)-5-fluoro-N-{4-[(4-methylpiperazin-1-yl)sulfonyl]phenyl}pyrimidin-2-amine hydrochloride;
5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride;
5-Fluoro-4-[2-methyl-1-(1-methylpiperidin-4-yl)-1H-imidazol-5-yl]-N-[4-(pyrrolidin-1-ylsulfonyl)phenyl]pyrimidin-2-amine hydrochloride;
5-Fluoro-4-[2-methyl-1-(tetrahydro-2H-pyran-4-yl)-1H-imidazol-5-yl]-N-[4-(trifluoromethyl)phenyl]pyrimidin-2-amine hydrochloride;
5-Fluoro-N-[3-(methylsulfonyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)phenyl]-4-[2-methyl-1-(tetrahydro-2H-pyran-4-yl)-1H-imidazol-5-yl]pyrimidin-2-amine hydrochloride;
3-({5-Fluoro-4-[2-methyl-1-(tetrahydro-2H-pyran-4-yl)-1H-imidazol-5-yl]pyrimidin-2-yl}amino)benzonitrile hydrochloride;
4-(1,2-Dimethyl-1H-imidazol-5-yl)-5-fluoro-N-[4-(morpholin-4-ylmethyl)phenyl]pyrimidin-2-amine hydrochloride;
4-(1,2-Dimethyl-1H-imidazol-5-yl)-5-fluoro-N-{4-[(4-methylpiperazin-1-yl)sulfonyl]phenyl}pyrimidin-2-amine;
4-(1,2-Dimethyl-1H-imidazol-5-yl)-5-fluoro-N-[4-(piperidin-1-ylcarbonyl)phenyl]pyrimidin-2-amine hydrochloride;
4-(1,2-Dimethyl-1H-imidazol-5-yl)-5-fluoro-N-{4-[(4-methylpiperazin-1-yl)carbonyl]phenyl}pyrimidin-2-amine hydrochloride;
4-(1,2-Dimethyl-1H-imidazol-5-yl)-5-fluoro-N-{4-[(4-methylpiperazin-1-yl)methyl]phenyl}pyrimidin-2-amine hydrochloride;
4-(1,2-Dimethyl-1H-imidazol-5-yl)-5-fluoro-N-{3-[(4-methylpiperazin-1-yl)carbonyl]phenyl}pyrimidin-2-amine hydrochloride;
(4-{[4-(1,2-Dimethyl-1H-imidazol-5-yl)-5-fluoropyrimidin-2-yl]amino}phenyl)(pyridin-2-yl)methanone hydrochloride;
4-({5-Fluoro-4-[2-methyl-1-(tetrahydro-2H-pyran-4-yl)-1H-imidazol-5-yl]pyrimidin-2-yl}amino)benzonitrile hydrochloride;
5-Fluoro-4-[2-methyl-1-(tetrahydro-2H-pyran-4-yl)-1H-imidazol-5-yl]-N-[4-(piperazin-1-ylsulfonyl)phenyl]pyrimidin-2-amine hydrochloride;
5-Fluoro-N-{4-[(4-methylpiperazin-1-yl)sulfonyl]phenyl}-4-[1-(tetrahydro-2H-pyran-4-yl)-2-(trifluoromethyl)-1H-imidazol-5-yl]pyrimidin-2-amine hydrochloride;
N-{4-[(Dimethylamino)methyl]phenyl}-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(1-morpholin-4-ylethyl)phenyl]pyrimidin-2-amine;
N-[4-(1-Azetidin-1-ylethyl)phenyl]-5-fluoro-4-[2-methyl-1-(tetrahydro-2H-pyran-4-yl)-1H-imidazol-5-yl]pyrimidin-2-amine;
5-Fluoro-4-[2-methyl-1-(tetrahydro-2H-pyran-4-yl)-1H-imidazol-5-yl]-N-[4-(2-morpholin-4-ylethyl)phenyl]pyrimidin-2-amine;
N-[4-(Methylsulfonyl)phenyl]-4-[2-methyl-1-(tetrahydro-2H-pyran-4-yl)-1H-imidazol-5-yl]pyrimidin-2-amine;
N-{4-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{4-[(4-Methylpiperazin-1-yl)carbonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(morpholin-4-ylmethyl)phenyl]pyrimidin-2-amine;
4-[2-methyl-1-(tetrahydro-2H-pyran-4-yl)-1H-imidazol-5-yl]-N-[4-(morpholin-4-ylsulfonyl)phenyl]pyrimidin-2-amine;
N-(4-{[4-(2-Methoxyethyl)piperazin-1-yl]sulfonyl}phenyl)-4-[2-methyl-1-(tetrahydro-2H-pyran-4-yl)-1H-imidazol-5-yl]pyrimidin-2-amine;
N-{4-[(4-Isopropylpiperazin-1-yl)sulfonyl]phenyl}-4-[2-methyl-1-(tetrahydro-2H-pyran-4-yl)-1H-imidazol-5-yl]pyrimidin-2-amine;
4-[2-Methyl-1-(tetrahydro-2H-pyran-4-yl)-1H-imidazol-5-yl]-N-[4-(pyrrolidin-1-ylsulfonyl)phenyl]pyrimidin-2-amine;
(N-(1-Methylpiperidin-4-yl)-4-({4-[2-methyl-1-(tetrahydro-2H-pyran-4-yl)-1H-imidazol-5-yl]pyrimidin-2-yl}amino)benzenesulfonamide;
N-{4-[(4-Methyl-1,4-diazepan-1-yl)sulfonyl]phenyl}-4-[2-methyl-1-(tetrahydro-2H-pyran-4-yl)-1H-imidazol-5-yl]pyrimidin-2-amine;
N,N-Diethyl-4-({4-[2-methyl-1-(tetrahydro-2H-pyran-4-yl)-1H-imidazol-5-yl]pyrimidin-2-yl}amino)benzenesulfonamide;
N-[4-(Azetidin-1-ylsulfonyl)phenyl]-4-[2-methyl-1-(tetrahydro-2H-pyran-4-yl)-1H-imidazol-5-yl]pyrimidin-2-amine;
N-{3-[(4-Methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Chloro-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
N-{3-Methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(3R)-3-methylmorpholin-4-yl]sulfonyl}phenyl)-4-[2-methyl-1-(tetrahydro-2H-pyran-4-yl)-1H-imidazol-5-yl]pyrimidin-2-amine;
5-Fluoro-N-{3-methyl-4-[(4-methylpiperazin-1-yl)sulfonyl]phenyl}-4-[2-methyl-1-(tetrahydro-2H-pyran-4-yl)-1H-imidazol-5-yl]pyrimidin-2-amine;
5-Fluoro-N-(4-{[(1S,4S)-5-methyl-2,5-diazabicyclo[2.2.1]hept-2-yl]sulfonyl}phenyl)-4-[2-methyl-1-(tetrahydro-2H-pyran-4-yl)-1H-imidazol-5-yl]pyrimidin-2-amine;
4-({5-Fluoro-4-[2-methyl-1-(tetrahydro-2H-pyran-4-yl)-1H-imidazol-5-yl]pyrimidin-2-yl}amino)-N,N-dimethylbenzenesulfonamide;
N-[4-(Azetidin-1-ylsulfonyl)phenyl]-5-fluoro-4-[2-methyl-1-(tetrahydro-2H-pyran-4-yl)-1H-imidazol-5-yl]pyrimidin-2-amine;
Methyl 3-{[4-(1,2-dimethyl-1H-imidazol-5-yl)-5-fluoropyrimidin-2-yl]amino}benzoate;
3-[[4-(2,3-Dimethylimidazol-4-yl)-5-fluoro-pyrimidin-2-yl]amino]-N-(3-methoxypropyl)benzamide hydrochloride;
[4-[[4-(2,3-Dimethylimidazol-4-yl)-5-fluoro-pyrimidin-2-yl]amino]-2-(trifluoromethoxy)phenyl]-(4-methylpiperazin-1-yl)methanone hydrochloride;
N-[4-(Azetidin-1-ylcarbonyl)phenyl]-5-fluoro-4-[2-methyl-1-(tetrahydro-2H-pyran-4-yl)-1H-imidazol-5-yl]pyrimidin-2-amine hydrochloride;
N-{4-[(3,3-Difluoroazetidin-1-yl)carbonyl]phenyl}-5-fluoro-4-[2-methyl-1-(tetrahydro-2H-pyran-4-yl)-1H-imidazol-5-yl]pyrimidin-2-amine hydrochloride;
5-Fluoro-N-[3-methyl-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-[4-(morpholin-4-ylmethyl)phenyl]-4-[3-oxan-4-yl-2-(trifluoromethyl)imidazol-4-yl]-pyrimidin-2-amine hydrochloride;
5-Fluoro-N-{4-[(4-fluoropiperidin-1-yl)carbonyl]phenyl}-4-[2-methyl-1-(tetrahydro-2H-pyran-4-yl)-1H-imidazol-5-yl]pyrimidin-2-amine hydrochloride;
Ethyl 4-({5-fluoro-4-[2-methyl-1-(tetrahydro-2H-pyran-4-yl)-1H-imidazol-5-yl]pyrimidin-2-yl}amino)benzoate;
N,N-Diethyl-4-({5-fluoro-4-[2-methyl-1-(tetrahydro-2H-pyran-4-yl)-1H-imidazol-5-yl]pyrimidin-2-yl}amino)benzamide hydrochloride;
4-[5-fluoro-4-(2-methyl-3-tetrahydropyran-4-yl-imidazol-4-yl)-pyrimidin-2-yl]amino-N-(3-methoxypropyl)benzamide hydrochloride;
[4-[5-fluoro-4-(2-methyl-3-tetrahydropyran-4-yl-imidazol-4-yl)-pyrimidin-2-yl]aminophenyl]-(1,4-oxazepan-4-yl)methanone hydrochloride;
(4-ethylpiperazin-1-yl)-[4-[5-fluoro-4-(2-methyl-3-tetrahydropyran-4-yl-imidazol-4-yl)-pyrimidin-2-yl]aminophenyl]-methanone hydrochloride;
(2,6-dimethylmorpholin-4-yl)-[4-[5-fluoro-4-(2-methyl-3-tetrahydropyran-4-yl-imidazol-4-yl)-pyrimidin-2-yl]aminophenyl]-methanone hydrochloride;
[4-[5-fluoro-4-(2-methyl-3-tetrahydropyran-4-yl-imidazol-4-yl)-pyrimidin-2-yl]aminophenyl]-(3-fluoropyrrolidin-1-yl)-methanone hydrochloride;
(3,3-difluoropyrrolidin-1-yl)-[4-[5-fluoro-4-(2-methyl-3-tetrahydropyran-4-yl-imidazol-4-yl)-pyrimidin-2-yl]aminophenyl]-methanone hydrochloride;
4-[5-fluoro-4-(2-methyl-3-tetrahydropyran-4-yl-imidazol-4-yl)-pyrimidin-2-yl]amino-N-methyl-benzamide hydrochloride;
4-[5-fluoro-4-(2-methyl-3-tetrahydropyran-4-yl-imidazol-4-yl)-pyrimidin-2-yl]amino-N-tetrahydropyran-4-yl-benzamide hydrochloride;
[4-[5-fluoro-4-(2-methyl-3-tetrahydropyran-4-yl-imidazol-4-yl)-pyrimidin-2-yl]aminophenyl]-(3-hydroxypyrrolidin-1-yl)-methanone hydrochloride;
N-(2-cyanoethyl)-4-[5-fluoro-4-(2-methyl-3-tetrahydropyran-4-yl-imidazol-4-yl)-pyrimidin-2-yl]amino-N-methyl-benzamide hydrochloride;
N-ethyl-4-[5-fluoro-4-(2-methyl-3-tetrahydropyran-4-yl-imidazol-4-yl)-pyrimidin-2-yl]amino-N-(2-hydroxyethyl)benzamide hydrochloride;
4-[5-fluoro-4-(2-methyl-3-tetrahydropyran-4-yl-imidazol-4-yl)-pyrimidin-2-yl]amino-N-(2-hydroxyethyl)-N-methyl-benzamide hydrochloride;
4-[5-fluoro-4-(2-methyl-3-tetrahydropyran-4-yl-imidazol-4-yl)-pyrimidin-2-yl]amino-N-(2-hydroxyethyl)benzamide hydrochloride;
N-(2-dimethylaminoethyl)-4-[5-fluoro-4-(2-methyl-3-tetrahydropyran-4-yl-imidazol-4-yl)-pyrimidin-2-yl]amino-benzamide hydrochloride;
(4-dimethylamino-1-piperidyl)-[4-[5-fluoro-4-(2-methyl-3-tetrahydropyran-4-yl-imidazol-4-yl)-pyrimidin-2-yl]aminophenyl]-methanone hydrochloride;
[4-[5-fluoro-4-(2-methyl-3-tetrahydropyran-4-yl-imidazol-4-yl)-pyrimidin-2-yl]aminophenyl]-[4-(2-methoxyethyl)piperazin-1-yl]-methanone hydrochloride;
4-[5-fluoro-4-(2-methyl-3-tetrahydropyran-4-yl-imidazol-4-yl)-pyrimidin-2-yl]amino-N-[2-(1-piperidyl)ethyl]benzamide hydrochloride;
4-[5-fluoro-4-(2-methyl-3-tetrahydropyran-4-yl-imidazol-4-yl)-pyrimidin-2-yl]amino-N-(2-morpholinoethyl)benzamide hydrochloride;
4-[5-fluoro-4-(2-methyl-3-tetrahydropyran-4-yl-imidazol-4-yl)-pyrimidin-2-yl]amino-N-isopropyl-benzamide hydrochloride;
N-[2-(3,3-difluoropyrrolidin-1-yl)ethyl]-4-[5-fluoro-4-(2-methyl-3-tetrahydropyran-4-yl-imidazol-4-yl)-pyrimidin-2-yl]amino-benzamide hydrochloride;
[4-[5-fluoro-4-(2-methyl-3-tetrahydropyran-4-yl-imidazol-4-yl)-pyrimidin-2-yl]aminophenyl]-(4-isopropylpiperazin-1-yl)-methanone hydrochloride;
[4-[5-fluoro-4-(2-methyl-3-tetrahydropyran-4-yl-imidazol-4-yl)-pyrimidin-2-yl]aminophenyl]-(4-methyl-1,4-diazepan-1-yl)-methanone hydrochloride;
4-[5-fluoro-4-(2-methyl-3-tetrahydropyran-4-yl-imidazol-4-yl)-pyrimidin-2-yl]amino-N-tetrahydrofuran-3-yl-benzamide hydrochloride;
5-Fluoro-N-[4-(methylsulfonyl)phenyl]-4-[1-(tetrahydro-2H-pyran-4-yl)-2-(trifluoromethyl)-1H-imidazol-5-yl]pyrimidin-2-amine;
N-[4-(Azetidin-1-ylcarbonyl)phenyl]-5-fluoro-4-[1-(tetrahydro-2H-pyran-4-yl)-2-(trifluoromethyl)-1H-imidazol-5-yl]pyrimidin-2-amine;
N-[4-(Azetidin-1-ylcarbonyl)-3-chlorophenyl]-4-(1,2-dimethyl-1H-imidazol-5-yl)-5-fluoropyrimidin-2-amine;
N-[4-(Azetidin-1-ylcarbonyl)-3-methylphenyl]-4-(1,2-dimethyl-1H-imidazol-5-yl)-5-fluoropyrimidin-2-amine;
N-[3-Chloro-4-(methylsulfonyl)phenyl]-4-(1,2-dimethyl-1H-imidazol-5-yl)-5-fluoropyrimidin-2-amine;
4-(1,2-Dimethyl-1H-imidazol-5-yl)-5-fluoro-N-[4-(methylsulfonyl)phenyl]pyrimidin-2-amine;
N-{3-Chloro-4-[(4-methylpiperazin-1-yl)sulfonyl]phenyl}-4-(1,2-dimethyl-1H-imidazol-5-yl)-5-fluoropyrimidin-2-amine;
4-(1,2-Dimethyl-1H-imidazol-5-yl)-5-fluoro-N-{3-methyl-4-[(4-methylpiperazin-1-yl)sulfonyl]phenyl}pyrimidin-2-amine;
N-[4-(Azetidin-1-ylcarbonyl)-3-(trifluoromethoxy)phenyl]-4-(1,2-dimethyl-1H-imidazol-5-yl)-5-fluoropyrimidin-2-amine;
5-Fluoro-N-[4-(4-methylpiperazin-1-yl)sulfonylphenyl]-4-[3-methyl-2-(trifluoromethyl)imidazol-4-yl]-pyrimidin-2-amine hydrochloride;
5-Fluoro-4-[3-methyl-2-(trifluoromethyl)imidazol-4-yl]-N-[4-(morpholin-4-ylmethyl)phenyl]-pyrimidin-2-amine hydrochloride;
[4-[5-Fluoro-4-[3-methyl-2-(trifluoromethyl)imidazol-4-yl]-pyrimidin-2-yl]aminophenyl]-(4-methylpiperazin-1-yl)-methanone hydrochloride;
[4-[5-Fluoro-4-[3-tetrahydropyran-4-yl-2-(trifluoromethyl)imidazol-4-yl]-pyrimidin-2-yl]aminophenyl]-(4-methylpiperazin-1-yl)-methanone hydrochloride;
5-Fluoro-N-[3-(methylsulfonyl)-4-(morpholin-4-ylmethyl)phenyl]-4-[2-methyl-1-(tetrahydro-2H-pyran-4-yl)-1H-imidazol-5-yl]pyrimidin-2-amine hydrochloride;
5-Fluoro-N-[4-(methylsulfonyl)-3-(trifluoromethyl)phenyl]-4-[2-methyl-1-(tetrahydro-2H-pyran-4-yl)-1H-imidazol-5-yl]pyrimidin-2-amine hydrochloride;
6-({5-Fluoro-4-[2-methyl-1-(tetrahydro-2H-pyran-4-yl)-1H-imidazol-5-yl]pyrimidin-2-yl}amino)-2,3-dihydro-4H-thiochromen-4-one 1,1-dioxide hydrochloride;
6-({5-Fluoro-4-[2-methyl-1-(tetrahydro-2H-pyran-4-yl)-1H-imidazol-5-yl]pyrimidin-2-yl}amino)thiochroman-4-ol 1,1-dioxide hydrochloride;
N-(3-Dimethylaminopropyl)-3-[[4-(2,3-dimethylimidazol-4-yl)-5-fluoro-pyrimidin-2-yl]amino]benzamide;
N-(3-Dimethylaminopropyl)-3-[[4-(2,3-dimethylimidazol-4-yl)-5-fluoro-pyrimidin-2-yl]amino]-N-methyl-benzamide hydrochloride;
[3-[[4-(2,3-Dimethylimidazol-4-yl)-5-fluoro-pyrimidin-2-yl]amino]phenyl]-[3-(hydroxymethyl)-1-piperidyl]methanone;
N-{3-Chloro-4-[(4-methylpiperazin-1-yl)carbonyl]phenyl}-5-fluoro-4-[2-methyl-1-(tetrahydro-2H-pyran-4-yl)-1H-imidazol-5-yl]pyrimidin-2-amine;
5-Fluoro-N-{3-[(4-methylpiperazin-1-yl)carbonyl]phenyl}-4-[2-methyl-1-(tetrahydro-2H-pyran-4-yl)-1H-imidazol-5-yl]pyrimidin-2-amine;
(4-{[4-(1,2-Dimethyl-1H-imidazol-5-yl)-5-fluoropyrimidin-2-yl]amino}phenyl)(pyridin-2-yl)methanol;
5-Fluoro-N-[4-(isopropylsulfonyl)phenyl]-4-[2-methyl-1-(tetrahydro-2H-pyran-4-yl)-1H-imidazol-5-yl]pyrimidin-2-amine;
N-[4-(Ethylsulfonyl)phenyl]-5-fluoro-4-[2-methyl-1-(tetrahydro-2H-pyran-4-yl)-1H-imidazol-5-yl]pyrimidin-2-amine;
5-Fluoro-N-{4-[(2-methoxyethyl)sulfonyl]phenyl}-4-[2-methyl-1-(tetrahydro-2H-pyran-4-yl)-1H-imidazol-5-yl]pyrimidin-2-amine;
N-(4-{[2-(Diethylamino)ethyl]sulfonyl}phenyl)-5-fluoro-4-[2-methyl-1-(tetrahydro-2H-pyran-4-yl)-1H-imidazol-5-yl]pyrimidin-2-amine;
2-{[4-({5-Fluoro-4-[2-methyl-1-(tetrahydro-2H-pyran-4-yl)-1H-imidazol-5-yl]pyrimidin-2-yl}amino)phenyl]sulfonyl}ethanol;
{5-Fluoro-4-[3-(tetrahydro-pyran-4-yl)-3H-imidazol-4-yl]-pyrimidin-2-yl}-[4-(4-methyl-piperazine-1-sulfonyl)-phenyl]-amine;
5-{5-Fluoro-2-[4-(4-methyl-piperazine-1-sulfonyl)-phenylamino]-pyrimidin-4-yl}-1-(tetrahydro-pyran-4-yl)-1H-imidazole-2-carbonitrile; or
{5-Fluoro-4-[2-methyl-3-(tetrahydro-pyran-4-yl)-3H-imidazol-4-yl]-pyrimidin-2-yl}-[4-(tetrahydro-pyran-2-ylmethanesulfonyl)-phenyl]-amine,
as a base or a pharmaceutically acceptable salt, solvate or solvate of a salt thereof.

86. A compound of formula J: 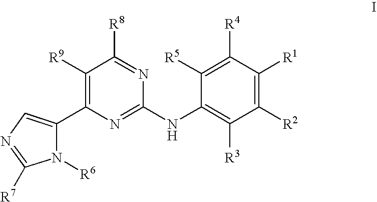
wherein
R¹ is C₀₋₂alkylC(O)NR^{b}R^{c} or C₁₋₄alkylNR^{b}R^{c};
R² and R⁴ are independently selected from hydrogen or C₁₋₄alkyl;
R³ and R⁵ are independently selected from hydrogen or halo;
R⁶ is a 6-membered heterocyclic ring containing one or more heteroatoms selected from N, O or S;
R⁷ is C₁₋₃alkyl;
R⁸ and R⁹ are independently selected from hydrogen and halo, as a base or a pharmaceutically acceptable salt, solvate or solvate of a salt thereof.

87. A compound selected from:
4-(1,2-Dimethyl-1H-imidazol-5-yl)-5-fluoro-N-[3-methoxy-5-(trifluoromethyl)phenyl]pyrimidin-2-amine;
N-(3,5-Dichlorophenyl)-4-(1,2-dimethyl-1H-imidazol-5-yl)-5-fluoropyrimidin-2-amine;
(4-{[4-(1,2-Dimethyl-1H-imidazol-5-yl)-5-fluoropyrimidin-2-yl]amino}phenyl)(phenyl)methanone;
4-(1,2-Dimethyl-1H-imidazol-5-yl)-5-fluoro-N-{2-methyl-4-[(4-methylpiperazin-1-yl)carbonyl]phenyl}pyrimidin-2-amine;
4-(1,2-Dimethyl-1H-imidazol-5-yl)-5-fluoro-N-{4-[(4-methylpiperazin-1-yl)carbonyl]-3-nitrophenyl}pyrimidin-2-amine;
4-(1,2-Dimethyl-1H-imidazol-5-yl)-5-fluoro-N-[4-[(4-methylpiperazin-1-yl)carbonyl]-2-(trifluoromethoxy)phenyl]pyrimidin-2-amine hydrochloride;
4-(1-Cyclohexyl-2-methyl-1H-imidazol-5-yl)-5-fluoro-N-{4-[(4-methylpiperazin-1-yl)carbonyl]phenyl}pyrimidin-2-amine hydrochloride;
4-(1-Cyclohexyl-2-methyl-1H-imidazol-5-yl)-5-fluoro-N-{4-[(4-methylpiperazin-1-yl)sulfonyl]phenyl}pyrimidin-2-amine hydrochloride;
5-Fluoro-4-[2-methyl-1-(1-methylpiperidin-4-yl)-1H-imidazol-5-yl]-N-{4-[(4-methylpiperazin-1-yl)carbonyl]phenyl}pyrimidin-2-amine hydrochloride;
5-Fluoro-4-[2-methyl-1-(1-methylpiperidin-4-yl)-1H-imidazol-5-yl]-N-[4-(pyrrolidin-1-ylsulfonyl)phenyl]pyrimidin-2-amine hydrochloride;
4-(1,2-Dimethyl-1H-imidazol-5-yl)-5-fluoro-N-[4-(morpholin-4-ylmethyl)phenyl]pyrimidin-2-amine hydrochloride;
4-(1,2-Dimethyl-1H-imidazol-5-yl)-5-fluoro-N-{4-[(4-methylpiperazin-1-yl)sulfonyl]phenyl}pyrimidin-2-amine;
4-(1,2-Dimethyl-1H-imidazol-5-yl)-5-fluoro-N-[4-(piperidin-1-ylcarbonyl)phenyl]pyrimidin-2-amine hydrochloride;
4-(1,2-Dimethyl-1H-imidazol-5-yl)-5-fluoro-N-{4-[(4-methylpiperazin-1-yl)carbonyl]phenyl}pyrimidin-2-amine hydrochloride;
4-(1,2-Dimethyl-1H-imidazol-5-yl)-5-fluoro-N-{4-[(4-methylpiperazin-1-yl)methyl]phenyl}pyrimidin-2-amine hydrochloride;
4-(1,2-Dimethyl-1H-imidazol-5-yl)-5-fluoro-N-{3-[(4-methylpiperazin-1-yl)carbonyl]phenyl}pyrimidin-2-amine hydrochloride, or
(4-{[4-(1,2-Dimethyl-1H-imidazol-5-yl)-5-fluoropyrimidin-2-yl]amino}phenyl)(pyridin-2-yl)methanone hydrochloride;
or as a base or alternative salt thereof.

88. A compound selected from:
Methyl 3-{[4-(1,2-dimethyl-1H-imidazol-5-yl)-5-fluoropyrimidin-2-yl]amino}benzoate;
N-[4-(Azetidin-1-ylcarbonyl)-3-chlorophenyl]-4-(1,2-dimethyl-1H-imidazol-5-yl)-5-fluoropyrimidin-2-amine;
N-[4-(Azetidin-1-ylcarbonyl)-3-methylphenyl]-4-(1,2-dimethyl-1H-imidazol-5-yl)-5-fluoropyrimidin-2-amine;
N-[3-Chloro-4-(methylsulfonyl)phenyl]-4-(1,2-dimethyl-1H-imidazol-5-yl)-5-fluoropyrimidin-2-amine;
4-(1,2-Dimethyl-1H-imidazol-5-yl)-5-fluoro-N-[4-(methylsulfonyl)phenyl]pyrimidin-2-amine;
N-{3-Chloro-4-[(4-methylpiperazin-1-yl)sulfonyl]phenyl}-4-(1,2-dimethyl-1H-imidazol-5-yl)-5-fluoropyrimidin-2-amine;
4-(1,2-Dimethyl-1H-imidazol-5-yl)-5-fluoro-N-{3-methyl-4-[(4-methylpiperazin-1-yl)sulfonyl]phenyl}pyrimidin-2-amine;
N-[4-(Azetidin-1-ylcarbonyl)-3-(trifluoromethoxy)phenyl]-4-(1,2-dimethyl-1H-imidazol-5-yl)-5-fluoropyrimidin-2-amine;
N-(3-Dimethylaminopropyl)-3-[[4-(2,3-dimethylimidazol-4-yl)-5-fluoro-pyrimidin-2-yl]amino]benzamide;
[3-[[4-(2,3-Dimethylimidazol-4-yl)-5-fluoro-pyrimidin-2-yl]amino]phenyl]-[3-(hydroxymethyl)-1-piperidyl]methanone, or
(4-{[4-(1,2-Dimethyl-1H-imidazol-5-yl)-5-fluoropyrimidin-2-yl]amino}phenyl)(pyridin-2-yl)methanol;
or a pharmaceutically acceptable salt thereof.

89. A compound selected from:
3-[[4-(2,3-Dimethylimidazol-4-yl)-5-fluoro-pyrimidin-2-yl]amino]-N-(3-methoxypropyl)benzamide hydrochloride;
[4-[[4-(2,3-Dimethylimidazol-4-yl)-5-fluoro-pyrimidin-2-yl]amino]-2-(trifluoromethoxy)phenyl]-(4-methylpiperazin-1-yl)methanone hydrochloride;
5-Fluoro-N-[4-(morpholin-4-ylmethyl)phenyl]-4-[3-oxan-4-yl-2-(trifluoromethyl)imidazol-4-yl]-pyrimidin-2-amine hydrochloride;
5-Fluoro-N-[4-(4-methylpiperazin-1-yl)sulfonylphenyl]-4-[3-methyl-2-(trifluoromethyl)imidazol-4-yl]-pyrimidin-2-amine hydrochloride;
5-Fluoro-4-[3-methyl-2-(trifluoromethyl)imidazol-4-yl]-N-[4-(morpholin-4-ylmethyl)phenyl]-pyrimidin-2-amine hydrochloride;
[4-[5-Fluoro-4-[3-methyl-2-(trifluoromethyl)imidazol-4-yl]-pyrimidin-2-yl]aminophenyl]-(4-methylpiperazin-1-yl)-methanone hydrochloride, or
N-(3-Dimethylaminopropyl)-3-[[4-(2,3-dimethylimidazol-4-yl)-5-fluoro-pyrimidin-2-yl]amino]-N-methyl-benzamide hydrochloride;
or as a base or alternative salt thereof.
